(19) United States
(12) Patent Application Publication
(10) Pub. No.: US 2026/0049088 A1
(43) Pub. Date: Feb. 19, 2026

(54) TRICYCLIC HETEROCYCLIC DERIVATIVES, COMPOSITIONS AND USES THEREOF

(71) Applicant: Danatlas Pharmaceuticals Co., Ltd., Beijing (CN)

(72) Inventors: Wenlai ZHOU, Beijing (CN); Jincong ZHUO, Beijing (CN); Yao ZHANG, Beijing (CN)

(21) Appl. No.: 19/367,857

(22) Filed: Oct. 24, 2025

Related U.S. Application Data
(63) Continuation of application No. PCT/CN2024/089971, filed on Apr. 26, 2024.

(30) Foreign Application Priority Data
Apr. 27, 2023 (WO) PCT/CN2023/091391
Jun. 19, 2023 (WO) PCT/CN2023/101168
Oct. 25, 2023 (WO) PCT/CN2023/126326

(51) Int. Cl. C07D 487/04; A61K 31/519; A61K 31/5377; A61K 31/541; A61K 45/06; C07D 519/00

(52) U.S. Cl. CPC C07D 487/04; A61K 31/519; A61K 31/5377; A61K 31/541; A61K 45/06; C07D 519/00

(57) ABSTRACT

The disclosure relates to tricyclic heterocyclic derivatives as shown in Formula (I), to pharmaceutical compositions comprising them, to a process for their preparation, and their use as therapeutic agents.

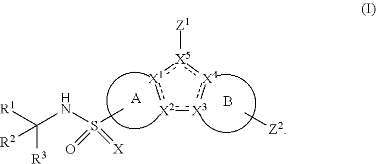

### CROSS REFERENCES TO RELATED APPLICATIONS

The present application is a continuation application of Internation Patent Application No. PCT/CN2024/089971, filed on Apr. 26, 2024, which claims the benefit of the priority of International Application No. PCT/CN2023/091391, filed Apr. 27, 2023, International Application No. PCT/CN2023/101168, filed Jun. 19, 2023, International Application No. PCT/CN2023/126326, filed Oct. 25, 2023, each of which is hereby incorporated in its entirety.

### FIELD

The present disclosure relates to tricyclic heterocyclic derivatives as PARG inhibitors. The present disclosure also relates to methods for preparing the tricyclic heterocyclic derivatives, pharmaceutical compositions, and their uses in the treatment of PARG-mediated diseases, e.g., cancers and other diseases.

### BACKGROUND

DNA damage repair (DDR) is a collection of processes by which a cell identifies and corrects damage to the DNA molecules that encode its genome. But once a cancer has formed, DNA repair pathways become a double-edged sword because they promote the repair and survival of cancer cells in response to chemotherapies and radiotherapies. As a result, cancers with compromised DNA repair are susceptible to DNA damage and depend on complementary repair pathways which can be exploited therapeutically.

An aberrant DDR often can sensitize cancer cells to specific types of DNA damage, thus defective DDR can be developed into targeted cancer therapies. Targeting DNA repair deficiencies have become a proven and effective strategy in cancer treatment. For example, the success of poly (ADP-ribose) polymerase (PARP) inhibitors in treating BRCA-deficient breast, ovarian, prostate and pancreatic cancers (Audeh M W et al., 2010).

Poly(ADP-ribosyl)ation (PARylation) is an unique posttranslational modification for maintaining genome stability through different molecular pathways, especially DNA repair (Kraus W L et al., 2015). The binding of PARP to the break and the rapid synthesis of poly ADP-ribose (PAR) on PARP itself is one of the earliest events during single strand DNA repair. Current PARP inhibitors primarily suppress PARP1 and PARP2 enzymatic activities, which inhibits PARP1/2-dependent DNA repair. Recently, clinical resistance to PARP inhibitors has been described (Drost and Jonkers, 2014) (Barber L J et al., 2013) (Tobalina L et al., 2021) and therefore alternative inhibitors targeting the DNA damage repair machinery are required.

PARylation is a transient posttranslational modification and is rapidly degraded by the enzyme PAR glycohydrolase (PARG) (Barkauskaite E et al., 2015). When PARP is bound to PAR, its catalytic activity is reduced and therefore PARG activity helps to restore PARP to its catalytically active form (Curtin and Szabo, 2013). Similar to PARPs, PARG also facilitates both DNA double-strand break (DSB) and single-strand break (SSB) repair (Mortusewicz O et al., 2011). Apart its primary role in DNA repair, PARG impacts PAR signaling in RNA splicing, transcriptional and epigenetic regulation (Ji and Tulin 2009) (Le May N et al., 2012) (Dahl M et al. 2014) (Guastafierro T et al., 2013) (Caiafa P et al., 2009). Some evidences suggest that PARG depletion inhibits SSB repair and reduces survival of BRCA2—deficient cells (Fathers C et al., 2012). However, other tumor mutations may give rise to deficiencies in DSB repair mechanisms (so-called “BRCA-ness”) may also cause sensitizing tumor cells to PARG inhibition.

However, as deficiency in PARG doesn't sensitize to all agents (e.g. gemcitabine, camptothecin), it indicates that a specificity for PARG function with certain pathways of DDR and chemo- and radiotherapies (Fujihara H et al., 2009) (Shirai H et al., 2013) (Zhou Y et al., 2011). In humans, PARG knock-down or depletion can sensitize lung, cervical and pancreatic cancer cells to irradiation or experimental DNA damaging agents (e.g. hydrogen peroxide, Methylmethanesulfonate) (Ame J C et al., 2009) (Nakadate Y et al., 2013) (Shirai H et al., 2013).

Some studies suggest that PARG inhibition may provide a therapeutic advantage in PARP inhibitor resistant cells (Fisher A E et al., 2007). Furthermore, depletion of PARG has been reported to lead to a markedly different gene expression pattern to that of PARP in breast cancer cells (Frizzell K M et al., 2009). Ovarian cancer cells respond differently to PARP inhibitor and PARG inhibitor and sensitivity to the latter is due to persistent fork stalling and replication catastrophe (Pillay N et al., 2019) (Coulson-Gilmer C et al., 2021).

Recent researches have also shown a mechanistic differentiation between PARG and PARP inhibition. Following a genotoxic stimulus depletion of PARG, in contrast to PARP depletion, leads to a drop in NAD levels and causes to lung cancer cell death that may be as a result of energy failure (Erdelyi K et al., 2009). PARG inhibition can also sequester NAD+ to potentiate the metabolic lethality of alkylating chemotherapy in IDH mutant tumor cells (Nagashima H et al., 2020).

Cell permeable PARG inhibitors have been limited to compounds such as Tannic acid or Gallotannin or PDD00017273 which have low specificity for PARG and limited bioavailability (Sun Y et al., 2012) (Fathers C et al., 2012) (Blenn C et al., 2011) (James D I et al., 2016).

An object of this invention is to provide cell permeable inhibitors of PARG.

### SUMMARY

Provided herein is a compound of Formula (I),

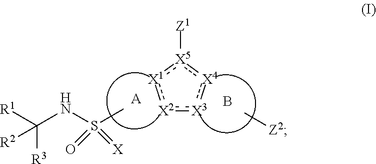

or a pharmaceutically acceptable salt, stereoisomer, solvate, N-oxide, tautomer, isotopic variant, prodrug, or deuterated compound thereof; wherein [symbol] , ring A, ring B, X, X¹, X², X³, X⁴, X⁵, Z¹, Z², R¹, R², R³ are each as defined herein.

Also provided herein is a pharmaceutical composition comprising a compound of formula (I), or pharmaceutically acceptable salt, stereoisomer, solvate, N-oxide, tautomer, isotopic variant, prodrug, or deuterated compound thereof; and a pharmaceutically acceptable carrier.

Additionally provided herein is a method of inhibiting PARG comprising: contacting a PARG with a compound of formula (I), or pharmaceutically acceptable salt, stereoisomer, solvate, N-oxide, tautomer, isotopic variant, prodrug, or deuterated compound thereof.

In another aspect, provided herein is a method of treating a cancer and/or a disease comprising administering to the subject in need thereof a therapeutically effective amount of a compound of formula (I), or pharmaceutically acceptable salt, stereoisomer, solvate, N-oxide, tautomer, isotopic variant, prodrug, or deuterated compound thereof.

### DETAILED DESCRIPTION

The present disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub-combination.

Before the present invention is further described, it is to be understood that the invention is not limited to the particular embodiments set forth herein, and it is also to be understood that the terminology used herein is for the purpose of describing particular embodiments only, and is not intended to be limiting.

In one embodiment, provided herein is a compound of formula (I):

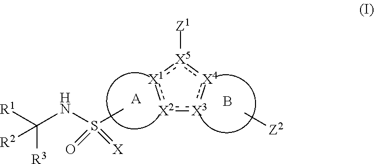

or a pharmaceutically acceptable salt, stereoisomer, solvate, N-oxide, tautomer, isotopic variant, prodrug, or deuterated compound thereof, wherein:
[symbol] is a single bond or double bond;
X is O or NR⁴;
X¹ is C or N;
X² is C or N;
X³ is C or N;
X⁴ is C or N;
X⁵ is C or N;
and

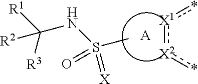

is

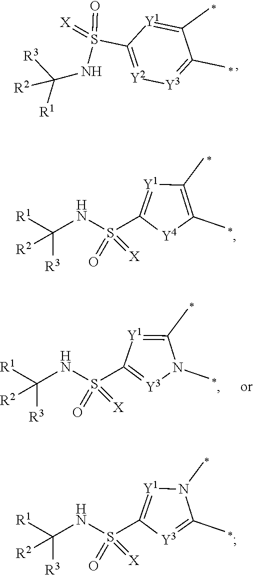

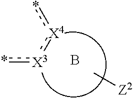

is

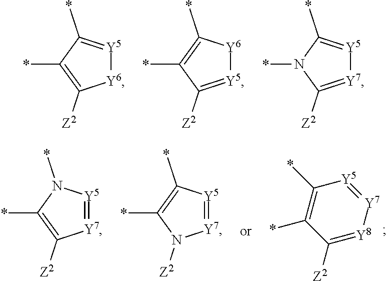

Y¹ and Y³ are each independently N or CR⁵;
Y² is N or CR⁶;
Y⁴ is S, O or NR⁷;
Y⁵, Y⁷ and Y⁸ are each independently N or CR⁸;
Y⁶ is S, O or NR⁹;
Z¹ is 5-10 membered heteroaryl optionally substituted by 1, 2, 3, 4, or 5 substituents independently selected from R¹⁰;
Z² is H, D, halo, CN, NO₂, SF₅, C₁-C₈ alkyl, C₂-C₈ alkenyl, C₂-C₈ alkynyl, C₆-C₁₀ aryl, C₃-C₁₀ cycloalkyl, 5-10 membered heteroaryl, 4-14 membered heterocycloalkyl, NRCR^{D}, OR^{A}, SR^{A}, NHOR^{A}, C(O)R^{B}, C(O)OR^{A}, C(O)NR^{C}R^{D}, OC(O)NR^{C}R^{D}, NRCC(O)R^{B}, NRCC(O)NR^{C}R^{D}NRCC(O)OR^{A}, NRCS(O)₂R^{B}, S(O)R^{B}, S(O)NR^{C}R^{D}, S(O)₂R^{B}, S(O)₂NR^{C}R^{D}, or NR^{C}S(O)₂NR^{C}R^{D}; wherein, the C₁-C₈ alkyl, C₂-C₈ alkenyl, C₂-C₈ alkynyl, C₆-C₁₀ aryl, C₃-C₁₀ cycloalkyl, 5-10 membered heteroaryl, or 4-14 membered heterocycloalkyl is optionally substituted by 1, 2, 3, 4, or 5 substituents independently selected from R¹¹;
R¹, R² and R³ are each independently H, D, CN, C₁-C₃ alkyl, C₂-C₃ alkenyl, C₂-C₃ alkynyl, C₃-C₇ cycloalkyl, or 4-7 membered heterocycloalkyl; wherein, the C₁-C₃ alkyl, C₂-C₃ alkenyl, C₂-C₃ alkynyl, C₃-C₇ cycloalkyl, or 4-7 membered heterocycloalkyl is optionally substituted by 1, 2, 3, 4 or 5 substituents independently selected from D, halo, CN, OH, C₁-C₆ alkyl, C₁-C₆ haloalkyl, —O—C₁-C₆ alkyl, —OC₁-C₆ haloalkyl;
or R² and R³ together with the carbon atom to which they are attached form C₃-C₇ cycloalkyl, or 4-7 membered heterocycloalkyl; wherein, the C₃-C₇ cycloalkyl, 4-7 membered heterocycloalkyl is optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl;
R⁴ is H, D, CN, OR^{B}, or C₁-C₄ alkyl optionally substituted with R^{4A}; wherein, each R^{4A} is independently D, F, Cl, CN, NH₂, OH, —O—C₁-C₆ alkyl, —OC₁-C₆ haloalkyl, optionally substituted C₃-C₇ cycloalkyl, or optionally substituted 4-7 membered heterocycloalkyl; wherein, optionally substituted is D, halo, CN, OH, C₁-C₄ alkyl, C₁-C₄haloalkyl, —O—C₁-C₄ alkyl, —OC₁-C₄haloalkyl;
or R¹ and R⁴ together with the atoms to which they are attached form 5-7 membered partially unsaturated heterocycloalkyl optionally substituted by 1, 2, 3 or 4 substituents independently selected from D, halogen, CN, CF₃, NO₂, oxo, OH, C₁-C₆ alkyl, C₁-C₆ haloalkyl, —O—C₁-C₆ alkyl, —OC₁-C₆ haloalkyl;
R⁵ is H, D, CN, halo, SF₅, OH, NH₂, CHO, COOH, C₁-C₃ alkyl, C₂-C₃ alkenyl, C₂-C₃ alkynyl, C₁-C₃ haloalkyl, C₁-C₃ cyanoalkyl, OR^{A}, NR^{C}R^{D} or C(O)R^{B};
R⁶ is H, D, CN, halo, OH, NH₂, SF₅, C₁-C₃ alkyl, C₁-C₃ haloalkyl, —O—C₁-C₃ alkyl, —OC₁-C₃ haloalkyl, C₁-C₃ cyanoalkyl;
R⁷ and R⁹ are each H, D, C₁-C₆ alkyl, C₁-C₆ haloalkyl, C₂-C₆ alkenyl, C₂-C₆ alkynyl, C₁-C₆ alkyl-OR^{A}, C₁-C₆ alkyl-CN, C₁-C₆ alkyl-NR^{C}R^{D}, C(O)R^{B}, C(O)NR^{C}R^{D};
each R⁸ is independently H, D, halogen, CN, NO₂, C₁-C₆ alkyl, C₁-C₆ haloalkyl, C₂-C₆ alkenyl, C₂-C₆ alkynyl, OR^{A}, SR^{A}, SF₅, NHOR^{A}, C(O)OR^{A}, C(O)R^{B}, C(O)NR^{C}R^{D}, OC(O)NR^{C}R^{D}, NR^{C}R^{D}, NR^{C}C(O)R^{B}, NR^{C}C(O)NR^{C}R^{D}, NR^{C}C(O)OR^{A}, NR^{C}S(O)₂R^{B} B(OR^{C})(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F}, P(O)OR^{E}OR^{F}, OP(O)OR^{E}OR^{F}, S(O)(═NR^{B})R^{B}, S(O)R^{B}, S(O)NR^{C}R^{D}, S(O)₂R^{B}, S(O)₂NR^{C}R^{D}, NR^{C}S(O)₂NR^{C}R^{D}, or NR^{C}S(O)(═NR^{B})R^{B}; wherein, the C₁-C₆ alkyl, C₂-C₆ alkenyl, or C₂-C₆ alkynyl is optionally substituted with 1, 2, or 3 substituents independently selected from R¹²;
each R¹⁰ is independently H, D, halo, C₁-C₆ alkyl, C₂-C₆ alkenyl, C₂-C₆ alkynyl, C₁-C₆ haloalkyl, OC₁-C₆ alkyl, OC₁-C₆ haloalkyl, OC₃-C₇ cycloalkyl, C₃-C₇ cycloalkyl, CN, NO₂, N₃, or SF₅; wherein, the C₁-C₆ alkyl, C₂-C₆ alkenyl, C₂-C₆ alkynyl or C₃-C₇ cycloalkyl is optionally substituted with 1, 2, or 3 substituents independently selected from R¹²;
two R¹⁰, together with the atom(s) to which they are attached form oxo, C₃-C₁₀ cycloalkyl or 4-10 membered heterocycloalkyl; wherein, the C₃-C₁₀ cycloalkyl or 4-10 membered heterocycloalkyl is optionally substituted by 1, 2, or 3 substituents independently selected from D, halo, C₁-C₆ alkyl, C₂-C₆ alkenyl, C₂-C₆ alkynyl, C₁-C₆ haloalkyl, C₁-C₆-cyanoalkyl, CN, NO₂, oxo, OR^{a}, SR^{a}, SF₅, NHOR^{a}, C(O)R^{b}, C(O)NR^{c}R^{D}, C(O)OR^{a}, OC(O)R^{b}, OC(O)NR^{c}R^{D}, NR^{C}R^{D}NR^{c}C(O)R^{b}, NR^{c}C(O)NR^{c}R^{d}, NR^{c}C(O)OR^{a}, B(OR^{c})(OR^{d}), C(═NR^{c})NR^{c}R^{d}, NR^{d}C(═NR^{c})NR^{c}R^{d}, NR^{d}C(═NR^{c})R^{b}, OP(O)OR^{e}OR^{f}, P(O)OR^{e}OR^{f}, S(O)(═NR^{b})R^{b}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d} NR^{c}S(O)(═NR^{b})R^{b}, Cy⁴; wherein, Cy⁴ is C₆-C₁₀ aryl, C₃-C₁₀ cycloalkyl, 5-10 membered heteroaryl, and 4-10 membered heterocycloalkyl; wherein Cy⁴ is optionally substituted by 1, 2, 3 or 4 substituents independently selected from D, halo, CN, NO₂, OH, oxo, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅;
each R¹¹ is independently H, D, halo, CN, NO₂, N₃, oxo, C₁-C₆ alkyl, C₂-C₆ alkenyl, C₂-C₆ alkynyl, OR^{A}, SR^{A}, SF₅, NR^{C}OR^{A}, C(O)R^{B}, C(═S)R^{B}, C(O)NR^{C}R^{D}, C(O)N(R^{C})OR^{A}, C(O)OR^{A}, OC(O)R^{B}, OC(O)NR^{C}R^{D}, NR^{C}R^{D}, NR^{C}C(O)R^{D}, NR^{C}C(O)NR^{C}R^{D}, NR^{C}C(O)OR^{A}, B(OR^{C})(OR^{D}), C(═NR^{C})NR^{C}R^{D} NR^{D}C(═NR^{C})NR^{C}R^{D}, NR^{D}C(═NR^{C})R^{B}, SiR^{G}R^{H}R¹, P(O)R^{E}R^{F}, P(O)OR^{E}OR^{F}, OP(O)OR^{E}OR^{F}, S(O)(═NR^{B})R^{B}, S(O)R^{B}, S(O)NR^{C}R^{D}, S(O)₂R^{B}, NR^{C}S(O)₂R^{B} S(O)₂NR^{C}R^{D}, NR^{C}S(O)₂NR^{C}R^{D}, NR^{C}S(O)(═NR^{B})R^{B} Cy³, C₁-C₆ alkyl-Cy³, OCy³, or O—C₁-C₆ alkyl-Cy³; wherein, the C₁-C₆ alkyl, C₂-C₆ alkenyl, C₂-C₆ alkynyl is optionally substituted with 1, 2, 3, 4 or 5 substituents independently selected from R¹²;
each R¹² is independently H, D, halo, CN, NO₂, N₃, C₁-C₆ alkyl, C₂-C₆ alkenyl, C₂-C₆ alkynyl, C₁-C₆ haloalkyl, OC₁-C₆ alkylOH, OC₁-C₆ alkyl-O—C₁-C₆ alkyl, OR^{a1}, SR^{a1}, SF₅, NHOR^{a1}, C(O)R^{b1}, C(O)NR^{c1}R^{d1}, C(O)OR^{a1}, OC(O)OR^{a1}, OC(O)R^{b1}, OC(O)NR^{c1}R^{d1}, NR^{c1}R^{d1} NR^{c1}C(O)R^{b1}, NR^{c1}C(O)NR^{c1}R^{d1} NR^{c1}C(O)OR^{a1}, B(OR^{c1})(OR^{d1}), C(═NR^{c1})NR^{c1}R^{d1} NR^{d1}C(═NR^{c1})NR^{c1}R^{d1}, NR^{d1}C(═NR^{c1})R^{b1}, P(O)OR^{e1}OR, OP(O)OR^{e1}OR^{f1}, S(O)(═NR^{b1})R^{b1}, S(O)R^{b1}, S(O)NR^{c1}R^{d1}, S(O)₂R^{b}1, NR^{c1}S(O)₂R^{b1}, S(O)₂NR^{c1}R^{d1} NR^{c1}S(O)₂NR^{c1}R^{d1} NR^{c1}S(O)(═NR^{b1})R^{b1}, C₆-C₁₀ aryl, C₃-C₁₀ cycloalkyl, 5-10 membered heteroaryl, and 4-10 membered heterocycloalkyl, wherein, the C₆-C₁₀ aryl, C₃-C₁₀ cycloalkyl, 5-10 membered heteroaryl, 4-10 membered heterocycloalkyl optionally substituted by 1, 2, 3, 4 or 5 substituents independently selected from D, halo, CN, NO₂, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅;
Cy³ is independently C₆-C₁₀ aryl, C₃-C₁₀ cycloalkyl, 5-10 membered heteroaryl, or 4-10 membered heterocycloalkyl; wherein Cy³ is optionally substituted with 1, 2, 3, 4 or 5 substituents independently selected from R¹³;
each R¹³ is independently D, halo, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{c1}R^{d1}, C(O)OR^{a1}, OC(O)R^{b1}, OC(O)NR^{c1}R^{d1} NR^{c1}R^{d1}, NR^{c1}C(O)R^{b1}, NR^{c1}C(O)NR^{c1}R^{d1} NR^{c1}C(O)OR^{a1}, B(OR^{c1})(OR^{d1}), C(═NR^{c1})NR^{c1}R^{d1} NR^{d1}C(═NR^{c1})NR^{c1}R^{d1} NR^{d1}C(═NR^{c1})R^{b1}, P(O)R^{e1}R^{f1}, P(O)OR^{e1}OR^{f1}, OP(O)OR^{e1}OR^{f1}, S(O)(═NR^{b1})R^{b1}, S(O)R^{b1}, S(O)NR^{c1}R^{d1}, S(O)₂R^{b1}, NR^{c1}S(O)₂R^{b1}, S(O)₂NR^{c1}R^{d1} NR^{c1}S(O)₂NR^{c1}R^{d1} NR^{c1}S(O)(═NR^{b1})R^{b}, C₆-C₁₀ aryl, C₃-C₁₀ cycloalkyl, 5-10 membered heteroaryl, and 4-10 membered heterocycloalkyl; wherein, the C₆-C₁₀ aryl, C₃-C₁₀ cycloalkyl, 5-10 membered heteroaryl, 4-10 membered heterocycloalkyl optionally substituted by 1, 2, 3, 4 or 5 substituents independently selected from D, halo, CN, NO₂, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅;
each R¹⁴ is independently H, D, NO₂, CN, halo, oxo, SF₅, C₁-C₈ alkyl, C₂-C₈ alkenyl, C₂-C₈ alkynyl, C₆-C₁₄ aryl, C₃-C₁₄ cycloalkyl, 5-14 membered heteroaryl, or 4-14 membered heterocycloalkyl, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d} NR^{c}C(O)OR^{a}, B(OR^{c})(OR^{d}), C(═NR^{c})NR^{c}R^{d}, NR^{d}C(═NR^{c})NR^{c}R^{d}, NR^{d}C(═NR^{c})R, P(O)R^{e}R^{f1}, P(O)OR^{c}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, or NR^{c}S(O)(═NR^{b})R^{b}; wherein, the C₁-C₈ alkyl, C₂-C₈ alkenyl, C₂-C₈ alkynyl, C₆-C₁₄ aryl, C₃-C₁₄ cycloalkyl, 5-14 membered heteroaryl, or 4-14 membered heterocycloalkyl is optionally substituted by 1, 2, 3, 4 or 5 substituents independently selected from D, NO₂, CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{c1}R^{d1}, C(O)OR^{a1}, OC(O)R^{b1}, OC(O)NR^{c1}R^{d1} NR^{c1}R^{d1} NR^{c1}C(O)R^{b1} NR^{c1} C(O)NR^{c1}R^{d1} NR^{c1}C(O)OR^{a1}, B(OR^{c1})(OR^{d1}), C(═NR^{c1})NR^{c1}R^{d1} NR^{d1}C(═NR^{c1})NR^{c1}R^{d1} NR^{d1}C(═NR^{c1})R^{b}, P(O)R^{e1}R^{f1}, P(O)OR^{e1}OR^{f1}, OP(O)OR^{e1}OR, S(O)(═NR^{b1})R^{b1}, S(O)R^{b1} S(O)NR^{c1}R^{d1}, S(O)₂R^{b1}, NR^{c1}S(O)₂R^{b}, S(O)₂NR^{c1}R^{d1} NR^{c1}S(O)₂NR^{c1}R^{d1} NR^{c1}S(O)(═NR^{b1})R^{b1}, C₆-C₁₀ aryl, C₃-C₁₀ cycloalkyl, 5-10 membered heteroaryl, and 4-10 membered heterocycloalkyl; each R^{A} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocyclalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-membered heterocycloalkyl-C₁-C₆ alkyl is optionally substituted with 1, 2, 3, 4 or 5 substituents independently selected from R¹⁴;
each R^{B} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-membered heterocycloalkyl-C₁-C₆ alkyl is optionally substituted with 1, 2, 3, 4 or 5 substituents independently selected from R¹⁴;
R^{C} and R^{D} are each independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-membered heterocycloalkyl-C₁-C₆ alkyl is optionally substituted with 1, 2, 3, 4 or 5 substituents independently selected from R¹⁴;
or R^{C} and R^{D} together with the N atom to which they are attached form 4-7 membered heterocycloalkyl optionally substituted with 1, 2, 3, 4 or 5 substituents independently selected from D, halo, oxo, CN, C₁-C₄ alkyl, C₁-C₄ haloalkyl, C₁-C₄ alkyl-CN, OR^{a}, SR^{a}, C(O)R^{b}, NR^{c}R^{d};
R^{a} and R^{a1} are each independently H, D, C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl; wherein the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄haloalkyl, or C₁-C₄haloalkoxy;
R^{b} and R^{b1} are each independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl; wherein the C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-membered heterocycloalkyl-C₁-C₄ alkyl is optionally substituted with 1, 2, 3, 4 or 5 substituents independently selected from D, OH, halo, CN, —NH₂, —NH(C₁-C₄ alkyl), —N(C₁-C₄ alkyl)₂, C₁-C₄ alkyl, C₁-C₄ haloalkyl, —OC₁-C₄ alkyl, —OC₁-C₄haloalkyl, C₆-C₁₀ aryl, C₃-C₁₀ cycloalkyl, 5-10 membered heteroaryl, or 4-10 membered heterocycloalkyl;
R^{c}, R^{d}, R^{c1} and R^{d1} are each independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, 4-10 membered heterocycloalkyl-C₁-C₄ alkyl, C₆-C₁₀ aryl-C₃-C₁₀ cycloalkyl, C₆-C₁₀ aryl-4-10 membered heterocycloalkyl, C₆-C₁₀ aryl-5-10 membered heteroaryl, bi(C₆-C₁₀ aryl), 5-10 membered heteroaryl-C₃-C₁₀ cycloalkyl, 5-10 membered heteroaryl-4-10 membered heterocycloalkyl, 5-10 membered heteroaryl-C₆-C₁₀ aryl, or bi(5-10 membered heteroaryl); wherein, the C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, 4-10 membered heterocycloalkyl-C₁-C₄ alkyl, C₆-C₁₀ aryl-C₃-C₁₀ cycloalkyl, C₆-C₁₀ aryl-4-10 membered heterocycloalkyl, C₆-C₁₀ aryl-5-membered heteroaryl, bi(C₆-C₁₀ aryl), 5-10 membered heteroaryl-C₃-C₁₀ cycloalkyl, 5-10 membered heteroaryl-4-10 membered heterocycloalkyl, 5-10 membered heteroaryl-C₆-C₁₀ aryl, or bi(5-10 membered heteroaryl) is optionally substituted with 1, 2, 3, 4 or 5 substituents independently selected from D, halo, OH, CN, —NH₂, —NH(C₁-C₄ alkyl), —N(C₁-C₄ alkyl)₂, C₁-C₄ alkyl, —O—C₁-C₄ alkyl, C₁-C₄ haloalkyl, —O—C₁-C₄ haloalkyl, C₁-C₄ alkyl-OH, C₁-C₄ alkyl-CN, C₆-C₁₀ aryl, 5-10 membered heteroaryl, —C(O)OR^{a1}, —C(O)R^{b1}, —S(O)₂R^{b1}, —C₁-C₄ alkyl-O—C₁-C₄ alkyl, and —C₁-C₄ alkyl-O—C₁-C₄ alkyl-O—;
or R^{c} and R^{d} together with the N atom to which they are attached form a 4-7 membered heterocycloalkyl optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkoxy-C₁-C₄ alkyl, and C₁-C₄ alkoxy-C₁-C₄ alkoxy;
or R^{c} and R^{d1} together with the N atom to which they are attached form a 4-7 membered heterocycloalkyl optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkoxy-C₁-C₄ alkyl, and C₁-C₄ alkoxy-C₁-C₄ alkoxy;
R^{E}, R^{e} and R^{e1} are each independently H, D, C₁-C₄ alkyl, C₁-C₄ haloalkyl, C₂-C₄ alkenyl, (C₁-C₄ alkoxy)-C₁-C₄ alkyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, C₃-C₁₀ cycloalkyl-C₁-C₄ alkyl, 5-10 membered heteroaryl-C₁-C₄ alkyl, or 4-10 membered heterocycloalkyl-C₁-C₄ alkyl;
R^{F}, R^{f} and R^{f1} are each independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, or 4-10 membered heterocycloalkyl;
R^{G}, R^{H} and R¹ are each independently C₁-C₄ alkyl or phenyl.

In another embodiment, provided herein is a compound of Formula (I′):

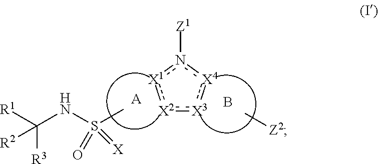

or a pharmaceutically acceptable salt, stereoisomer, solvate, N-oxide, tautomer, isotopic variant, prodrug, or deuterated compound thereof,
wherein, [symbol] , ring A, ring B, R¹, R², R³, X, X¹, X², X³, X⁴, Z¹ and Z² are each as defined herein.

In yet another embodiment, provided herein is a compound of Formula (IA), (IB), (IC), (ID), (Ia), (Ib), (Ic), (Id), (Ie), (If), (Ig), (Ih), (Ii), (Ij), (Ik), (Il), (Im), (In), (Io), (Ip), (Iq), (Ir):

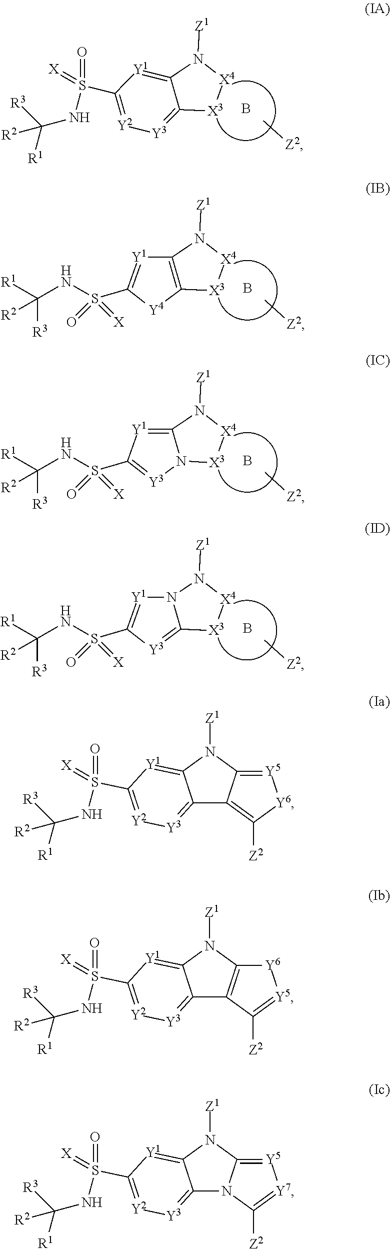

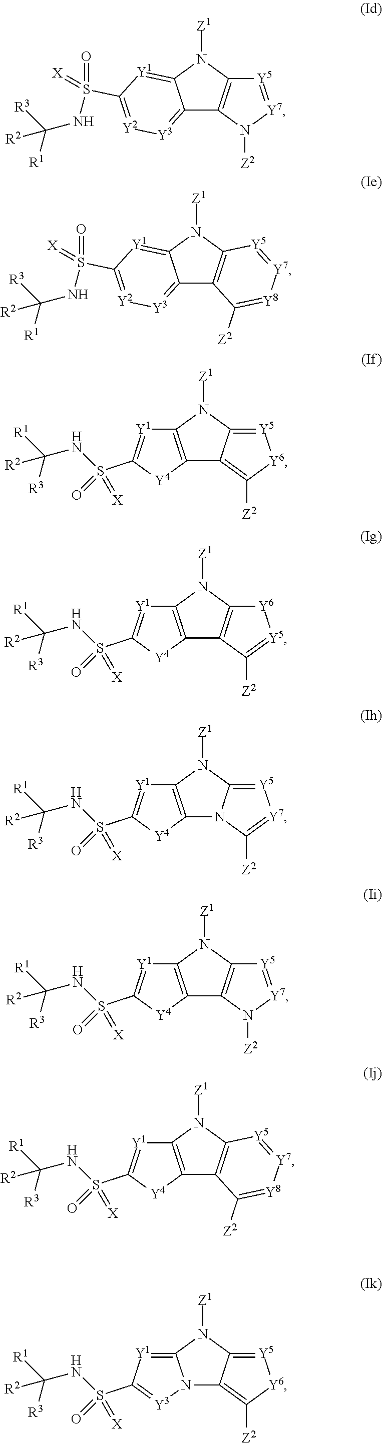

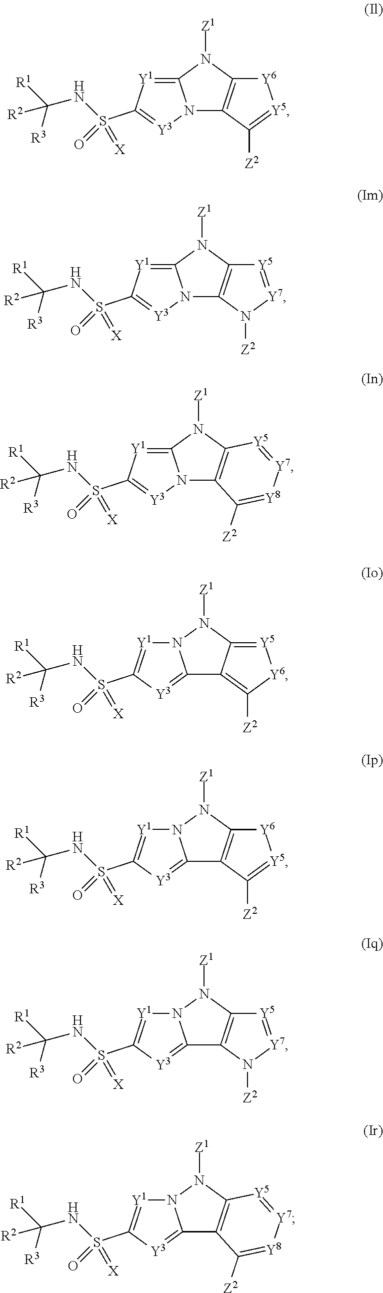

or a pharmaceutically acceptable salt, stereoisomer, solvate, N-oxide, tautomer, isotopic variant, prodrug, or deuterated compound thereof,
wherein, ring B, R¹, R², R³, X, X³, X⁴, Y¹, Y², Y³, Y⁴, Y⁵, Y⁶, Y⁷, Y⁸, Z¹, and Z² are each as defined herein.

In yet another embodiment, provided herein is a compound of Formula (IA), (Ib), (Ic), (Id), (Ie):

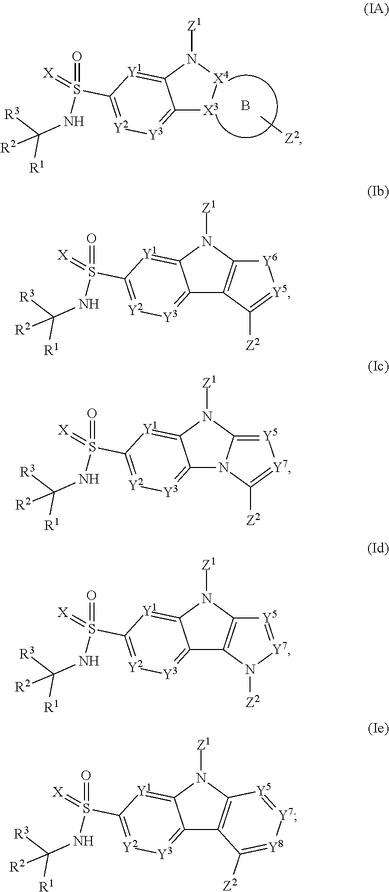

or a pharmaceutically acceptable salt, stereoisomer, solvate, N-oxide, tautomer, isotopic variant, prodrug, or deuterated compound thereof,
wherein, ring B, R¹, R², R³, X, X³, X⁴, Y, Y¹, Y², Y³, Y⁴, Y⁵, Y⁶, Y⁸, Z¹, and Z² are each as defined herein.

In yet another embodiment, provided herein is a compound of Formula (IB), (If), (Ig), (Ih), (Ii), (Ij):

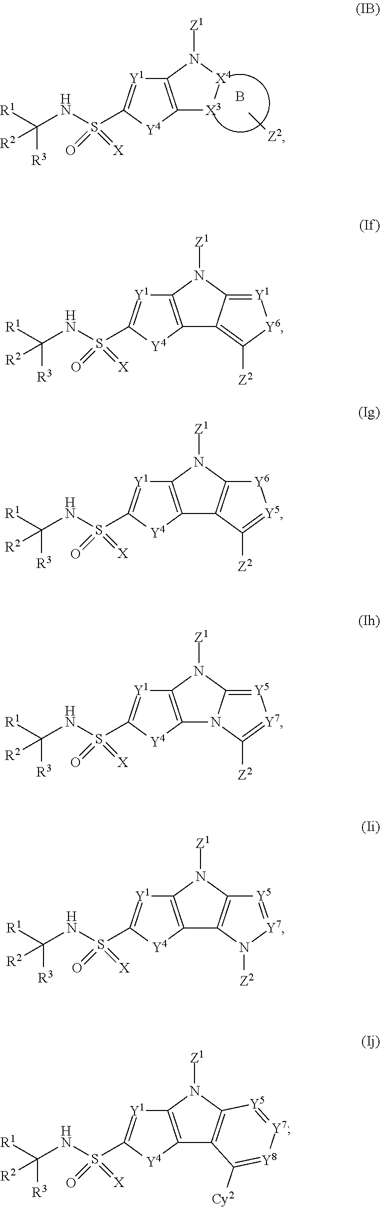

or a pharmaceutically acceptable salt, stereoisomer, solvate, N-oxide, tautomer, isotopic variant, prodrug, or deuterated compound thereof,
wherein, ring B, R¹, R², R³, X, X³, X⁴, Y¹, Y⁴, Y⁵, Y⁶, Y⁷, Y⁸, Z¹, and Z² are each as defined herein.

In some embodiments, Y¹ is N, and Y⁴ is S.

In some embodiments, Y¹ is N, and Y⁴ is O.

In some embodiments, Y¹ is N, and Y⁴ is NR⁷.

In some embodiments, Y¹ is CR⁵, and Y⁴ is S.

In some embodiments, Y¹ is CR⁵, and Y⁴ is O.

In some embodiments, Y¹ is CR⁵, and Y⁴ is NR⁷.

In some embodiments, R⁷ is H, D, C₁-C₆ alkyl, C₁-C₆ haloalkyl, C₂-C₆ alkenyl, C₂-C₆ alkynyl, C₁-C₆ alkyl-OR^{A}, C₁-C₆ alkyl-CN, C₁-C₆ alkyl-NR^{C}R^{D}, C(O)R^{B}, C(O)NR^{C}R^{D}.

In some embodiments, R⁷ is H. In some embodiments, R⁷ is D.

In some embodiments, R⁷ is C₁-C₆ alkyl. In some embodiments, R⁷ is C₁-C₆ haloalkyl. In some embodiments, R⁷ is C₂-C₆ alkenyl. In some embodiments, R⁷ is C₂-C₆ alkynyl. In some embodiments, R⁷ is C₁-C₆ alkyl-OR^{A}. In some embodiments, R⁷ is C₁-C₆ alkyl-CN. In some embodiments, R⁷ is C₁-C₆ alkyl-NR^{C}R^{D}. In some embodiments, R⁷ is C(O)R^{B}. In some embodiments, R⁷ is C(O)NR^{C}R^{D}.

In some embodiments, R⁷ is H, D, C₁-C₆ alkyl. In some embodiments, R⁷ is H, D, CH₃.

In yet another embodiment, provided herein is a compound of Formula (IC), (Ik), (Il), (Im), (In):

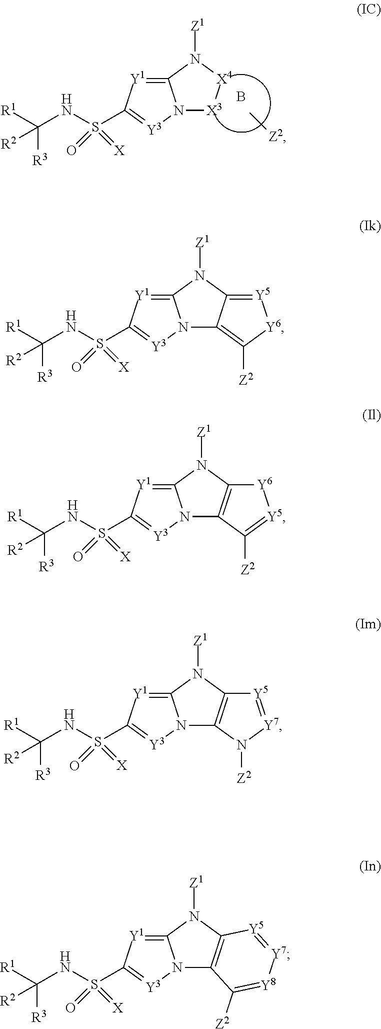

or a pharmaceutically acceptable salt, stereoisomer, solvate, N-oxide, tautomer, isotopic variant, prodrug, or deuterated compound thereof;
wherein, ring B, R¹, R², R³, X, X³, X⁴, Y¹, Y³, Y⁵, Y⁶, Y⁷, Y⁸, Z¹, and Z² are each as defined herein.

In yet another embodiment, provided herein is a compound of Formula (ID), (Io), (Ip), (Iq), (Ir):

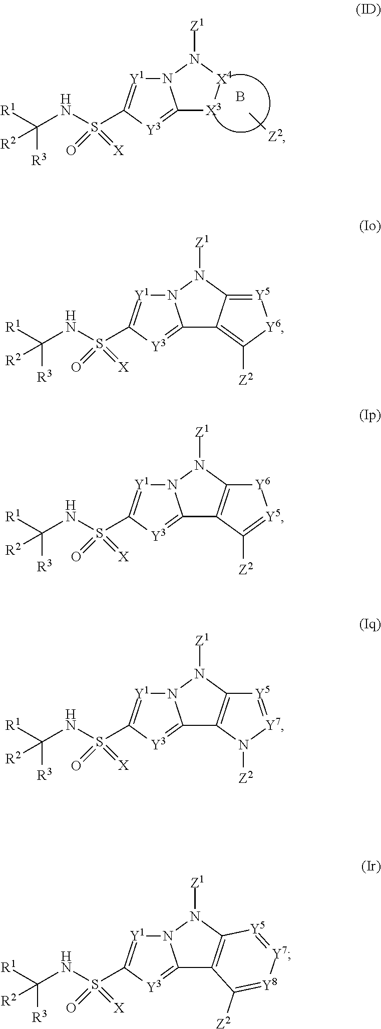

or a pharmaceutically acceptable salt, stereoisomer, solvate, N-oxide, tautomer, isotopic variant, prodrug, or deuterated compound thereof;
wherein, ring B, R¹, R², R³, X, X³, X⁴, Y¹, Y³, Y⁵, Y⁶, Y⁷, Y⁸, Z¹, and Z² are each as defined herein.

In some embodiments, Y¹ is N, and Y³ is N.

In some embodiments, Y¹ is N, and Y³ is CR⁵.

In some embodiments, Y¹ is CR⁵, and Y³ is N.

In some embodiments, Y¹ is CR⁵, and Y³ is CR⁵.

In some embodiments, Y⁵ is N, and Y⁶ is S.

In some embodiments, Y⁵ is N, and Y⁶ is O.

In some embodiments, Y⁵ is N, and Y⁶ is NR⁹.

In some embodiments, Y⁵ is CR⁸, and Y⁶ is S.

In some embodiments, Y⁵ is CR⁸, and Y⁶ is O.

In some embodiments, Y⁵ is CR⁸, and Y⁶ is NR⁹.

In some embodiments, Y⁵ is N, and Y⁷ is N.

In some embodiments, Y⁵ is N, and Y⁷ is CR⁸.

In some embodiments, Y⁵ is CR⁸, and Y⁷ is N.

In some embodiments, Y⁵ is CR⁸, and Y⁷ is CR⁸.

In some embodiments, R⁹ is H, D, C₁-C₆ alkyl, C₁-C₆ haloalkyl, C₂-C₆ alkenyl, C₂-C₆ alkynyl, C₁-C₆ alkyl-OR^{A}, C₁-C₆ alkyl-CN, C₁-C₆ alkyl-NR^{C}R^{D}, C(O)R^{B}, C(O)NR^{C}R^{D}.

In some embodiments, R⁹ is H. In some embodiments, R⁹ is D.

In some embodiments, R⁹ is C₁-C₆ alkyl. In some embodiments, R⁹ is C₁-C₆ haloalkyl. In some embodiments, R⁹ is C₂-C₆ alkenyl. In some embodiments, R⁹ is C₂-C₆ alkynyl. In some embodiments, R⁹ is C₁-C₆ alkyl-OR^{A}. In some embodiments, R⁹ is C₁-C₆ alkyl-CN. In some embodiments, R⁹ is C₁-C₆ alkyl-NR^{C}R^{D}. In some embodiments, R⁹ is C(O)R^{B}. In some embodiments, R⁹ is C(O)NR^{C}R^{D}.

In yet another embodiment, provided herein is a compound of Formula (IA) or (le):

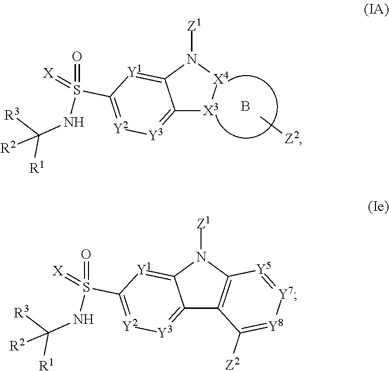

or a pharmaceutically acceptable salt, stereoisomer, solvate, N-oxide, tautomer, isotopic variant, prodrug, or deuterated compound thereof,
wherein, ring B, R¹, R², R³, X, X³, X⁴, Y¹, Y², Y³, Y⁵, Y⁷, Y⁸, Z¹, and Z² are each as defined herein.

In yet another embodiment, provided herein is a compound of Formula (Ie):

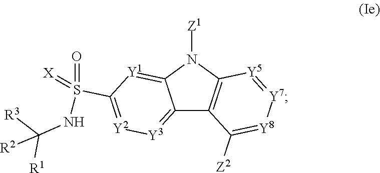

or a pharmaceutically acceptable salt, stereoisomer, solvate, N-oxide, tautomer, isotopic variant, prodrug, or deuterated compound thereof,
wherein, R¹, R², R³, X, Y¹, Y², Y³, Y⁵, Y⁷, Y⁸, Z¹, and Z² are each as defined herein.

In some embodiments, X is O or NR⁴.

In some embodiments, X is O.

In other embodiments, X is NR⁴.

In some embodiments, R⁴ is H, D, CN, OR^{B}, C₁-C₄ alkyl optionally substituted with at least one of R^{4A}; wherein, each R⁴ is independently D, F, Cl, CN, NH₂, OH, —O—C₁-C₆ alkyl, —OC₁-C₆ haloalkyl, optionally substituted C₃-C₇ cycloalkyl, or optionally substituted 4-7 membered heterocycloalkyl; wherein, optionally substituted is D, halo, CN, OH, C₁-C₄ alkyl, C₁-C₄ haloalkyl, —O—C₁-C₄ alkyl, —OC₁-C₄haloalkyl.

In some embodiments, R⁴ is H. In some embodiments, R⁴ is D. In some embodiments, R⁴ is CN. In some embodiments, R⁴ is OR^{B}.

In some embodiments, R⁴ is C₁-C₄ alkyl optionally substituted with R⁴; wherein, each R^{4A} is independently D, F, Cl, CN, NH₂, OH, —O—C₁-C₆ alkyl, —OC₁-C₆ haloalkyl, optionally substituted C₃-C₇ cycloalkyl, or optionally substituted 4-7 membered heterocycloalkyl; wherein, optionally substituted is D, halo, CN, OH, C₁-C₄ alkyl, C₁-C₄haloalkyl, —O—C₁-C₄ alkyl, —OC₁-C₄ haloalkyl.

In yet another embodiment, provided herein is a compound of Formula (IIa) and (IIb):

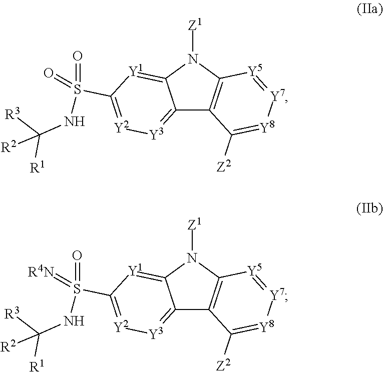

or pharmaceutically acceptable salt, stereoisomer, solvate, N-oxide, tautomer, isotopic variant, prodrug, or deuterated compound thereof, wherein, R¹, R², R³, R⁴, Y¹, Y², Y³, Y⁵, Y⁷, Y⁸, Z¹, and Z² are each as defined herein.

In some embodiments, Z¹ is 5-10 membered heteroaryl optionally substituted by 1, 2, 3, 4, or 5 substituents independently selected from R¹⁰. In some embodiments, Z¹ is 5-10 membered heteroaryl having 1, 2, 3, 4 heteroatoms independently selected from N, O, S which is optionally substituted by 1, 2, 3, 4, or 5 substituents independently selected from R¹⁰.

In some embodiments, Z¹ is 5-6 membered heteroaryl optionally substituted by 1, 2, 3, or 4 substituents independently selected from R¹⁰. In some embodiments, Z¹ is 5-6 membered heteroaryl having 1, 2, or 3 heteroatoms independently selected from N, O, S which is optionally substituted by 1, 2, 3, 4 substituents independently selected from R¹⁰.

In some embodiments, Z¹ is 5 membered heteroaryl optionally substituted by 1, 2, or 3 substituents independently selected from R¹⁰. In some embodiments, Z¹ is 5 membered heteroaryl having 1, 2, or 3 heteroatoms independently selected from N, O, S which is optionally substituted by 1, 2, or 3 substituents independently selected from R¹⁰.

In some embodiments, Z¹ is

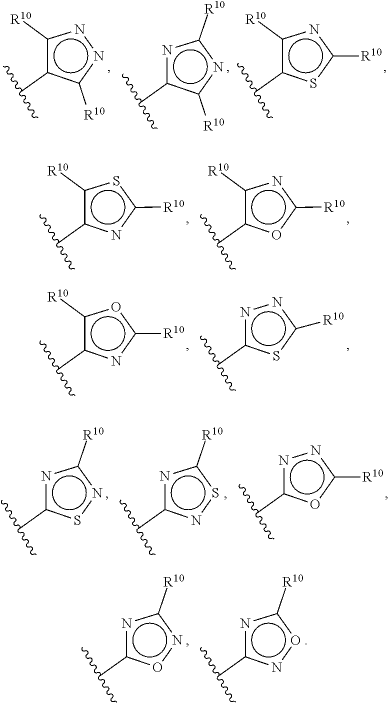

In some embodiments, Z¹ is

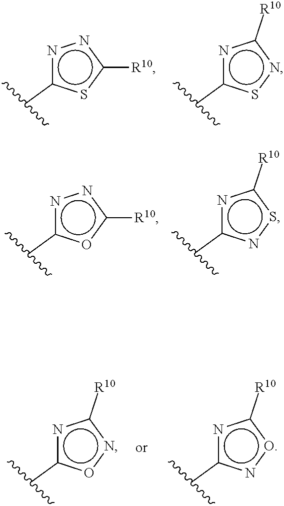

In some embodiments, Z¹ is

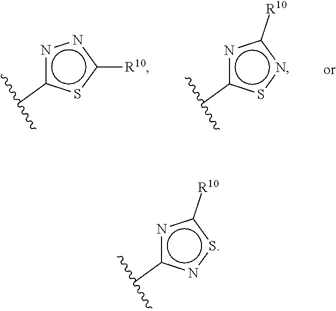

In some embodiments, Z¹ is

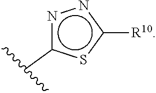

In some embodiments, provided herein is a compound of Formula (III):

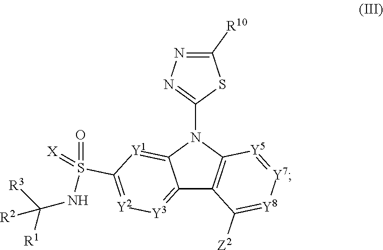

or pharmaceutically acceptable salt, stereoisomer, solvate, N-oxide, tautomer, isotopic variant, prodrug, or deuterated compound thereof, wherein R¹, R², R³, R¹⁰, X, Y¹, Y², Y³, Y⁵, Y⁷, Y⁸, and Z² are each as defined herein.

In some embodiments, R¹, R² and R³ are each independently H, D, CN, C₁-C₃ alkyl, C₂-C₃ alkenyl, C₂-C₃ alkynyl, C₃-C₇ cycloalkyl, or 4-7 membered heterocycloalkyl; wherein, the C₁-C₃ alkyl, C₂-C₃ alkenyl, C₂-C₃ alkynyl, C₃-C₇ cycloalkyl, 4-7 membered heterocycloalkyl is optionally substituted by 1, 2, 3, 4 or 5 substituents independently selected from D, halo, CN, OH, C₁-C₆ alkyl, C₁-C₆ haloalkyl, —O—C₁-C₆ alkyl, —OC₁-C₆ haloalkyl.

In some embodiments, R¹ is H, D, CN, C₁-C₃ alkyl, C₂-C₃ alkenyl, C₂-C₃ alkynyl, C₃-C₇ cycloalkyl, or 4-7 membered heterocycloalkyl; wherein, the C₁-C₃ alkyl, C₂-C₃ alkenyl, C₂-C₃ alkynyl, C₃-C₇ cycloalkyl, 4-7 membered heterocycloalkyl is optionally substituted by 1, 2, 3, 4 or 5 substituents independently selected from D, halo, CN, OH, C₁-C₆ alkyl, C₁-C₆ haloalkyl, —O—C₁-C₆ alkyl, —OC₁-C₆ haloalkyl.

In some embodiments, R¹ is H, D, CN, C₁-C₃ alkyl, C₂-C₃ alkenyl, C₂-C₃ alkynyl, C₃-C₇ cycloalkyl, or 4-7 membered heterocycloalkyl; wherein, the C₁-C₃ alkyl, C₂-C₃ alkenyl, C₂-C₃ alkynyl, C₃-C₇ cycloalkyl, or 4-7 membered heterocycloalkyl is optionally substituted by 1, 2, 3, 4 or 5 substituents independently selected from D, halo (such as F, Cl, Br or I), CN, OH, Me, CF₃, OMe, OCF₃, OEt.

In some embodiments, R¹ is H, D, CN, C₁-C₃ alkyl optionally substituted by 1, 2, 3, 4 or 5 substituents independently selected from D, halo (such as F, Cl, Br or I), CN, OH, Me, CF₃, OMe, OCF₃, OEt.

In some embodiments, R¹ is H, D, CN, CH₃, CD₃, CH₂CH₃, CF₃, CHF₂, CH₂F, CH₂CH₂F, CH₂OH, CH₂OCH₃ or CH₂CN.

In some embodiments, R¹ is CN, CH₃, CD₃, CF₃, CHF₂, CD₂F or CH₂F. In some embodiments, R¹ is CF₃. In some embodiments, R¹ is CHF₂. In some embodiments, R¹ is CH₂F.

In some embodiments, R¹ is CD₂F. In some embodiments, R¹ is CH₃. In some embodiments, R¹ is CN.

In some embodiments, R¹ and R⁴ together with the atoms to which they are attached form 5- to 7-membered partially saturated heterocycloalkyl optionally substituted by 1, 2, 3 or 4 substituents independently selected from D, halogen, CN, CF₃, NO₂, oxo, OH, C₁-C₃ alkyl, C₁-C₃ haloalkyl, —O—C₁-C₃ alkyl, —OC₁-C₃ haloalkyl.

In some embodiments, R¹ and R⁴ together with the atoms to which they are attached form 5- to 7-membered partially saturated heterocycloalkyl optionally substituted by 1, 2, 3 or 4 substituents independently selected from D, halogen, CN, CF₃, NO₂, oxo, OH, Me, CF₃, OMe, OCF₃, OEt.

In some embodiments, R² is H, D, CN, C₁-C₃ alkyl, C₂-C₃ alkenyl, C₂-C₃ alkynyl, C₃-C₇ cycloalkyl, or 4-7 membered heterocycloalkyl; wherein, the C₁-C₃ alkyl, C₂-C₃ alkenyl, C₂-C₃ alkynyl, C₃-C₇ cycloalkyl, 4-7 membered heterocycloalkyl is optionally substituted by 1, 2, 3, 4 or 5 substituents independently selected from D, halo, CN, OH, C₁-C₆ alkyl, C₁-C₆ haloalkyl, —O—C₁-C₆ alkyl, —OC₁-C₆ haloalkyl.

In some embodiments, R² is H, D, CN, C₁-C₃ alkyl, C₂-C₃ alkenyl, C₂-C₃ alkynyl, C₃-C₇ cycloalkyl, or 4-7 membered heterocycloalkyl; wherein, the C₁-C₃ alkyl, C₂-C₃ alkenyl, C₂-C₃ alkynyl, C₃-C₇ cycloalkyl, or 4-7 membered heterocycloalkyl is optionally substituted by 1, 2, 3, 4 or 5 substituents independently selected from D, halo, CN, OH, Me, CF₃, OMe, OCF₃, OEt.

In some embodiments, R² is C₁-C₃ alkyl, C₂-C₃ alkenyl, C₂-C₃ alkynyl, C₃-C₇ cycloalkyl, or 4-7 membered heterocycloalkyl; wherein, the C₁-C₃ alkyl, C₂-C₃ alkenyl, C₂-C₃ alkynyl, C₃-C₇ cycloalkyl, 4-7 membered heterocycloalkyl is optionally substituted by 1, 2, 3, 4 or 5 substituents independently selected from D, halo, CN, OH, C₁-C₆ alkyl, C₁-C₆ haloalkyl, —O—C₁-C₆ alkyl, —OC₁-C₆ haloalkyl.

In some embodiments, R³ is H, D, CN, C₁-C₃ alkyl, C₂-C₃ alkenyl, C₂-C₃ alkynyl, C₃-C₇ cycloalkyl, or 4-7 membered heterocycloalkyl; wherein, the C₁-C₃ alkyl, C₂-C₃ alkenyl, C₂-C₃ alkynyl, C₃-C₇ cycloalkyl, 4-7 membered heterocycloalkyl is optionally substituted by 1, 2, 3, 4 or 5 substituents independently selected from D, halo, CN, OH, C₁-C₆ alkyl, C₁-C₆ haloalkyl, —O—C₁-C₆ alkyl, —OC₁-C₆ haloalkyl.

In some embodiments, R³ is H, D, CN, C₁-C₃ alkyl, C₂-C₃ alkenyl, C₂-C₃ alkynyl, C₃-C₇ cycloalkyl, or 4-7 membered heterocycloalkyl; wherein, the C₁-C₃ alkyl, C₂-C₃ alkenyl, C₂-C₃ alkynyl, C₃-C₇ cycloalkyl, or 4-7 membered heterocycloalkyl is optionally substituted by 1, 2, 3, 4 or 5 substituents independently selected from D, halo, CN, OH, Me, CF₃, OMe, OCF₃, OEt.

In some embodiments, R³ is C₁-C₃ alkyl, C₂-C₃ alkenyl, C₂-C₃ alkynyl, C₃-C₇ cycloalkyl, or 4-7 membered heterocycloalkyl; wherein the C₁-C₃ alkyl, C₂-C₃ alkenyl, C₂-C₃ alkynyl, C₃-C₇ cycloalkyl, 4-7 membered heterocycloalkyl is optionally substituted by 1, 2, 3, 4 or 5 substituents independently selected from D, halo, CN, OH, C₁-C₆ alkyl, C₁-C₆ haloalkyl, —O—C₁-C₆ alkyl, —OC₁-C₆ haloalkyl.

In some embodiments, R² and R³ together with the carbon atom to which they are attached form C₃-C₇ cycloalkyl, or 4-7 membered heterocycloalkyl; wherein, the C₃-C₇ cycloalkyl, or 4-7 membered heterocycloalkyl is optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl.

In some embodiments, R² and R³ together with the carbon atom to which they are attached form cyclopropyl, cyclobutyl, oxetanyl, or siletanyl; each is optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl.

In some embodiments, R² and R³ together with the carbon atom to which they are attached form cyclopropyl. In some embodiments, R² and R³ together with the carbon atom to which they are attached form cyclobutyl. In some embodiments, R² and R³ together with the carbon atom to which they are attached form oxetanyl. In some embodiments, R² and R³ together with the carbon atom to which they are attached form siletanyl optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl.

In some embodiments, the moiety

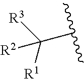

has the structure of

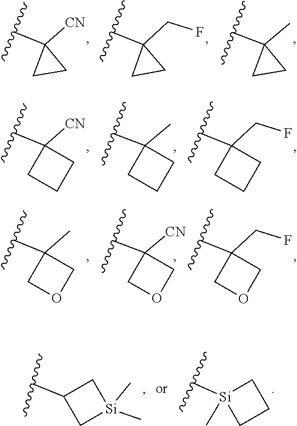

In yet another embodiment, provided herein is a compound of Formula (IV):

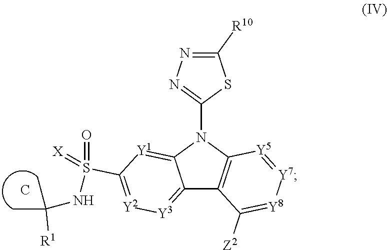

or pharmaceutically acceptable salt, stereoisomer, solvate, N-oxide, tautomer, isotopic variant, prodrug, or deuterated compound thereof, wherein,
ring C is C₃-C₇ cycloalkylene, or 4-7 membered heterocycloalkylene; wherein, the C₃-C₇ cycloalkylene, 4-7 membered heterocycloalkylene is optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl;
R¹, R¹⁰, X, Y¹, Y², Y³, Y⁵, Y⁷, Y⁸, and Z² are each as defined herein.

In some embodiments, ring C is 4-7 membered heterocycloalkylene optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl. In some embodiments, ring C is azetidinylene, oxetanylene, siletanylene, pyrrolidinylene, or tetrahydrofuranylene; each is optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl. In some embodiments, ring C is oxetanylene optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl.

In some embodiments, ring C is C₃-C₇ cycloalkylene optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl.

In some embodiments, ring C is cyclobutylene optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl.

In some embodiments, ring C is cyclopropylene optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl.

In some embodiments, ring C is cyclobutylene. In some embodiments, ring C is cyclopropyl.

In yet another embodiment, provided herein is a compound of Formula (IVa):

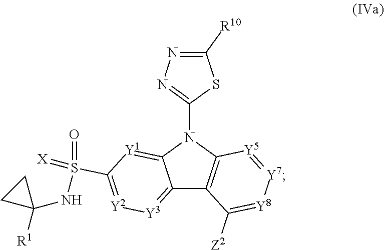

or pharmaceutically acceptable salt, stereoisomer, solvate, N-oxide, tautomer, isotopic variant, prodrug, or deuterated compound thereof, wherein R¹, R₁₀, X, Y¹, Y³, Y⁵, Y⁷, Y⁸ and Z² are each as defined herein.

In some embodiments, R¹ is H, D, CN, C₁-C₃ alkyl optionally substituted by 1, 2, 3, 4 or 5 substituents independently selected from D, halo, CN, OH, Me, CF₃, OMe, OCF₃, OEt.

In some embodiments, R¹ is H, D, CN, CH₃, CD₃, CH₂CH₃, CF₃, CHF₂, CH₂F, CD₂F, CH₂CH₂F, CH₂OH, CH₂OCH₃ or CH₂CN.

In some embodiments, R¹ is CN, CH₃, CD₃, CF₃, CHF₂, CD₂F, or CH₂F. In some embodiments, R¹ is CF₃. In some embodiments, R¹ is CHF₂. In some embodiments, R¹ is CH₂F.

In some embodiments, R¹ is CD₂F. In some embodiments, R¹ is CH₃. In some embodiments, R¹ is CD₃. In some embodiments, R¹ is CN.

In some embodiments, X is NR⁴. In some embodiments, X is O.

In some embodiments, Y¹ is N, Y² is N, and Y³ is CR⁵.

In some embodiments, Y¹ is N, Y² is N, and Y³ is N.

In some embodiments, Y¹ is N, Y² is CR⁶, and Y³ is CR⁵.

In some embodiments, Y¹ is N, Y² is CR⁶, and Y³ is N.

In some embodiments, Y¹ is CR⁵, Y² is N, and Y³ is CR⁵.

In some embodiments, Y¹ is CR⁵, Y² is N, and Y³ is N.

In some embodiments, Y¹ is CR⁵, Y² is CR⁶, and Y³ is N.

In some embodiments, Y¹ is CR⁵, Y² is CR⁶, and Y³ is CR⁵. In some embodiments, Y¹ is CH, Y² is CH, and Y³ is CH. In some embodiments, Y¹ is CH, Y² is CF, and Y³ is CH.

In some embodiments, each R⁵ is independently H, D, CN, halo, SF₅, OH, NH₂, CHO, COOH, C₁-C₃ alkyl, C₂-C₃ alkenyl, C₂-C₃ alkynyl, C₁-C₃ haloalkyl, C₁-C₃ cyanoalkyl, OR^{A}, NR^{C}R^{D} or C(O)R^{B}.

In some embodiments, each R⁵ is independently H, D, OH, CN, NO₂, SF₅, halo, C₁-C₃ haloalkyl, C₁-C₃ cyanoalkyl.

In some embodiments, each R⁵ is independently H, D, halo (such as F, Cl, Br or I), C₁-C₃ alkyl.

In some embodiments, each R⁵ is independently H, D, F, Cl or CH₃. In some embodiments, each R⁵ is independently H.

In some embodiments, R⁶ is H, D, CN, halo, OH, NH₂, C₁-C₃ alkyl (such as, CH₃, CH₂CH₃, CH₂CH₂CH₃, CH(CH₃)₂), C₁-C₃ haloalkyl (such as CF₃, CHF₂, CH₂F), —O—C₁-C₃ alkyl (such as, OCH₃, OCH₂CH₃, OCH₂CH₂CH₃, OCH(CH₃)₂), —OC₁-C₃ haloalkyl, C₁-C₃ cyanoalkyl, or SF₅.

In other embodiments, R⁶ is H, D, F, Cl, OH, NH₂, CN, CH₃, CF₃, OMe, OCF₃, or SF₅. In other embodiments, R⁶ is H, D, F, Cl, OH, NH₂, or CN.

In some embodiments, R⁶ is H. In some embodiments, R⁶ is D. In some embodiments, R⁶ is F. In some embodiments, R⁶ is Cl. In some embodiments, R⁶ is OH. In some embodiments, R⁶ is CN.

In other embodiments, R⁶ is H, D, or F.

In some embodiments, R⁶ is C₁-C₃ alkyl, for example, CH₃. In some embodiments, R⁶ is C₁-C₃ haloalkyl, for example, CHF₂, CF₃. In some embodiments, R⁶ is —O—C₁-C₃ alkyl, for example, OMe. In some embodiments, R⁶ is —OC₁-C₃ haloalkyl, for example, OCF₃. In some embodiments, R⁶ is SF₅.

In some embodiments, Y⁵ is CR⁸, Y⁷ is CR⁸, and Y⁸ is CR⁸.

In some embodiments, Y⁵ is CR⁸, Y⁷ is CR⁸, and Y⁸ is N.

In some embodiments, Y⁵ is CR⁸, Y⁷ is N, and Y⁸ is CR⁸.

In some embodiments, Y⁵ is CR⁸, Y⁷ is N, and Y⁸ is N.

In some embodiments, Y⁵ is N, Y⁷ is CR⁸, and Y⁸ is CR⁸.

In some embodiments, Y⁵ is N, Y⁷ is CH, and Y⁸ is CH. In some embodiments, Y⁵ is N, Y⁷ is CCH₃, and Y⁸ is CH.

In some embodiments, Y⁵ is N, Y⁷ is CR⁸, and Y⁸ is N.

In some embodiments, Y⁵ is N, Y⁷ is CH, and Y⁸ is N. In some embodiments, Y⁵ is N, Y⁷ is CCH₃, and Y⁸ is N.

In some embodiments, Y⁵ is N, Y⁷ is N, and Y⁸ is CR⁸.

In some embodiments, Y⁵ is N, Y⁷ is N, and Y⁸ is N.

In some embodiments, each R⁸ is independently H, D, halogen, CN, NO₂, C₁-C₆ alkyl, C₁-C₆ haloalkyl, C₂-C₆ alkenyl, C₂-C₆ alkynyl, OR^{A}, SR^{A}, SF₅, NHOR^{A}, C(O)OR^{A}, C(O)R^{B}C(O)NR^{C}R^{D}, OC(O)NR^{C}R^{D}, NR^{C}R^{D}, NR^{C}C(O)R^{B}, NR^{C}C(O)NR^{C}R^{D}, NR^{C}C(O)OR^{A}, NR^{C}S(O)₂R^{B}, B(OR^{C})(OR^{D}), C(═NR^{C})NR^{C}R^{D}, NR^{D}C(═NR^{C})NR^{C}R^{D} NR^{D}C(═NR^{C})R^{B} P(O)R^{E}R^{F}, P(O)OR^{E}OR^{F}, OP(O)OR^{E}OR^{F}, S(O)(═NR^{B})R^{B}, S(O)R^{B}, S(O)NR^{C}R^{D}, S(O)₂R^{B}, S(O)₂NR^{C}R^{D}, NR^{C}S(O)₂NR^{C}R^{D}, or NR^{C}S(O)(═NR^{B})R^{B}; wherein, the C₁-C₆ alkyl, C₂-C₆ alkenyl, or C₂-C₆ alkynyl is optionally substituted with 1, 2, or 3 substituents independently selected from R¹²

In some embodiments, each R⁸ is independently H, D, halogen, CN, NO₂, OR^{A}, SR^{A}, SF₅, C₁-C₆ alkyl or C₁-C₆ haloalkyl. In some embodiments, R⁸ is H. In some embodiments, R⁸ is D. In some embodiments, R⁸ is halogen. In some embodiments, R⁸ is F. In some embodiments, R⁸ is Cl. In some embodiments, R⁸ is Br. In some embodiments, R⁸ is I. In some embodiments, R⁸ is CN. In some embodiments, R⁸ is NO₂. In some embodiments, R⁸ is SF₅. In some embodiments, R⁸ is OR^{A}, for example, —OH, —OMe, —OCF₃. In some embodiments, R⁸ is SR^{A}, for example, —SMe.

In some embodiments, R⁸ is C₁-C₆ alkyl optionally substituted with 1, 2, or 3 substituents independently selected from R¹², for example, —CH₃, —CH₂CH₃. In some embodiments, R⁶ is C₁-C₆ haloalkyl, for example, —CF₃, —CHF₂, —CH₂F.

In some embodiments, R⁸ is C₂-C₆ alkenyl optionally substituted with 1, 2, or 3 substituents independently selected from R¹². In some embodiments, R¹ is C₂-C₆ alkynyl optionally substituted with 1, 2, or 3 substituents independently selected from R¹².

In some embodiments, R⁸ is B(OR^{C})(OR^{D}), for example, B(OH)₂. In some embodiments, R⁸ is NHOR^{A}, for example, NHOH. In some embodiments, R⁸ is NR^{C}R^{D}, for example, —NH₂, —NHCH₃, —N(CH₃)₂.

In some embodiments, R⁸ is H, D, C₁-C₆ alkyl. In some embodiments, R⁸ is H, D, CH₃.

In some embodiments, R¹⁰ is H, D, halo, C₁-C₆ alkyl, C₂-C₆ alkenyl, C₂-C₆ alkynyl, C₁-C₆ haloalkyl, OC₁-C₆ alkyl, OC₁-C₆ haloalkyl, OC₃-C₇ cycloalkyl, C₃-C₇ cycloalkyl, CN, NO₂, N₃, or SF₅; wherein the C₁-C₆ alkyl, C₂-C₆ alkenyl, C₂-C₆ alkynyl or C₃-C₇ cycloalkyl is optionally substituted with 1, 2, or 3 substituents independently selected from R¹².

In some embodiments, R¹⁰ is H. In some embodiments, R¹⁰ is D. In some embodiments, R¹⁰ is halo. In some embodiments, R¹⁰ is F, Cl, Br, I. In some embodiments, R¹⁰ is CN. In some embodiments, R¹⁰ is NO₂. In some embodiments, R¹⁰ is N₃. In some embodiments, R¹⁰ is SF₅.

In yet other embodiments, R¹⁰ is C₁-C₆ alkyl, C₂-C₆ alkenyl, C₂-C₆ alkynyl, C₁-C₆ haloalkyl; wherein the C₁-C₆ alkyl, C₂-C₆ alkenyl, or C₂-C₆ alkynyl is optionally substituted with 1, 2, or 3 substituents independently selected from R¹².

In yet other embodiments, R¹⁰ is C₁-C₆ alkyl, C₂-C₆ alkenyl, C₂-C₆ alkynyl, C₁-C₆ haloalkyl. In some embodiments, R¹⁰ is C₁-C₆ alkyl. In some embodiments, R¹⁰ is methyl. In some embodiments, R¹⁰ is ethyl. In some embodiments, R¹⁰ is isopropyl. In some embodiments, R¹⁰ is t-butyl.

In other embodiments, R¹⁰ is C₂-C₆ alkenyl. In yet other embodiments, R¹⁰ is C₂-C₆ alkynyl.

In yet other embodiments, R¹⁰ is C₁-C₆ haloalkyl. In some embodiments, R¹⁰ is CF₃. In some embodiments, R¹⁰ is CHF₂. In some embodiments, R¹⁰ is CH₂F. In some embodiments, R¹⁰ is CDF₂.

In yet other embodiments, R¹⁰ is C₃-C₇ cycloalkyl optionally substituted with 1, 2, or 3 substituents independently selected from R¹². In yet other embodiments, R¹⁰ is C₃-C₇ cycloalkyl. In yet other embodiments, R¹⁰ is cyclobutyl. In yet other embodiments, R¹⁰ is cyclopropyl.

In yet other embodiments, R¹⁰ is OC₁-C₆ alkyl, OC₁-C₆ haloalkyl, OC₃-C₇ cycloalkyl; wherein, the C₁-C₆ alkyl or C₃-C₇ cycloalkyl is optionally substituted with 1, 2, or 3 substituents independently selected from R¹². In some embodiments, R¹⁰ is optionally substituted OC₁-C₆ alkyl. In some embodiments, R¹⁰ is OC₁-C₆ haloalkyl. In some embodiments, R¹⁰ is OC₃-C₇ cycloalkyl.

In some embodiments, R¹⁰ is C₁-C₆ haloalkyl. In some embodiments, R¹⁰ is CHF₂.

In some embodiments, Z² is H, D, halo, CN, NO₂, SF₅, C₁-C₅ alkyl, C₂-C₅ alkenyl, C₂-C₅ alkynyl, C₆-C₁₀ aryl, C₃-C₁₀ cycloalkyl, 5-10 membered heteroaryl, or 4-14 membered heterocycloalkyl, NR^{C}R^{D}, OR^{A}, SR^{A}, NHOR^{A}, C(O)R^{B}, C(O)OR^{A}, C(O)NR^{C}R^{D}, OC(O)NR^{C}R^{D}NR^{C}C(O)R^{B}, NR^{C}C(O)NR^{C}R^{D}, NR^{C}C(O)OR^{A}, NR^{C}S(O)₂R^{B}, S(O)R^{B}, S(O)NR^{C}R^{D}, S(O)₂R^{B}, S(O)₂NR^{C}R^{D}, or NR^{C}S(O)₂NR^{C}R^{D}; wherein, the C₁-C₅ alkyl, C₂-C₅ alkenyl, C₂-C₅ alkynyl, C₆-C₁₀ aryl, C₃-C₁₀ cycloalkyl, 5-10 membered heteroaryl, or 4-14 membered heterocycloalkyl is optionally substituted by 1, 2, 3, 4, or 5 substituents independently selected from R¹¹.

In some embodiments, Z² is H. In some embodiments, Z² is D. In some embodiments, Z² is halo (such as F, Cl, Br or I). In some embodiments, Z² is CN. In some embodiments, Z² is NO₂. In some embodiments, Z² is SF₅.

In some embodiments, Z² is NR^{C}R^{D}, OR^{A}, SR^{A}, NR^{C}C(O)R^{B} C₁-C₈ alkyl, C₂-C₈ alkenyl, C₂-C₈ alkynyl; wherein, the C₁-C₅ alkyl, C₂-C₅ alkenyl, C₂-C₅ alkynyl is optionally substituted by 1, 2, 3, 4, or 5 substituents independently selected from R¹¹.

In some embodiments, each R^{A} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocyclalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In some embodiments, each R^{A} is independently H, D, C₁-C₆ alkyl, C₃-C₁₀ cycloalkyl, 4-10 membered heterocycloalkyl, C₃-C₁₀ cycloalkyl-C₁-C₆ alkyl, or 4-10 membered heterocycloalkyl-C₁-C₆ alkyl; wherein, the C₁-C₆ alkyl, C₃-C₁₀ cycloalkyl, 4-10 membered heterocyclalkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In some embodiments, R^{B} is independently C₁-C₆ alkyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In some embodiments, R^{B} is independently C₁-C₆ alkyl, C₃-C₁₀ cycloalkyl-C₁-C₆ alkyl, or 4-membered heterocycloalkyl-C₁-C₆ alkyl; wherein, the C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or substituents independently selected from R¹⁴.

In some embodiments, each R^{C} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In some embodiments, each R^{C} is independently H, D, C₁-C₆ alkyl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In some embodiments, each R^{C} is independently H, D, C₁-C₆ alkyl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In some embodiments, each R^{C} is independently H, D, C₁-C₆ alkyl. In some embodiments, each R^{C} is independently H, D, CH₃, CD₃, CH₂CH₃.

In some embodiments, R^{D} is H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-membered heterocycloalkyl-C₁-C₆ alkyl is optionally substituted with 1, 2, 3, 4 or 5 substituents independently selected from R¹⁴.

In some embodiments, R^{D} is H, D, C₁-C₆ alkyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In some embodiments, each R¹⁴ is independently H, D, CN, halo, oxo, SF₅, C₁-C₈ alkyl, C₂-C₈ alkenyl, C₂-C₈ alkynyl, C₆-C₁₄ aryl, C₃-C₁₄ cycloalkyl, 5-14 membered heteroaryl, or 4-14 membered heterocycloalkyl, OR^{a}, SR^{a}, SF₅, NHOR^{a}, C(O)R^{b}, C(O)NR^{c}R^{d}, C(O)OR^{a}, NR^{c}R^{d}, or NR^{c}C(O)R; wherein, the C₁-C₈ alkyl, C₂-C₈ alkenyl, C₂-C₈ alkynyl, C₆-C₁₄ aryl, C₃-C₁₄ cycloalkyl, 5-14 membered heteroaryl, or 4-14 membered heterocycloalkyl is optionally substituted by 1, 2, 3, 4 or 5 substituents independently selected from D, NO₂, CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{c1}R^{d1}, C(O)OR^{a1}, OC(O)R^{b1}, OC(O)NR^{c1}R^{d1} NR^{c1}R^{d1} NR^{c1}C(O)R^{b1}.

In some embodiments, each R¹⁴ is independently H, D, CN, halo, oxo, SF₅, C₁-C₈ alkyl, C₆-C₁₄ aryl, C₃-C₁₄ cycloalkyl, 5-14 membered heteroaryl, or 4-14 membered heterocycloalkyl, OR^{a}, SR^{a}, SF₅, NHOR^{a}, C(O)R^{b}, C(O)NR^{c}R^{d}, C(O)OR^{a}, NR^{c}R^{d}, or NR^{c}C(O)R^{b}; wherein, the C₁-C₈ alkyl, C₆-C₁₄ aryl, C₃-C₁₄ cycloalkyl, 5-14 membered heteroaryl, or 4-14 membered heterocycloalkyl is optionally substituted by 1, 2, 3, 4 or 5 substituents independently selected from D, NO₂, CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1} NR^{c1}C(O)R^{b1}.

In some embodiments, each R¹⁴ is independently H, D, CN, halo, oxo, SF₅, —CH₃, —CD₃, —CH₂CH₃, —OH, —OCH₃, —OCH₂CH₃, —NH₂, —N(CH₃)₂, —N(CH₂CH₃)₂, —C(O)CH(CH₃)₂, —COOH, —C(O)N(CH₃)₂, or

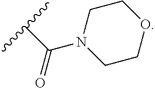 [SMILES: *C(=O)N1CCOCC1]

In some embodiments, each R^{A} is independently H, D, —CH₃, —CD₃, —CH₂CH₃, —CF₃, —CH₂CH₂OH, —CH₂CH₂OCH₃, tetrahydrofuranyl,

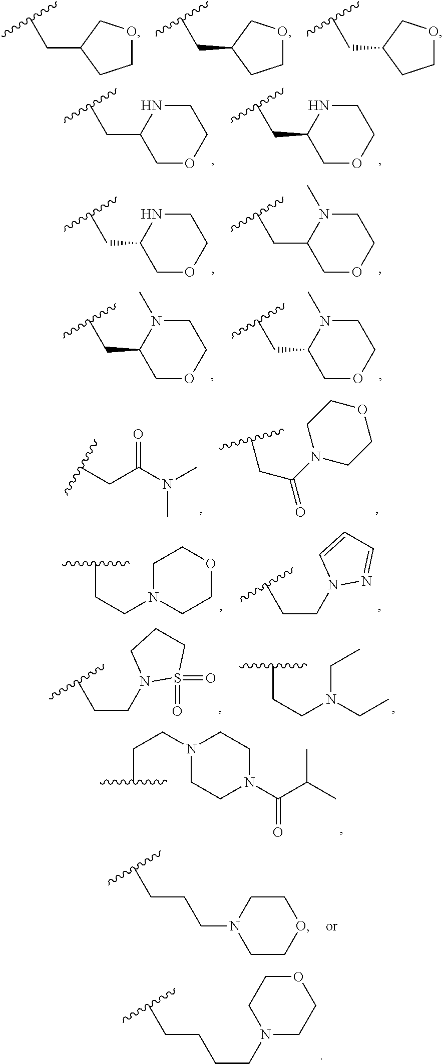

In some embodiments, R^{B} is

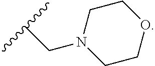 [SMILES: *CN1CCOCC1]

In some embodiments, R^{D} is H, D, —CH₃, —CD₃, —CH₂CH₃,

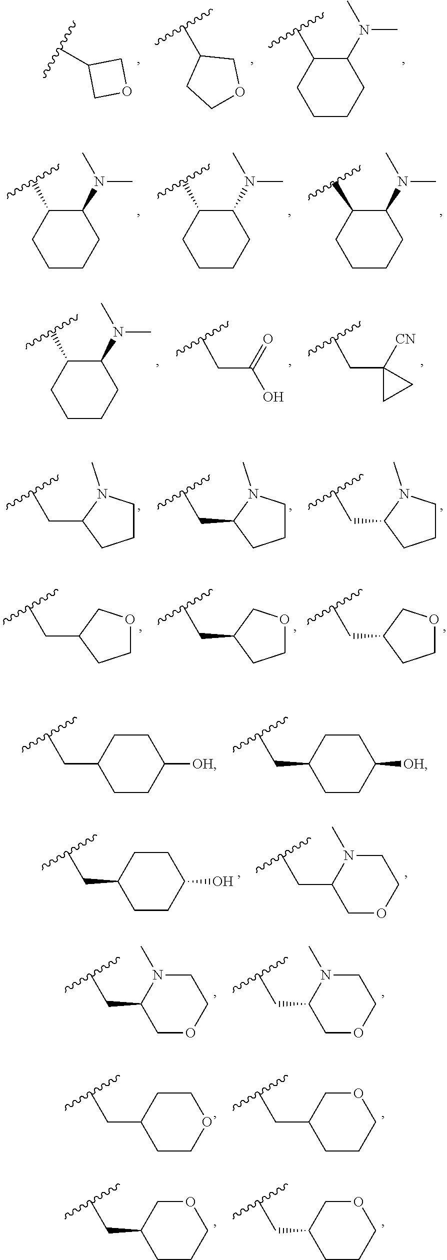

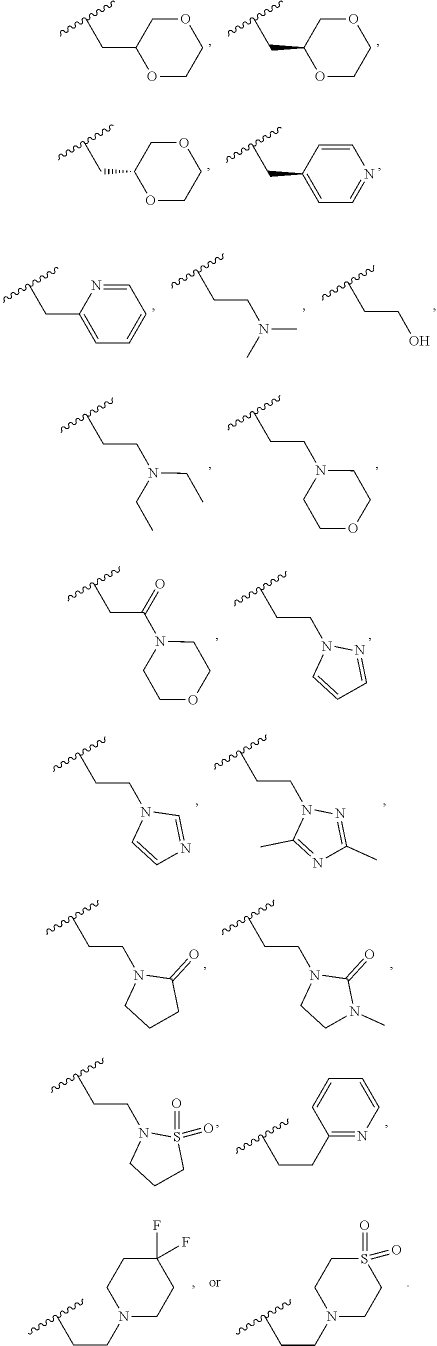

In some embodiments, Z² is C₁-C₈ alkyl, C₂-C₈ alkenyl, C₂-C₈ alkynyl; wherein, the C₁-C₈ alkyl, C₂-C₈ alkenyl, C₂-C₈ alkynyl is optionally substituted by 1, 2, 3, 4, or 5 substituents independently selected from R^{D}.

In some embodiments, Z² is C₁-C₈ alkyl optionally substituted by 1, 2, 3, 4, or 5 substituents independently selected from R¹¹.

In some embodiments, the moiety Z² has the structure of

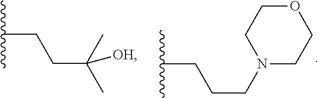

In some embodiments, Z² is C₂-C₈ alkenyl optionally substituted by 1, 2, 3, 4, or 5 substituents independently selected from R¹¹.

In some embodiments, the moiety Z² has the structure of

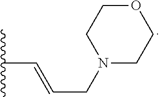 [SMILES: */C=C/CN1CCOCC1]

In some embodiments, Z² is C₂-C₈ alkynyl optionally substituted by 1, 2, 3, 4, or 5 substituents independently selected from R¹¹.

In some embodiments, the moiety Z² has the structure of

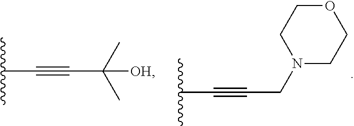

In some embodiments, each R¹¹ is independently H, D, halo, CN, N₃, oxo, OR^{A}, SR^{A}, SF₅, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D} Cy³.

In some embodiments, each R¹¹ is independently H, D, halo, CN, —OH, —OCH₃, —OCH₂CH₃, —NH₂, —NHCH₃, —N(CH₃)₂, —N(CH₂CH₃)₂, morpholinyl, or pyrazolyl.

In some embodiments, the moiety Z² has the structure of SH, OCF₂CF₃,

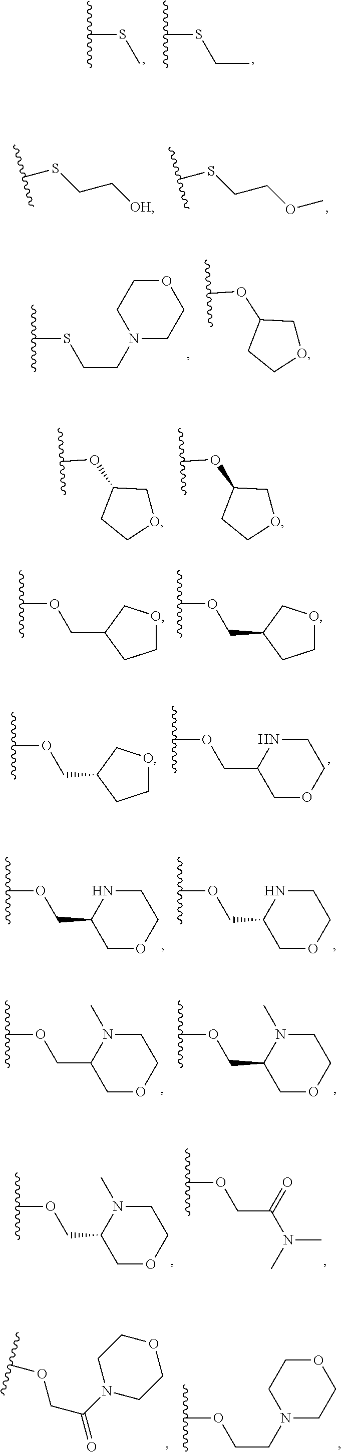

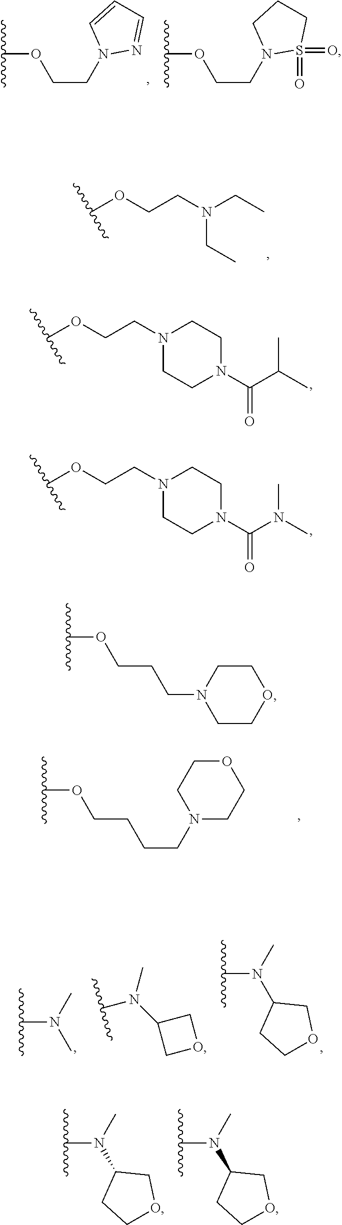

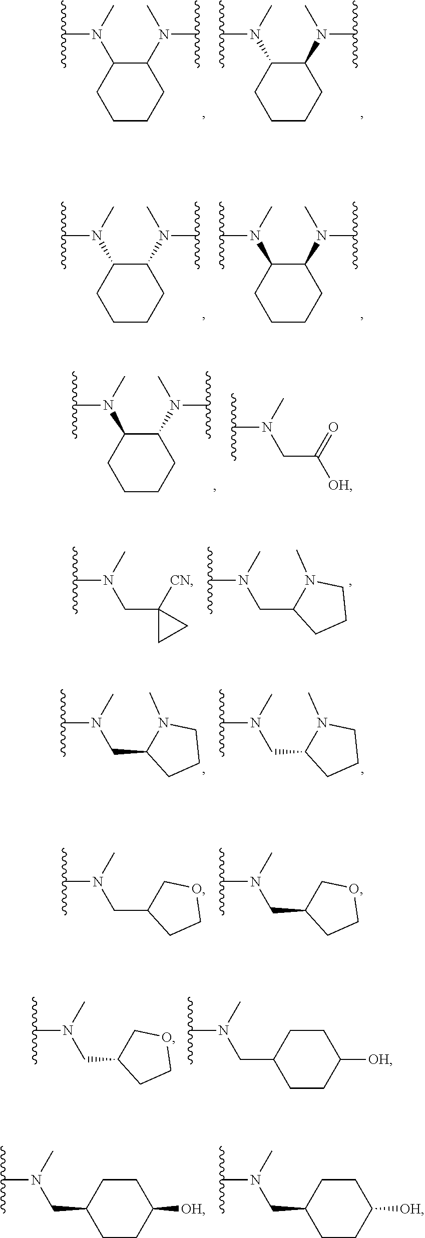

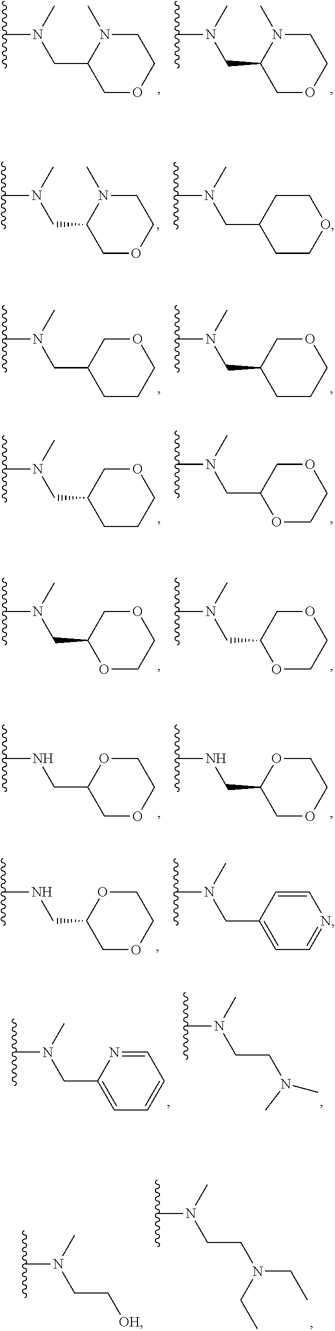

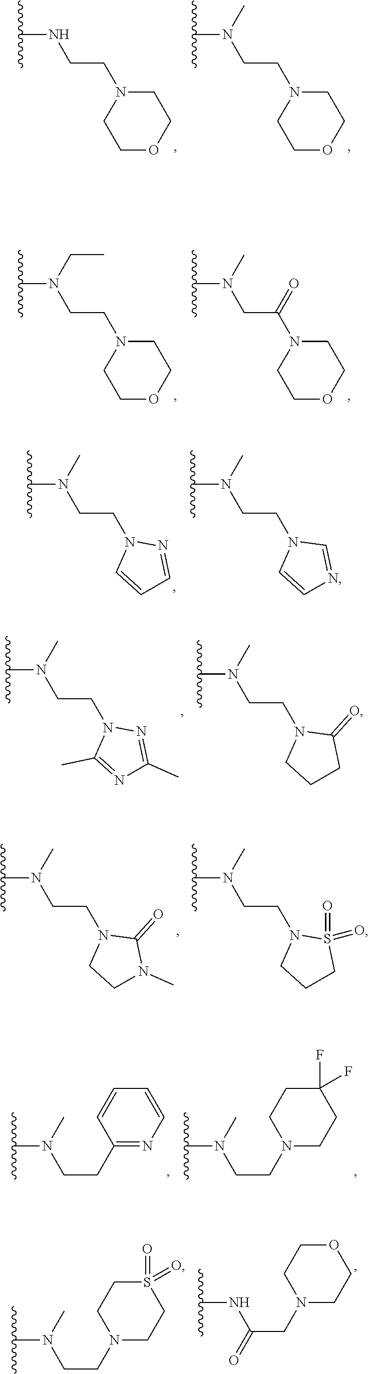

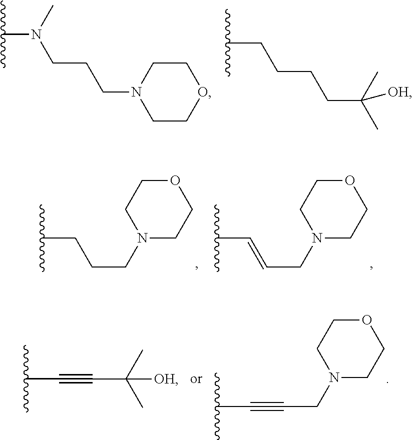

In some embodiments, Z² is NHOR^{A}. In some embodiments, Z² is C(O)R^{B}. In some embodiments, Z² is C(O)OR^{A}. In some embodiments, Z² is C(O)NR^{C}R^{D}. In some embodiments, Z² is OC(O)NR^{C}R^{D}. In some embodiments, Z² is NR^{C}C(O)NR^{C}R^{D}. In some embodiments, Z² is NR^{C}C(O)OR^{A}. In some embodiments, Z² is NR^{C}S(o)₂R^{B}. In some embodiments, Z² is S(O)R^{B}. In some embodiments, Z² is S(O)NR^{C}R^{D}. In some embodiments, Z² is S(O)₂R^{B}. In some embodiments, Z² is S(O)₂NR^{C}R^{D}. In some embodiments, Z² is NR^{C}S(O)₂NR^{C}R^{D}.

In some embodiments, Z² is C₆-C₁₀ aryl, C₃-C₁₀ cycloalkyl, 5-10 membered heteroaryl, or 4-14 membered heterocycloalkyl; wherein, the C₆-C₁₀ aryl, C₃-C₁₀ cycloalkyl, 5-10 membered heteroaryl, or 4-14 membered heterocycloalkyl optionally substituted by 1, 2, 3, 4 or 5 substituents independently selected from R¹¹.

In some embodiments, Z² is C₆-C₁₀ aryl, C₃-C₁₀ cycloalkyl, 5-10 membered heteroaryl, or 4-membered heterocycloalkyl; wherein, the C₆-C₁₀ aryl, C₃-C₁₀ cycloalkyl, 5-10 membered heteroaryl, or 4-10 membered heterocycloalkyl optionally substituted by 1, 2, 3, 4 or 5 substituents independently selected from R^{D}.

In some embodiments, Z² is C₆-C₁₀ aryl optionally substituted by 1, 2, 3, 4 or 5 substituents independently selected from R¹⁰. In some embodiments, Z² is phenyl optionally substituted by 1, 2, 3, 4 or 5 R¹¹.

In some embodiments, Z² is 5-10 membered heteroaryl optionally substituted by 1, 2, 3, 4 or 5 substituents independently selected from R^{D}.

In some embodiments, for example, Z² is pyrrolyl, furanyl, thiophenyl, imidazolyl, oxazolyl, thiazolyl, pyrazolyl, triazolyl, thiadiazolyl, oxadiazolyl, pyridinyl, pyrimidinyl, pyrazinyl, pyridazinyl, indolyl, indazolyl, benzo[d]imidazolyl, quinolinyl, quinoxalinyl, pyrrolo[3,2-b]pyridinyl, indolizinyl, each ring optionally substituted by 1, 2, 3, 4 or 5 substituents independently selected from R¹¹.

In some embodiments, Z² is C₃-C₁₀ cycloalkyl optionally substituted by 1, 2, 3, 4 or 5 substituents independently selected from R^{D}.

In some embodiments, Z² is saturated C₃-C₁₀ cycloalkyl or partially unsaturated C₅-C₁₀ cycloalkyl; each is optionally substituted by 1, 2, 3, 4 or 5 substituents independently selected from R¹¹.

In some embodiments, Z² is cyclopropyl, cyclobutyl, cyclopentyl, cyclohexyl, or cycloheptyl, each ring optionally substituted by 1, 2, 3, 4 or 5 substituents independently selected from R¹¹.

In some embodiments, Z² is saturated 4-14 membered heterocycloalkyl optionally substituted by 1, 2, 3, 4 or 5 substituents independently selected from R¹¹. In some embodiments, Z² is saturated 4-14 membered heterocycloalkyl having 1, 2, 3, 4 heteroatoms independently selected from N, O, S, P, Si and heteroatoms can be optionally substituted by one or more oxo or sulfido (e.g., S(O), S(O)₂, or P(O)), wherein, the 4-14 membered heterocycloalkyl is optionally substituted by 1, 2, 3, 4 or 5 substituents independently selected from R^{D}.

In some embodiments, Z² is partially unsaturated 5-14 membered mono-heterocycloalkyl optionally substituted by 1, 2, 3, 4 or 5 substituents independently selected from R^{1D}.

In some embodiments, Z² is piperidinyl, piperazinyl, morpholinyl, octahydropyrrolo[3,4-c]pyrrolyl, octahydro-1H-pyrrolo[3,2-c]pyridinyl, 2-azabicyclo[2.2.1]heptanyl, 2,5-diazabicyclo[2.2.1]heptanyl, 5,6,7,8-tetrahydroimidazo[1,5-a]pyrazinyl, 5,6,7,8-tetrahydroimidazo[1,2-a]pyrazinyl, 5,6,7,8-tetrahydro-[1,2,4]triazolo[4,3-a]pyrazinyl, 5,6,7,8-tetrahydro-[1,2,4]triazolo[1,5-a]pyrazinyl, 4,7-diazaspiro[2.5]octanyl, 1,8-diazaspiro[4.5]decanyl; each is optionally substituted by 1, 2, 3, 4 or 5 substituents independently selected from R¹¹.

In some embodiments, each R¹¹ is independently H, D, halo, CN, N₃, oxo, C₁-C₆ alkyl, OR^{A}, SR^{A}, SF₅, NR^{C}OR^{A}, C(O)R^{B}, NR^{C}R^{D} Cy³; wherein, the C₁-C₆ alkyl is optionally substituted 1, 2, 3, 4 or 5 substituents independently selected from R¹².

In some embodiments, each R¹¹ is independently H, D, halo, CN, N₃, oxo, —CH₃, —CD₃, CH₂F, CHF₂, CF₃, —CH₂CH₃, —CH(CH₃)₂, —CH₂OH, —CH₂OCH₃, —CH₂NH₂, —CH₂NHCH₃, —CH₂N(CH₃)₂, —OH, —OCH₃, —OCH₂CH₃, —NH₂, —NHCH₃, —N(CH₃)₂, —NHCH₂CH₃, —N(CH₂CH₃)₂, morpholinyl, pyrazolyl, or 4,4-difluoro-1-piperidinyl.

In some embodiments, the moiety Z² has the structure of

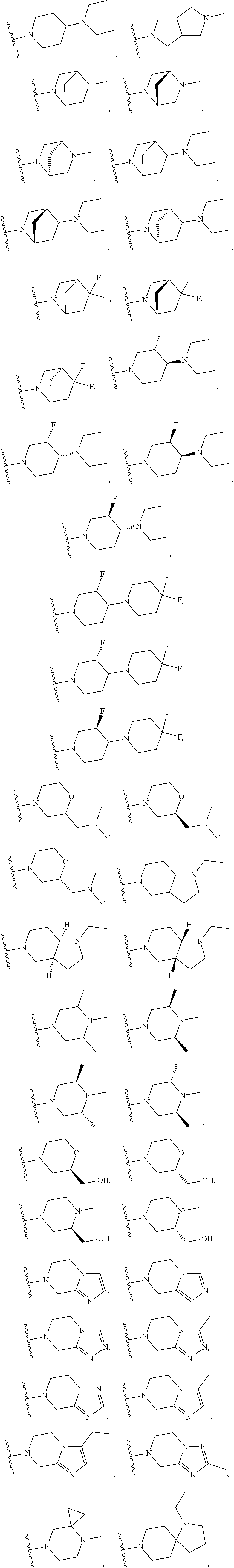

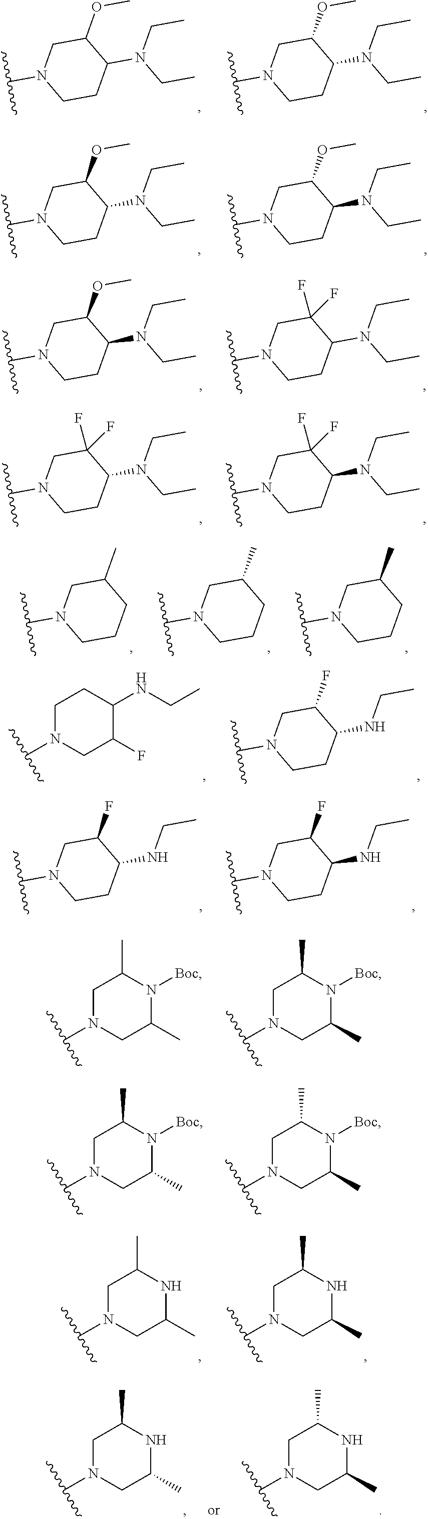

In some embodiments, each R¹¹ is independently H, D, halo, CN, NO₂, N₃, oxo, C₁-C₆ alkyl, C₂-C₆ alkenyl, C₂-C₆ alkynyl, OR^{A}, SR^{A}, SF₅, NR^{C}OR^{A}, C(O)R^{B}, C(═S)R^{B}, C(O)NR^{C}R^{D}, C(O)NR^{C}OR^{A}, C(O)OR^{A}, C(═NR^{C})NR^{C}R^{D}, OC(O)R^{B}, OC(O)NR^{C}R^{D}, NR^{C}R^{D}, NR^{C}C(O)R^{D} NR^{C}C(O)NR^{C}R^{D}, NR^{C}C(O)OR^{A}, B(OR^{C})(OR^{D}), NR^{D}C(═NR^{C})NR^{C}R^{D}, NR^{D}C(═NR^{C})R^{B} SiR^{G}R^{H}R^{I}, P(O)R^{E}R^{F}, P(O)OR^{E}OR^{F}, OP(O)OR^{E}OR^{F}, S(O)(═NR^{B})R^{B}, S(O)R^{B}, S(O)NR^{C}R^{D}, S(O)₂R^{B}, NR^{C}S(O)₂R^{B}, S(O)₂NR^{C}R^{D}, NR^{C}S(O)₂NR^{C}R^{D}, NR^{C}S(O)(═NR^{B})R^{B} Cy³, C_-C₆ alkyl-Cy³, OCy³, O—C₁-C₆ alkyl-Cy³; wherein, the C₁-C₆ alkyl, C₂-C₆ alkenyl, C₂-C₆ alkynyl is optionally substituted 1, 2, 3, 4 or 5 R¹².

In some embodiments, each R¹¹ is independently H, D, halo, CN, NO₂, N₃, oxo, SF₅. In some embodiments, each R¹¹ is independently H. In some embodiments, each R¹¹ is independently D. In some embodiments, each R¹¹ is independently halo (such as F, Cl, Br, I). In some embodiments, each R¹¹ is independently CN. In some embodiments, each R¹¹ is independently NO₂. In some embodiments, each R¹¹ is independently N₃. In some embodiments, each R¹¹ is independently oxo, such as carbon atoms and heteroatoms can be optionally substituted by one or more oxo or sulfido (e.g., C(O), S(O), C(S), or S(O)₂, or P(O), etc.). In some embodiments, each R¹¹ is independently SF₅.

In some embodiments, each R¹¹ is independently OR^{A}. In some embodiments, for example, each R¹¹ is independently OH, OCH₃, OCH₂CH₃, OCF₃, OCH₂CF₃.

In some embodiments, each R¹¹ is independently SR^{A}. In some embodiments, each R¹¹ is independently NHOR^{A}, such as NHOH.

In some embodiments, each R¹¹ is independently C(O)R^{B}. In some embodiments, each R¹¹ is independently C(O)R^{B}, and R^{B} is H, D. In some embodiments, each R¹¹ is CHO.

In some embodiments, each R¹¹ is independently C(O)R^{B}, and R^{B} is C_-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, arylalkyl, heteroarylalkyl, cycloalkylalkyl, or heterocycloalkylalkyl; each is optionally substituted with 1, 2, 3, 4 or 5 substituents independently selected from R¹⁴.

In some embodiments, each R¹¹ is independently C(O)R^{B}, and R^{B} is C_-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; each is optionally substituted with 1, 2, 3, 4 or 5 substituents independently selected from R¹⁴.

In some embodiments, each R¹¹ is independently C(═S)R^{B}. In some embodiments, each R¹¹ is independently C(═S)CH₃, C(═S)CH₂CH₃, C(═S)CH₂CH₂CH₃, C(═S)CH(CH₃)₂, C(═S)C(CH₃)₃.

In some embodiments, each R¹¹ is independently C(O)NR^{C}R^{D}. In some embodiments, each R¹¹ is independently C(O)NR^{C}OR^{A}. In some embodiments, each R¹¹ is independently C(O)OR^{A}.

In some embodiments, each R¹¹ is independently C(═NR^{C})NR^{C}R^{D}. In other embodiments, each R¹¹ is independently OC(O)R^{B}. In other embodiments, each R¹¹ is independently OC(O)NR^{C}R^{D}.

In other embodiments, each R¹¹ is independently NR^{C}R^{D}, and R^{C} and R^{D} are each independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴. In other embodiments, each R¹¹ is independently NH₂, NHCH₃, N(CH₃)₂, NHCH₂CH₃, N(CH₂CH₃)₂, NHCH₂CH₂CH₃, N(CH₂CH₂CH₃)₂, NHCH(CH₃)₂, NHCH₂CH₂OH, N(CH₃)CH₂CH₂OH, NHCH₂CH₂OCH₃, N(CH₃)CH₂CH₂OCH₃.

In other embodiments, each R¹¹ is independently NR^{C}C(O)R^{D}. In other embodiments, each R¹¹ is independently NR^{C}C(O)NR^{C}R^{D}. In other embodiments, each R¹¹ is independently NR^{C}C(O)OR^{A}. In other embodiments, each R¹¹ is independently NR^{D}C(═NR^{C})NR^{C}R^{D}. In other embodiments, each R¹¹ is independently NR^{D}C(═NR^{C})R^{B}.

In other embodiments, each R¹¹ is independently B(OR^{C})(OR^{D}). In other embodiments, each R¹¹ is independently SiR^{G}R^{H}R^{I}, for example, each R¹¹ is independently Si(CH₃)₃. In other embodiments, each R¹¹ is independently P(O)R^{E}R^{F}, for example, each R¹¹ is independently P(O)(CH₃)₂. In other embodiments, each R¹¹ is independently P(O)OR^{E}OR^{F}. In other embodiments, each R¹¹ is independently OP(O)OR^{E}OR^{F}. In other embodiments, each R¹¹ is independently S(O)(═NR^{B})R^{B}.

In other embodiments, each R¹¹ is independently S(O)R^{B}. In other embodiments, each R¹¹ is independently S(O)NR^{C}R^{D}. In other embodiments, each R¹¹ is independently S(O)₂R^{B}. In other embodiments, each R¹¹ is independently NR^{C}S(O)₂R^{B}. In other embodiments, each R¹¹ is independently S(O)₂NR^{C}R^{D}. In other embodiments, each R¹¹ is independently NR^{C}S(O)₂NR^{C}R^{D}. In other embodiments, each R¹¹ is independently NR^{C}S(O)(═NR^{B})R^{B}.

In other embodiments, each R¹¹ is independently Cy³, Cy³ is phenyl, naphthalenyl, pyrrolyl, furanyl, thiophenyl, imidazolyl, oxazolyl, thiazolyl, tetrazolyl, pyrazolyl, triazolyl, thiadiazolyl, oxadiazolyl, pyridinyl, pyrimidinyl, pyrazinyl, pyridazinyl, indolyl, isoindolyl, indolizinyl, benzofuranyl, isobenzofuranyl, benzo[b]thiophenyl, benzo[c]thiophenyl, indazolyl, benzo[d]imidazolyl, pyrrolo[3,2-b]pyridinyl, pyrrolo[3,2-c]pyridinyl, pyrrolo[2,3-c]pyridinyl, pyrrolo[2,3-b]pyridinyl, pyrrolo[3,4-b]pyridinyl, pyrrolo[3,4-c]pyridinyl, benzo[d]isoxazolyl, benzo[d]oxazolyl, furo[3,2-b]pyridinyl, furo[3,2-c]pyridinyl, furo[2,3-c]pyridinyl, furo[2,3-b]pyridinyl, benzo[c]isoxazolyl, furo[3,4-b]pyridinyl, furo[3,4-c]pyridinyl, benzo[d]isothiazolyl, benzo[d]thiazolyl, thieno[3,2-b]pyridinyl, thieno[3,4-c]pyridinyl, benzo[d][1,2,3]triazolyl, pyrazolo[4,3-b]pyridinyl, pyrazolo[4,3-c]pyridinyl, pyrazolo[3,4-c]pyridinyl, pyrazolo[3,4-b]pyridinyl, imidazo[4,5-b]pyridinyl, imidazo[4,5-c]pyridinyl, imidazo[4,5-c]pyridinyl, imidazo[4,5-b]pyridinyl, pyrrolo[3,2-c]pyridazinyl, pyrrolo[3,2-d]pyrimidinyl, pyrrolo[2,3-b]pyrazinyl, pyrrolo[2,3-d]pyridazinyl, pyrrolo[2,3-d]pyrimidinyl, pyrrolo[2,3-c]pyridazinyl, pyrrolo[3,4-c]pyridazinyl, pyrrolo[3,4-d]pyrimidinyl, pyrrolo[3,4-b]pyrazinyl, pyrrolo[3,4-d]pyridazinyl, pyrrolo[3,4-d]pyrimidinyl, 6H-pyrrolo[3,4-c]pyridazinyl, azetidinyl, oxetanyl, thietanyl, pyrrolidinyl, tetrahydrofuranyl, tetrahydrothiophenyl, piperidinyl, dioxanyl tetrahydropyranyl, tetrahydrothiopyranyl, piperazinyl, morpholinyl, azepanyl, diazocanyl, diazepanyl, azepanyl, cyclopropyl, cyclobutyl, cyclopentyl, cyclohexyl, cycloheptyl, each ring is optionally substituted with 1, 2, 3, 4 or 5 R¹³.

In other embodiments, each R¹¹ is independently C₁-C₆ alkyl-Cy³. In other embodiments, each R¹¹ is independently C₁ alkyl-Cy³ In other embodiments, each R¹¹ is independently C₂ alkyl-Cy³. In other embodiments, each R¹¹ is independently C₃ alkyl-Cy³. In other embodiments, each R¹¹ is independently C₄ alkyl-Cy³. In other embodiments, each R¹¹ is independently C₅ alkyl-Cy³. In other embodiments, each R¹¹ is independently C₆ alkyl-Cy³.

In other embodiments, each R¹¹ is independently OCy³. In other embodiments, each R¹¹ is independently OC₆-C₁₀ aryl. In other embodiments, each R¹¹ is independently OC₃-C₁₀ cycloalkyl. In other embodiments, each R¹¹ is independently O-5-10 membered heteroaryl. In other embodiments, each R¹¹ is independently O-4-10 membered heterocycloalkyl.

In other embodiments, each R¹¹ is independently O—C₁-C₆ alkyl-Cy³. In other embodiments, each R¹¹ is independently O—C₁ alkyl-Cy³. In other embodiments, each R¹¹ is independently O—C₂ alkyl-Cy³. In other embodiments, each R¹¹ is independently O—C₃ alkyl-Cy³. In other embodiments, each R¹¹ is independently O—C₄ alkyl-Cy³. In other embodiments, each R¹¹ is independently O—C₅ alkyl-Cy³. In other embodiments, each R¹¹ is independently O—C₆ alkyl-Cy³.

In some embodiments, each R¹¹ is independently C₁-C₆ alkyl (such as C₁-C₆ alkyl, C₁-C₅ alkyl, C₁-C₄ alkyl, C₁-C₃ alkyl, C₁-C₂ alkyl) optionally substituted by 1, 2, 3, 4 or 5 R¹². In some embodiments, each R¹¹ is independently CH₃, CH₂CH₃, CH₂CH₂CH₃, CH(CH₃)₂, CH₂CH₂CH₂CH₃, CH₂CH(CH₃)₂, C(CH₃)₃, CH₂F, CHF₂, CF₃, CH₂CH₂F, CH₂CHF₂, CH₂CF₃, CF₂CH₃, CF₂CF₃, CF₂CH₂CH₃, CH₂OH, CH₂CH₂OH, CH(OH)CH₃, CH₂CH₂CH₂OH, CH(OH)CH₂CH₂OH, CH₂NH₂, CH₂CH₂NH₂, CH₂NH₂, CH₂CH₂NHCH₃, CH₂CH₂N(CH₃)₂, CH₂CN, CH₂CH₂CN, CH₂CH₂CH₂CN.

In some embodiments, each R¹¹ is independently C₂-C₆ alkenyl (such as C₂-C₆ alkenyl, C₂-C₅ alkenyl, C₂-C₄ alkenyl, C₂-C₃ alkenyl) optionally substituted by 1, 2, 3, 4 or 5 R¹².

In some embodiments, each R¹¹ is independently C₂-C₆ alkynyl (such as C₂-C₆ alkynyl, C₂-C₅ alkynyl, C₂-C₄ alkynyl, C₂-C₃ alkynyl) optionally substituted by 1, 2, 3, 4 or 5 substituents independently selected from R¹².

In some embodiments, each R¹¹ is independently H, D, halo, CN, OR^{A}, NR^{C}R^{D} Cy³, C₁-C₆ alkyl optionally substituted 1, 2, 3, 4 or 5 substituents independently selected from R¹².

In some embodiments, each R¹¹ is independently H, D, halo, CN, OH, OCH₃, OCH₂CH₃, NH₂, NHCH₃, N(CH₃)₂, NCH₂CH₃, N(CH₂CH₃)₂, CH₃, CH₂F, CHF₂, CF₃, CH₂CH₃, CH₂OH, CH₂N(CH₃)₂, morpholinyl, 4,4-difluoro-1-piperidinyl.

In some embodiments, two R¹¹, together with the atom to which they are attached form oxo.

In some embodiments, two adjacent R¹¹ together with the atoms to which they are attached form C₃-C₁₀ membered cycloalkyl or 4-10 membered heterocycloalkyl, wherein, the C₃-C₁₀ membered cycloalkyl or 4-10 membered heterocycloalkyl optionally substituted by 1, 2, or 3 substituents independently selected from D, halo, C₁-C₆ alkyl, C₂-C₆ alkenyl, C₂-C₆ alkynyl, C₁-C₆ haloalkyl, C₁-C₆-cyanoalkyl, CN, NO₂, oxo, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{e}R^{d} NR^{c}C(O)R^{b}, NR^{c}C(O)NR^{c}R^{d}, NR^{c}C(O)OR^{a}, B(OR^{c})(OR^{d}), C(═NR^{c})NR^{c}R^{d}, NR^{d}C(═NR^{c})NR^{c}R^{d} NR^{d}C(═NR^{c})R^{b}, OP(O)OR^{e}OR^{f}, P(O)OR^{e}OR^{f}, S(O)(═NR^{b})R^{b}, S(O)R^{b}, S(O)NR^{c}R^{d}, S(O)₂R^{b}, NRS(O)₂R^{b}, S(O)₂NR^{c}R^{d}, NR^{c}S(O)₂NR^{C}R^{d} NR^{c}S(O)(═NR^{b})R^{b}, C₆-C₁₀ aryl, C₃-C₁₀ cycloalkyl, 5-10 membered heteroaryl, and 4-10 membered heterocycloalkyl, wherein the C₆-C₁₀ aryl, C₃-C₁₀ cycloalkyl, 5-10 membered heteroaryl, 4-10 membered heterocycloalkyl is optionally substituted by 1, 2, 3 or 4 substituents independently selected from D, halo, CN, NO₂, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅.

In some embodiments, two adjacent R¹¹ together with the atoms to which they are attached form C₃-C₁₀ membered cycloalkyl optionally substituted by 1, 2, or 3 substituents independently selected from D, halo, C₁-C₆ alkyl, C₂-C₆ alkenyl, C₂-C₆ alkynyl, C₁-C₆ haloalkyl, C₁-C₆-cyanoalkyl, CN, NO₂, oxo, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d} NR^{c}C(O)OR^{a}, B(OR^{c})(OR^{d}), C(═NR^{c})NR^{c}R^{d}, NR^{d}C(═NR^{c})NR^{c}R^{d}, NR^{d}C(═NR^{c})R^{b}, OP(O)OR^{e}OR^{f}, P(O)OR^{e}OR^{f}, S(O)(═NR^{b})R^{b}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}NR^{c}S(O)(═NR^{b})R^{b}, Cy⁴; wherein, Cy⁴ is C₆-C₁₀ aryl, C₃-C₁₀ cycloalkyl, 5-10 membered heteroaryl, and 4-10 membered heterocycloalkyl; wherein, Cy⁴ is optionally substituted by 1, 2, 3 or 4 substituents independently selected from D, halo, CN, NO₂, OH, oxo, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅.

In some embodiments, two adjacent R¹¹ together with the atoms to which they are attached form 4-10 membered heterocycloalkyl optionally substituted by 1, 2, or 3 substituents independently selected from D, halo, C₁-C₆ alkyl, C₂-C₆ alkenyl, C₂-C₆ alkynyl, C₁-C₆ haloalkyl, C₁-C₆-cyanoalkyl, CN, NO₂, oxo, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{c}R^{d} NR^{c}C(O)R^{b}, NR^{c}C(O)NR^{c}R^{d} NR^{c}C(O)OR^{a}, B(OR^{c})(OR^{d}), C(═NR^{c})NR^{c}R^{d}, NR^{d}C(═NR^{c})NR^{c}R^{d}, NR^{d}C(═NR^{c})R^{b}, OP(O)OR^{e}OR^{f}, P(O)OR^{e}OR^{f}, S(O)(═NR^{b})R^{b}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}NR^{c}S(O)(═NR^{b})R^{b}, Cy⁴; wherein, Cy⁴ is C₆-C₁₀ aryl, C₃-C₁₀ cycloalkyl, 5-10 membered heteroaryl, and 4-10 membered heterocycloalkyl; wherein, Cy⁴ is optionally substituted by 1, 2, 3 or 4 substituents independently selected from D, halo, CN, NO₂, OH, oxo, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅.

In some embodiments, each Cy³ is independently optionally substituted C₆-C₁₀ aryl, C₃-C₁₀ cycloalkyl, 5-10 membered heteroaryl, or 4-10 membered heterocycloalkyl; each Cy³ is optionally substituted with 1, 2, 3, 4 or 5 substituents independently selected from R¹³.

In some embodiments, Cy³ is C₆-C₁₀ aryl optionally substituted with 1, 2, 3, 4 or 5 substituents independently selected from R¹². In some embodiments, Cy³ is phenyl, naphthalenyl optionally substituted with 1, 2, 3, 4 or 5 substituents independently selected from R¹². In some embodiments, Cy³ is phenyl optionally substituted with 1, 2, 3, 4 or 5 substituents independently selected from R¹³.

In some embodiments, Cy³ is 5-10 membered heteroaryl optionally substituted with 1, 2, 3, 4 or 5 substituents independently selected from R¹³.

In some embodiments, Cy³ is pyrrolyl, furanyl, thiophenyl, imidazolyl, oxazolyl, thiazolyl, tetrazolyl, pyrazolyl, triazolyl, thiadiazolyl, oxadiazolyl, pyridinyl, pyrimidinyl, pyrazinyl, pyridazinyl, indolyl, isoindolyl, indolizinyl, benzofuranyl, isobenzofuranyl, benzo[b]thiophenyl, benzo[c]thiophenyl, indazolyl, benzo[d]imidazolyl, pyrrolo[3,2-b]pyridinyl, pyrrolo[3,2-c]pyridinyl, pyrrolo[2,3-c]pyridinyl, pyrrolo[2,3-b]pyridinyl, pyrrolo[3,4-b]pyridinyl, pyrrolo[3,4-c]pyridinyl, benzo[d]isoxazolyl, benzo[d]oxazolyl, furo[3,2-b]pyridinyl, furo[3,2-c]pyridinyl, furo[2,3-c]pyridinyl, furo[2,3-b]pyridinyl, benzo[c]isoxazolyl, furo[3,4-b]pyridinyl, furo[3,4-c]pyridinyl, benzo[d]isothiazolyl, benzo[d]thiazolyl, thieno[3,2-b]pyridinyl, thieno[3,4-c]pyridinyl, benzo[d][1,2,3]triazolyl, pyrazolo[4,3-b]pyridinyl, pyrazolo[4,3-c]pyridinyl, pyrazolo[3,4-c]pyridinyl, pyrazolo[3,4-b]pyridinyl, imidazo[4,5-b]pyridinyl, imidazo[4,5-c]pyridinyl, imidazo[4,5-c]pyridinyl, imidazo[4,5-b]pyridinyl, pyrrolo[3,2-c]pyridazinyl, pyrrolo[3,2-d]pyrimidinyl, pyrrolo[2,3-b]pyrazinyl, pyrrolo[2,3-d]pyridazinyl, pyrrolo[2,3-d]pyrimidinyl, pyrrolo[2,3-c]pyridazinyl, pyrrolo[3,4-c]pyridazinyl, pyrrolo[3,4-d]pyrimidinyl, pyrrolo[3,4-b]pyrazinyl, pyrrolo[3,4-d]pyridazinyl, pyrrolo[3,4-d]pyrimidinyl, 6H-pyrrolo[3,4-c]pyridazinyl; each ring is optionally substituted with 1, 2, 3, 4 or 5 substituents independently selected from R¹³.

In some embodiments, Cy³ is pyrimidinyl optionally substituted with 1, 2, 3, 4 or 5 substituents independently selected from R¹³. In some embodiments, Cy³ is pyridazinyl optionally substituted with 1, 2, 3, 4 or 5 substituents independently selected from R¹³. In some embodiments, Cy³ is pyrazinyl optionally substituted with 1, 2, 3, 4 or 5 substituents independently selected from R¹³. In other embodiments, Cy³ is pyrazolyl optionally substituted with 1, 2, 3, 4 or 5 substituents independently selected from R¹³.

In other embodiments, Cy³ is C₃-C₁₀ cycloalkyl optionally substituted with 1, 2, 3, 4 or 5 substituents independently selected from R¹³.

In other embodiments, Cy³ is 4-10 membered heterocycloalkyl optionally substituted with 1, 2, 3, 4 or 5 substituents independently selected from R¹³.

In some embodiments, Cy³ is azetidinyl, oxetanyl, thietanyl, pyrrolidinyl, tetrahydrofuranyl, tetrahydrothiophenyl, piperidinyl, dioxanyl tetrahydropyranyl, tetrahydrothiopyranyl, piperazinyl, morpholinyl, azepanyl, diazocanyl, diazepanyl, azepanyl; each ring is optionally substituted with 1, 2, 3, 4 or 5 R¹³.

In other embodiments, each R¹² is independently H, D, halo, CN, NO₂, N₃, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, OR^{a1}, SR^{a1}, SF₅, NHOR^{a1}, C(O)R^{b1}, C(O)NR^{c1}R^{d1}, C(O)OR^{a1}, OC(O)OR^{a1}, OC(O)R^{b1}, OC(O)NR^{c1}R^{d1} NR^{c1}R^{d1} NR^{c1}C(O)R^{b1}, NR^{c1}C(O)NR^{c1}R^{d1} NR^{c1}C(O)OR^{a1}, B(OR^{c1})(OR^{d1}), C(═NR^{c1})NR^{c1}R^{d1} NR^{d1}C(═NR^{c})NR^{c1}R^{d1} NR^{d1}C(═NR^{c1})R¹, P(O)OR^{e1}OR, OP(O)OR^{e1}OR^{f1}, S(O)(═NR^{b1})R^{b1}, S(O)R^{b1}, S(O)NR^{c1}R^{d1}, S(O)₂R^{b1}, NR^{c1}S(O)₂R^{b}1, S(O)₂NR^{c1}R^{d1} NR^{c1} S(O)₂NR^{c1}R^{d1} NR^{c1}S(O)(═NR^{b1})R^{b}, C₆-C₁₀ aryl, C₃-C₁₀ cycloalkyl, 5-10 membered heteroaryl, and 4-10 membered heterocycloalkyl.

In some embodiments, each R¹² is independently H, D, halo, CN, NO₂, N₃, SF₅, C₁-C₆ alkyl, C₂-C₆ alkenyl, C₂-C₆ alkynyl, C₁-C₆ haloalkyl, OC₁-C₆ alkylOH, OC₁-C₆ alkyl-O—C₁-C₆ alkyl, OR^{a1}, SR^{a1}, C(O)R^{b1}, C(O)NR^{c1}R^{d1}, S(O)₂R^{b1}, C₆-C₁₀ aryl, C₃-C₁₀ cycloalkyl, 5-10 membered heteroaryl, and 4-10 membered heterocycloalkyl; wherein, the C₆-C₁₀ aryl, C₃-C₁₀ cycloalkyl, 5-10 membered heteroaryl, 4-10 membered heterocycloalkyl can be unsubstituted or substituted independently selected from D, halo, CN, NO₂, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅.

In some embodiments, each R¹² is independently H, D, halo, CN, NO₂, N₃, SF₅. In some embodiments, each R¹² is independently H. In some embodiments, each R¹² is independently D. In some embodiments, each R¹² is independently halo (such as F, Cl, Br, I). In some embodiments, each R¹² is independently CN. In some embodiments, each R¹² is independently NO₂. In some embodiments, each R¹² is independently N₃. In some embodiments, each R¹² is independently SF₅.

In some embodiments, each R¹² is independently C₁-C₆ alkyl, C₂-C₆ alkenyl, C₂-C₆ alkynyl, C₁-C₆ haloalkyl, OC₁-C₆ alkylOH, OC₁-C₆ alkyl-O—C₁-C₆ alkyl.

In some embodiments, each R¹² is independently OR^{a1}.

In some embodiments, each R¹² is independently SR^{a1}.

In some embodiments, each R¹² is independently NHOR^{a1}. In some embodiments, each R¹² is independently C(O)R^{b1}. In some embodiments, each R¹² is independently C(O)NR^{c1}R^{d1} In some embodiments, each R¹² is independently C(O)OR^{a1}. In some embodiments, each R¹² is independently OC(O)OR^{a1}. In some embodiments, each R¹² is independently OC(O)R^{b1}. In some embodiments, each R¹² is independently OC(O)NR^{c1}R^{d1}.

In some embodiments, each R¹² is independently NR^{c1}R^{d1}. In some embodiments, each R¹² is independently NR^{c1}C(O)R^{b1}. In some embodiments, each R¹² is independently NR^{c1}C(O)NR^{c1}R^{d1}. In some embodiments, each R¹² is independently NR^{c1}C(O)OR^{a1}.

In some embodiments, each R¹² is independently B(OR^{c1})(OR^{d1}). In some embodiments, each R¹² is independently C(═NR^{cl})NR^{c1}R^{d1}. In some embodiments, each R¹² is independently NR^{d1}C(═NR^{c1})NR^{c1}R^{d1} In some embodiments, each R¹² is independently NR^{d1}C(═NR^{c1})R^{b1}.

In some embodiments, each R¹² is independently P(O)OR^{e1}OR^{f1}. In some embodiments, each R¹² is independently OP(O)OR^{e1}OR^{f1}.

In some embodiments, each R¹² is independently S(O)(═NR^{b1})R^{b1}. In some embodiments, each R¹² is independently S(O)R^{b1}. In some embodiments, each R¹² is independently S(O)NR^{c1}R^{d1}.

In some embodiments, each R¹² is independently S(O)₂R^{b1}. In some embodiments, each R¹² is independently NR^{c1}S(O)₂R^{b1}. In some embodiments, each R¹² is independently S(O)₂NR^{c1}R^{d1}.

In some embodiments, each R¹² is independently NR^{c1}S(O)₂NR^{c1}R^{d1}. In some embodiments, each R¹² is independently NR^{c1}S(O)(═NR^{b1})R^{b1}.

In some embodiments, each R¹² is independently C₆-C₁₀ aryl, C₃-C₁₀ cycloalkyl, 5-10 membered heteroaryl, and 4-10 membered heterocycloalkyl; wherein, the C₆-C₁₀ aryl, C₃-C₁₀ cycloalkyl, 5-10 membered heteroaryl, 4-10 membered heterocycloalkyl can be unsubstituted or substituted by 1, 2, 3, 4 or 5 substituents independently selected from D, halo, CN, NO₂, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅.

In some embodiments, each R¹³ is independently D, halo, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{c1}R^{d1}, C(O)OR^{a1}, OC(O)R^{b1}, OC(O)NR^{c1}R^{d1} N^{c1}R^{d1}, NR^{c1}C(O)R^{b1} NR^{C}C(O)NR^{c1}R^{d1} NR^{c1}C(O)OR^{a1}, B(OR^{c1})(OR^{d1}), C(═NR^{c1})NR^{c}R^{d1} NR^{d1}C(═NR^{c1})NR^{c1}R^{d1} NR^{d1}C(═NR^{c1})R^{b1}, P(O)R^{e1}R^{f1}, P(O)OR^{e1}OR^{f1}, OP(O)OR^{e1}OR^{f1}, S(O)(═NR^{b1})R^{b}1, S(O)R^{b1}, S(O)NR^{c1}R^{d1}, S(O)₂R^{b1}, NR^{c1} S(O)₂R^{b1}, S(O)₂NR^{c1}R^{d1} NR^{c}S(O)₂NR^{c1}R^{d1} NR^{c1}S(O)(═NR¹)R^{b1}, C₆-C₁₀ aryl, C₃-C₁₀ cycloalkyl, 5-10 membered heteroaryl, and 4-10 membered heterocycloalkyl, wherein the C₆-C₁₀ aryl, C₃-C₁₀ cycloalkyl, 5-membered heteroaryl, 4-10 membered heterocycloalkyl can be unsubstituted or substituted independently selected from D, halo, CN, NO₂, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅.

In some embodiments, each R¹³ is independently D, halo, CN, NO₂, N₃, OR^{a1}, SR^{a1}, SF₅, or NHOR^{a1}. In some embodiments, each R¹³ is independently D. In some embodiments, each R¹³ is independently halo (such as F, Cl, Br, I). In some embodiments, each R¹³ is independently CN. In some embodiments, each R¹³ is independently NO₂. In some embodiments, each R¹³ is independently N₃. In some embodiments, each R¹³ is independently OR^{a1} (such as OH, OCH₃, OCH₂CH₃, OCH₂F, OCHF₂, OCF₃). In some embodiments, each R¹³ is independently SR^{a1} (such as SCH₃). In some embodiments, each R¹³ is independently SF₅. In some embodiments, each R¹³ is independently NHOR^{a1}.

In some embodiments, each R¹³ is independently C(O)R^{b1}. In some embodiments, each R¹³ is independently C(O)NR^{c1}R^{d1}. In some embodiments, each R¹³ is independently C(O)OR^{a1}.

In some embodiments, each R¹³ is independently OC(O)R^{b1}. In some embodiments, each R¹³ is independently OC(O)NR^{c1}R^{d1}.

In some embodiments, each R¹³ is independently NR^{c1}R^{d1} (such as NH₂, NHCH₃, N(CH₃)₂).

In some embodiments, each R¹³ is independently NR^{c1}C(O)R^{b1}. In some embodiments, each R¹³ is independently NR^{c1}C(O)NR^{c1}R^{d1}. In some embodiments, each R¹³ is independently NR^{c1}C(O)OR^{a1}.

In some embodiments, each R¹³ is independently B(OR^{c1})(OR^{d1}). In some embodiments, each R¹³ is independently C(═NR^{cl})NR^{c1}R^{d1}. In some embodiments, each R¹³ is independently NR^{d1}C(═NR^{c1})NR^{c1}R^{d1} In some embodiments, each R¹³ is independently NR^{d1}C(═NR^{c1})R^{b1}.

In some embodiments, each R¹³ is independently P(O)R^{e1}R^{f1}. In some embodiments, each R¹³ is independently P(O)OR^{e1}OR^{f1}. In some embodiments, each R¹³ is independently OP(O)OR^{e1}OR^{f1}.

In some embodiments, each R¹³ is independently S(O)(═NR^{b1})R^{b1}. In some embodiments, each R¹³ is independently S(O)R^{b1}. In some embodiments, each R¹³ is independently S(O)NR^{c1}R^{d1}.

In some embodiments, each R¹³ is independently S(O)₂R^{b1}. In some embodiments, each R¹³ is independently NR^{c1}S(O)₂R^{b1}. In some embodiments, each R¹³ is independently S(O)₂NR^{c1}R^{d1}.

In some embodiments, each R¹³ is independently NR^{c1}S(O)₂NR^{c1}R^{d1}. In some embodiments, each R¹³ is independently NR^{c1}S(O)(═NR^{b1})R^{b1}In other embodiments, each R¹³ is independently C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl.

In other embodiments, each R¹³ is independently C₆-C₁₀ aryl, C₃-C₁₀ cycloalkyl, 5-10 membered heteroaryl, and 4-10 membered heterocycloalkyl; wherein, the C₆-C₁₀ aryl, C₃-C₁₀ cycloalkyl, 5-10 membered heteroaryl, 4-10 membered heterocycloalkyl can be unsubstituted or substituted by 1, 2, 3, 4 or 5 substituents independently selected from D, halo, CN, NO₂, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅.

In some embodiments, each R¹⁴ is independently H, D, NO₂, CN, halo, oxo, SF₅, C₁-C₈ alkyl, C₂-C₈ alkenyl, C₂-C₈ alkynyl, C₆-C₁₄ aryl, C₃-C₁₄ cycloalkyl, 5-14 membered heteroaryl, or 4-14 membered heterocycloalkyl, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{c}R^{d} NR^{c}C(O)R^{b}, NR^{c}C(O)NR^{c}R^{d} NR^{c}C(O)OR^{a}, B(OR^{c})(OR^{d}), C(═NR^{c})NR^{c}R^{d}, NR^{d}C(═NR^{c})NR^{c}R^{d}, NR^{d}C(═NR^{c})R, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{C}R^{d}, or NR^{c}S(O)(═NR^{b})R^{b}; wherein, the C₁-C₈ alkyl, C₂-C₈ alkenyl, C₂-C₈ alkynyl, C₆-C₁₄ aryl, C₃-C₁₄ cycloalkyl, 5-14 membered heteroaryl, or 4-14 membered heterocycloalkyl is optionally substituted by 1, 2, 3, 4 or 5 substituents independently selected from D, NO₂, CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{c1}R^{d1}, C(O)OR^{a1}, OC(O)R^{b}1, OC(O)NR^{c1}R^{d1} NR^{c1}R^{d1} NR^{c1}C(O)R^{b}, NR^{c1}C(O)NR^{c1}R^{d1} NR^{c1}C(O)OR^{a1}, B(OR^{c1})(OR^{d1}), C(═NR^{c1})NR^{c1}R^{d1} NR^{d1}C(═NR^{c1})NR^{c1}R^{d1} NR^{d1}C(═NR^{c1})R^{b}, P(O)R^{e1}R^{f1}, P(O)OR^{e1}OR^{f1}, OP(O)OR^{e1}OR^{f1}, S(O)(═NR^{b1})R^{b1}, S(O)R^{b1}, S(O)NR^{c1}R^{d1}, S(O)₂R^{b}1, NR^{c1}S(O)₂R^{b}, S(O)₂NR^{c1}R^{d1} NR^{c1}S(O)₂NR^{c1}R^{d1} NR^{c1}S(O)(═NR^{b1})R^{b}, C₆-C₁₀ aryl, C₃-C₁₀ cycloalkyl, 5-10 membered heteroaryl, and 4-10 membered heterocycloalkyl.

In some embodiments, each R¹⁴ is independently H. In some embodiments, each R¹⁴ is independently D. In some embodiments, each R¹⁴ is independently NO₂. In some embodiments, each R¹⁴ is independently CN. In some embodiments, each R¹⁴ is independently halo (such as F, Cl, Br or I). In some embodiments, each R¹⁴ is independently oxo. In some embodiments, each R¹⁴ is independently SF₅.

In some embodiments, each R¹⁴ is independently C₁-C₈ alkyl optionally substituted by 1, 2, 3, 4 or 5 substituents independently selected from D, NO₂, CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{c1}R^{d1}, C(O)OR^{a1}, OC(O)R^{b}1, OC(O)NR^{c1}R^{d1} NR^{c1}R^{d1} NR^{c1}C(O)R^{b1}, NR^{c1}C(O)NR^{c1}R^{d1} NR^{c1}C(O)OR^{a1}, B(OR^{c1})(OR^{d1}), C(═NR^{c1})NR^{c1}R^{d1} NR^{d1}C(═NR^{c1})NR^{c1}R^{d1} NR^{d1}C(═NR^{c1})R^{b}, P(O)R^{e1}R^{f1}, P(O)OR^{e1}OR^{f1}, OP(O)OR^{e1}OR^{f1}, S(O)(═NR^{b1})R^{b1}, S(O)R¹, S(O)NR^{c1}R^{d1}, S(O)₂R^{b1}, NR^{C}S(O)₂R^{b1}, S(O)₂NR^{c1}R^{d1} NR^{c1}S(O)₂NR^{c1}R^{d1} NR^{c1}S(O)(═NR^{b1})R^{b1}, C₆-C₁₀ aryl, C₃-C₁₀ cycloalkyl, 5-10 membered heteroaryl, and 4-10 membered heterocycloalkyl.

In some embodiments, each R¹⁴ is independently C₂-C₈ alkenyl optionally substituted by 1, 2, 3, 4 or 5 substituents independently selected from D, NO₂, CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{c1}R^{d1}, C(O)OR^{a1}, OC(O)R^{b}1, OC(O)NR^{c1}R^{d1} NR^{c1}R^{d1} NR^{c1}C(O)R^{b}, NR^{c1}C(O)NR^{c1}R^{d1} NR^{c1}C(O)OR^{a1}, B(OR^{c1})(OR^{d1}), C(═NR^{c1})NR^{c1}R^{d1} NR^{d1}C(═NR^{c1})NR^{c1}R^{d1} NR^{d1}C(═NR^{c1})R^{b} P(O)R^{e1}R^{f1}, P(O)OR^{e1}OR^{f1}, OP(O)OR^{e1}OR^{f1}, S(O)(═NR^{b1})R^{b1}, S(O)R^{b1}, S(O)NR^{c1}R^{d1}, S(O)₂R^{b}1, NR^{c1}S(O)₂R^{b1}, S(O)₂NR^{c1}R^{d1} NR^{c1}S(O)₂NR^{c1}R^{d1} NR^{c1}S(O)(═NR^{b1})R^{b1}, C₆-C₁₀ aryl, C₃-C₁₀ cycloalkyl, 5-10 membered heteroaryl, and 4-10 membered heterocycloalkyl.

In some embodiments, each R¹⁴ is independently C₂-C₈ alkynyl optionally substituted by 1, 2, 3, 4 or 5 substituents independently selected from D, NO₂, CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{c1}R^{d1}, C(O)OR^{a1}, OC(O)R^{b1}, OC(O)NR^{c1}R^{d1}, NR¹R^{d1} NR^{C}C(O)R^{b1}, NR^{C}C(O)NR^{c1}R^{d1} NR^{c1}C(O)OR^{a1}, B(OR^{c1})(OR^{d1}), C(═NR^{c1})NR^{c1}R^{d1} NR^{d1}C(═NR^{c1})NR^{c1}R^{d1} NR^{d1}C(═NR^{c1})R^{b1}, P(O)R^{e1}R^{b1}, P(O)OR^{e1}OR^{f1}, OP(O)OR^{e1}OR^{f1}, S(O)(═NR^{b1})R^{b1}, S(O)R^{b1}, S(O)NR^{c1}R^{d1}, S(O)₂R^{b1}, NR^{c1}S(O)₂R^{b1}, S(O)₂NR^{c1}R^{d1} NR^{c1}S(O)₂NR^{c1}R^{d1} NR^{c1}S(O)(═NR^{b1})R^{b1}, C₆-C₁₀ aryl, C₃-C₁₀ cycloalkyl, 5-10 membered heteroaryl, and 4-10 membered heterocycloalkyl.

In some embodiments, each R¹⁴ is independently C₆-C₁₄ aryl optionally substituted by 1, 2, 3, 4 or 5 substituents independently selected from D, NO₂, CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{c1}R^{d1}, C(O)OR^{a1}, OC(O)R^{b1}, OC(O)NR^{c1}R^{d1}, NR¹R^{d1} NR^{c1}C(O)R^{b1} NR^{c1}C(O)NR^{c1}R^{d1} NR^{c1}C(O)OR^{a1}, B(OR^{c1})(OR^{d1}), C(═NR^{c1})NR^{c1}R^{d1} NR^{d1}C(═NR^{c1})NR^{c1}R^{d1} NR^{d1}C(═NR^{c1})R^{b1}, P(O)R^{e1}R^{f1}, P(O)OR^{e1}OR^{f}, OP(O)OR^{e1}OR^{f1}, S(O)(═NR¹)R^{b1}, S(O)R¹, S(O)NR^{c1}R^{d1}, S(O)₂R^{b}1, NR^{c1}S(O)₂R^{b1}, S(O)₂NR^{c1}R^{d1} NR^{c1}S(O)₂NR^{c1}R^{d1} NR^{c1}S(O)(═NR^{b1})R^{b1}, C₆-C₁₀ aryl, C₃-C₁₀ cycloalkyl, 5-10 membered heteroaryl, and 4-10 membered heterocycloalkyl.

In some embodiments, each R¹⁴ is independently C₃-C₁₄ cycloalkyl optionally substituted by 1, 2, 3, 4 or 5 substituents independently selected from D, NO₂, CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{c1}R^{d1}, C(O)OR^{a1}, OC(O)R^{b1}, OC(O)NR^{c1}R^{d1} NR^{c1}R^{d1} NR^{c1}C(O)R^{b1} NR^{c1}C(O)NR^{c1}R^{d1} NR^{c1}C(O)OR^{a1}, B(OR^{c1})(OR^{d1}), C(═NR^{c1})NR^{c1}R^{d1} NR^{d1}C(═NR^{c1})NR^{c1}R^{d1} NR^{d1}C(═NR^{c1})R^{b1}, P(O)R^{e1}R^{f1}, P(O)OR^{e1}OR^{f1}, OP(O)OR^{e1}OR^{f1}, S(O)(═NR^{b1})R^{b1}, S(O)R¹, S(O)NR^{c1}R^{d1}, S(O)₂R^{b}1, NR^{c1}S(O)₂R^{b1}, S(O)₂NR^{c1}R^{d1} NR^{c1}S(O)₂NR^{c1}R^{d1} NR^{c1}S(O)(═NR^{b1})R^{b1}, C₆-C₁₀ aryl, C₃-C₁₀ cycloalkyl, 5-10 membered heteroaryl, and 4-10 membered heterocycloalkyl.

In some embodiments, each R¹⁴ is independently 5-14 membered heteroaryl optionally substituted by 1, 2, 3, 4 or 5 substituents independently selected from D, NO₂, CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{c1}R^{d1}, C(O)OR^{a1}, OC(O)R¹, OC(O)NR^{c1}R^{d1} NR^{c1}R^{d1} NR^{c1}C(O)R^{b1}, NR^{c1}C(O)NR^{c1}R^{d1} NR^{c1}C(O)OR^{a1} B(OR^{c1})(OR^{d1}), C(═NR^{c1})NR^{c1}R^{d1} NR^{d1}C(═NR^{c1})NR^{c1}R^{d1} NR^{d1}C(═NR^{c1})R^{b}, P(O)R^{e1}R^{f1}, P(O)OR^{e1}OR^{f}1, OP(O)OR^{e1}OR, S(O)(═NR^{b1})R^{b1}, S(O)R^{b1}, S(O)NR^{c1}R^{d1}, S(O)₂R^{b1}, NR^{c1}S(O)₂R^{b1}, S(O)₂NR^{c1}R^{d1} NR^{c1} S(O)₂NR^{c1}R^{d1} NR^{c1}S(O)(═NR^{b1})R^{b}, C₆-C₁₀ aryl, C₃-C₁₀ cycloalkyl, 5-10 membered heteroaryl, and 4-10 membered heterocycloalkyl.

In some embodiments, each R¹⁴ is independently 4-14 membered heterocycloalkyl optionally substituted by 1, 2, 3, 4 or 5 substituents independently selected from D, NO₂, CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R^{b1}, C(O)NR^{cl}R^{dl}, C(O)OR^{a1}, OC(O)R^{b1}, OC(O)NR^{c1}R^{d1} NR^{c1}R^{d1} NR^{c1}C(O)R^{b1} NR^{c1}C(O)NR^{c1}R^{d1} NR^{c1}C(O)OR^{a}, B(OR^{c1})(OR^{d1}), C(═NR^{c1})NR^{c1}R^{d1} NR^{d1}C(═NR^{c1})NR^{c1}R^{d1} NR^{d1}C(═NR^{c1})R^{b1}, P(O)R^{e1}R^{f1}, P(O)OR^{e1}OR^{f}1, OP(O)OR^{e1}OR, S(O)(═NR^{b1})R^{b1}, S(O)R^{b1}, S(O)NR^{c1}R^{d1} S(O)₂R^{b}1, NR^{c1}S(O)₂R^{b1}, S(O)₂NR^{c1}R^{d1} NR^{c1}S(O)₂NR^{c1}R^{d1} NR^{c1}S(O)(═NR^{b1})R^{b1}, C₆-C₁₀ aryl, C₃-C₁₀ cycloalkyl, 5-10 membered heteroaryl, and 4-10 membered heterocycloalkyl.

In some embodiments, each R¹⁴ is independently OR^{a}. In some embodiments, each R¹⁴ is independently SR^{a}. In some embodiments, each R¹⁴ is independently SF₅. In some embodiments, each R¹⁴ is independently NHOR^{a}.

In some embodiments, each R¹⁴ is independently C(O)R^{b}. In some embodiments, each R¹⁴ is independently C(O)NR^{c}R^{d}. In some embodiments, each R¹⁴ is independently C(O)OR^{a}. In some embodiments, each R¹⁴ is independently OC(O)R^{b}. In some embodiments, each R¹⁴ is independently OC(O)NR^{c}R^{d}.

In some embodiments, each R¹⁴ is independently NR^{c}R^{d}. In some embodiments, each R¹⁴ is independently NR^{c}C(O)R^{b}. In some embodiments, each R¹⁴ is independently NR^{c}C(O)NR^{c}R^{d}. In some embodiments, each R¹⁴ is independently NR^{c}C(O)OR^{a}.

In some embodiments, each R¹⁴ is independently B(OR^{c})(OR^{d}). In some embodiments, each R¹⁴ is independently C(═NR^{c})NR^{c}R^{d}. In some embodiments, each R¹⁴ is independently NR^{d}C(═NR^{c})NR^{c}R^{d}. In some embodiments, each R¹⁴ is independently NR^{d}C(═NR^{c})R^{b}.

In some embodiments, each R¹⁴ is independently P(O)R^{e}R^{f}. In some embodiments, each R¹⁴ is independently P(O)OR^{e}OR^{f}. In some embodiments, each R¹⁴ is independently OP(O)OR^{e}OR^{f}.

In some embodiments, each R¹⁴ is independently S(O)R^{b}. In some embodiments, each R¹⁴ is independently S(O)NR^{c}R^{d}. In some embodiments, each R¹⁴ is independently S(O)₂R^{b}. In some embodiments, each R¹⁴ is independently NR^{c}S(O)₂R^{b}. In some embodiments, each R¹⁴ is independently S(O)₂NR^{c}R^{d}. In some embodiments, each R¹⁴ is independently NR^{c}S(O)₂NR^{c}R^{d}. In some embodiments, each R¹⁴ is independently NR^{c}S(O)(═NR^{b})R^{b}.

In compounds of Formula I, each R^{A} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocyclalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In some embodiments, each R^{A} is independently H. In some embodiments, each R^{A} is independently D.

In other embodiments, each R^{A} is independently C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocyclalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In compounds of Formula I, each R^{B} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In some embodiments, each R^{B} is independently H. In some embodiments, R^{B} is independently D.

In some embodiments, each R^{B} is independently C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁—C₆ alkyl, or 4-10 membered heterocycloalkyl-C₁-C₆ alkyl is optionally substituted with 1, 2, 3, 4 or 5 substituents independently selected from R¹⁴.

In some embodiments, each R⁸ is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In some embodiments, each R⁸ is independently H. In some embodiments, each R⁸ is independently D.

In some embodiments, each R⁸ is independently C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-membered heterocycloalkyl-C₁-C₆ alkyl; wherein the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In some embodiments, each R^{D} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In some embodiments, each R^{D} is independently H. In some embodiments, each R^{D} is independently D.

In some embodiments, each R^{D} is independently C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-membered heterocycloalkyl-C₁-C₆ alkyl; wherein the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴.

In other embodiments, R^{C} and R^{D} together with the N atom to which they are attached form form 4-7 membered heterocycloalkyl optionally substituted with 1, 2, 3, 4 or 5 substituents independently selected from D, halo, oxo, CN, C₁-C₄ alkyl, C₁-C₄haloalkyl, C₁-C₄ alkyl-CN, OR^{a}, SR^{a}, C(O)R^{b}, NR^{c}R^{d}.

In some embodiments, each R^{E} is independently H, D, C₁-C₄ alkyl, C₁-C₄ haloalkyl, C₂-C₄ alkenyl, (C₁-C₄ alkoxy)-C₁-C₄ alkyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, C₃-C₁₀ cycloalkyl-C₁-C₄ alkyl, 5-10 membered heteroaryl-C₁-C₄ alkyl, or 4-10 membered heterocycloalkyl-C₁-C₄ alkyl.

In some embodiments, each RFis independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl.

In some embodiments, each R^{a} is independently H, D, C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl, wherein the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄haloalkyl, or C₁-C₄haloalkoxy.

In some embodiments, each R^{a} is independently H, D.

In some embodiments, each R^{a} is independently C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl, wherein the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄haloalkyl, or C₁-C₄haloalkoxy.

In some embodiments, each R^{b} is independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl; wherein the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl is optionally substituted with 1, 2, 3, 4 or 5 substituents independently selected from D, OH, halo, CN, —NH₂, —NH(C₁-C₄ alkyl), —N(C₁-C₄ alkyl)₂, C₁-C₄ alkyl, C₁-C₄ haloalkyl, —OC₁-C₄ alkyl or —OC₁-C₄ haloalkyl, C₆-C₁₀ aryl, C₃-C₁₀ cycloalkyl, 5-10 membered heteroaryl, or 4-10 membered heterocycloalkyl.

In some embodiments, each R^{b} is independently H, D.

In some embodiments, each R^{b} is independently C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-membered heterocycloalkyl-C₁-C₄ alkyl; wherein the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl is optionally substituted with 1, 2, 3, 4 or 5 substituents independently selected from D, OH, halo, CN, —NH₂, —NH(C₁-C₄ alkyl), —N(C₁-C₄ alkyl)₂, C₁-C₄ alkyl, C₁-C₄ haloalkyl, —OC₁-C₄ alkyl or —OC₁-C₄haloalkyl, C₆-C₁₀ aryl, C₃-C₁₀ cycloalkyl, 5-10 membered heteroaryl, or 4-10 membered heterocycloalkyl.

In some embodiments, R^{c} and R^{d} are each independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, 4-10 membered heterocycloalkyl-C₁-C₄ alkyl, C₆-C₁₀ aryl-C₃-C₁₀ cycloalkyl, C₆-C₁₀ aryl-4-10 membered heterocycloalkyl, C₆-C₁₀ aryl-5-10 membered heteroaryl, bi(C₆-C₁₀ aryl), 5-10 membered heteroaryl-C₃-C₁₀ cycloalkyl, 5-10 membered heteroaryl-4-10 membered heterocycloalkyl, 5-10 membered heteroaryl-C₆-C₁₀ aryl, or bi(5-10 membered heteroaryl); wherein, the C₁₋₄ alkyl, C₂₋₄ alkenyl, C₂₋₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, 4-10 membered heterocycloalkyl-C₁-C₄ alkyl, C₆-C₁₀ aryl-C₃-C₁₀ cycloalkyl, C₆-C₁₀ aryl-4-10 membered heterocycloalkyl, C₆-C₁₀ aryl-5-10 membered heteroaryl, bi(C₆-C₁₀ aryl), 5-10 membered heteroaryl-C₃-C₁₀ cycloalkyl, 5-10 membered heteroaryl-4-10 membered heterocycloalkyl, 5-10 membered heteroaryl-C₆-C₁₀ aryl, or bi(5-10 membered heteroaryl) is optionally substituted with 1, 2, 3, 4 or 5 substituents independently selected from D, halo, OH, CN, —NH₂, —NH(C₁-C₄ alkyl), —N(C₁-C₄ alkyl)₂, C₁-C₄ alkyl, O—C₁-C₄ alkyl, C₁-C₄ haloalkyl, O—C₁-C₄ haloalkyl, C₁-C₄ alkyl-OH, C₁-C₄ alkyl-CN, C₆-C₁₀ aryl, 5-10 membered heteroaryl, C(O)OR^{a1}, C(O)R^{b1}, S(O)₂R^{b1}, C₁-C₄ alkyl-O—C₁-C₄ alkyl, and C₁-C₄ alkyl-O—C₁-C₄ alkyl-O—.

In some embodiments, R^{c} and R^{d} together with the N atom to which they are attached form 4-7 membered heterocycloalkyl (such as 4-membered heterocycloalkyl, 5-membered heterocycloalkyl, 6-membered heterocycloalkyl, 7-membered heterocycloalkyl) optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkoxy-C₁-C₄ alkyl, and C₁-C₄ alkoxy-C₁-C₄ alkoxy.

In some embodiments, each R^{e} is independently H. In some embodiments, each R^{e} is independently D.

In some embodiments, each R^{e} is independently C₁-C₄ alkyl. In some embodiments, each R^{e} is independently C₁-C₄ haloalkyl. In some embodiments, each R^{e} is independently C₂-C₄ alkenyl.

In some embodiments, each R^{e} is independently (C₁-C₄ alkoxy)-C₁-C₄ alkyl. In some embodiments, each R^{e} is independently C₂-C₄ alkynyl.

In some embodiments, each R^{e} is independently C₆-C₁₀ aryl. In some embodiments, each R^{e} is independently 5-10 membered heteroaryl. In some embodiments, each R^{e} is independently C₃-C₁₀ cycloalkyl. In some embodiments, each R^{e} is independently 4-10 membered heterocycloalkyl.

In some embodiments, each R^{e} is independently C₆-C₁₀ aryl-C₁-C₄ alkyl. In some embodiments, each R^{e} is independently C₃-C₁₀ cycloalkyl-C₁-C₄ alkyl. In some embodiments, each R^{e} is independently 5-10 membered heteroaryl-C₁-C₄ alkyl. In some embodiments, each R^{e} is independently 4-10 membered heterocycloalkyl-C₁-C₄ alkyl.

In some embodiments, each R^{f} is independently H, D.

In some embodiments, each R^{f} is independently C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl.

In some embodiments, each R^{a1} is independently H, D.

In some embodiments, each R^{a1} is independently C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl, wherein the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄haloalkyl, or C₁-C₄haloalkoxy.

In some embodiments, each R^{b1} is independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl; wherein the C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl is optionally substituted with 1, 2, 3, 4 or 5 substituents independently selected from D, OH, halo, CN, —NH₂, —NH(C₁-C₄ alkyl), —N(C₁-C₄ alkyl)₂, C₁-C₄ alkyl, C₁-C₄ haloalkyl, —OC₁-C₄ alkyl or —OC₁-C₄ haloalkyl, C₆-C₁₀ aryl, C₃-C₁₀ cycloalkyl, 5-10 membered heteroaryl, or 4-10 membered heterocycloalkyl.

In some embodiments, R^{c1} and R^{d1} are each independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, 4-10 membered heterocycloalkyl-C₁-C₄ alkyl, C₆-C₁₀ aryl-C₃-C₁₀ cycloalkyl, C₆-C₁₀ aryl-4-10 membered heterocycloalkyl, C₆-C₁₀ aryl-5-10 membered heteroaryl, bi(C₆-C₁₀ aryl), 5-10 membered heteroaryl-C₃-C₁₀ cycloalkyl, 5-10 membered heteroaryl-4-10 membered heterocycloalkyl, 5-10 membered heteroaryl-C₆-C₁₀ aryl, or bi(5-10 membered heteroaryl); wherein, the C₁₋₄ alkyl, C₂₋₄ alkenyl, C₂₋₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, 4-10 membered heterocycloalkyl-C₁-C₄ alkyl, C₆-C₁₀ aryl-C₃-C₁₀ cycloalkyl, C₆-C₁₀ aryl-4-10 membered heterocycloalkyl, C₆-C₁₀ aryl-5-10 membered heteroaryl, bi(C₆-C₁₀ aryl), 5-10 membered heteroaryl-C₃-C₁₀ cycloalkyl, 5-10 membered heteroaryl-4-10 membered heterocycloalkyl, 5-10 membered heteroaryl-C₆-C₁₀ aryl, or bi(5-10 membered heteroaryl) is optionally substituted with 1, 2, 3, 4 or 5 substituents independently selected from D, halo, OH, CN, —NH₂, —NH(C₁-C₄ alkyl), —N(C₁-C₄ alkyl)₂, C₁-C₄ alkyl, 0-C₁-C₄ alkyl, C₁-C₄ haloalkyl, 0-C₁-C₄ haloalkyl, C₁-C₄ alkyl-OH, C₁-C₄ alkyl-CN, C₆-C₁₀ aryl, 5-10 membered heteroaryl, C(O)OR^{a1}, C(O)R^{b1}, S(O)₂R^{b1}, C₁-C₄ alkyl-O—C₁-C₄ alkyl, and C₁-C₄ alkyl-O—C₁-C₄ alkyl-O—.

In some embodiments, R^{c1} and R^{d1} together with the N atom to which they are attached form a 4-7 membered heterocycloalkyl optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkoxy-C₁-C₄ alkyl, and C₁-C₄ alkoxy-C₁-C₄ alkoxy.

In some embodiments, each R^{e1} is independently H, D.

In some embodiments, each R^{e1} is independently C₁-C₄ alkyl, C₁-C₄ haloalkyl, C₂-C₄ alkenyl, (C₁-C₄ alkoxy)-C₁-C₄ alkyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, C₃-C₁₀ cycloalkyl-C₁-C₄ alkyl, 5-10 membered heteroaryl-C₁-C₄ alkyl, or 4-10 membered heterocycloalkyl-C₁-C₄ alkyl.

In some embodiments, each R^{f1} is independently H, D.

In some embodiments, each R^{f1} is independently C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl.

In some embodiments, R^{G}, R^{H} and R¹ are each independently C₁-C₄ alkyl or phenyl.

In some embodiments, R^{G} is C₁-C₄ alkyl or phenyl. In some embodiments, R^{G} is methyl, ethyl, n-propyl, i-propyl, n-butyl, i-butyl, t-butyl, or phenyl.

In some embodiments, R^{H} is C₁-C₄ alkyl or phenyl. In some embodiments, R^{G} is methyl, ethyl, n-propyl, i-propyl, n-butyl, i-butyl, t-butyl, or phenyl.

In some embodiments, R¹ is C₁-C₄ alkyl or phenyl. In some embodiments, R^{G} is methyl, ethyl, n-propyl, i-propyl, n-butyl, i-butyl, t-butyl, or phenyl.

In some embodiments, the compounds of Formula (I) are the pharmaceutically acceptable salts. In some embodiments, the compounds of Formula (I) are stereoisomers. In some embodiments, the compounds of Formula (I) are solvates. In some embodiments, the compounds of Formula (I) are N-oxides of the compounds of Formula (I).

Stereoisomers of the compounds of Formula I, and the pharmaceutical salts and solvates thereof, are also contemplated, described, and encompassed herein. Methods of using compounds of Formula I are described, as well as pharmaceutical compositions including the compounds of Formula I.

In one embodiment, provided herein is a compound of Formula (I):

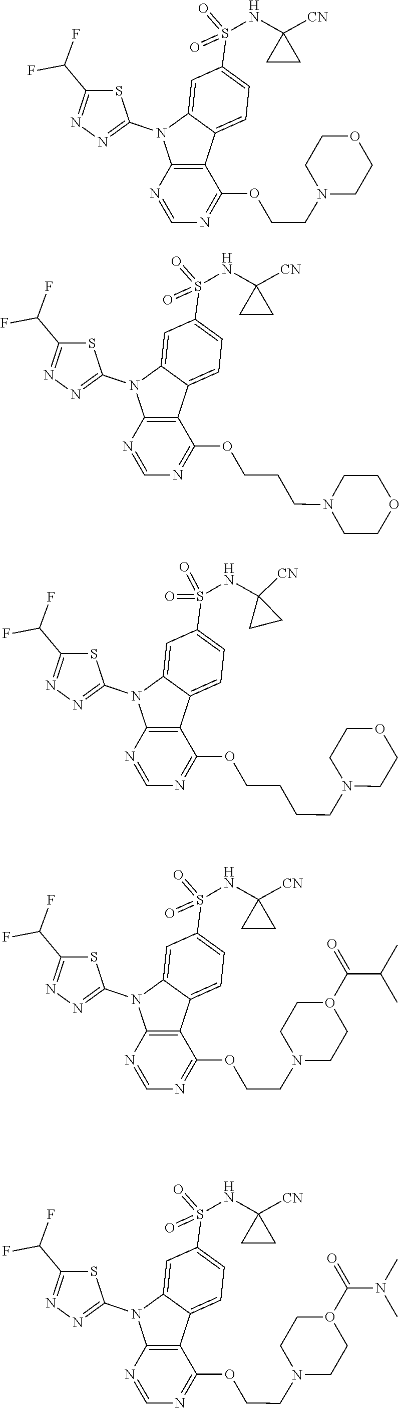

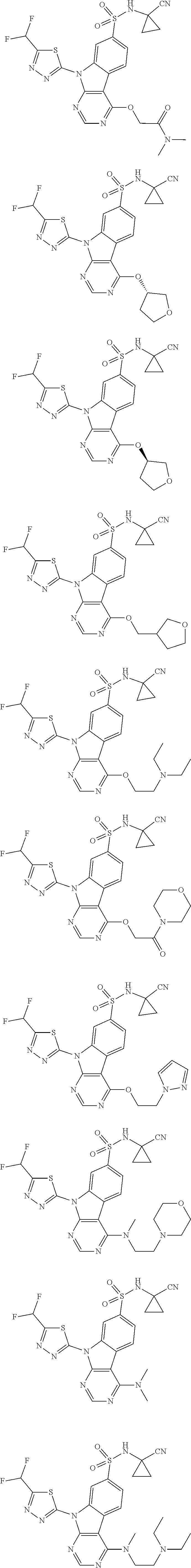

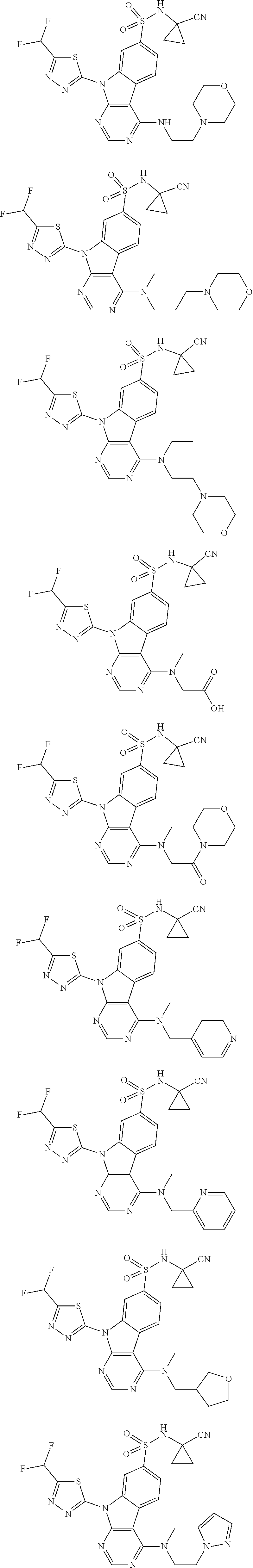

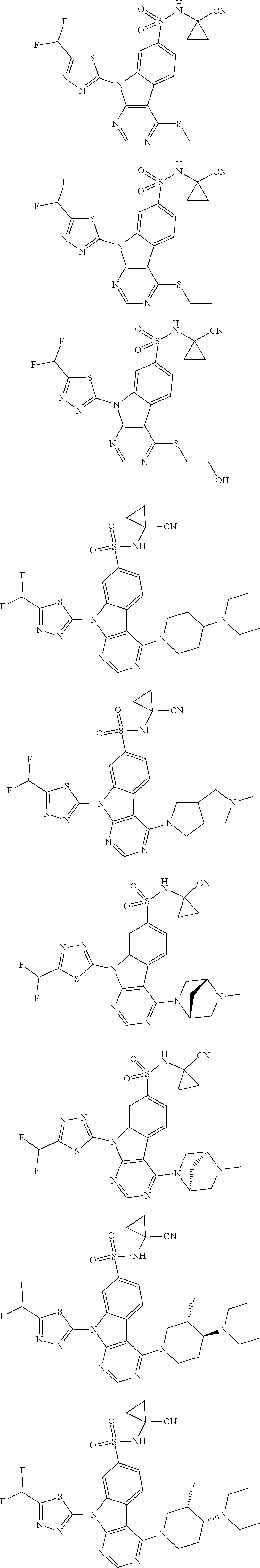

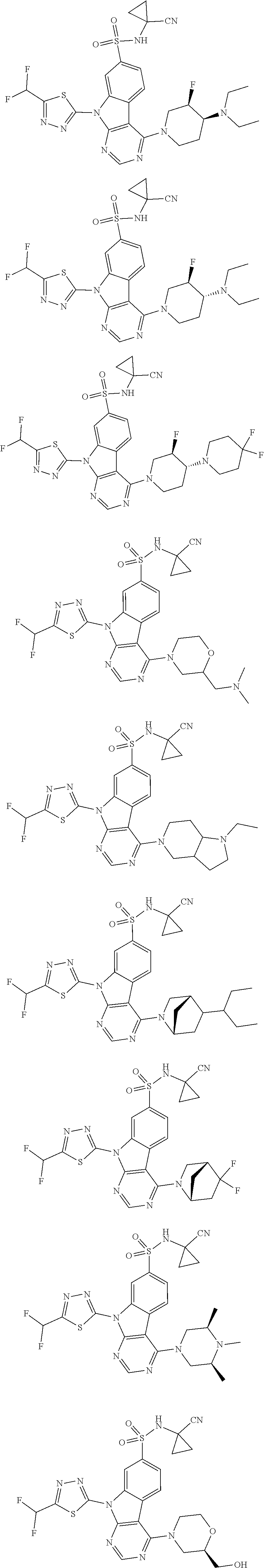

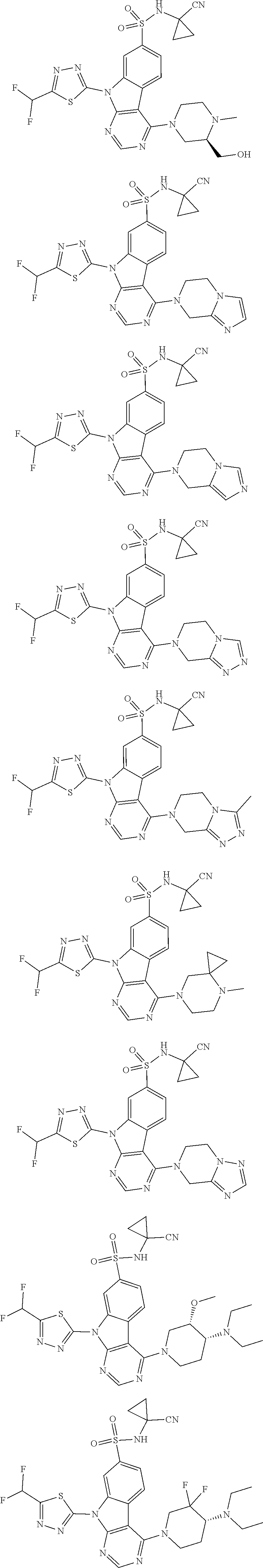

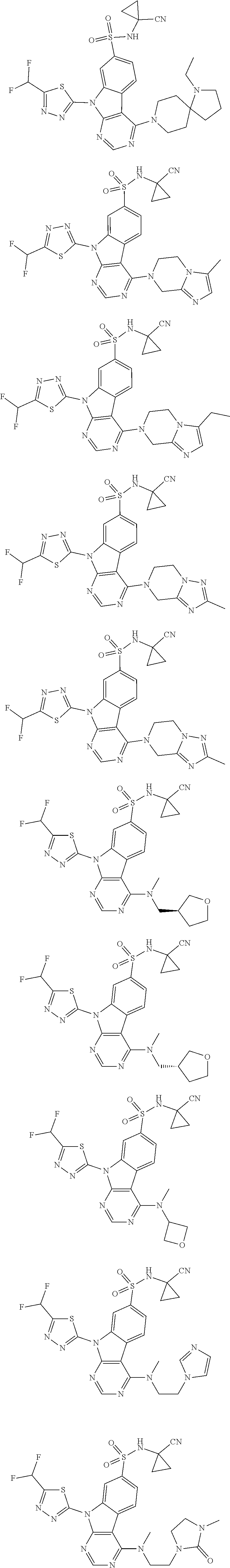

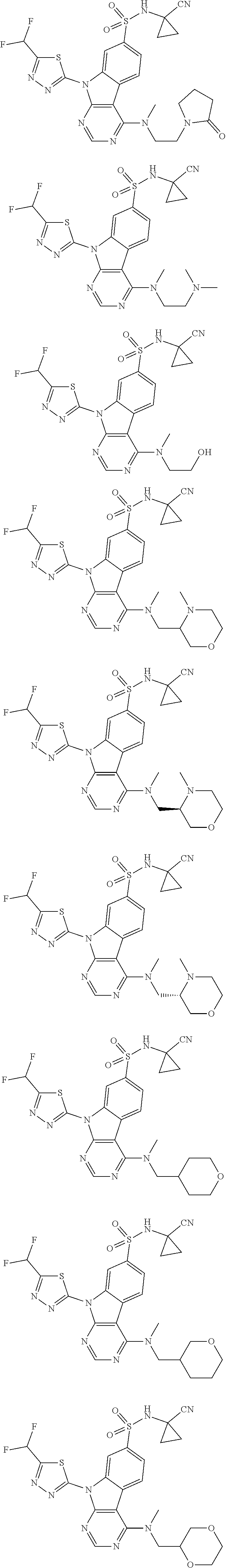

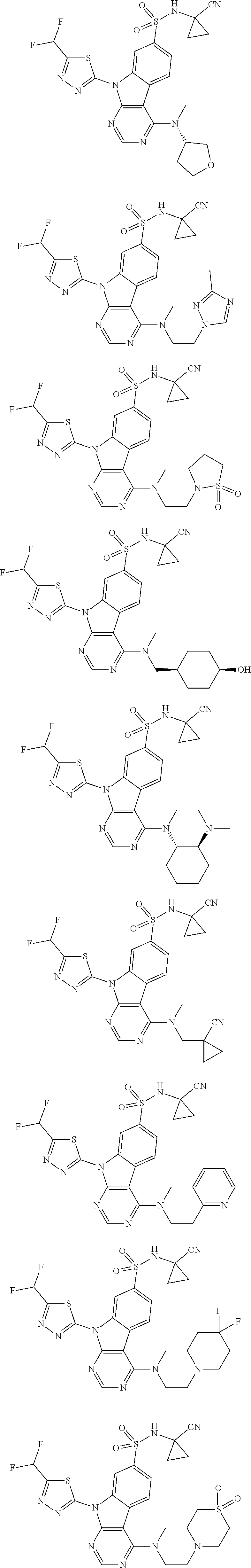

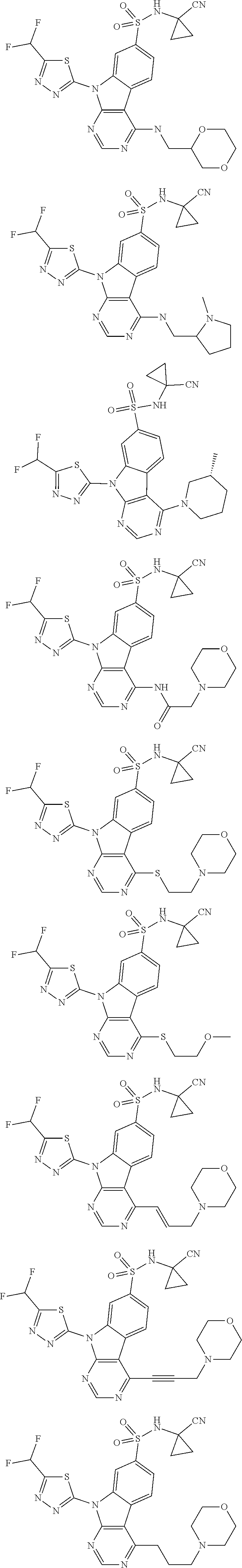

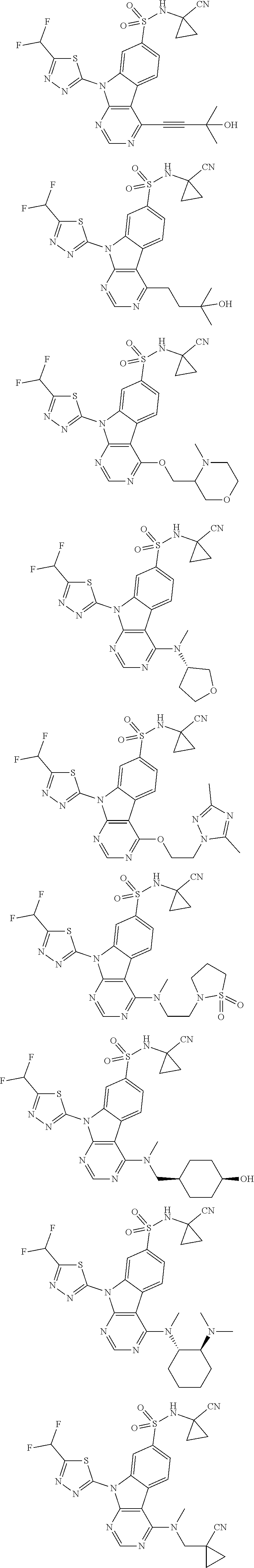

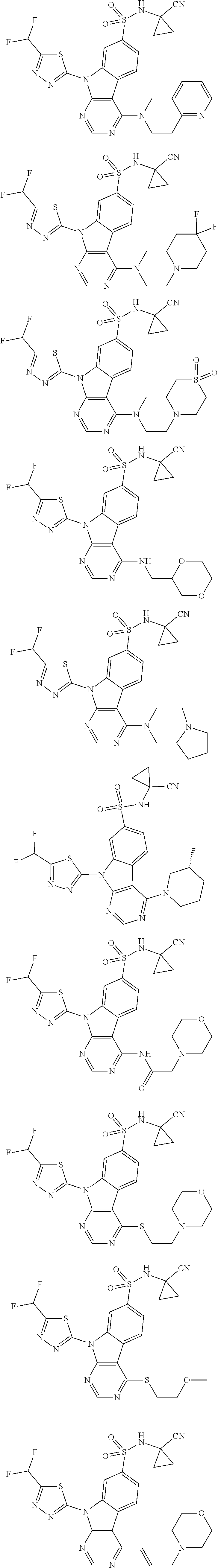

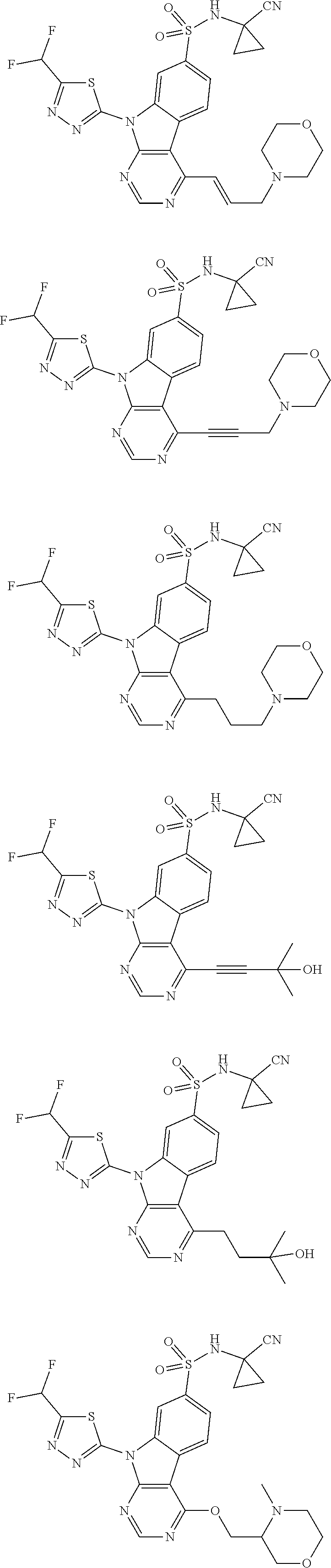 [SMILES: N#CC1(NS(=O)(=O)c2ccc3c4c(/C=C/CN5CCOCC5)ncnc4n(-c4nnc(C(F)F)s4)c3c2)CC1 ; N#CC1(NS(=O)(=O)c2ccc3c4c(C#CCN5CCOCC5)ncnc4n(-c4nnc(C(F)F)s4)c3c2)CC1 ; N#CC1(NS(=O)(=O)c2ccc3c4c(CCCN5CCOCC5)ncnc4n(-c4nnc(C(F)F)s4)c3c2)CC1 ; CC(C)(O)C#Cc1ncnc2c1c1ccc(S(=O)(=O)NC3(C#N)CC3)cc1n2-c1nnc(C(F)F)s1 ; CC(C)(O)CCc1ncnc2c1c1ccc(S(=O)(=O)NC3(C#N)CC3)cc1n2-c1nnc(C(F)F)s1 ; CN1CCOCC1COc1ncnc2c1c1ccc(S(=O)(=O)NC3(C#N)CC3)cc1n2-c1nnc(C(F)F)s1]

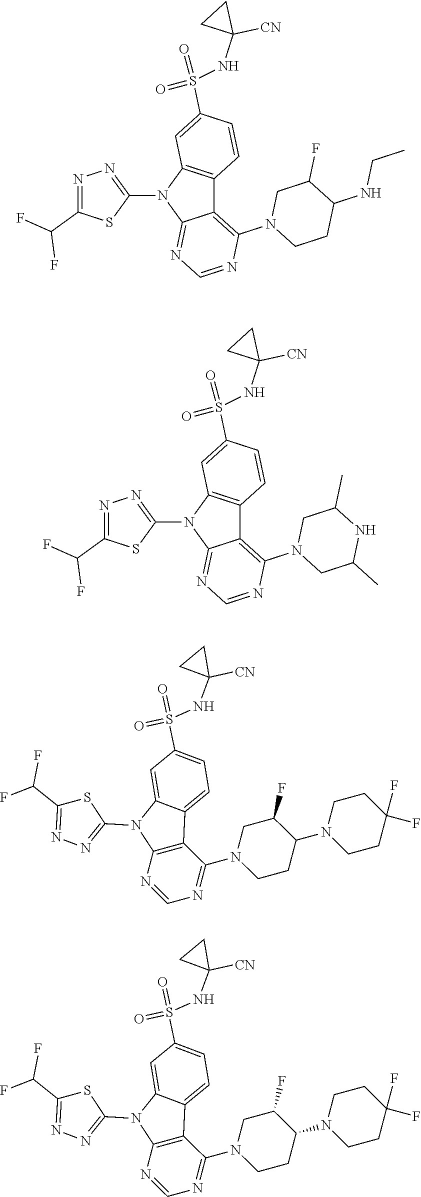 [SMILES: CCNC1CCN(c2ncnc3c2c2ccc(S(=O)(=O)NC4(C#N)CC4)cc2n3-c2nnc(C(F)F)s2)CC1F ; CC1CN(c2ncnc3c2c2ccc(S(=O)(=O)NC4(C#N)CC4)cc2n3-c2nnc(C(F)F)s2)CC(C)N1 ; N#CC1(NS(=O)(=O)c2ccc3c4c(N5CCC(N6CCC(F)(F)CC6)[C@H](F)C5)ncnc4n(-c4nnc(C(F)F)s4)c3c2)CC1 ; N#CC1(NS(=O)(=O)c2ccc3c4c(N5CC[C@@H](N6CCC(F)(F)CC6)[C@@H](F)C5)ncnc4n(-c4nnc(C(F)F)s4)c3c2)CC1]

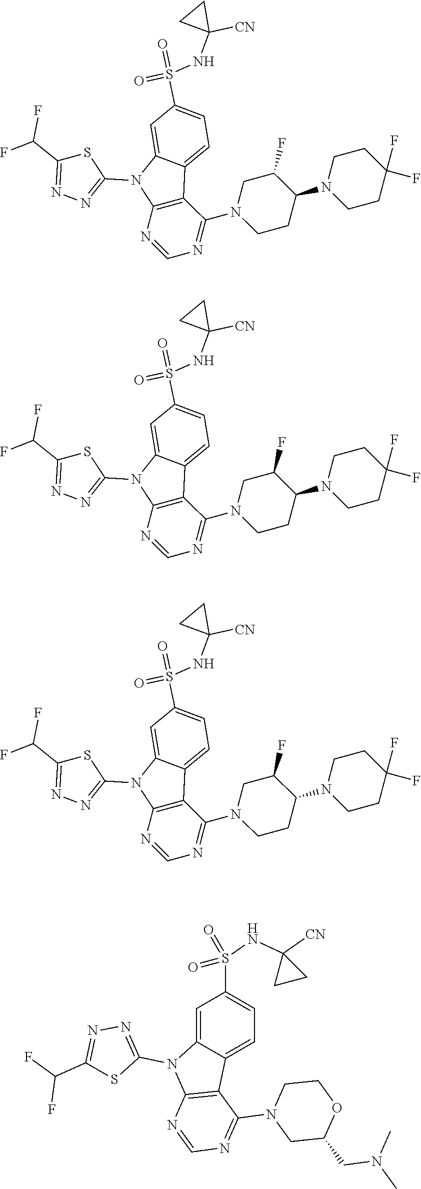 [SMILES: N#CC1(NS(=O)(=O)c2ccc3c4c(N5CC[C@H](N6CCC(F)(F)CC6)[C@@H](F)C5)ncnc4n(-c4nnc(C(F)F)s4)c3c2)CC1 ; N#CC1(NS(=O)(=O)c2ccc3c4c(N5CC[C@H](N6CCC(F)(F)CC6)[C@H](F)C5)ncnc4n(-c4nnc(C(F)F)s4)c3c2)CC1 ; N#CC1(NS(=O)(=O)c2ccc3c4c(N5CC[C@@H](N6CCC(F)(F)CC6)[C@H](F)C5)ncnc4n(-c4nnc(C(F)F)s4)c3c2)CC1 ; CN(C)C[C@@H]1CN(c2ncnc3c2c2ccc(S(=O)(=O)NC4(C#N)CC4)cc2n3-c2nnc(C(F)F)s2)CCO1]

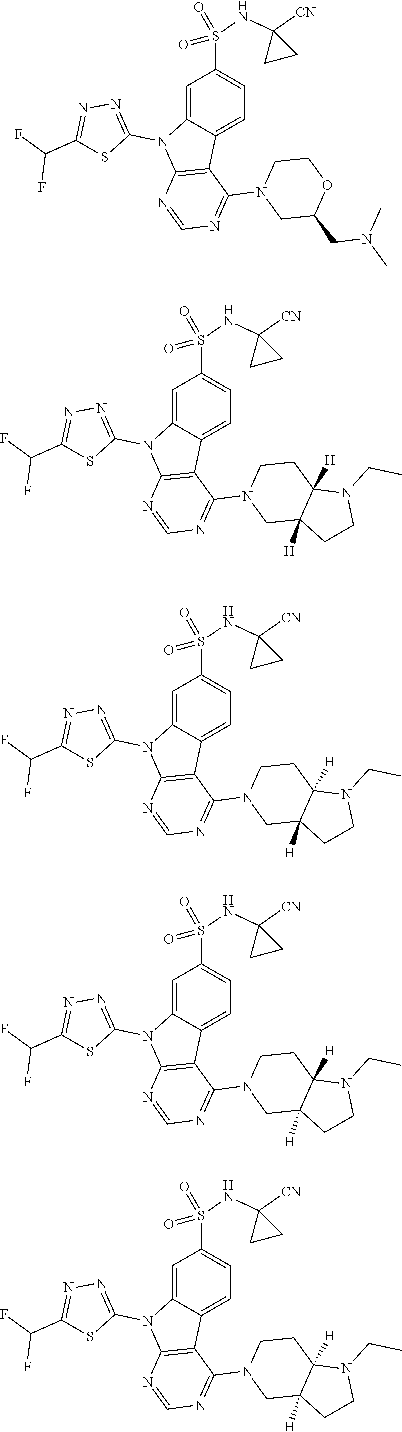 [SMILES: CN(C)C[C@H]1CN(c2ncnc3c2c2ccc(S(=O)(=O)NC4(C#N)CC4)cc2n3-c2nnc(C(F)F)s2)CCO1 ; CCN1CC[C@@H]2CN(c3ncnc4c3c3ccc(S(=O)(=O)NC5(C#N)CC5)cc3n4-c3nnc(C(F)F)s3)CC[C@@H]21 ; CCN1CC[C@@H]2CN(c3ncnc4c3c3ccc(S(=O)(=O)NC5(C#N)CC5)cc3n4-c3nnc(C(F)F)s3)CC[C@H]21 ; CCN1CC[C@H]2CN(c3ncnc4c3c3ccc(S(=O)(=O)NC5(C#N)CC5)cc3n4-c3nnc(C(F)F)s3)CC[C@@H]21 ; CCN1CC[C@H]2CN(c3ncnc4c3c3ccc(S(=O)(=O)NC5(C#N)CC5)cc3n4-c3nnc(C(F)F)s3)CC[C@H]21]

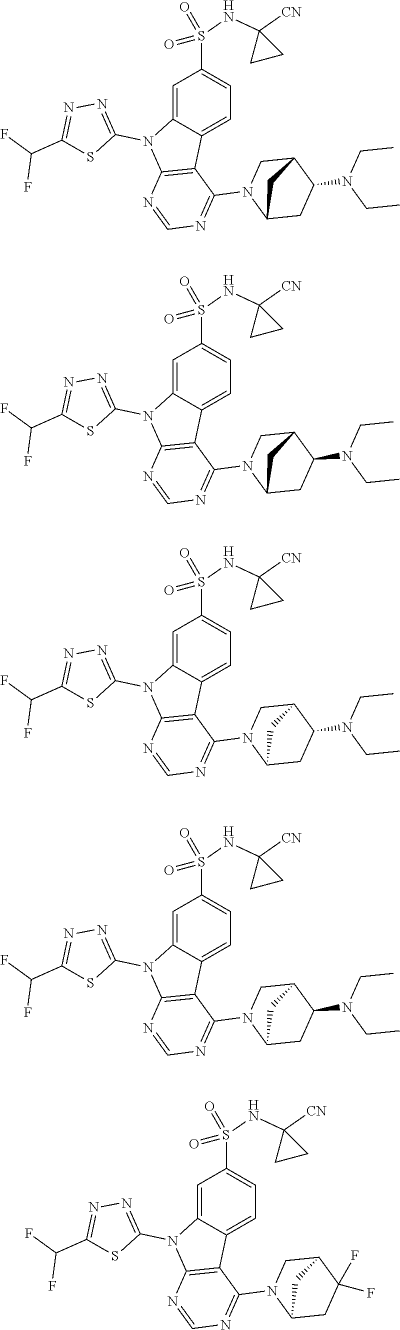 [SMILES: CCN(CC)[C@@H]1C[C@H]2C[C@@H]1CN2c1ncnc2c1c1ccc(S(=O)(=O)NC3(C#N)CC3)cc1n2-c1nnc(C(F)F)s1 ; CCN(CC)[C@H]1C[C@H]2C[C@@H]1CN2c1ncnc2c1c1ccc(S(=O)(=O)NC3(C#N)CC3)cc1n2-c1nnc(C(F)F)s1 ; CCN(CC)[C@@H]1C[C@@H]2C[C@H]1CN2c1ncnc2c1c1ccc(S(=O)(=O)NC3(C#N)CC3)cc1n2-c1nnc(C(F)F)s1 ; CCN(CC)[C@H]1C[C@@H]2C[C@H]1CN2c1ncnc2c1c1ccc(S(=O)(=O)NC3(C#N)CC3)cc1n2-c1nnc(C(F)F)s1 ; N#CC1(NS(=O)(=O)c2ccc3c4c(N5C[C@@H]6C[C@H]5CC6(F)F)ncnc4n(-c4nnc(C(F)F)s4)c3c2)CC1]

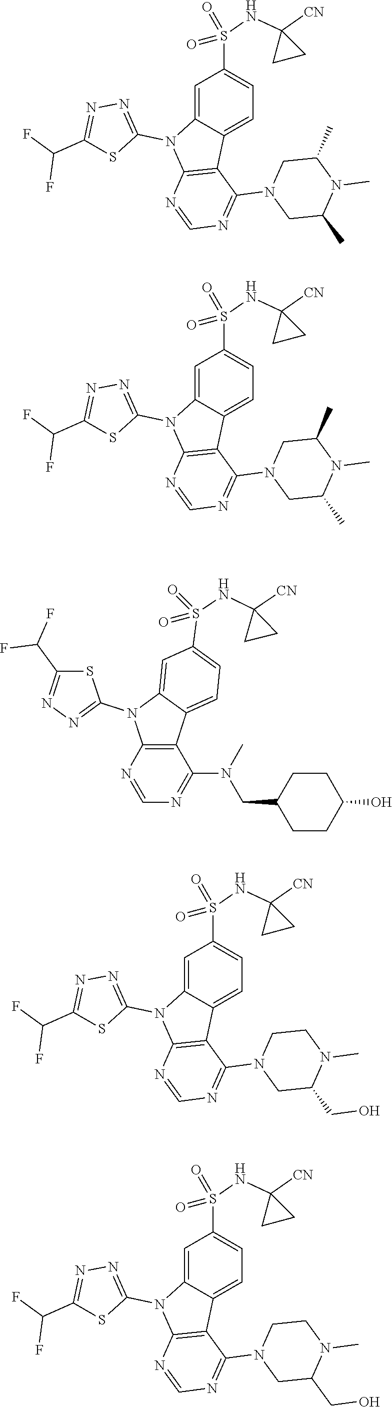

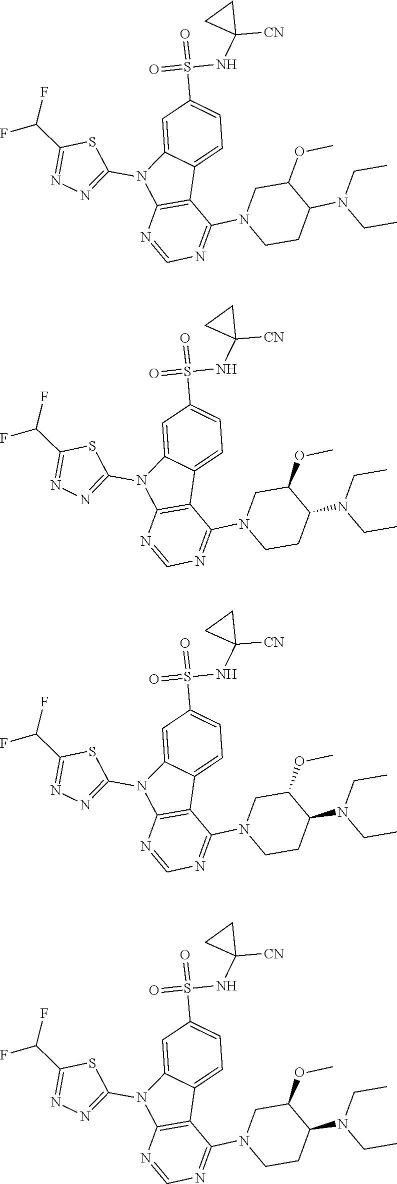 [SMILES: CCN(CC)C1CCN(c2ncnc3c2c2ccc(S(=O)(=O)NC4(C#N)CC4)cc2n3-c2nnc(C(F)F)s2)CC1OC ; CCN(CC)[C@@H]1CCN(c2ncnc3c2c2ccc(S(=O)(=O)NC4(C#N)CC4)cc2n3-c2nnc(C(F)F)s2)C[C@H]1OC ; CCN(CC)[C@H]1CCN(c2ncnc3c2c2ccc(S(=O)(=O)NC4(C#N)CC4)cc2n3-c2nnc(C(F)F)s2)C[C@@H]1OC ; CCN(CC)[C@H]1CCN(c2ncnc3c2c2ccc(S(=O)(=O)NC4(C#N)CC4)cc2n3-c2nnc(C(F)F)s2)C[C@H]1OC]

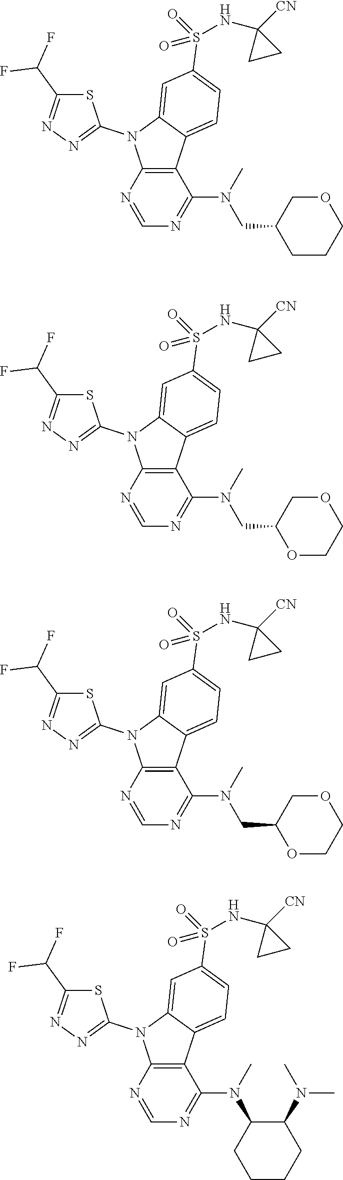 [SMILES: CN(C[C@H]1CCCOC1)c1ncnc2c1c1ccc(S(=O)(=O)NC3(C#N)CC3)cc1n2-c1nnc(C(F)F)s1 ; CN(C[C@@H]1COCCO1)c1ncnc2c1c1ccc(S(=O)(=O)NC3(C#N)CC3)cc1n2-c1nnc(C(F)F)s1 ; CN(C[C@H]1COCCO1)c1ncnc2c1c1ccc(S(=O)(=O)NC3(C#N)CC3)cc1n2-c1nnc(C(F)F)s1 ; CN(C)[C@H]1CCCC[C@H]1N(C)c1ncnc2c1c1ccc(S(=O)(=O)NC3(C#N)CC3)cc1n2-c1nnc(C(F)F)s1]

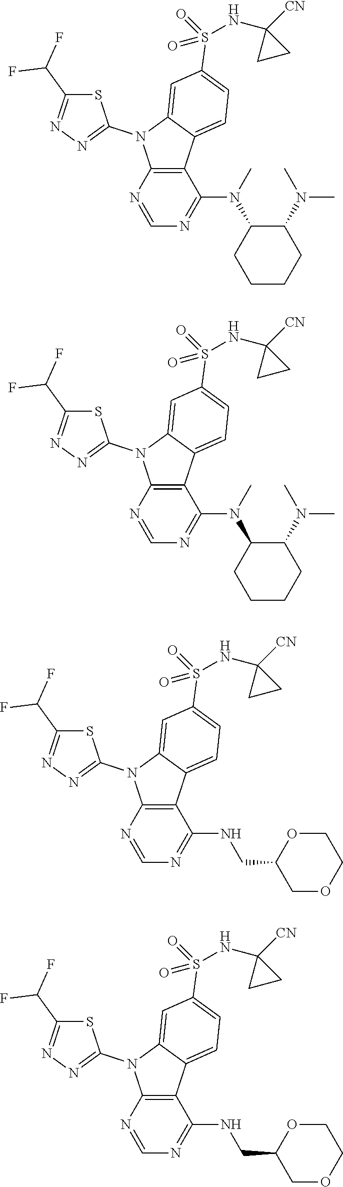

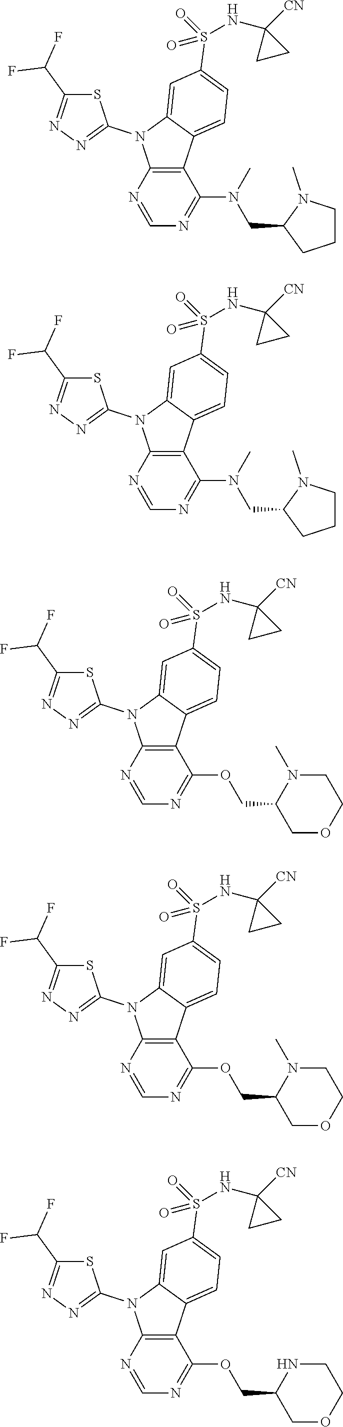

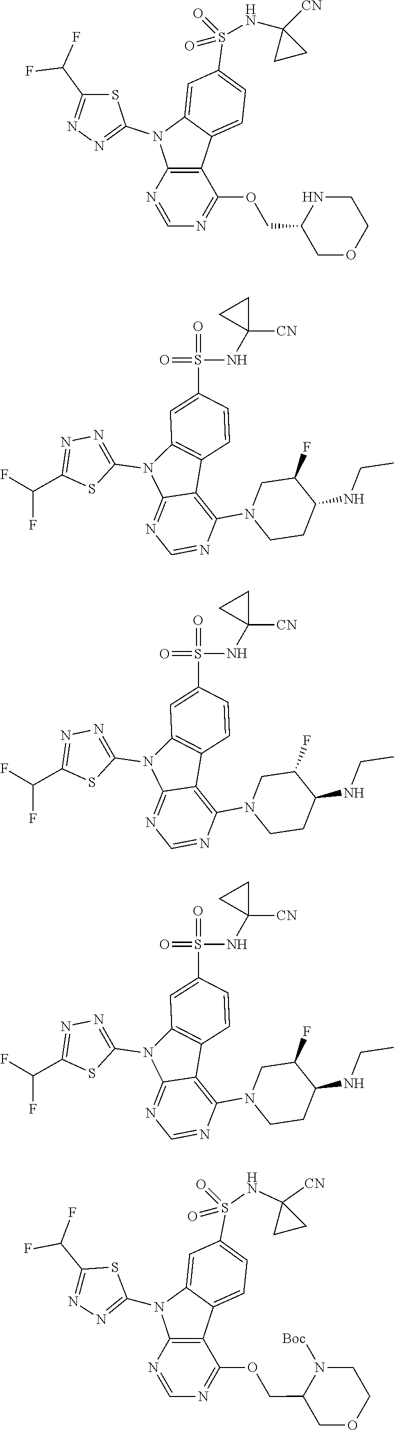 [SMILES: N#CC1(NS(=O)(=O)c2ccc3c4c(OC[C@H]5COCCN5)ncnc4n(-c4nnc(C(F)F)s4)c3c2)CC1 ; CCN[C@@H]1CCN(c2ncnc3c2c2ccc(S(=O)(=O)NC4(C#N)CC4)cc2n3-c2nnc(C(F)F)s2)C[C@H]1F ; CCN[C@H]1CCN(c2ncnc3c2c2ccc(S(=O)(=O)NC4(C#N)CC4)cc2n3-c2nnc(C(F)F)s2)C[C@@H]1F ; CCN[C@H]1CCN(c2ncnc3c2c2ccc(S(=O)(=O)NC4(C#N)CC4)cc2n3-c2nnc(C(F)F)s2)C[C@H]1F ; CC(C)(C)OC(=O)N1CCOCC1COc1ncnc2c1c1ccc(S(=O)(=O)NC3(C#N)CC3)cc1n2-c1nnc(C(F)F)s1]

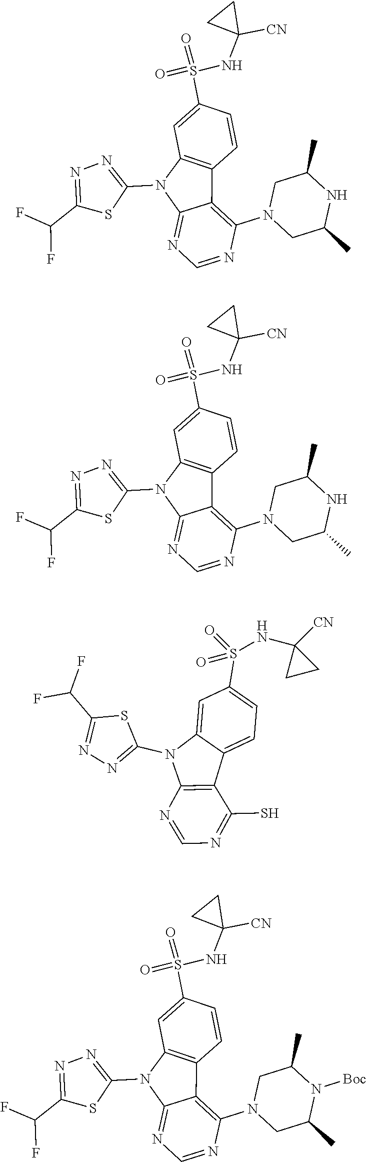 [SMILES: C[C@@H]1CN(c2ncnc3c2c2ccc(S(=O)(=O)NC4(C#N)CC4)cc2n3-c2nnc(C(F)F)s2)C[C@H](C)N1 ; C[C@@H]1CN(c2ncnc3c2c2ccc(S(=O)(=O)NC4(C#N)CC4)cc2n3-c2nnc(C(F)F)s2)C[C@@H](C)N1 ; N#CC1(NS(=O)(=O)c2ccc3c4c(S)ncnc4n(-c4nnc(C(F)F)s4)c3c2)CC1 ; C[C@@H]1CN(c2ncnc3c2c2ccc(S(=O)(=O)NC4(C#N)CC4)cc2n3-c2nnc(C(F)F)s2)C[C@H](C)N1C(=O)OC(C)(C)C]

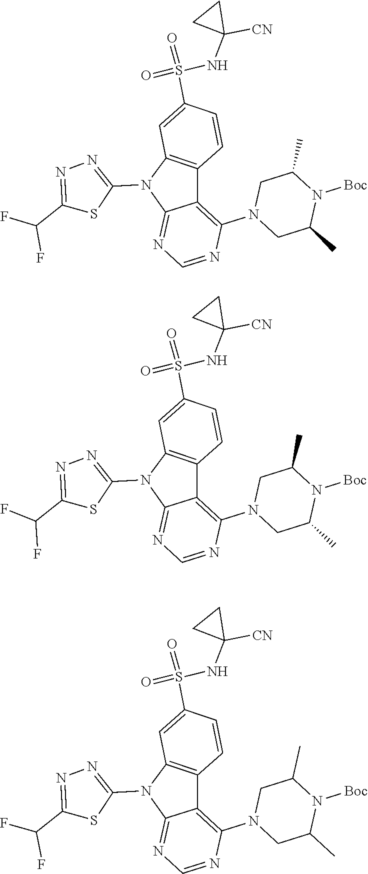

or a pharmaceutically acceptable salt thereof.

In another embodiment, provided herein is a compound of Formula (I):

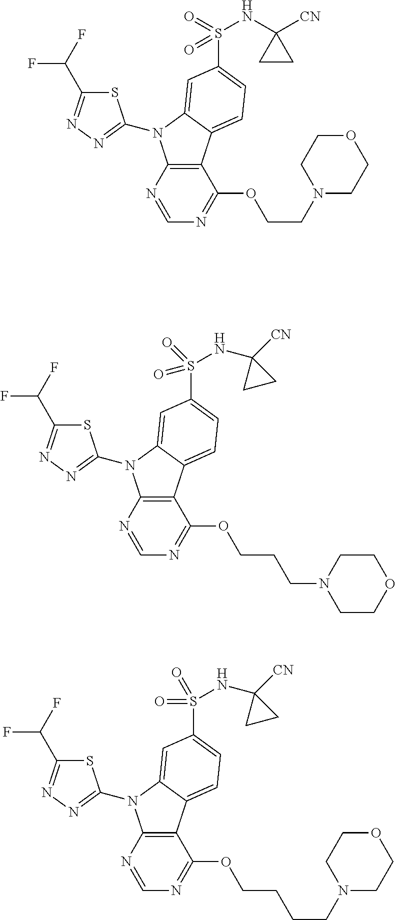 [SMILES: N#CC1(NS(=O)(=O)c2ccc3c4c(OCCN5CCOCC5)ncnc4n(-c4nnc(C(F)F)s4)c3c2)CC1 ; N#CC1(NS(=O)(=O)c2ccc3c4c(OCCCN5CCOCC5)ncnc4n(-c4nnc(C(F)F)s4)c3c2)CC1 ; N#CC1(NS(=O)(=O)c2ccc3c4c(OCCCCN5CCOCC5)ncnc4n(-c4nnc(C(F)F)s4)c3c2)CC1]

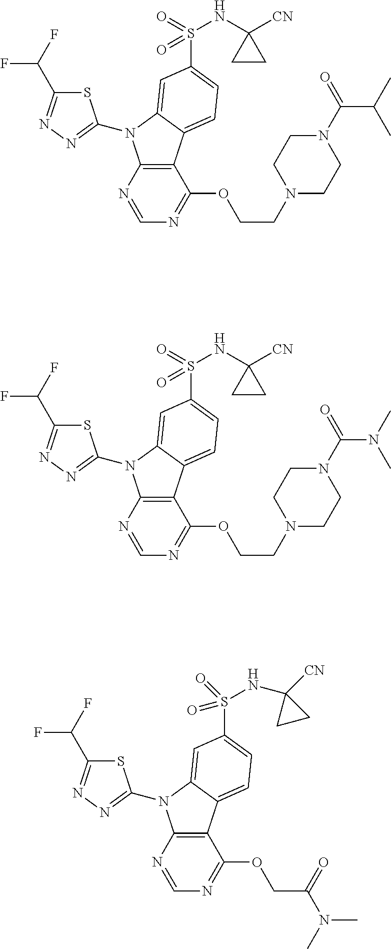 [SMILES: CC(C)C(=O)N1CCN(CCOc2ncnc3c2c2ccc(S(=O)(=O)NC4(C#N)CC4)cc2n3-c2nnc(C(F)F)s2)CC1 ; CN(C)C(=O)N1CCN(CCOc2ncnc3c2c2ccc(S(=O)(=O)NC4(C#N)CC4)cc2n3-c2nnc(C(F)F)s2)CC1 ; CN(C)C(=O)COc1ncnc2c1c1ccc(S(=O)(=O)NC3(C#N)CC3)cc1n2-c1nnc(C(F)F)s1]

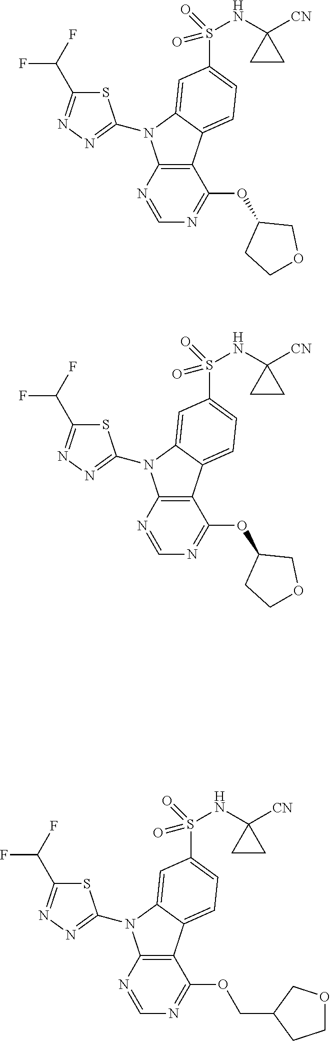 [SMILES: N#CC1(NS(=O)(=O)c2ccc3c4c(O[C@H]5CCOC5)ncnc4n(-c4nnc(C(F)F)s4)c3c2)CC1 ; N#CC1(NS(=O)(=O)c2ccc3c4c(O[C@@H]5CCOC5)ncnc4n(-c4nnc(C(F)F)s4)c3c2)CC1 ; N#CC1(NS(=O)(=O)c2ccc3c4c(OCC5CCOC5)ncnc4n(-c4nnc(C(F)F)s4)c3c2)CC1]

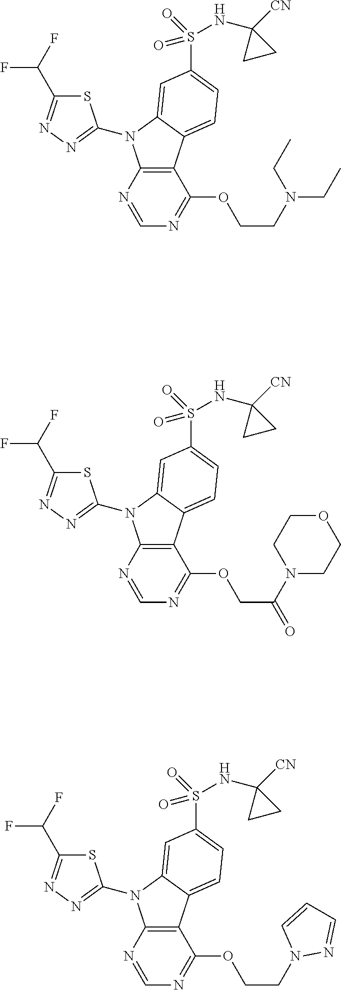 [SMILES: CCN(CC)CCOc1ncnc2c1c1ccc(S(=O)(=O)NC3(C#N)CC3)cc1n2-c1nnc(C(F)F)s1 ; N#CC1(NS(=O)(=O)c2ccc3c4c(OCC(=O)N5CCOCC5)ncnc4n(-c4nnc(C(F)F)s4)c3c2)CC1 ; N#CC1(NS(=O)(=O)c2ccc3c4c(OCCn5cccn5)ncnc4n(-c4nnc(C(F)F)s4)c3c2)CC1]

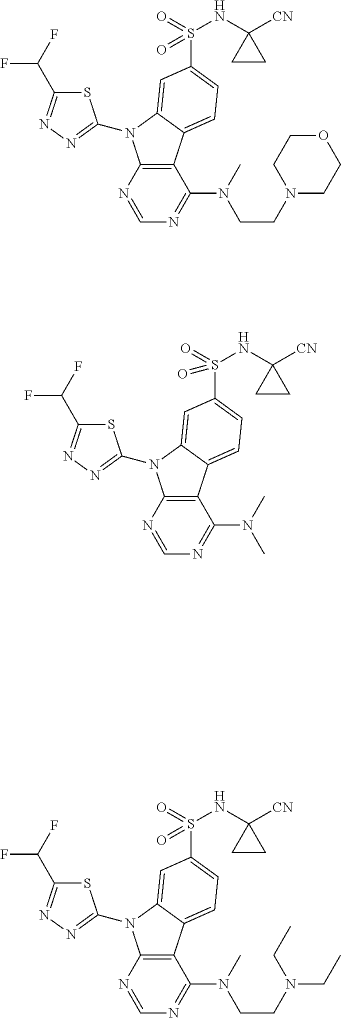 [SMILES: CN(CCN1CCOCC1)c1ncnc2c1c1ccc(S(=O)(=O)NC3(C#N)CC3)cc1n2-c1nnc(C(F)F)s1 ; CN(C)c1ncnc2c1c1ccc(S(=O)(=O)NC3(C#N)CC3)cc1n2-c1nnc(C(F)F)s1 ; CCN(CC)CCN(C)c1ncnc2c1c1ccc(S(=O)(=O)NC3(C#N)CC3)cc1n2-c1nnc(C(F)F)s1]

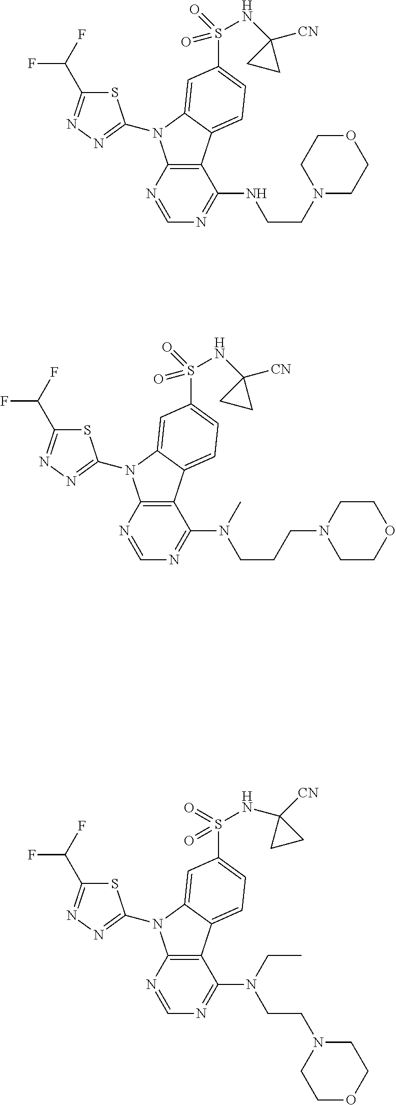 [SMILES: N#CC1(NS(=O)(=O)c2ccc3c4c(NCCN5CCOCC5)ncnc4n(-c4nnc(C(F)F)s4)c3c2)CC1 ; CN(CCCN1CCOCC1)c1ncnc2c1c1ccc(S(=O)(=O)NC3(C#N)CC3)cc1n2-c1nnc(C(F)F)s1 ; CCN(CCN1CCOCC1)c1ncnc2c1c1ccc(S(=O)(=O)NC3(C#N)CC3)cc1n2-c1nnc(C(F)F)s1]

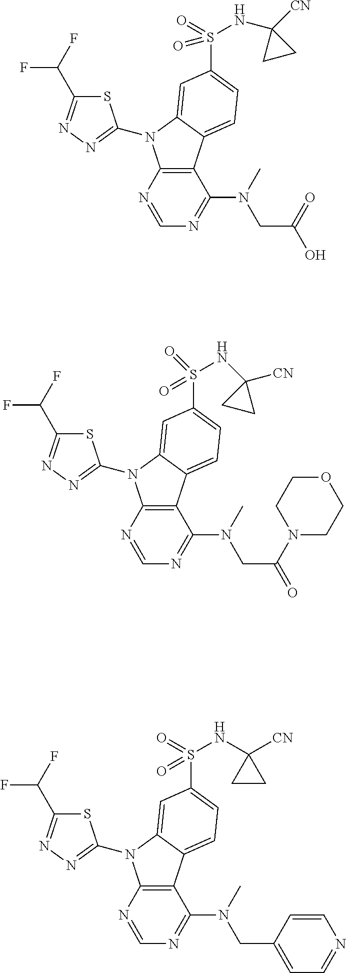 [SMILES: CN(CC(=O)O)c1ncnc2c1c1ccc(S(=O)(=O)NC3(C#N)CC3)cc1n2-c1nnc(C(F)F)s1 ; CN(CC(=O)N1CCOCC1)c1ncnc2c1c1ccc(S(=O)(=O)NC3(C#N)CC3)cc1n2-c1nnc(C(F)F)s1 ; CN(Cc1ccncc1)c1ncnc2c1c1ccc(S(=O)(=O)NC3(C#N)CC3)cc1n2-c1nnc(C(F)F)s1]

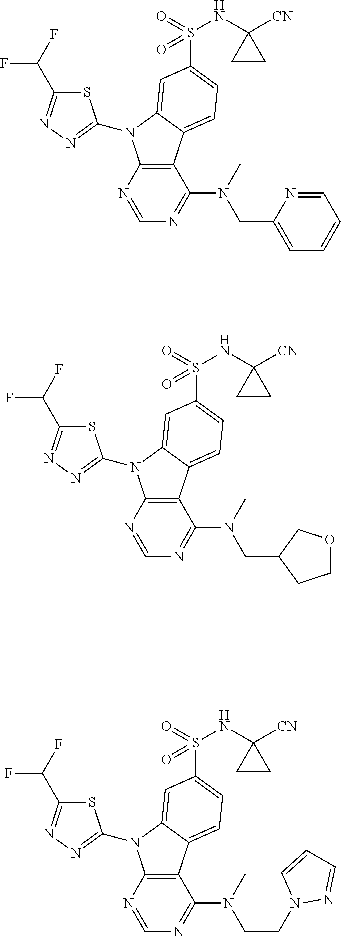 [SMILES: CN(Cc1ccccn1)c1ncnc2c1c1ccc(S(=O)(=O)NC3(C#N)CC3)cc1n2-c1nnc(C(F)F)s1 ; CN(CC1CCOC1)c1ncnc2c1c1ccc(S(=O)(=O)NC3(C#N)CC3)cc1n2-c1nnc(C(F)F)s1 ; CN(CCn1cccn1)c1ncnc2c1c1ccc(S(=O)(=O)NC3(C#N)CC3)cc1n2-c1nnc(C(F)F)s1]

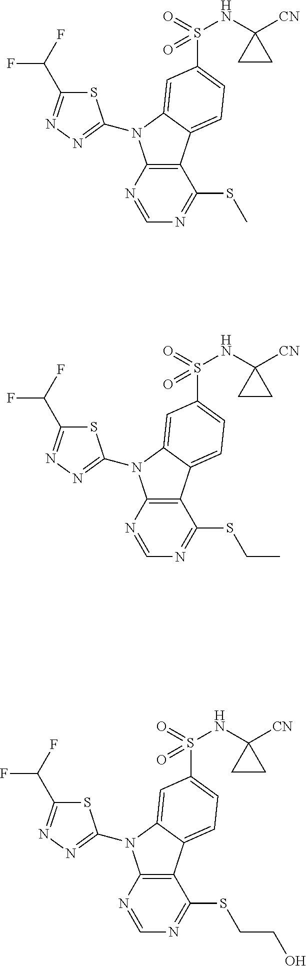 [SMILES: CSc1ncnc2c1c1ccc(S(=O)(=O)NC3(C#N)CC3)cc1n2-c1nnc(C(F)F)s1 ; CCSc1ncnc2c1c1ccc(S(=O)(=O)NC3(C#N)CC3)cc1n2-c1nnc(C(F)F)s1 ; N#CC1(NS(=O)(=O)c2ccc3c4c(SCCO)ncnc4n(-c4nnc(C(F)F)s4)c3c2)CC1]

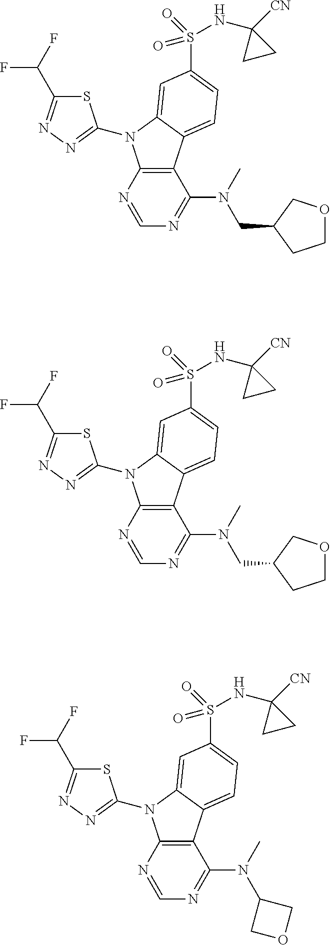 [SMILES: CN(C[C@@H]1CCOC1)c1ncnc2c1c1ccc(S(=O)(=O)NC3(C#N)CC3)cc1n2-c1nnc(C(F)F)s1 ; CN(C[C@H]1CCOC1)c1ncnc2c1c1ccc(S(=O)(=O)NC3(C#N)CC3)cc1n2-c1nnc(C(F)F)s1 ; CN(c1ncnc2c1c1ccc(S(=O)(=O)NC3(C#N)CC3)cc1n2-c1nnc(C(F)F)s1)C1COC1]

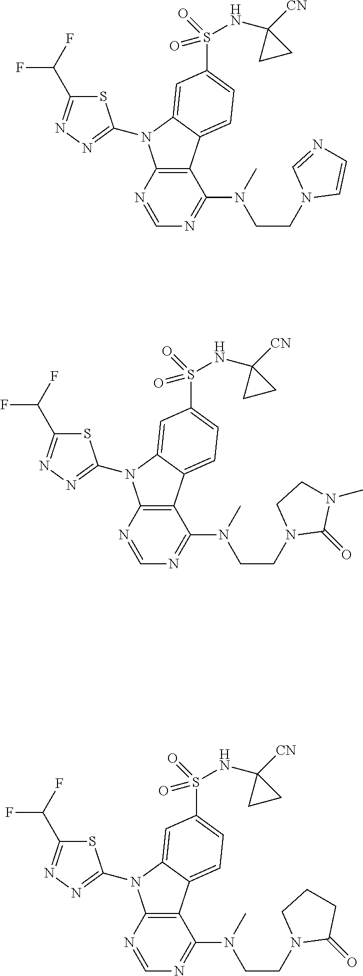 [SMILES: CN(CCn1ccnc1)c1ncnc2c1c1ccc(S(=O)(=O)NC3(C#N)CC3)cc1n2-c1nnc(C(F)F)s1 ; CN1CCN(CCN(C)c2ncnc3c2c2ccc(S(=O)(=O)NC4(C#N)CC4)cc2n3-c2nnc(C(F)F)s2)C1=O ; CN(CCN1CCCC1=O)c1ncnc2c1c1ccc(S(=O)(=O)NC3(C#N)CC3)cc1n2-c1nnc(C(F)F)s1]

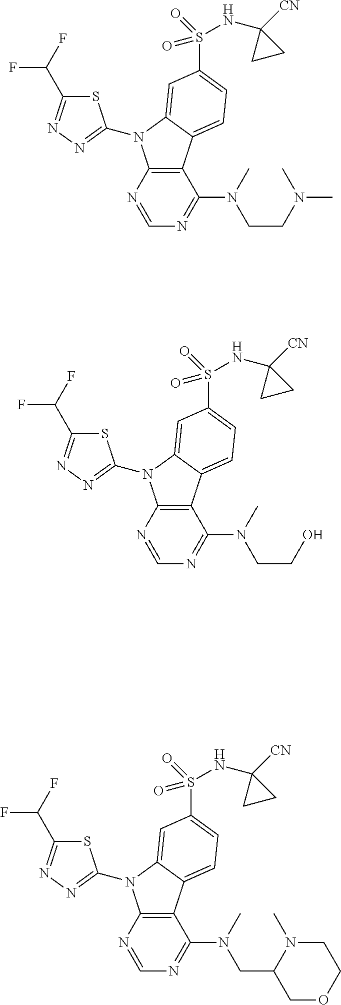 [SMILES: CN(C)CCN(C)c1ncnc2c1c1ccc(S(=O)(=O)NC3(C#N)CC3)cc1n2-c1nnc(C(F)F)s1 ; CN(CCO)c1ncnc2c1c1ccc(S(=O)(=O)NC3(C#N)CC3)cc1n2-c1nnc(C(F)F)s1 ; CN(CC1COCCN1C)c1ncnc2c1c1ccc(S(=O)(=O)NC3(C#N)CC3)cc1n2-c1nnc(C(F)F)s1]

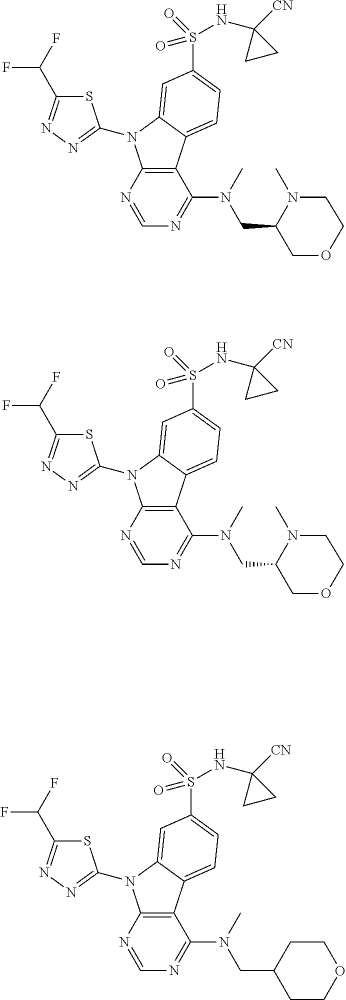 [SMILES: CN(C[C@@H]1COCCN1C)c1ncnc2c1c1ccc(S(=O)(=O)NC3(C#N)CC3)cc1n2-c1nnc(C(F)F)s1 ; CN(C[C@H]1COCCN1C)c1ncnc2c1c1ccc(S(=O)(=O)NC3(C#N)CC3)cc1n2-c1nnc(C(F)F)s1 ; CN(CC1CCOCC1)c1ncnc2c1c1ccc(S(=O)(=O)NC3(C#N)CC3)cc1n2-c1nnc(C(F)F)s1]

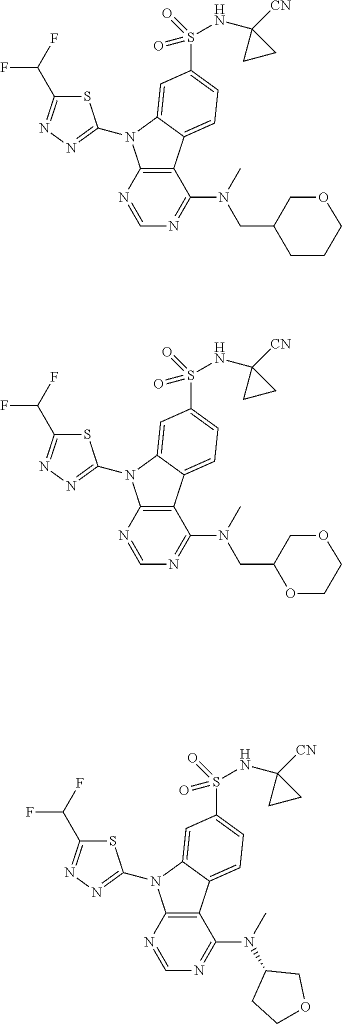 [SMILES: CN(CC1CCCOC1)c1ncnc2c1c1ccc(S(=O)(=O)NC3(C#N)CC3)cc1n2-c1nnc(C(F)F)s1 ; CN(CC1COCCO1)c1ncnc2c1c1ccc(S(=O)(=O)NC3(C#N)CC3)cc1n2-c1nnc(C(F)F)s1 ; CN(c1ncnc2c1c1ccc(S(=O)(=O)NC3(C#N)CC3)cc1n2-c1nnc(C(F)F)s1)[C@H]1CCOC1]

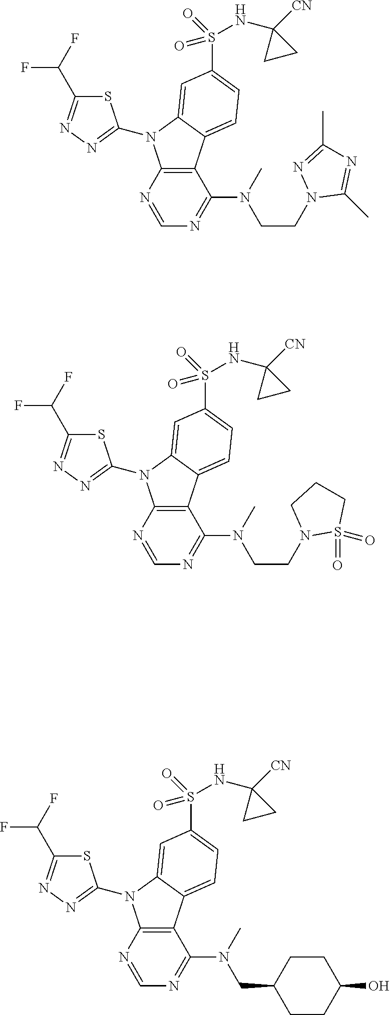 [SMILES: Cc1nc(C)n(CCN(C)c2ncnc3c2c2ccc(S(=O)(=O)NC4(C#N)CC4)cc2n3-c2nnc(C(F)F)s2)n1 ; CN(CCN1CCCS1(=O)=O)c1ncnc2c1c1ccc(S(=O)(=O)NC3(C#N)CC3)cc1n2-c1nnc(C(F)F)s1 ; CN(C[C@H]1CC[C@@H](O)CC1)c1ncnc2c1c1ccc(S(=O)(=O)NC3(C#N)CC3)cc1n2-c1nnc(C(F)F)s1]

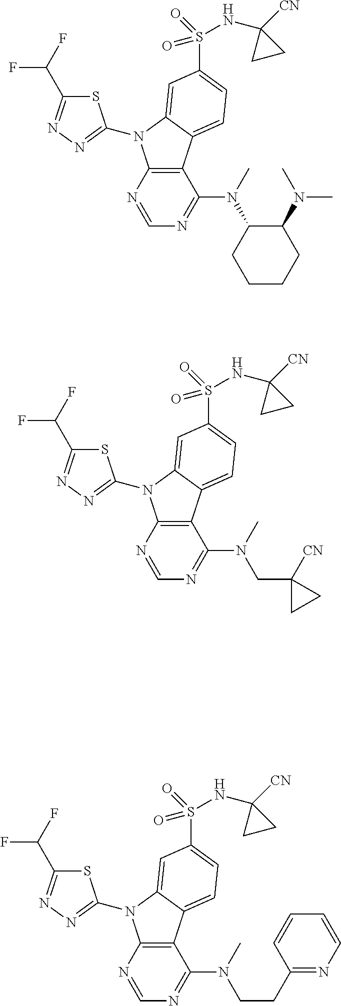 [SMILES: CN(C)[C@H]1CCCC[C@@H]1N(C)c1ncnc2c1c1ccc(S(=O)(=O)NC3(C#N)CC3)cc1n2-c1nnc(C(F)F)s1 ; CN(CC1(C#N)CC1)c1ncnc2c1c1ccc(S(=O)(=O)NC3(C#N)CC3)cc1n2-c1nnc(C(F)F)s1 ; CN(CCc1ccccn1)c1ncnc2c1c1ccc(S(=O)(=O)NC3(C#N)CC3)cc1n2-c1nnc(C(F)F)s1]

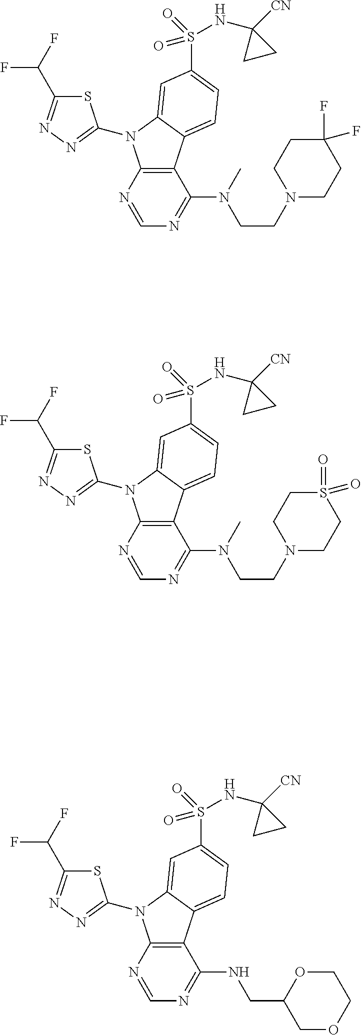 [SMILES: CN(CCN1CCC(F)(F)CC1)c1ncnc2c1c1ccc(S(=O)(=O)NC3(C#N)CC3)cc1n2-c1nnc(C(F)F)s1 ; CN(CCN1CCS(=O)(=O)CC1)c1ncnc2c1c1ccc(S(=O)(=O)NC3(C#N)CC3)cc1n2-c1nnc(C(F)F)s1 ; N#CC1(NS(=O)(=O)c2ccc3c4c(NCC5COCCO5)ncnc4n(-c4nnc(C(F)F)s4)c3c2)CC1]

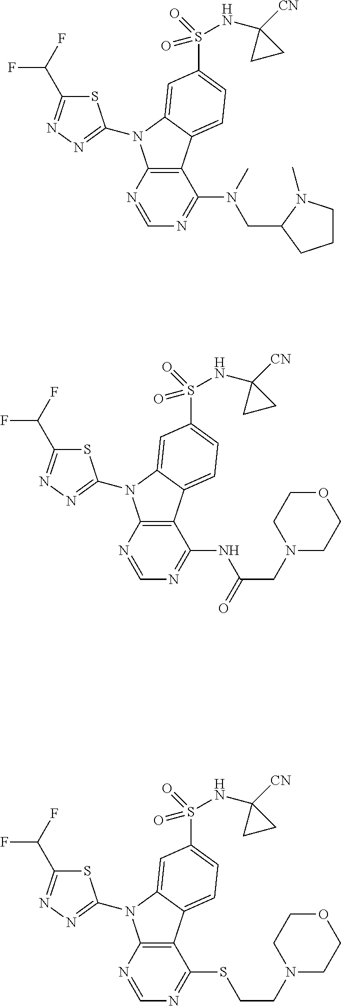 [SMILES: CN(CC1CCCN1C)c1ncnc2c1c1ccc(S(=O)(=O)NC3(C#N)CC3)cc1n2-c1nnc(C(F)F)s1 ; N#CC1(NS(=O)(=O)c2ccc3c4c(NC(=O)CN5CCOCC5)ncnc4n(-c4nnc(C(F)F)s4)c3c2)CC1 ; N#CC1(NS(=O)(=O)c2ccc3c4c(SCCN5CCOCC5)ncnc4n(-c4nnc(C(F)F)s4)c3c2)CC1]

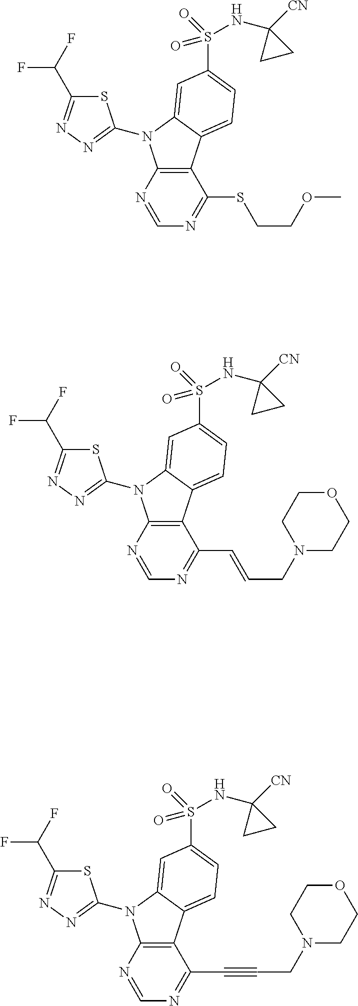 [SMILES: COCCSc1ncnc2c1c1ccc(S(=O)(=O)NC3(C#N)CC3)cc1n2-c1nnc(C(F)F)s1 ; N#CC1(NS(=O)(=O)c2ccc3c4c(/C=C/CN5CCOCC5)ncnc4n(-c4nnc(C(F)F)s4)c3c2)CC1 ; N#CC1(NS(=O)(=O)c2ccc3c4c(C#CCN5CCOCC5)ncnc4n(-c4nnc(C(F)F)s4)c3c2)CC1]

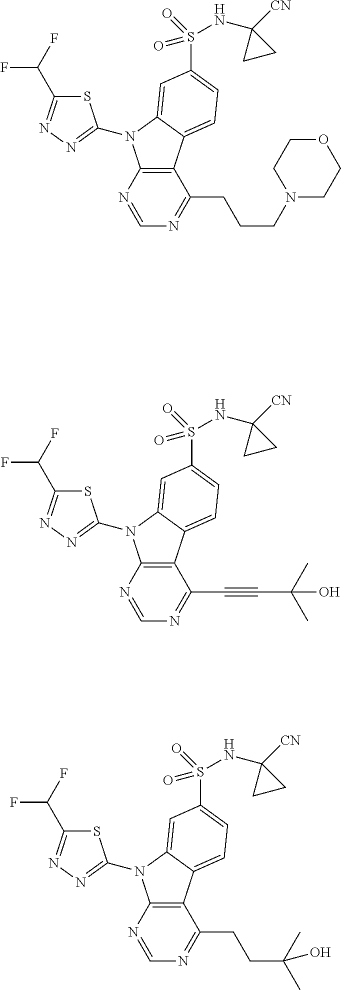 [SMILES: N#CC1(NS(=O)(=O)c2ccc3c4c(CCCN5CCOCC5)ncnc4n(-c4nnc(C(F)F)s4)c3c2)CC1 ; CC(C)(O)C#Cc1ncnc2c1c1ccc(S(=O)(=O)NC3(C#N)CC3)cc1n2-c1nnc(C(F)F)s1 ; CC(C)(O)CCc1ncnc2c1c1ccc(S(=O)(=O)NC3(C#N)CC3)cc1n2-c1nnc(C(F)F)s1]

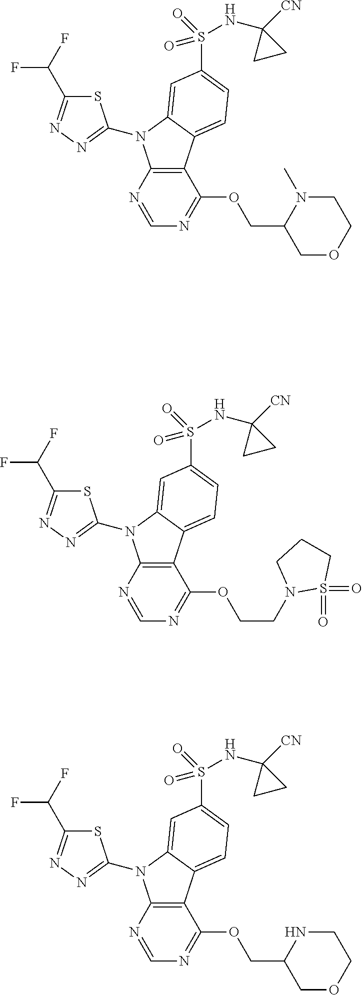 [SMILES: CN1CCOCC1COc1ncnc2c1c1ccc(S(=O)(=O)NC3(C#N)CC3)cc1n2-c1nnc(C(F)F)s1 ; N#CC1(NS(=O)(=O)c2ccc3c4c(OCCN5CCCS5(=O)=O)ncnc4n(-c4nnc(C(F)F)s4)c3c2)CC1 ; N#CC1(NS(=O)(=O)c2ccc3c4c(OCC5COCCN5)ncnc4n(-c4nnc(C(F)F)s4)c3c2)CC1]

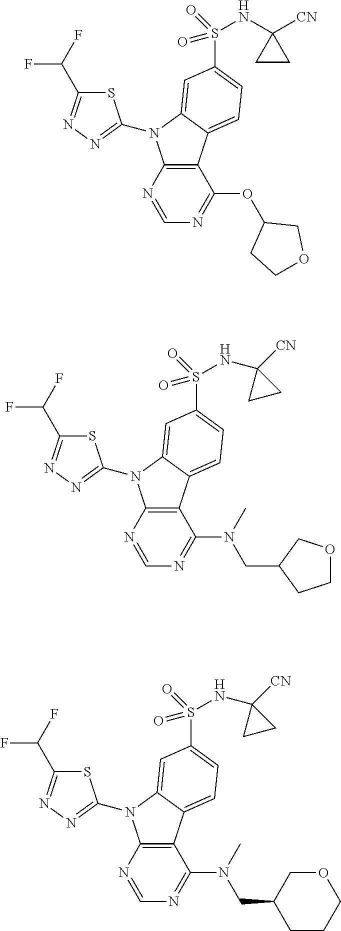 [SMILES: N#CC1(NS(=O)(=O)c2ccc3c4c(OC5CCOC5)ncnc4n(-c4nnc(C(F)F)s4)c3c2)CC1 ; CN(CC1CCOC1)c1ncnc2c1c1ccc(S(=O)(=O)NC3(C#N)CC3)cc1n2-c1nnc(C(F)F)s1 ; CN(C[C@@H]1CCCOC1)c1ncnc2c1c1ccc(S(=O)(=O)NC3(C#N)CC3)cc1n2-c1nnc(C(F)F)s1]

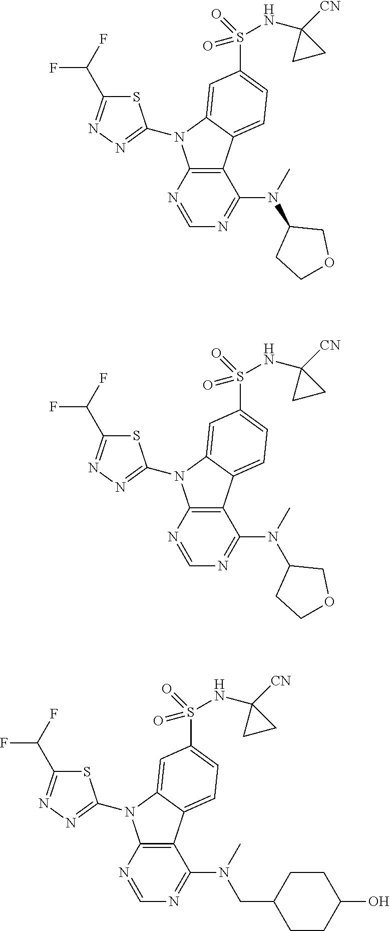 [SMILES: CN(c1ncnc2c1c1ccc(S(=O)(=O)NC3(C#N)CC3)cc1n2-c1nnc(C(F)F)s1)[C@@H]1CCOC1 ; CN(c1ncnc2c1c1ccc(S(=O)(=O)NC3(C#N)CC3)cc1n2-c1nnc(C(F)F)s1)C1CCOC1 ; CN(CC1CCC(O)CC1)c1ncnc2c1c1ccc(S(=O)(=O)NC3(C#N)CC3)cc1n2-c1nnc(C(F)F)s1]

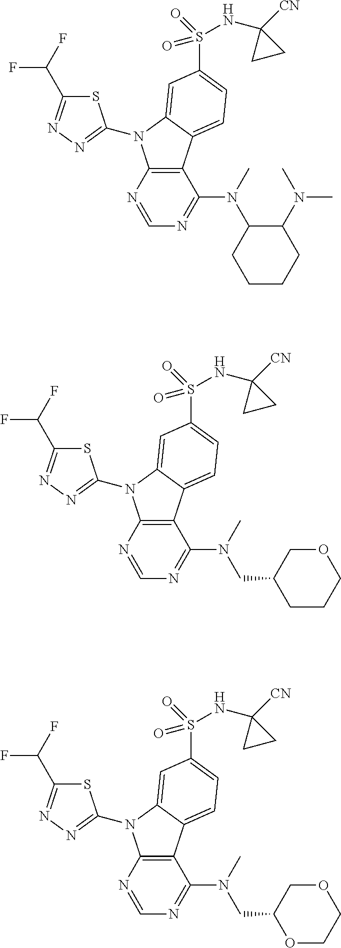 [SMILES: CN(C)C1CCCCC1N(C)c1ncnc2c1c1ccc(S(=O)(=O)NC3(C#N)CC3)cc1n2-c1nnc(C(F)F)s1 ; CN(C[C@H]1CCCOC1)c1ncnc2c1c1ccc(S(=O)(=O)NC3(C#N)CC3)cc1n2-c1nnc(C(F)F)s1 ; CN(C[C@@H]1COCCO1)c1ncnc2c1c1ccc(S(=O)(=O)NC3(C#N)CC3)cc1n2-c1nnc(C(F)F)s1]

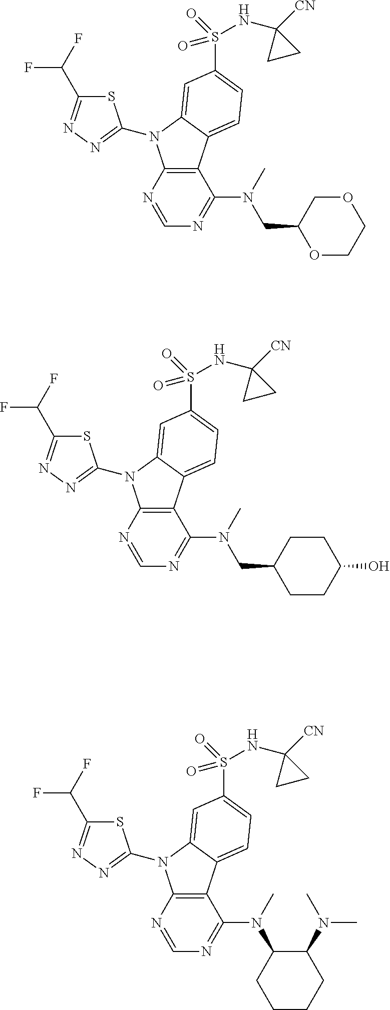 [SMILES: CN(C[C@H]1COCCO1)c1ncnc2c1c1ccc(S(=O)(=O)NC3(C#N)CC3)cc1n2-c1nnc(C(F)F)s1 ; CN(C[C@H]1CC[C@H](O)CC1)c1ncnc2c1c1ccc(S(=O)(=O)NC3(C#N)CC3)cc1n2-c1nnc(C(F)F)s1 ; CN(C)[C@H]1CCCC[C@H]1N(C)c1ncnc2c1c1ccc(S(=O)(=O)NC3(C#N)CC3)cc1n2-c1nnc(C(F)F)s1]

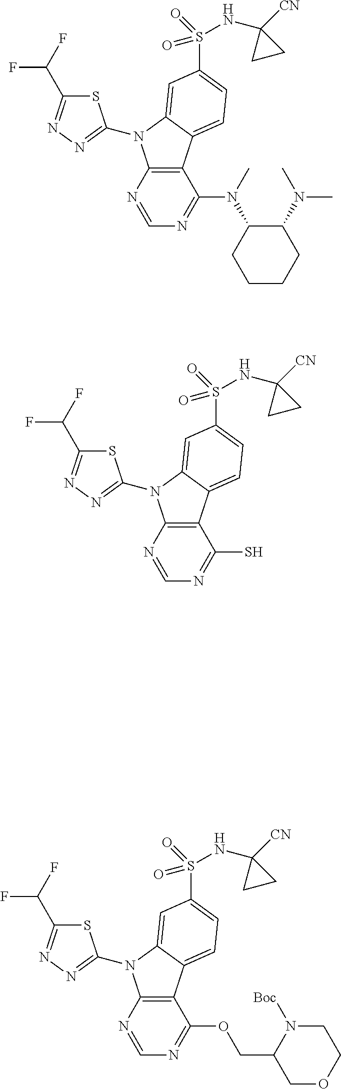 [SMILES: CN(C)[C@@H]1CCCC[C@@H]1N(C)c1ncnc2c1c1ccc(S(=O)(=O)NC3(C#N)CC3)cc1n2-c1nnc(C(F)F)s1 ; N#CC1(NS(=O)(=O)c2ccc3c4c(S)ncnc4n(-c4nnc(C(F)F)s4)c3c2)CC1 ; CC(C)(C)OC(=O)N1CCOCC1COc1ncnc2c1c1ccc(S(=O)(=O)NC3(C#N)CC3)cc1n2-c1nnc(C(F)F)s1]

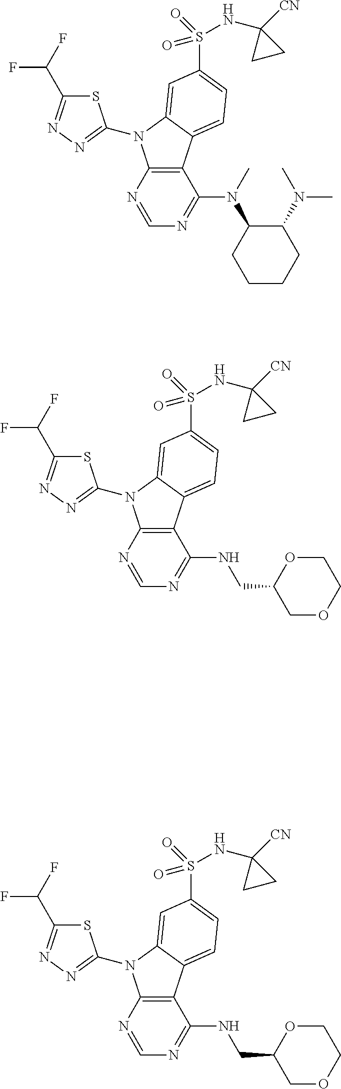 [SMILES: CN(C)[C@@H]1CCCC[C@H]1N(C)c1ncnc2c1c1ccc(S(=O)(=O)NC3(C#N)CC3)cc1n2-c1nnc(C(F)F)s1 ; N#CC1(NS(=O)(=O)c2ccc3c4c(NC[C@H]5COCCO5)ncnc4n(-c4nnc(C(F)F)s4)c3c2)CC1 ; N#CC1(NS(=O)(=O)c2ccc3c4c(NC[C@@H]5COCCO5)ncnc4n(-c4nnc(C(F)F)s4)c3c2)CC1]

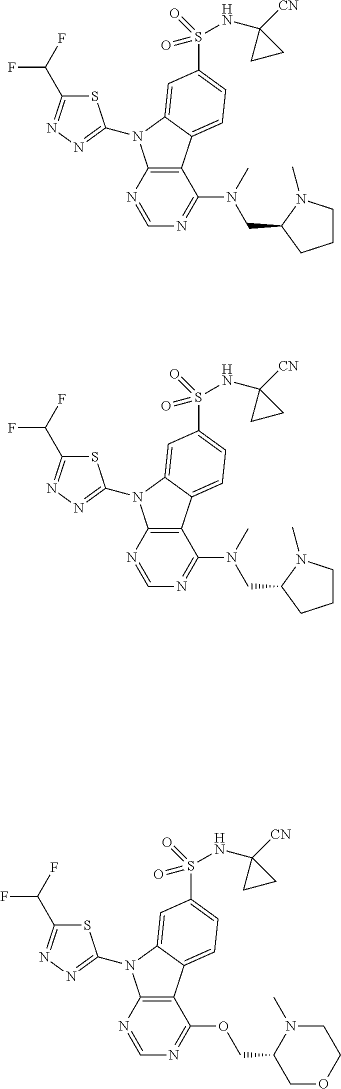 [SMILES: CN(C[C@@H]1CCCN1C)c1ncnc2c1c1ccc(S(=O)(=O)NC3(C#N)CC3)cc1n2-c1nnc(C(F)F)s1 ; CN(C[C@H]1CCCN1C)c1ncnc2c1c1ccc(S(=O)(=O)NC3(C#N)CC3)cc1n2-c1nnc(C(F)F)s1 ; CN1CCOC[C@@H]1COc1ncnc2c1c1ccc(S(=O)(=O)NC3(C#N)CC3)cc1n2-c1nnc(C(F)F)s1]

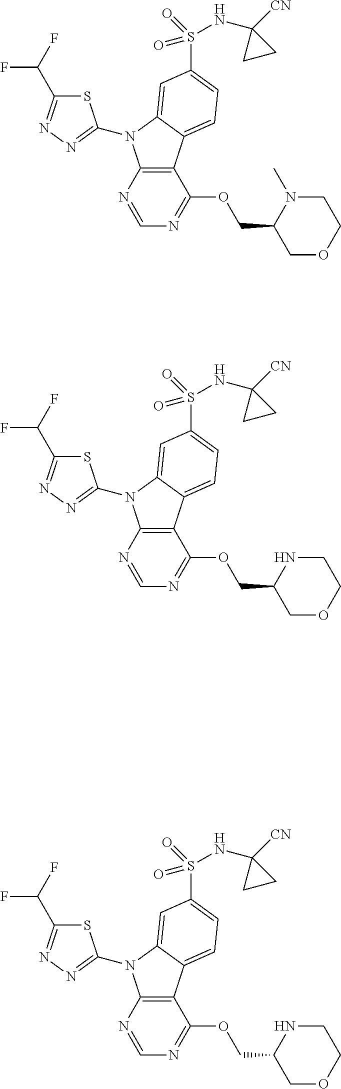 [SMILES: CN1CCOC[C@H]1COc1ncnc2c1c1ccc(S(=O)(=O)NC3(C#N)CC3)cc1n2-c1nnc(C(F)F)s1 ; N#CC1(NS(=O)(=O)c2ccc3c4c(OC[C@@H]5COCCN5)ncnc4n(-c4nnc(C(F)F)s4)c3c2)CC1 ; N#CC1(NS(=O)(=O)c2ccc3c4c(OC[C@H]5COCCN5)ncnc4n(-c4nnc(C(F)F)s4)c3c2)CC1]

or a pharmaceutically acceptable salt thereof.

In yet another embodiment, provided herein is a compound of Formula (I):

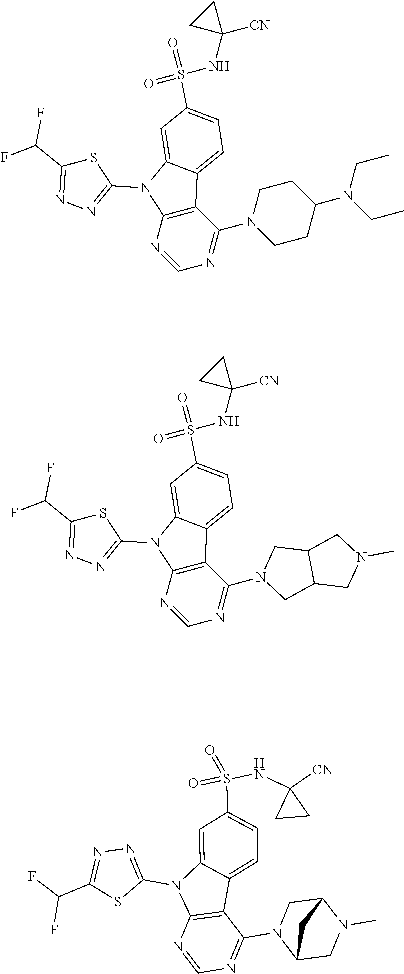 [SMILES: CCN(CC)C1CCN(c2ncnc3c2c2ccc(S(=O)(=O)NC4(C#N)CC4)cc2n3-c2nnc(C(F)F)s2)CC1 ; CN1CC2CN(c3ncnc4c3c3ccc(S(=O)(=O)NC5(C#N)CC5)cc3n4-c3nnc(C(F)F)s3)CC2C1 ; CN1C[C@H]2C[C@@H]1CN2c1ncnc2c1c1ccc(S(=O)(=O)NC3(C#N)CC3)cc1n2-c1nnc(C(F)F)s1]

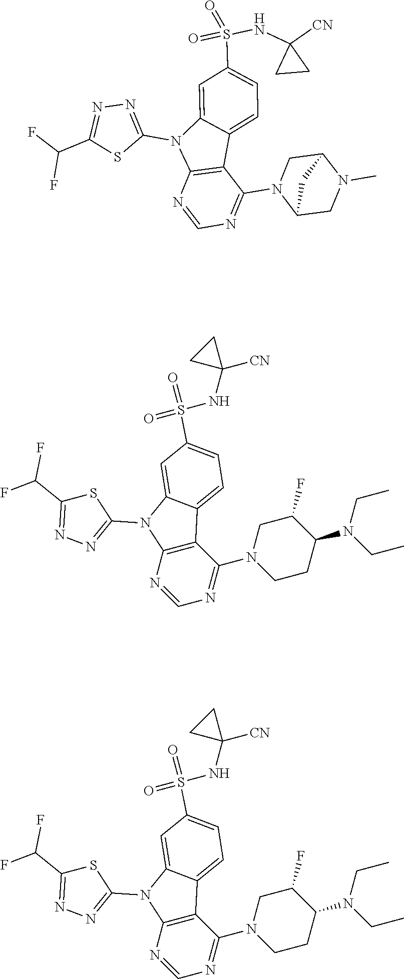 [SMILES: CN1C[C@@H]2C[C@H]1CN2c1ncnc2c1c1ccc(S(=O)(=O)NC3(C#N)CC3)cc1n2-c1nnc(C(F)F)s1 ; CCN(CC)[C@H]1CCN(c2ncnc3c2c2ccc(S(=O)(=O)NC4(C#N)CC4)cc2n3-c2nnc(C(F)F)s2)C[C@@H]1F ; CCN(CC)[C@@H]1CCN(c2ncnc3c2c2ccc(S(=O)(=O)NC4(C#N)CC4)cc2n3-c2nnc(C(F)F)s2)C[C@@H]1F]

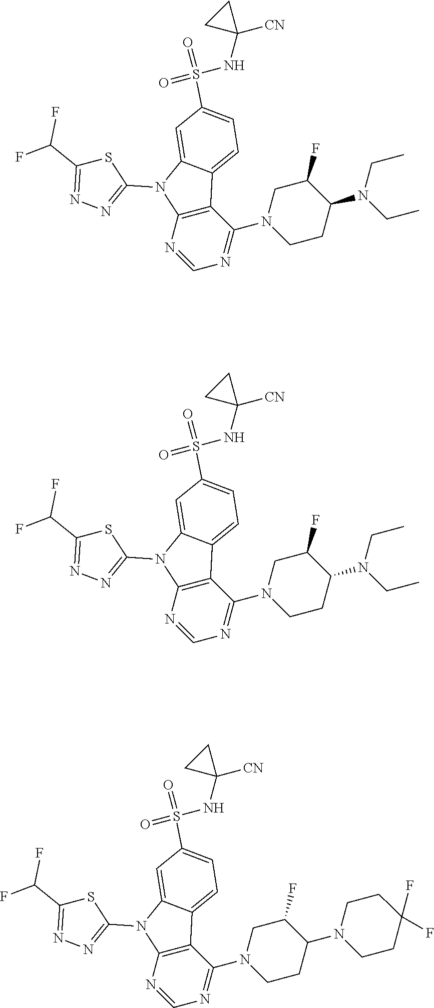 [SMILES: CCN(CC)[C@H]1CCN(c2ncnc3c2c2ccc(S(=O)(=O)NC4(C#N)CC4)cc2n3-c2nnc(C(F)F)s2)C[C@H]1F ; CCN(CC)[C@@H]1CCN(c2ncnc3c2c2ccc(S(=O)(=O)NC4(C#N)CC4)cc2n3-c2nnc(C(F)F)s2)C[C@H]1F ; N#CC1(NS(=O)(=O)c2ccc3c4c(N5CCC(N6CCC(F)(F)CC6)[C@@H](F)C5)ncnc4n(-c4nnc(C(F)F)s4)c3c2)CC1]

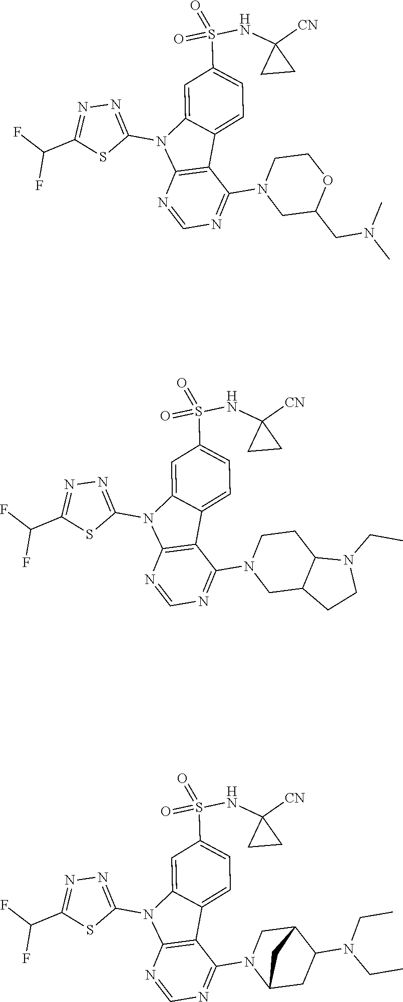 [SMILES: CN(C)CC1CN(c2ncnc3c2c2ccc(S(=O)(=O)NC4(C#N)CC4)cc2n3-c2nnc(C(F)F)s2)CCO1 ; CCN1CCC2CN(c3ncnc4c3c3ccc(S(=O)(=O)NC5(C#N)CC5)cc3n4-c3nnc(C(F)F)s3)CCC21 ; CCN(CC)C1C[C@H]2C[C@@H]1CN2c1ncnc2c1c1ccc(S(=O)(=O)NC3(C#N)CC3)cc1n2-c1nnc(C(F)F)s1]

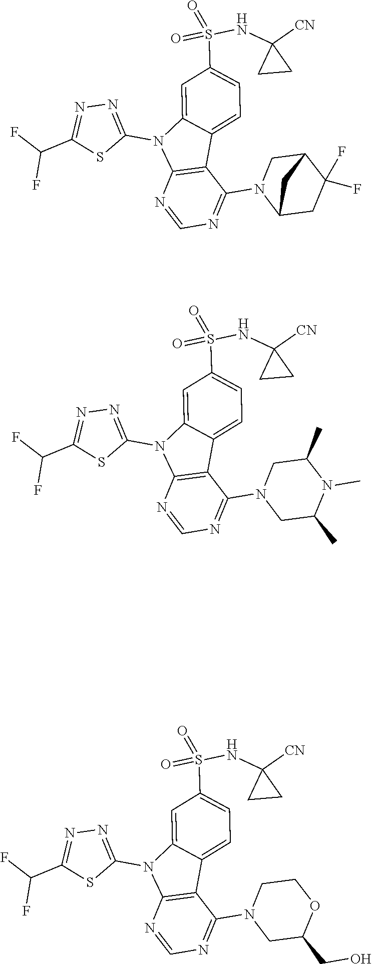 [SMILES: N#CC1(NS(=O)(=O)c2ccc3c4c(N5C[C@H]6C[C@@H]5CC6(F)F)ncnc4n(-c4nnc(C(F)F)s4)c3c2)CC1 ; C[C@@H]1CN(c2ncnc3c2c2ccc(S(=O)(=O)NC4(C#N)CC4)cc2n3-c2nnc(C(F)F)s2)C[C@H](C)N1C ; N#CC1(NS(=O)(=O)c2ccc3c4c(N5CCO[C@@H](CO)C5)ncnc4n(-c4nnc(C(F)F)s4)c3c2)CC1]

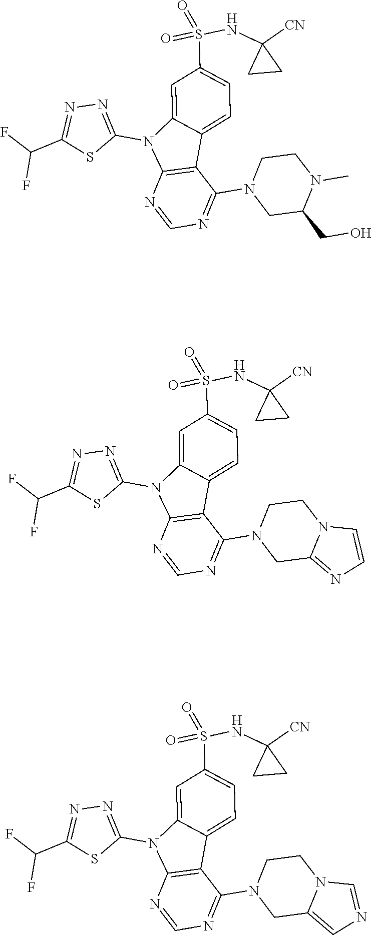 [SMILES: CN1CCN(c2ncnc3c2c2ccc(S(=O)(=O)NC4(C#N)CC4)cc2n3-c2nnc(C(F)F)s2)C[C@@H]1CO ; N#CC1(NS(=O)(=O)c2ccc3c4c(N5CCn6ccnc6C5)ncnc4n(-c4nnc(C(F)F)s4)c3c2)CC1 ; N#CC1(NS(=O)(=O)c2ccc3c4c(N5CCn6cncc6C5)ncnc4n(-c4nnc(C(F)F)s4)c3c2)CC1]

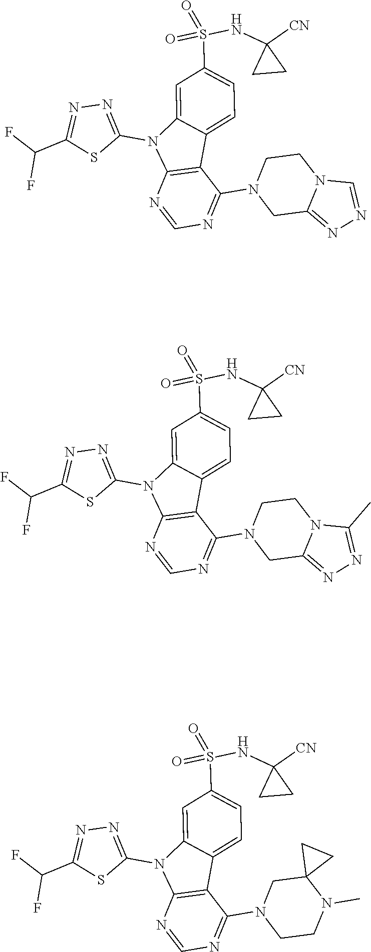 [SMILES: N#CC1(NS(=O)(=O)c2ccc3c4c(N5CCn6cnnc6C5)ncnc4n(-c4nnc(C(F)F)s4)c3c2)CC1 ; Cc1nnc2n1CCN(c1ncnc3c1c1ccc(S(=O)(=O)NC4(C#N)CC4)cc1n3-c1nnc(C(F)F)s1)C2 ; CN1CCN(c2ncnc3c2c2ccc(S(=O)(=O)NC4(C#N)CC4)cc2n3-c2nnc(C(F)F)s2)CC12CC2]

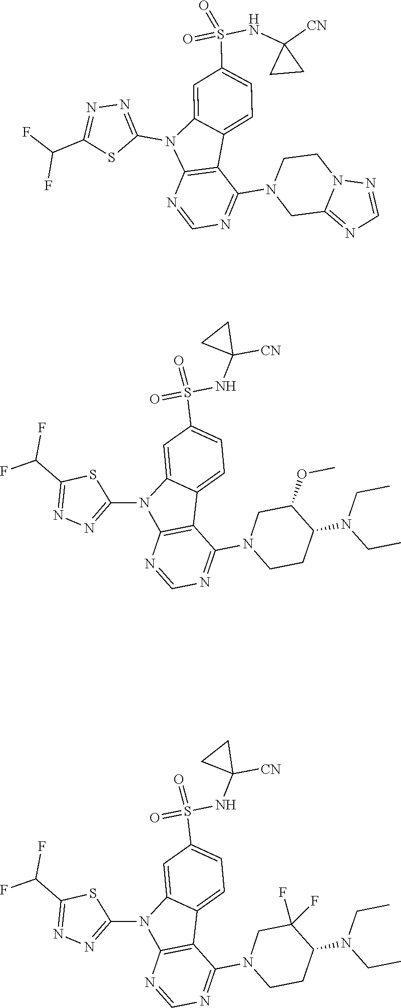 [SMILES: N#CC1(NS(=O)(=O)c2ccc3c4c(N5CCn6ncnc6C5)ncnc4n(-c4nnc(C(F)F)s4)c3c2)CC1 ; CCN(CC)[C@@H]1CCN(c2ncnc3c2c2ccc(S(=O)(=O)NC4(C#N)CC4)cc2n3-c2nnc(C(F)F)s2)C[C@@H]1OC ; CCN(CC)[C@@H]1CCN(c2ncnc3c2c2ccc(S(=O)(=O)NC4(C#N)CC4)cc2n3-c2nnc(C(F)F)s2)CC1(F)F]

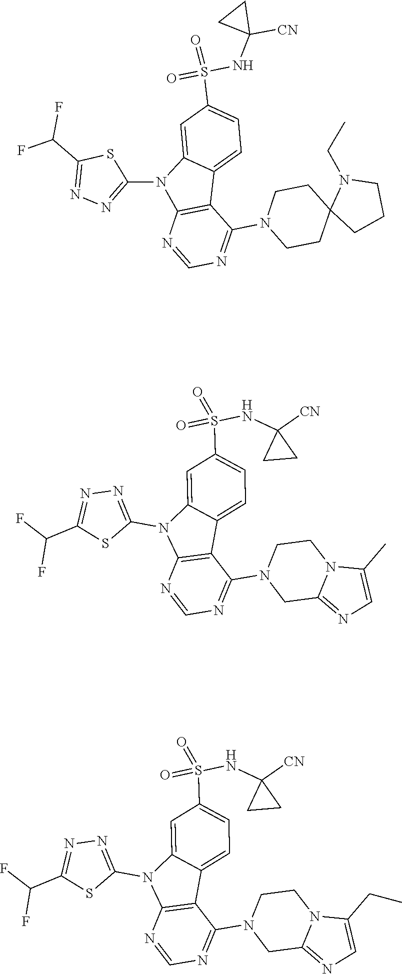 [SMILES: CCN1CCCC12CCN(c1ncnc3c1c1ccc(S(=O)(=O)NC4(C#N)CC4)cc1n3-c1nnc(C(F)F)s1)CC2 ; Cc1cnc2n1CCN(c1ncnc3c1c1ccc(S(=O)(=O)NC4(C#N)CC4)cc1n3-c1nnc(C(F)F)s1)C2 ; CCc1cnc2n1CCN(c1ncnc3c1c1ccc(S(=O)(=O)NC4(C#N)CC4)cc1n3-c1nnc(C(F)F)s1)C2]

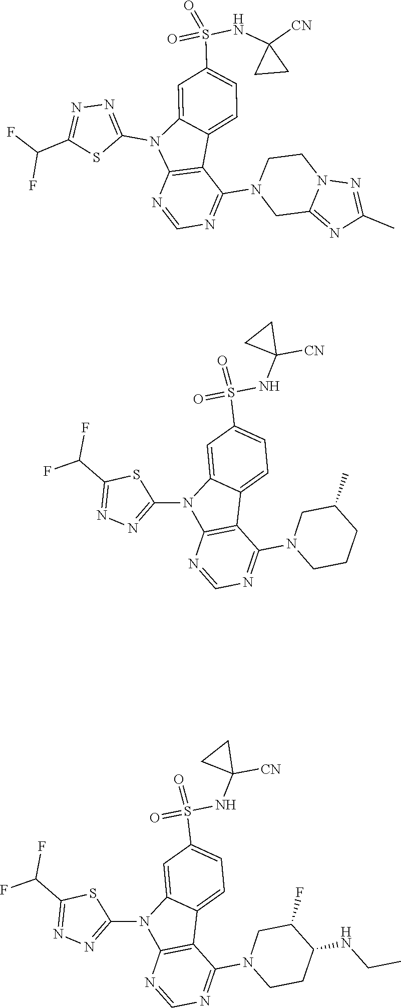 [SMILES: Cc1nc2n(n1)CCN(c1ncnc3c1c1ccc(S(=O)(=O)NC4(C#N)CC4)cc1n3-c1nnc(C(F)F)s1)C2 ; C[C@@H]1CCCN(c2ncnc3c2c2ccc(S(=O)(=O)NC4(C#N)CC4)cc2n3-c2nnc(C(F)F)s2)C1 ; CCN[C@@H]1CCN(c2ncnc3c2c2ccc(S(=O)(=O)NC4(C#N)CC4)cc2n3-c2nnc(C(F)F)s2)C[C@@H]1F]

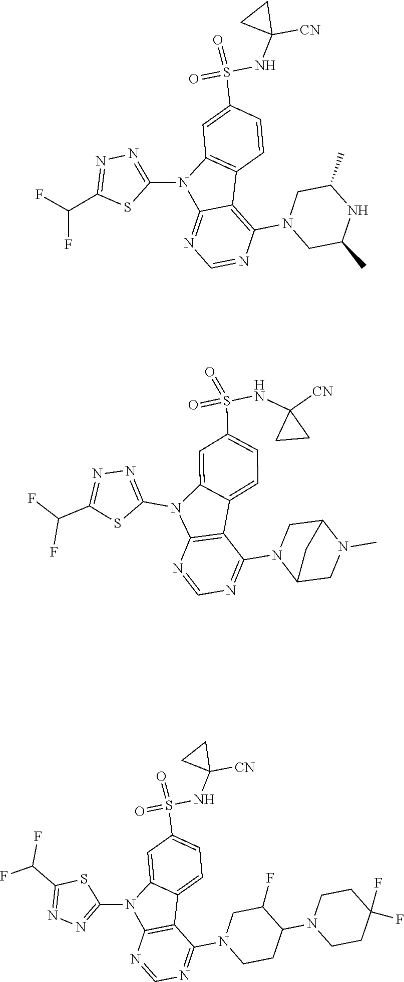 [SMILES: C[C@H]1CN(c2ncnc3c2c2ccc(S(=O)(=O)NC4(C#N)CC4)cc2n3-c2nnc(C(F)F)s2)C[C@H](C)N1 ; CN1CC2CC1CN2c1ncnc2c1c1ccc(S(=O)(=O)NC3(C#N)CC3)cc1n2-c1nnc(C(F)F)s1 ; N#CC1(NS(=O)(=O)c2ccc3c4c(N5CCC(N6CCC(F)(F)CC6)C(F)C5)ncnc4n(-c4nnc(C(F)F)s4)c3c2)CC1]

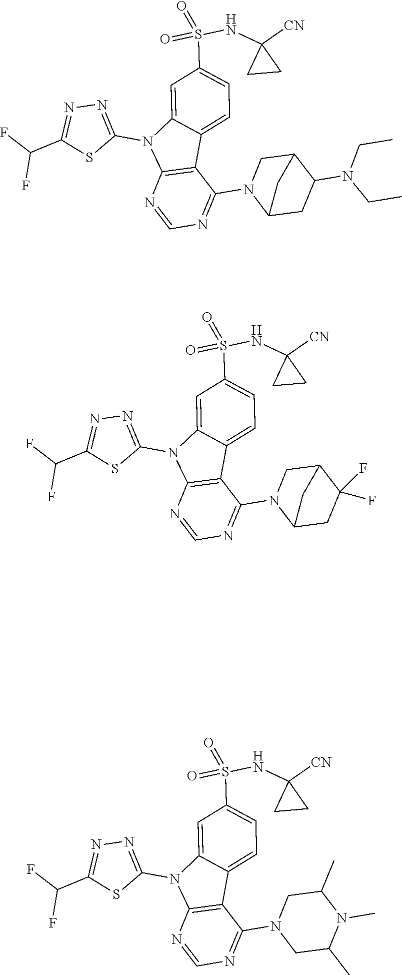 [SMILES: CCN(CC)C1CC2CC1CN2c1ncnc2c1c1ccc(S(=O)(=O)NC3(C#N)CC3)cc1n2-c1nnc(C(F)F)s1 ; N#CC1(NS(=O)(=O)c2ccc3c4c(N5CC6CC5CC6(F)F)ncnc4n(-c4nnc(C(F)F)s4)c3c2)CC1 ; CC1CN(c2ncnc3c2c2ccc(S(=O)(=O)NC4(C#N)CC4)cc2n3-c2nnc(C(F)F)s2)CC(C)N1C]

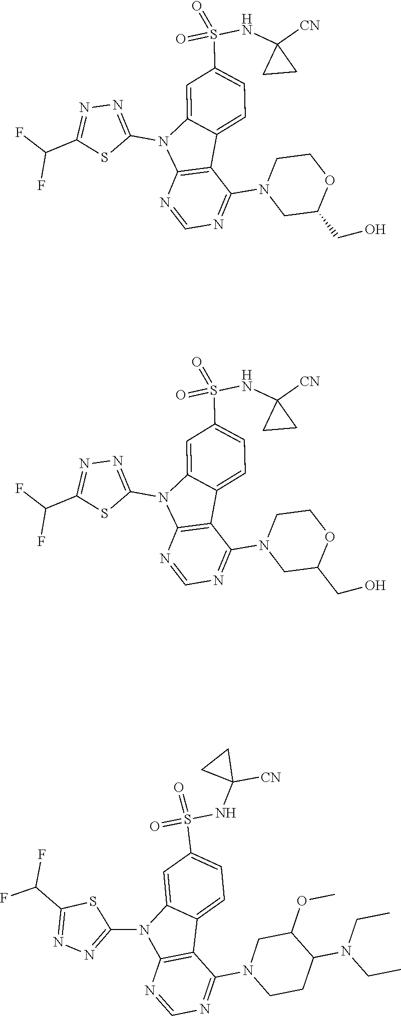 [SMILES: N#CC1(NS(=O)(=O)c2ccc3c4c(N5CCO[C@H](CO)C5)ncnc4n(-c4nnc(C(F)F)s4)c3c2)CC1 ; N#CC1(NS(=O)(=O)c2ccc3c4c(N5CCOC(CO)C5)ncnc4n(-c4nnc(C(F)F)s4)c3c2)CC1 ; CCN(CC)C1CCN(c2ncnc3c2c2ccc(S(=O)(=O)NC4(C#N)CC4)cc2n3-c2nnc(C(F)F)s2)CC1OC]

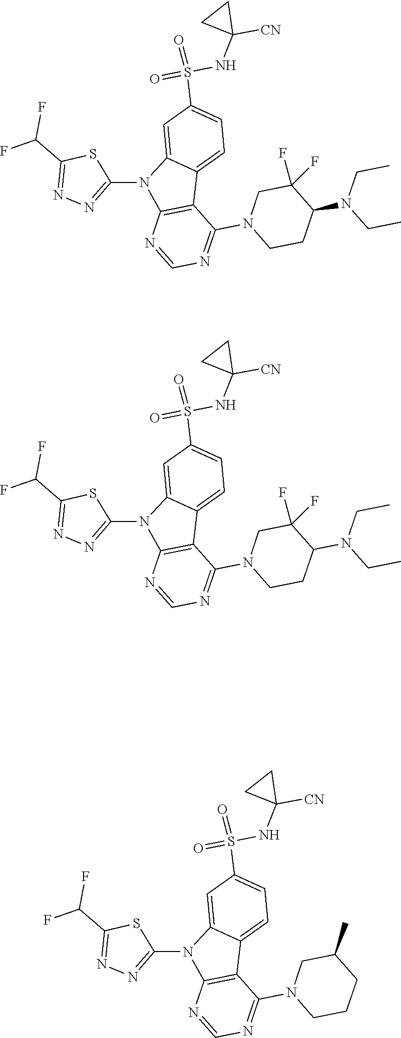 [SMILES: CCN(CC)[C@H]1CCN(c2ncnc3c2c2ccc(S(=O)(=O)NC4(C#N)CC4)cc2n3-c2nnc(C(F)F)s2)CC1(F)F ; CCN(CC)C1CCN(c2ncnc3c2c2ccc(S(=O)(=O)NC4(C#N)CC4)cc2n3-c2nnc(C(F)F)s2)CC1(F)F ; C[C@H]1CCCN(c2ncnc3c2c2ccc(S(=O)(=O)NC4(C#N)CC4)cc2n3-c2nnc(C(F)F)s2)C1]

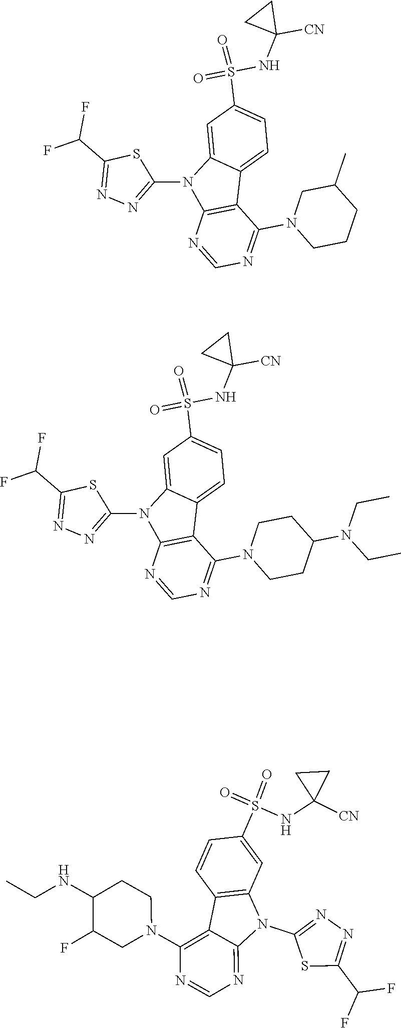

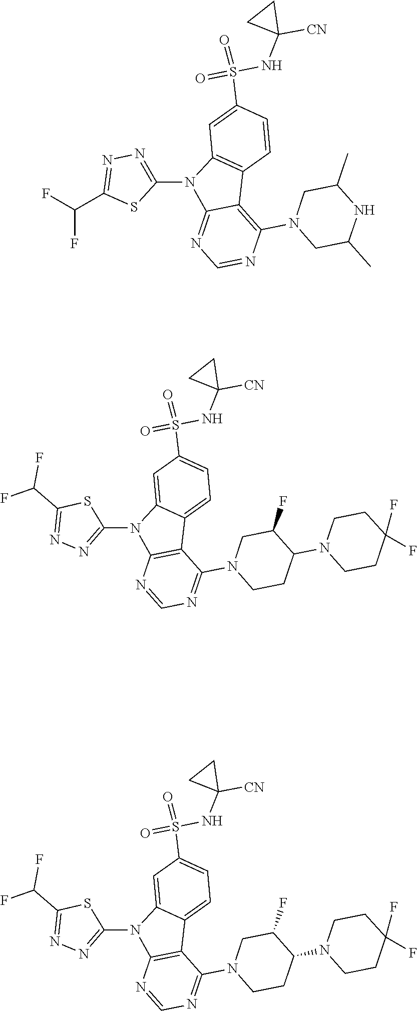 [SMILES: CC1CN(c2ncnc3c2c2ccc(S(=O)(=O)NC4(C#N)CC4)cc2n3-c2nnc(C(F)F)s2)CC(C)N1 ; N#CC1(NS(=O)(=O)c2ccc3c4c(N5CCC(N6CCC(F)(F)CC6)[C@H](F)C5)ncnc4n(-c4nnc(C(F)F)s4)c3c2)CC1 ; N#CC1(NS(=O)(=O)c2ccc3c4c(N5CC[C@@H](N6CCC(F)(F)CC6)[C@@H](F)C5)ncnc4n(-c4nnc(C(F)F)s4)c3c2)CC1]

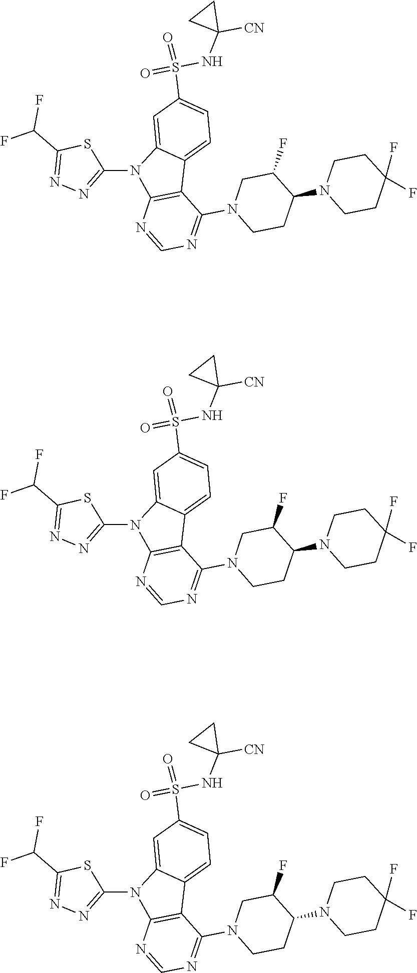 [SMILES: N#CC1(NS(=O)(=O)c2ccc3c4c(N5CC[C@H](N6CCC(F)(F)CC6)[C@@H](F)C5)ncnc4n(-c4nnc(C(F)F)s4)c3c2)CC1 ; N#CC1(NS(=O)(=O)c2ccc3c4c(N5CC[C@H](N6CCC(F)(F)CC6)[C@H](F)C5)ncnc4n(-c4nnc(C(F)F)s4)c3c2)CC1 ; N#CC1(NS(=O)(=O)c2ccc3c4c(N5CC[C@@H](N6CCC(F)(F)CC6)[C@H](F)C5)ncnc4n(-c4nnc(C(F)F)s4)c3c2)CC1]

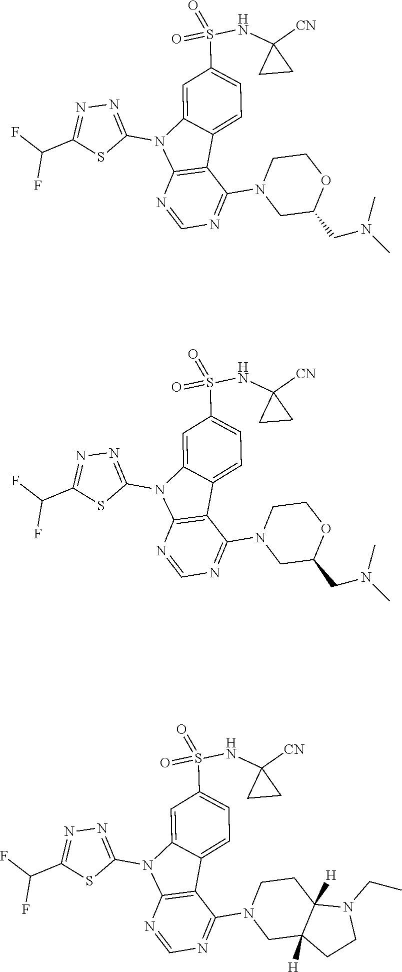 [SMILES: CN(C)C[C@@H]1CN(c2ncnc3c2c2ccc(S(=O)(=O)NC4(C#N)CC4)cc2n3-c2nnc(C(F)F)s2)CCO1 ; CN(C)C[C@H]1CN(c2ncnc3c2c2ccc(S(=O)(=O)NC4(C#N)CC4)cc2n3-c2nnc(C(F)F)s2)CCO1 ; CCN1CC[C@@H]2CN(c3ncnc4c3c3ccc(S(=O)(=O)NC5(C#N)CC5)cc3n4-c3nnc(C(F)F)s3)CC[C@@H]21]

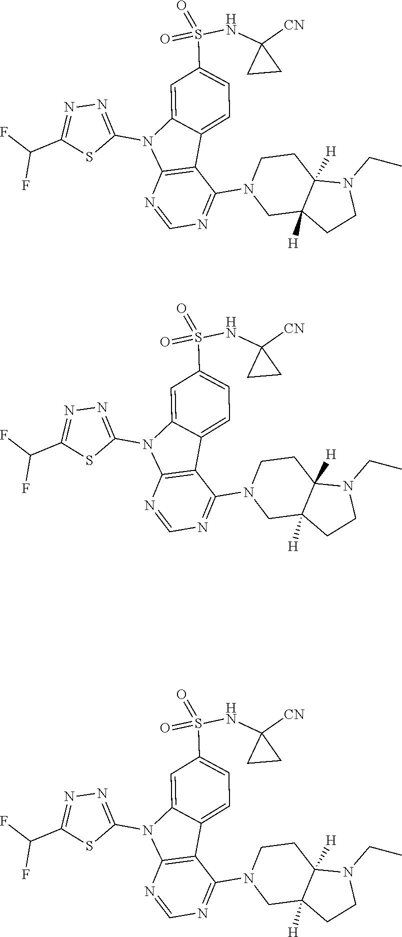 [SMILES: CCN1CC[C@@H]2CN(c3ncnc4c3c3ccc(S(=O)(=O)NC5(C#N)CC5)cc3n4-c3nnc(C(F)F)s3)CC[C@H]21 ; CCN1CC[C@H]2CN(c3ncnc4c3c3ccc(S(=O)(=O)NC5(C#N)CC5)cc3n4-c3nnc(C(F)F)s3)CC[C@@H]21 ; CCN1CC[C@H]2CN(c3ncnc4c3c3ccc(S(=O)(=O)NC5(C#N)CC5)cc3n4-c3nnc(C(F)F)s3)CC[C@H]21]

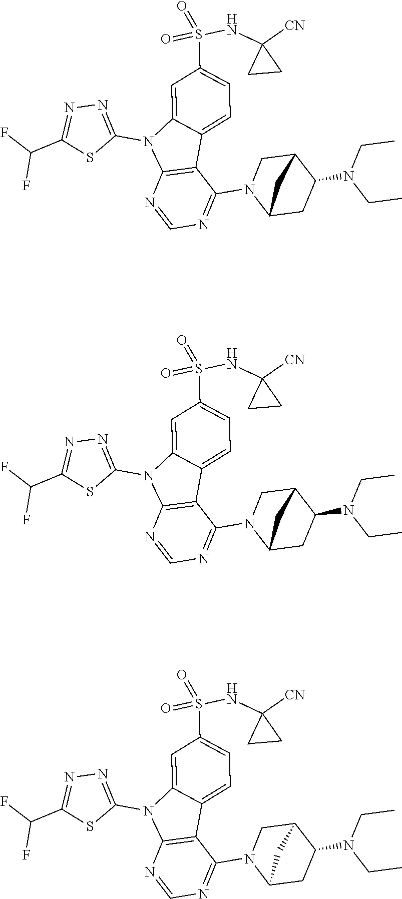 [SMILES: CCN(CC)[C@@H]1C[C@H]2C[C@@H]1CN2c1ncnc2c1c1ccc(S(=O)(=O)NC3(C#N)CC3)cc1n2-c1nnc(C(F)F)s1 ; CCN(CC)[C@H]1C[C@H]2C[C@@H]1CN2c1ncnc2c1c1ccc(S(=O)(=O)NC3(C#N)CC3)cc1n2-c1nnc(C(F)F)s1 ; CCN(CC)[C@@H]1C[C@@H]2C[C@H]1CN2c1ncnc2c1c1ccc(S(=O)(=O)NC3(C#N)CC3)cc1n2-c1nnc(C(F)F)s1]

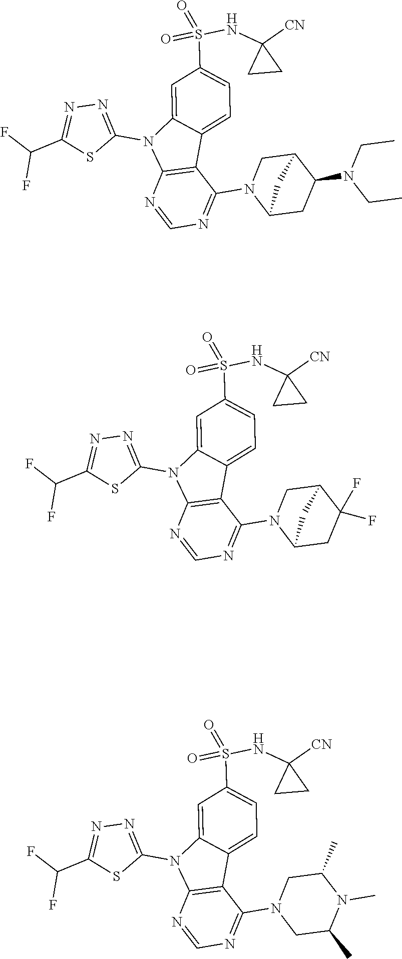 [SMILES: CCN(CC)[C@H]1C[C@@H]2C[C@H]1CN2c1ncnc2c1c1ccc(S(=O)(=O)NC3(C#N)CC3)cc1n2-c1nnc(C(F)F)s1 ; N#CC1(NS(=O)(=O)c2ccc3c4c(N5C[C@@H]6C[C@H]5CC6(F)F)ncnc4n(-c4nnc(C(F)F)s4)c3c2)CC1 ; C[C@H]1CN(c2ncnc3c2c2ccc(S(=O)(=O)NC4(C#N)CC4)cc2n3-c2nnc(C(F)F)s2)C[C@H](C)N1C]

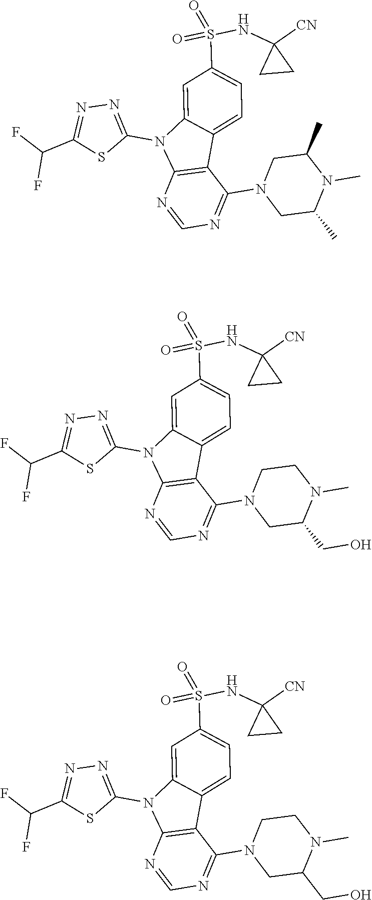 [SMILES: C[C@@H]1CN(c2ncnc3c2c2ccc(S(=O)(=O)NC4(C#N)CC4)cc2n3-c2nnc(C(F)F)s2)C[C@@H](C)N1C ; CN1CCN(c2ncnc3c2c2ccc(S(=O)(=O)NC4(C#N)CC4)cc2n3-c2nnc(C(F)F)s2)C[C@H]1CO ; CN1CCN(c2ncnc3c2c2ccc(S(=O)(=O)NC4(C#N)CC4)cc2n3-c2nnc(C(F)F)s2)CC1CO]

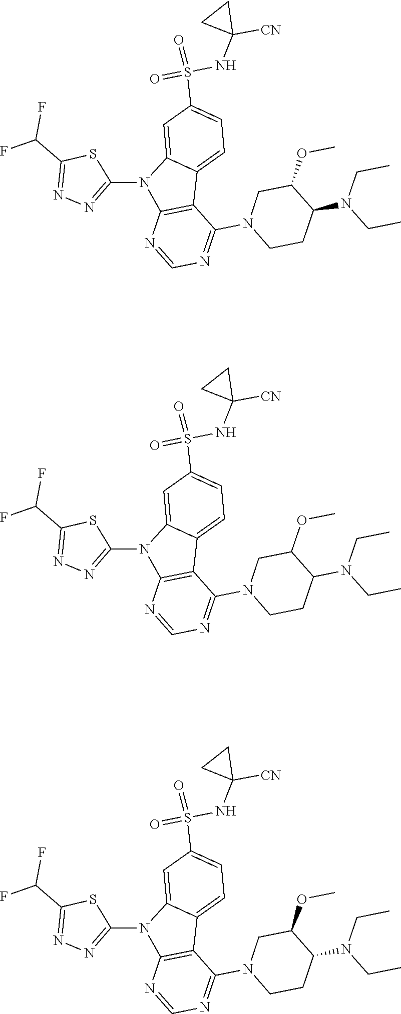 [SMILES: CCN(CC)[C@H]1CCN(c2ncnc3c2c2ccc(S(=O)(=O)NC4(C#N)CC4)cc2n3-c2nnc(C(F)F)s2)C[C@@H]1OC ; CCN(CC)C1CCN(c2ncnc3c2c2ccc(S(=O)(=O)NC4(C#N)CC4)cc2n3-c2nnc(C(F)F)s2)CC1OC ; CCN(CC)[C@@H]1CCN(c2ncnc3c2c2ccc(S(=O)(=O)NC4(C#N)CC4)cc2n3-c2nnc(C(F)F)s2)C[C@H]1OC]

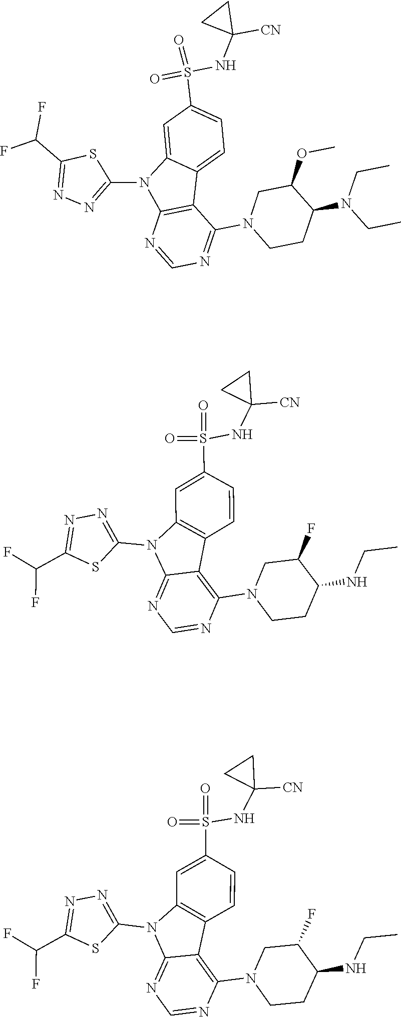 [SMILES: CCN(CC)[C@H]1CCN(c2ncnc3c2c2ccc(S(=O)(=O)NC4(C#N)CC4)cc2n3-c2nnc(C(F)F)s2)C[C@H]1OC ; CCN[C@@H]1CCN(c2ncnc3c2c2ccc(S(=O)(=O)NC4(C#N)CC4)cc2n3-c2nnc(C(F)F)s2)C[C@H]1F ; CCN[C@H]1CCN(c2ncnc3c2c2ccc(S(=O)(=O)NC4(C#N)CC4)cc2n3-c2nnc(C(F)F)s2)C[C@@H]1F]

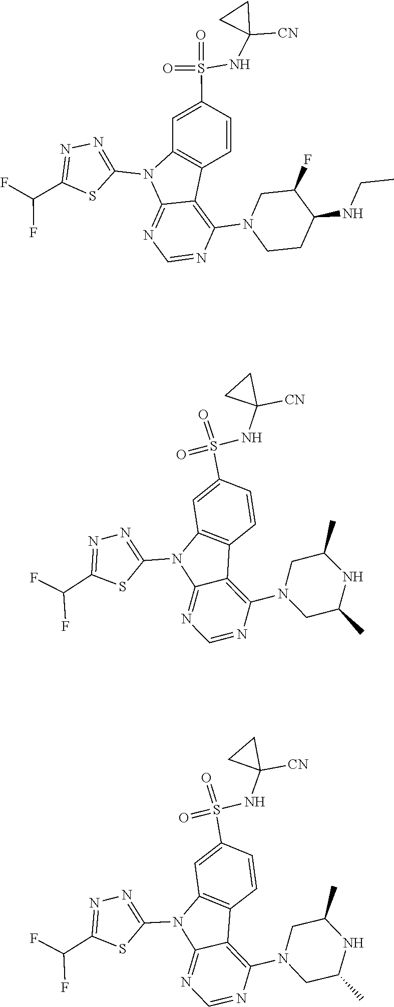 [SMILES: CCN[C@H]1CCN(c2ncnc3c2c2ccc(S(=O)(=O)NC4(C#N)CC4)cc2n3-c2nnc(C(F)F)s2)C[C@H]1F ; C[C@@H]1CN(c2ncnc3c2c2ccc(S(=O)(=O)NC4(C#N)CC4)cc2n3-c2nnc(C(F)F)s2)C[C@H](C)N1 ; C[C@@H]1CN(c2ncnc3c2c2ccc(S(=O)(=O)NC4(C#N)CC4)cc2n3-c2nnc(C(F)F)s2)C[C@@H](C)N1]

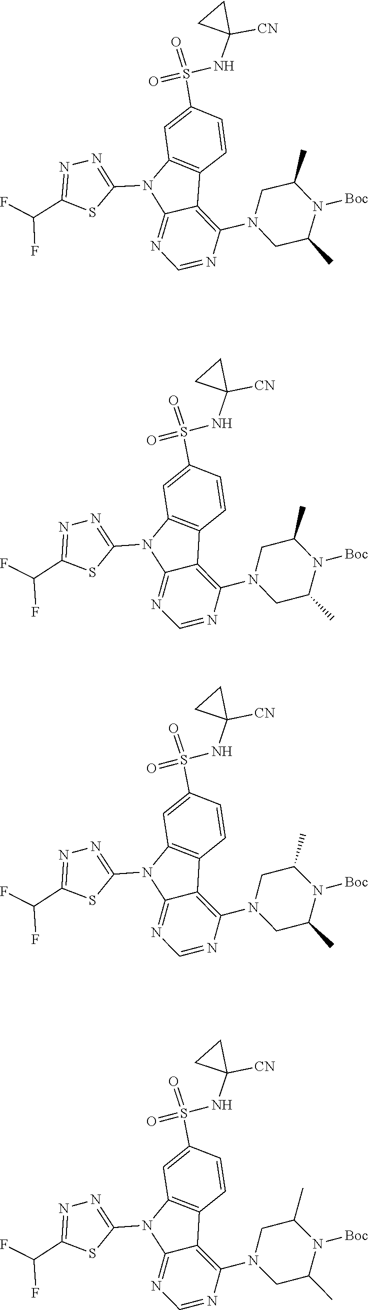 [SMILES: C[C@@H]1CN(c2ncnc3c2c2ccc(S(=O)(=O)NC4(C#N)CC4)cc2n3-c2nnc(C(F)F)s2)C[C@H](C)N1C(=O)OC(C)(C)C ; C[C@@H]1CN(c2ncnc3c2c2ccc(S(=O)(=O)NC4(C#N)CC4)cc2n3-c2nnc(C(F)F)s2)C[C@@H](C)N1C(=O)OC(C)(C)C ; C[C@H]1CN(c2ncnc3c2c2ccc(S(=O)(=O)NC4(C#N)CC4)cc2n3-c2nnc(C(F)F)s2)C[C@H](C)N1C(=O)OC(C)(C)C ; CC1CN(c2ncnc3c2c2ccc(S(=O)(=O)NC4(C#N)CC4)cc2n3-c2nnc(C(F)F)s2)CC(C)N1C(=O)OC(C)(C)C]

or a pharmaceutically acceptable salt thereof.

It will be apparent that the compounds of Formula I, including all subgenera described herein, may have multiple stereogenic centers. As a result, there exist multiple stereoisomers (enantiomers and diastereomers) of the compounds of Formula I (and subgenera described herein). The present disclosure contemplates and encompasses each stereoisomer of any compound of Formula I (and subgenera described herein), as well as mixtures of said stereoisomers.

Pharmaceutically acceptable salts and solvates of the compounds of Formula I (including all subgenera described herein) are also within the scope of the disclosure.

Isotopic variants of the compounds of Formula I (including all subgenera described herein) are also contemplated by the present disclosure.

The present disclosure further provides compounds described herein, or a pharmaceutically acceptable salt thereof, for use in any of the methods described herein. The present disclosure further provides uses of a compound described herein, or a pharmaceutically acceptable salt thereof, for the preparation of a medicament for use in any of the methods described herein.

The present disclosure further provides pharmaceutical compositions comprising a compound described herein, or a pharmaceutically acceptable salt thereof, and a pharmaceutically acceptable carrier.

The PARG inhibitors of the present disclosure may be useful in the treatment of various types of cancer, including but not limited to breast, ovarian, gastric, prostate, pancreatic, uterine, cervical, endometrial, lung, brain, bile duct and hematological cancers.

Routs of administration for the compounds in the present disclosure include, but not limited to oral, injection, topical and inhalation.

The compounds of the present disclosure may be used as single agent or combined with other treatments. Such treatment may include one or more of the following categories of cancer therapies: such as surgery, chemotherapies, radiation therapies, targeted therapy (for example kinase inhibitors, growth factor inhibitors, cyclin dependent kinase inhibitors and so on), other DDR modulators (for example DNA-PK inhibitor, ATM inhibitor, ATR inhibitor, CHK1 inhibitor, WEE1 inhibitor, CDK1 inhibitor, LIG4 inhibitor, HIF-1 inhibitor, HDAC inhibitor, RAD51 inhibitor, Polθ inhibitor, WRN inhibitor, PRMT5 inhibitor, MAT2A inhibitor and PKMYT1 inhibitor and so on), immunotherapies, and gene and cell therapy approaches.

In some embodiments, provided herein is an intermediate compound of Formula (A):

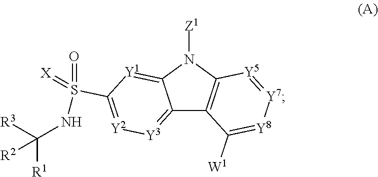

W¹ is a leaving group (such as halogen (e.g., Cl, Br, or I), pseudohalogen (e.g., C₁-C₃ alkyl-SO₂—, phenyl-SO₂— (such as OTf, OTs or OMs), —SC₁-C₄ alkyl (e.g., —SCH₃, —SCH₂CH₃), —S— phenyl, —OC₁-C₄ alkyl, —OC₁-C₄ haloalkyl (e.g., —OCF₂CF₃); wherein, the C₁-C₄ alkyl, phenyl is optionally substituted by halo, CN, NO₂, SF₅, OH, C₁-C₄ alkyl, C₁-C₄haloalkyl, —O—C₁-C₄ alkyl, —OC₁-C₄haloalkyl);
wherein, R¹, R², R³, X, Y¹, Y², Y³, Y⁵, Y⁷, Y⁸, and Z¹ are each as defined herein.

In some embodiments, W¹ is halogen, C₁-C₃ alkyl-SO₂—, phenyl-SO₂—, —SC₁-C₄ alkyl, —S— phenyl, —OC₁-C₄ alkyl, —OC₁-C₄ haloalkyl; wherein, the C₁-C₄ alkyl, phenyl is optionally substituted by halo, CN, NO₂, SF₅, OH, C₁-C₄ alkyl, C₁-C₄haloalkyl, —O—C₁-C₄ alkyl, —OC₁-C₄ haloalkyl.

In some embodiments, W¹ is F, Cl, Br, I, OTf, OTs, OMs, —SCH₃, —SCH₂CH₃, —S-phenyl, —OCF₂CF₃.

In another embodiments, provided herein is the intermediate compound of Formula (Aa):

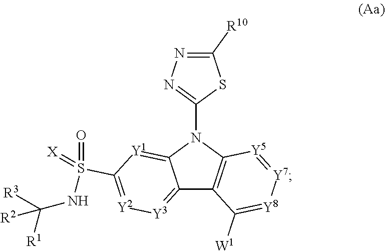

wherein, W¹, R¹, R², R³, R¹⁰, X, Y¹, Y^{Z}, Y³, Y⁵, Y⁷, Y⁸, and Z¹ are each as defined herein.

In another embodiments, provided herein is the intermediate compound of Formula (Ab):

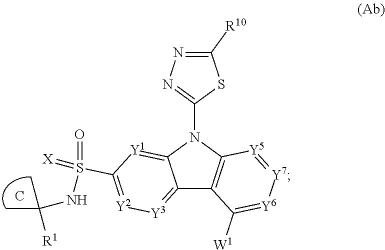

wherein, W¹, ring C, R R¹⁰, X, Y y, Y³, Y⁵, Y⁷, Y⁸, and Z¹ are each as defined herein.

In one embodiment, provided herein the intermediate compound is:

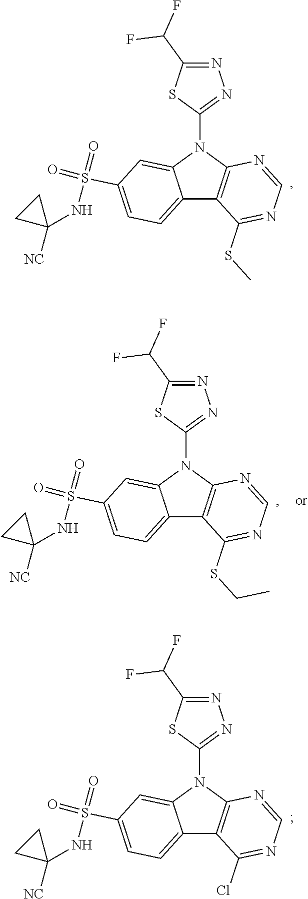 [SMILES: CSc1ncnc2c1c1ccc(S(=O)(=O)NC3(C#N)CC3)cc1n2-c1nnc(C(F)F)s1 ; CCSc1ncnc2c1c1ccc(S(=O)(=O)NC3(C#N)CC3)cc1n2-c1nnc(C(F)F)s1 ; N#CC1(NS(=O)(=O)c2ccc3c4c(Cl)ncnc4n(-c4nnc(C(F)F)s4)c3c2)CC1]

or salts thereof.

In some embodiments, provided herein is an intermediate compound of Formula (B):

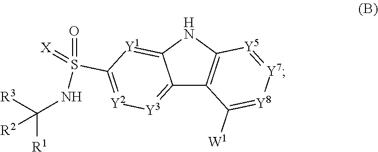

W² is a leaving group (such as, —SC₁-C₄ alkyl (e.g., —SCH₃, —SCH₂CH₃), —S-phenyl, —OC₁-C₄ alkyl, —OC₁-C₄ haloalkyl (e.g., —OCF₂CF₃); wherein, the C₁-C₄ alkyl, phenyl is optionally substituted by halo, CN, NO₂, SF₅, OH, C₁-C₄ alkyl, C₁-C₄haloalkyl, —O—C₁-C₄ alkyl, —OC₁-C₄ haloalkyl);
wherein, R¹, R², R³, X, Y¹, Y², Y³, Y⁵, Y⁷, and Y⁸ are each as defined herein.

In some embodiments, W² is —SC₁-C₄ alkyl, —S-phenyl, —OC₁-C₄ alkyl, or —OC₁-C₄ haloalkyl; wherein, the C₁-C₄ alkyl, phenyl is optionally substituted by halo, CN, NO₂, SF₅, OH, C₁-C₄ alkyl, C₁-C₄haloalkyl, —O—C₁-C₄ alkyl, —OC₁-C₄haloalkyl.

In some embodiments, W² is —SCH₃, —SCH₂CH₃, —S-phenyl, or —OCF₂CF₃.

In another embodiments, provided herein is the intermediate compound of Formula (Ba):

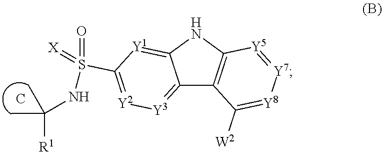

wherein, W², ring C, R¹, X, Y¹, Y², Y³, Y⁵, Y⁷, and Y⁸ are each as defined herein.

In one embodiment, provided herein the intermediate compound is:

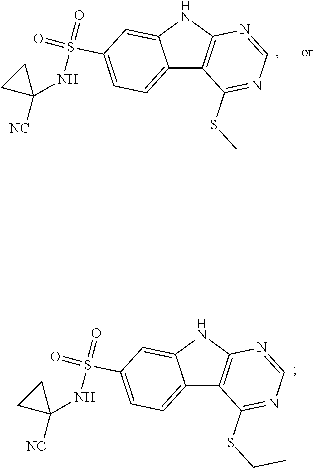 [SMILES: CSc1ncnc2[nH]c3cc(S(=O)(=O)NC4(C#N)CC4)ccc3c12 ; CCSc1ncnc2[nH]c3cc(S(=O)(=O)NC4(C#N)CC4)ccc3c12]

or salts thereof.

### Definitions

Unless other indicated, the following terms are intended to have the meaning set forth below. Other terms are defined elsewhere throughout the specification.

As used herein, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology such as “solely”, “only” and the like in connection with the recitation of claim elements, or use of a “negative” limitation.

At various places in the present specification, variables defining divalent linking groups are described. It is specifically intended that each linking substituent include both the forward and backward forms of the linking substituent. For example, —NR(CR′R″)— includes both —NR(CR′R″)— and —(CR′R″)NR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cn-Cm” indicates a range which includes the endpoints, wherein n and m are integers and indicate the number of carbons. For example, the term “C₁-C₆ alkyl” is specifically intended to individually disclose methyl, ethyl, C₃ alkyl, C₄ alkyl, C₅ alkyl, and C₆ alkyl. “C₀ alkyl” refers to a covalent bond.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

As used herein, unless otherwise indicated, the term “alkyl”, by itself or as part of another substituent, is meant to refer to a saturated hydrocarbon group which is straight-chained or branched. An alkyl group can contain from 1 to about 20, from 2 to about 20, from 1 to about 10, from 1 to about 8, from 1 to about 6, from 1 to about 4, or from 1 to about 3 carbon atoms. Similarly, C₁₋₈, as in C₁₋₈ alkyl is defined to identify the group as having 1, 2, 3, 4, 5, 6, 7 or 8 carbon atoms in a linear or branched arrangement. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like.

As used herein, unless otherwise indicated, “alkenyl” refers to an alkyl group having one or more double carbon-carbon bonds. Example alkenyl groups include, but are not limited to, ethenyl, propenyl, and the like.

As used herein, unless otherwise indicated, “alkynyl” refers to an alkyl group having one or more triple carbon-carbon bonds. Example alkynyl groups include, but are not limited to, ethynyl, propynyl, and the like.

As used herein, unless otherwise indicated, “haloalkyl” refers to an alkyl group having one or more halogen substituents. Example haloalkyl groups include, but are not limited to, CF₃, C₂F₅, CHF₂, CH₂F, CCl₃, CHCl₂, C₂Cl₅, and the like.

As used herein, unless otherwise indicated, “aryl” refers to an unsubstituted or substituted monocyclic or polycyclic (e.g., having 2, 3 or 4 fused rings) aromatic hydrocarbons. In some embodiments, aryl groups have from 6 to about 20 carbon atoms. In some embodiments, aryl groups have from 6 to about 14 carbon atoms. In some embodiments, aryl groups have from 6 to about 10 carbon atoms. Example aryl groups include, but are not limited to, phenyl, naphthyl, anthracenyl, phenanthrenyl, indanyl, indenyl, and the like.

As used herein, unless otherwise indicated, “cycloalkyl” refers to an unsubstituted or substituted non-aromatic carbocycles (saturated or partially unsaturated ring) including cyclized alkyl, alkenyl, and alkynyl groups. Cycloalkyl groups can include mono- or polycyclic (e.g., having 2, 3 or 4 fused rings) ring systems, including fused rings, spirocyclic rings, and bridged rings (e.g., a bridged bicycloalkyl group). In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Cycloalkyl groups can be optionally substituted by oxo or sulfido (e.g., —C(O)— or —C(S)—). Also included in the definition of cycloalkyl are moieties that have one or more aromatic rings fused (i.e., having a bond in common with) to the cycloalkyl ring, for example, benzo derivatives of pentane, pentene, hexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent. In some embodiments, the cycloalkyl is a C₃-C₇ monocyclic cycloalkyl. In some embodiments, the cycloalkyl is a C₄-C₁₀ spirocycle or bridged cycloalkyl. Example cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl. In some embodiments, cycloalkyl are cyclic-containing, non-aromatic hydrocarbon groups having from 3 to 12 carbon atoms (“C₃-C₁₂”), preferably from 3 to 6 carbon atoms (“C₃-C₆”). Examples of cycloalkyl groups include, for example, cyclopropyl (C₃; 3-membered), cyclobutyl (C₄; 4-membered), cyclopropylmethyl (C₄), cyclopentyl (C₅), cyclohexyl (C₆), 1-methylcyclopropyl (C₄), 2-methylcyclopentyl (C₄), adamantanyl (C₁₀), and the like.

The term “spirocycloalkyl” when used alone or as part of a substituent group refers to a non-aromatic hydrocarbon group containing two cycloalkyl rings, and wherein the two cycloalyl rings share a single carbon atom in common.

As used herein, unless otherwise indicated, a “heteroaryl” group refers to an unsubstituted or substituted aromatic heterocycle having at least one heteroatom ring member such as boron, sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, unless otherwise indicated, “heterocycloalkyl” refers to an unsubstituted or substituted monocyclic (saturated or partially unsaturated ring) or polycyclic heterocycles having at least one non-aromatic ring (saturated or partially unsaturated ring), wherein one or more of the ring-forming carbon atoms of the heterocycloalkyl is replaced by a heteroatom selected from N, O, S, Si, P and B, and wherein the ring-forming carbon atoms and heteroatoms of the heterocycloalkyl group can be optionally substituted by one or more oxo or sulfido (e.g., C(0), S(O), C(S), S(O)₂, or P(O), etc.). Heterocycloalkyl groups include monocyclic and polycyclic (e.g., having 2 fused rings) systems. Included in heterocycloalkyl are monocyclic and polycyclic 3-10, 4-10, 3-7, 4-7, and 5-6 membered heterocycloalkyl groups. Heterocycloalkyl groups can also include spirocycles and bridged rings (e.g., a 5-10 membered bridged biheterocycloalkyl ring having one or more of the ring-forming carbon atoms replaced by a heteroatom independently selected from N, O, S, Si, P and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S, Si and B and having one or more oxidized ring members.

Example heterocycloalkyl groups include, but are not limited to, pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, 1,2,3,4-tetrahydroisoquinoline, azabicyclo[3.1.0]hexanyl, diazabicyclo[3.1.0]hexanyl, oxabicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, diazabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, oxa-azaspiro[3.5]nonanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl, octahydropyrrolo[3,4-c]pyrrolyl and the like.

In some embodiments, heterocycloalkyl refers to any three to ten membered monocyclic or bicyclic, saturated ring structure containing at least one heteroatom selected from the group consisting of O, N and S. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, and the like.

In some embodiments, the term “spiroheterocycloalkyl” when used alone or as part of a substituent group refers to a non-aromatic group containing two rings, at least one of which is a heterocycloalkyl ring, and wherein the two rings share a single carbon atom in common.

As used herein, unless otherwise indicated, “arylcycloalkyl” refers to cycloalkyl group substituted by an aryl group.

As used herein, unless otherwise indicated, “arylheterocycloalkyl” refers to a heterocycloalkyl group substituted by an aryl group.

As used herein, unless otherwise indicated, “arylheteroaryl” refers to a heteroaryl group substituted by an aryl group.

As used herein, unless otherwise indicated, “biaryl” refers to an aryl group substituted by another aryl group.

As used herein, unless otherwise indicated, “heteroarylcycloalkyl” refers to a cycloalkyl group substituted by a heteroaryl group.

As used herein, unless otherwise indicated, “heteroarylheterocycloalkyl” refers to a heterocycloalkyl group substituted by a heteroaryl group.

As used herein, unless otherwise indicated, “heteroarylaryl” refers to an aryl group substituted by a heteroaryl group.

As used herein, unless otherwise indicated, “biheteroaryl” refers to a heteroaryl group substituted by another heteroaryl group.

As used herein, “halo” or “halogen” includes fluoro, chloro, bromo, and iodo.

As used herein, unless otherwise indicated, “alkoxy” refers to an —O-alkyl group. Example alkoxy groups include methoxy, ethoxy, propoxy (e.g., n-propoxy and isopropoxy), t-butoxy, and the like.

As used herein, unless otherwise indicated, “hydroxylalkyl” refers to an alkyl group substituted by OH.

As used herein, unless otherwise indicated, “cyanoalkyl” refers to an alkyl group substituted by CN.

As used herein, unless otherwise indicated, “alkoxyalkyl” refers to an alkyl group substituted by an alkoxy group.

As used herein, unless otherwise indicated, “alkoxyalkoxy” refers to an alkoxy group substituted by alkoxy.

As used herein, unless otherwise indicated, “haloalkoxy” refers to an —O-(haloalkyl) group.

As used herein, unless otherwise indicated, “arylalkyl” refers to alkyl substituted by aryl and “cycloalkylalkyl” refers to alkyl substituted by cycloalkyl. An example arylalkyl group is benzyl.

As used herein, unless otherwise indicated, “heteroarylalkyl” refers to alkyl substituted by heteroaryl and “heterocycloalkylalkyl” refers to alkyl substituted by heterocycloalkyl.

As used herein, unless otherwise indicated, “oxo” refers to an oxygen substituent that is connected by a double bond (i.e., ═O).

As used herein, unless otherwise indicated, the phrase “optionally substituted” means unsubstituted or substituted.

As used herein, unless otherwise indicated, the term “substituted” refers to a group in which one or more hydrogen atoms are each independently replaced with the same or different substituent(s). Typical substituents include, but are not limited to, D, halo, oxo, C₁-C₆ alkyl, C₂-C₆ alkenyl, C₂-C₆ alkynyl, C₁-C₆ haloalkyl, C₁-C₆ alkyl-NR^{c}R^{d}, —(CH₂CH₂O), C₁-C₆alkyl wherein o is 1-10; C₂₋₆ alkenyl-NR^{c}R^{d}, C₂₋₆ alkynyl-NR^{c}R^{d}, OC₂₋₆ alkyl-NR^{c}R^{d}, CN, NO₂, N₃, OR^{a}, SR^{a}, C(O)R, C(O)NR^{c}R^{d}, —CH₂C(O)NR^{c}R^{d}, C(O)OR^{a}, OC(O)R^{b}, OC(O)NR^{c}R^{d}, —NR^{c}R^{d}, NR^{c}C(O)R^{b}, NR^{c}C(O)NR^{c}R^{d}, NR^{c}C(O)OR^{a}, C(═NR⁹)NR^{c}R^{d}, NR^{e}iC(═NR^{g})NR^{c}R^{d}, P(Rt)₂, P(OR^{e})₂, P(O)R^{e}R^{f} P(O)OR^{e}OR^{f}, S(O)R^{b}, —SO(═NR^{b}), S(O)NR^{c}R^{d}, S(O)₂R^{b}, NR^{c}S(O)₂R^{b}, S(O)₂NR^{c}R^{d}, aryl, heteroaryl, spirocycloalkyl, spiroheterocycloalkyl, cycloalkyl, or heterocycloalkyl, wherein the aryl, heteroaryl, spirocycloalkyl, spiroheterocycloalkyl, cycloalkyl, or heterocycloalkyl are optionally substituted with D, halo, oxo, C₁-C₆ alkyl, C₂-C₆ alkenyl, C₂-C₆ alkynyl, C₁-C₆ haloalkyl, C₁-C₆ alkyl-NR^{c}R^{d}, C₂₋₆ alkenyl-NR^{c1}R^{d1}, C₂₋₆ alkynyl-NR^{c1}R^{d1}OC₂₋₆ alkyl-NR^{c1}R^{d1}, CN, NO₂, N₃, OR^{a1}, SR^{a1}, C(O)R¹, C(O)NR^{c}R^{d1}, —CH₂C(O)NR^{c1}R^{d1}, C(O)OR^{a1}, OC(O)R^{b1}, OC(O)NR^{c1}R^{d1}, —NR^{c}R^{d}1, NR^{C}C(O)R^{b1}, NR^{c1}C(O)NR^{c1}R^{d1} NR^{c1} C(O)OR^{a1}, C(═NR^{g1})NR^{c1}R^{d1} NR^{c1}C(═NR^{g1})NR^{c1}R^{d1}, P(R^{f1})₂, P(OR^{e1})₂, P(O)R^{e1}R^{f1}, P(O)OR^{e1}OR^{f1}, S(O)R^{b1}, S(O)NR^{c1}R^{d1}, S(O)₂R^{b1}, NR^{c1}S(O)₂R^{b1}, S(O)₂NR^{c1}R^{d1} The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some cases, the compounds of the present disclosure may exist as rotational isomers. Descriptions of a compound of the invention that do not indicate a particular rotational isomer are intended to encompass any individual rotational isomers, as well as mixtures of rotational isomers in any proportion. Depiction of a particular rotational isomer is meant to refer to the depicted rotational isomer, substantially free of other rotational isomers.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the compounds of the invention, and salts thereof, are substantially isolated. By “substantially isolated” is meant that the compound is at least partially or substantially separated from the environment in which was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

A “leaving group” refers to an atom or a group of atoms that is displaced in a chemical reaction as stable species taking with it the bonding electrons, e.g., typically forming an anion. Preferably, a leaving group is the group comprising: halogen, in particular a fluoro, chloro, bromo, iodo, methylthio-, ethylthio-, —S-phenyl, —OCF₂CF₃, (methylsulfonyl)oxy-, [(4-methylphenyl)sulfonyl]oxy-, [(trifluoromethyl)sulfonyl]oxy-, [(nonafluorobutyl)sulfonyl]oxy-, [(4-bromophenyl)sulfonyl]oxy-, [(4-nitrophenyl)sulfonyl]oxy-, [(2-nitrophenyl)sulfonyl]oxy-, [(4-isopropylphenyl)sulfonyl]oxy-, [(2,4,6-triisopropylphenyl)sulfonyl]oxy-, [(2,4,6-trimethylphenyl)sulfonyl]oxy-, [(4-tert-butylphenyl)sulfonyl]oxy-, (phenylsulfonyl)oxy-, and a [(4-methoxyphenyl)sulfonyl]oxy group.

“Subject” includes humans. The terms “human”, “patient”, and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure”, and equivalent expressions, are meant to embrace compounds of Formula I as described herein, as well as its subgenera, which expression includes the stereoisomers (e.g., entaniomers, diastereomers) and constitutional isomers (e.g., tautomers) of compounds of Formula I as well as the pharmaceutically acceptable salts, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be 13, or any nitrogen may be ¹⁵N, and that the presence and placement of such atoms may be determined within the art.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”, for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

### Pharmaceutical Compositions

Also provided are pharmaceutical compositions comprising compounds of Formula (I), or a pharmaceutically acceptable salt, stereoisomer, solvate, N-oxide, tautomer, isotopic variant, prodrug, or deuterated compound thereof, and a pharmaceutically acceptable carrier.

The compositions may be in a form suitable for oral use (for example as tablets, lozenges, hard or soft capsules, aqueous or oily suspensions, emulsions, dispersible powders or granules, syrups or elixirs), for injection use (for example as aqueous or oil suspensions, or emulsions, with sesame oil, corn oil, cottonseed oil, or peanut oil, as well as elixirs, mannitol, dextrose, or a sterile aqueous solution, and similar pharmaceutical vehicles), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions may be obtained by conventional procedures using conventional pharmaceutical excipients, well known in the art. Thus, compositions intended for oral use may contain, for example, one or more coloring, sweetening, flavoring and/or preservative agents.

An effective amount of a compound of Formula (I) or a pharmaceutically salt thereof for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.1 mg to 1000 mg of Formula (I) or a pharmaceutically salt thereof with an appropriate and convenient amount of excipients which may vary from about 5 to about 98 percent by weight of the total composition.

The size of the dose for therapeutic or prophylactic purposes of a compound of the Formula (I) will naturally vary according to the nature and severity of the conditions, the age and sex of the animal or patient and the route of administration, according to well-known principles of medicine.

Described below are non-limiting exemplary pharmaceutical compositions and methods for preparing the same.

### Methods of Administration

The compounds of Formula (I) or a pharmaceutically salt thereof or pharmaceutical compositions comprising these compounds may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrastemal; by implant of a depot or reservoir, for example, subcutaneously or intramuscularly.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

The subject methods are useful for treating a disease condition associated with PARG. Any disease condition that results directly or indirectly from an abnormal activity or expression level of PARG can be an intended disease condition.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

In other methods, the compounds of the disclosure, as well as pharmaceutical compositions comprising them, can be administered in combination with an anti-proliferative agent.

### Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith el ah, March's Advanced Organic Chemistry: Reactions, Mechanisms, and Structure, 8th Ed. (Wiley, 2019); Peturssion et al, “Protecting Groups in Carbohydrate Chemistry”, J Chem. Educ., 1997, 74(11), 1297; and Wuts et al., Protective Groups in Organic Synthesis, 5th Ed., (Wiley, 2014).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high-performance liquid chromatography (HPLC) or thin layer chromatography.

The expressions, “ambient temperature”, “room temperature”, and “r.t.” as used herein, are understood in the art, and refer generally to a temperature, e.g. a reaction temperature, that is about the temperature of the room in which the reaction is carried out, for example, a temperature from about 20° C. to about 30° C.

Compounds of the invention can be prepared according to numerous preparatory routes known in the literatures. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The following Examples are provided to illustrate some of the concepts described within this disclosure. While the Examples are considered to provide an embodiment, it should not be considered to limit the more general embodiments described herein.

### Abbreviations

| | |
|---|---|
| aq. | Aqueous |
| BINAP | (2,2′-Bis(diphenylphosphino)-1,1′-binaphthyl) |
| Bippyphos | 5-(Di-tert-butylphosphino)-1′,3′,5′-triphenyl-1′H-[1,4′]bipyrazole |
| BrettPhos Pd G3 | [(2-Di-cyclohexylphosphino-3,6-dimethoxy-2′,4′,6′-triisopropyl-1,1′- |
| | biphenyl)-2-(2′-amino-1,1′-biphenyl)]palladium(II) methanesulfonate |
| | methanesulfonate |
| brine | Saturated solution of sodium chloride |
| BSA | Bovine albumin |
| DAST | Diethylaminosulfur trifluoride |
| DCM | Dichloromethane |
| DIEA or DIPEA | N,N-diisopropylethylamine |
| DMAc | Dimethylacetamide |
| DMAP | 4-Dimethylaminopyridine |
| DMF | N,N-dimethylformamide |
| DMSO | Dimethylsulfoxide |
| DTT | DL-Dithiothreitol |
| EDTA | Ethylene diamine tetra-acetic acid |
| EGTA | Ethylenebis(oxyethylenenitrilo)tetra-acetic acid |
| EtOAc or EA | Ethyl acetate |
| EtOH | Ethanol |
| FBS | Fetal Bovine Serum |
| FcP^{t}Bu₂ | Di-tert-butylphosphanylferrocene |
| HATU | 2-(7-Azabenzotriazol-1-yl)-N,N,N′,N′-tetramethyluronium |
| | hexafluorophosphate |
| ¹H NMR | Hydrogen-1 nuclear magnetic resonance spectroscopy |
| KHMDS | Potassium bis(trimethylsilyl)amide |
| LCMS | Liquid chromatography-mass spectrometry |
| m-CPBA | m-Chloroperbenzoic Acid |
| MeCN/ACN | Acetonitrile |
| MeNH₂ | Methylamine |
| MeOH | Methanol |
| MsCl | Methanesulfonyl Chloride |
| MTBE | Methyl tert-butyl ether |
| n-BuLi | n-Butyllithium |
| NaHMDS | Sodium bis(trimethylsilyl)amide |
| NaOAc | Sodium acetate |
| NaOMe | Sodium methoxide |
| NaOEt | Sodium ethoxide |
| NaSMe | Sodium methanethiolate |
| NaBH(OAc)₃ | Sodium Triacetoxyborohydride |
| Pd(dppf)Cl₂ | [1,1′-Bis(diphenylphosphino)ferrocene]dichloropalladium(II) |
| Pd(OAc)₂ | Palladium (II) Acetate |
| Pd(PPh₃)₄ | Tetrakis(triphenylphosphine)palladium(0) |
| Pd(PPh₃)₂Cl₂ | Bis(triphenylphosphine)palladium(II) chloride |
| Pd₂(dba)₃ | Bis(dibenzylideneacetone)palladium(0) |
| PE | Petroleum ether |
| p-TsOH | p-Toluenesulfonic acid |
| r.t. | Room temperature |
| RockPhos | 2-(Di-t-butylphosphino)-3-methoxy-6-methyl-2′,4′,6′-tri-i-propyl-1,1′- |
| | biphenyl |
| RuPhos Pd G3 | (2-Dicyclohexylphosphino-2′,6′-diisopropoxy-1,1′-biphenyl)[2-(2′- |
| | amino-1,1′-biphenyl)]palladium(II) methanesulfonate |
| t-BuOK | Potassium tert-butoxide |
| t-BuONa | Sodium tert-butoxide |
| t-BuXphos Pd G3 | [(2-Di-tert-butylphosphino-2′,4′,6′-triisopropyl-1,1′-biphenyl)-2-(2′- |
| | amino-1,1′-biphenyl)]palladium(II) methanesulfonate |
| TBSCl | t-Butyldimethylchlorosilane |
| TEA or Et₃N | Triethylamine |
| TFA | Trifluoroacetic acid |
| TfCl | Trifluoromethanesulfonyl chloride |
| THF | Tetrahydrofuran |
| UHP | Hydrogen peroxide-Urea adduct |
| xantphos | 9,9-Dimethyl-4,5-bis(diphenylphosphino)xanthene |
| XantPhos Pd G3 | [(4,5-Bis(diphenylphosphino)-9,9-dimethylxanthene)-2-(2′-amino-1,1′- |
| | biphenyl)]palladium(II) methanesulfonate |

1M or 1N = 1 mol/L, 2M or 2N = 2 mol/L etc.

### General Synthetic Procedures

A series of tricyclic amino-derivatives of formula 1-5 can be prepared by the methods outlined in Scheme 1. Compounds 1-3 can be prepared by reactions of compounds 1-1 where W¹ is halogen (e.g., Cl, Br, or I) or pseudohalogen (e.g., OTf or OMs) with appropriate amine derivatives 1-2 in the presence of a base such as Hunig's base. Coupling of compounds 1-3 with difluoromethyl-1,3,4-thiadiazol derivatives 1-4 where W is halogen (e.g., Cl, Br, or I) or pseudohalogen (e.g., OTf or OMs) under standard Buchwald coupling conditions (e.g., in the presence of a palladium catalyst, such as BrettPhos Pd G3, t-BuXphos Pd G3, RuPhos Pd G3 or XantPhos Pd G3 and a base, such as t-BuOK, t-BuONa, C⁵₂CO₃, or K₂CO₃) or under Ullmann coupling conditions (e.g., in the presence of a catalyst, such as CuI, CsF and ligand such as N₁,N₂-dimethylcyclohexane-1,2-diamine or N-methylimidazole) can provide compounds 1-5. Nucleophilic aromatic substitution reaction of compounds 1-3 with difluoromethyl-1,3,4-thiadiazol derivatives 1-4 in the presence of a base (e.g., C⁵₂CO₃, K₂CO₃) can also provide the compounds 1-5.

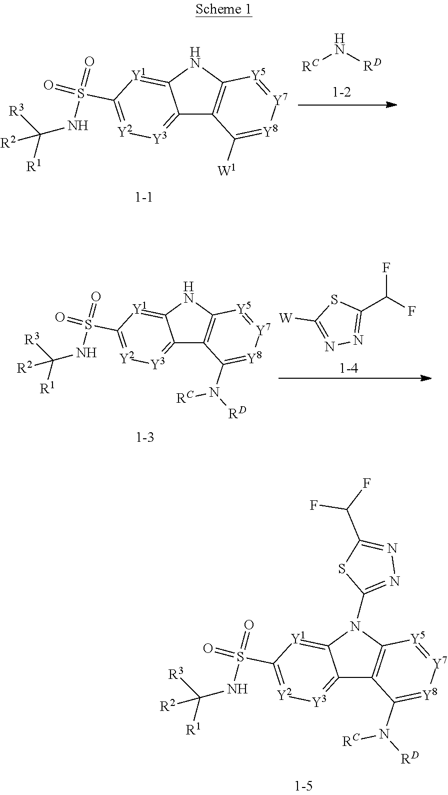

A series of tricyclic ether derivatives of formula 2-5 can be prepared by the methods outlined in Scheme 2. Compounds 2-3 can be prepared by reactions of compounds 2-1 where W¹ is halogen (e.g., Cl, Br, or I) or pseudohalogen (e.g., OTf or OMs) with a suitable alcohol derivatives 2-2 in the presence of a base such as KOH, NaH, NaHMDS, KHMDS or n-BuLi. Alternatively, coupling of compounds 2-1 with appropriate alcohol derivatives 2-2 under Ullmann coupling conditions (e.g., in the presence of a catalyst, such as CuI, K₃PO₄ and ligand such as proline derivatives, BINAP et. al) or Buchwald-Hartwig coupling conditions (e.g., in the presence of a palladium catalyst and ligand, such as Pd₂(dba)₃, Bippyphos, RockPhos, FcP^{t}Bu₂ and a base, such as t-BuOK, K₃PO₄, C⁵₂CO₃, or K₂CO₃) can also provide compounds 2-3. Nucleophilic aromatic substitution reaction of compounds 2-3 with difluoromethyl-1,3,4-thiadiazol derivatives 2-4 can also provide the compounds 2-5 in the presence of a base (e.g., Cs₂CO₃, K₂CO₃).

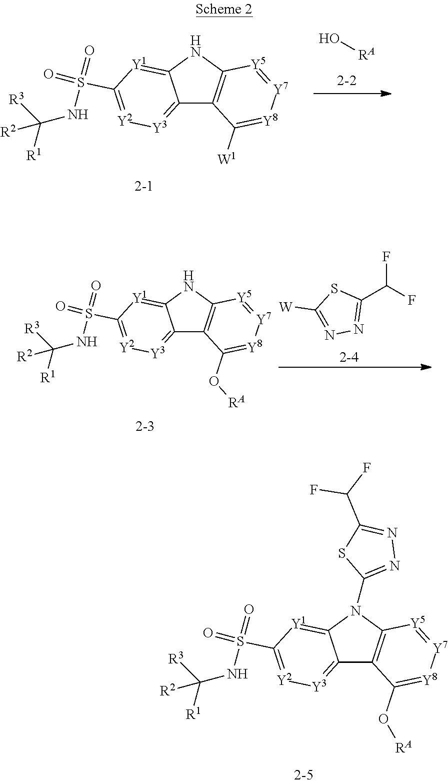

A series of tricyclic derivatives of formula 3-7 and 3-8 can be prepared by the methods outlined in Scheme 3. Vinyl compounds 3-4 can be prepared by reaction of compounds 3-1 where W¹ is halogen (e.g., Cl, Br, or I) or pseudohalogen (e.g., OTf or OMs) with vinyl boronic acid or boronate ester 3-2 where n is 0, 1, 2, 3, 4, 5, 6 can afford vinyl compounds 3-4 under standard Suzuki conditions (e.g., in the presence of a palladium catalyst, such as Pd(dppf)Cl₂ and a base, such as K₂CO₃) or with olefin derivatives 3-3 where n is 0, 1, 2, 3, 4, 5, 6 under standard Heck coupling conditions (e.g., in the presence of a palladium catalyst, such as Pd(OAc)₂, a base, such as TEA and a ligand such as tris(2-methoxyphenyl)phosphine). Hydrogenation of the vinyl compounds 3-4 can produce the corresponding compounds 3-5 in the presence of a palladium catalyst such as Pd/C or Pd(OH)₂/C. Coupling of compounds 3-4 or compounds 3-5 with difluoromethyl-1,3,4-thiadiazol derivatives 3-6 can provide corresponding compounds 3-7 or compounds 3-8, respectively, by using procedures analogous to those described in Scheme 1.

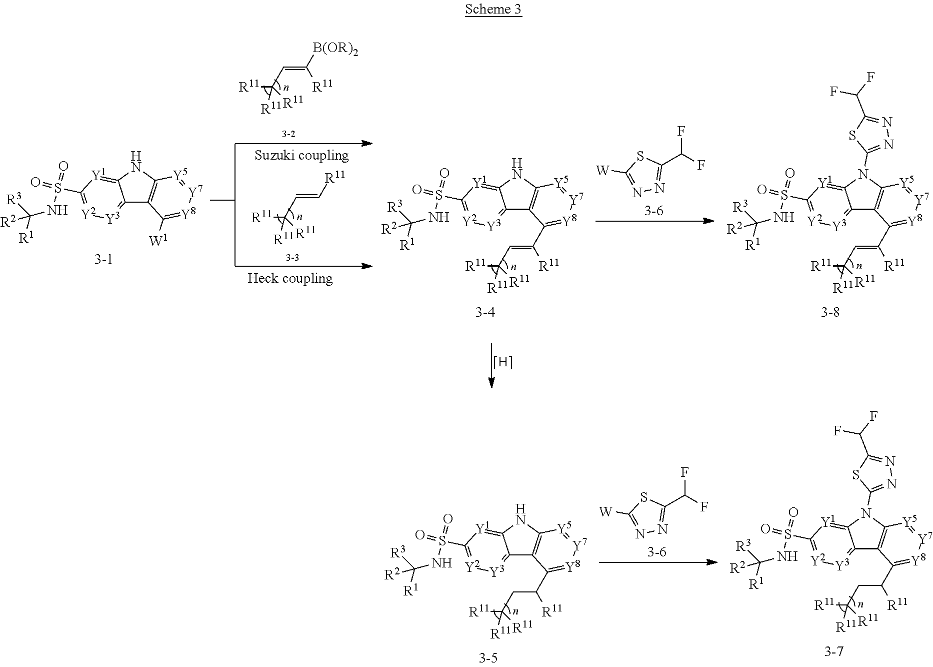

A series of tricyclic derivatives of formula 4-5 can be prepared by the methods outlined in Scheme 4. Compounds 4-3 can be prepared by reactions of compounds 4-1 where W¹ is halogen (e.g., Cl, Br, or I) or pseudohalogen (e.g., OTf or OMs) with a suitable terminal acetylene derivative 4-2 where n is 0, 1, 2, 3, 4, 5, 6 under Sonogashira coupling conditions (e.g., in the presence of a catalyst, Pd(PPh₃)₂Cl₂, CuI, and a base, such as TEA and DIPA). Coupling of compounds 4-3 with difluoromethyl-1,3,4-thiadiazol derivatives 4-4 can provide compounds 4-5 using procedures analogous to those described in Scheme 1.

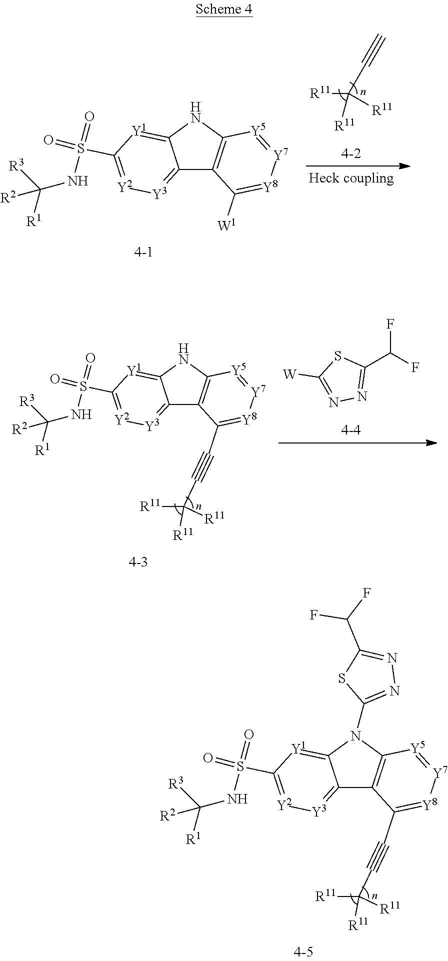

A series of tricyclic thia-ether derivatives of formula 5-5 can be prepared by the methods outlined in Scheme 5. Compounds 5-3 can be prepared by reactions of compounds 5-1 where W¹ is halogen (e.g., Cl, Br, or I) or pseudohalogen (e.g., OTf or OMs) with appropriate thioalcohols 5-2 in the presence of a base such as KOH, or NaH. Coupling of compounds 5-3 with difluoromethyl-1,3,4-thiadiazol derivatives 5-4 can provide compounds 5-5 using procedures analogous to those described in Scheme 1.

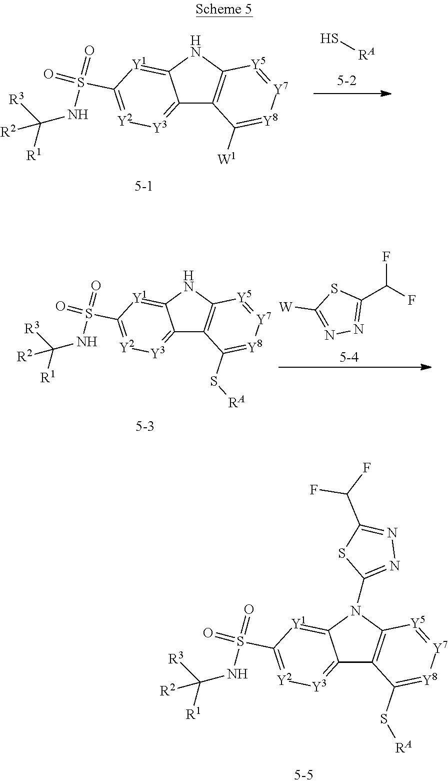

A series of tricyclic derivatives of formula 6-4, 6-6, 6-8 and 6-11 can be prepared by the methods outlined in Scheme 6. The tricyclic thia-ether derivatives 6-1 can be transformed into key intermediates tricyclic derivatives 6-2 where W¹ is halogen (e.g., Cl) or pseudohalogen (e.g., SO₂R^{A} or SOR^{A}) by treatment with a suitable halogenation reagent such as SO₂Cl₂ or by oxidation with a suitable oxidant such as m-CPBA, NaClO, NaIO₄, UHP (Hydrogen peroxide-Urea adduct) or oxone. The tricyclic derivatives 6-2 (where W¹ is Cl) can be furtherly reacted with appropriate amine derivatives 6-3 to afford the tricyclic derivatives of formula 6-4 in the presence of a base such as Hunig's base, or under standard Buchwald coupling conditions (e.g., in the presence of a palladium catalyst, such as BrettPhos Pd G3, t-BuXphos Pd G3, RuPhos Pd G3 or XantPhos Pd G3 and a base, such as t-BuOK, t-BuONa, C⁵₂CO₃, or K₂CO₃) or under Ullmann coupling conditions (e.g., CuI, CsF and ligand such as N₁,N₂-dimethylcyclohexane-1,2-diamine or N-methylimidazole); or with suitable alcohol derivatives 6-5 to afford the tricyclic derivatives of formula 6-6 in the presence of a base such as KOH, NaH, NaHMIDS, KHIVIDS or n-BuLi.

Reactions of the tricyclic chloride derivatives 6-2 (where W¹ is Cl) with a suitable terminal acetylene derivative 6-7 where n is 0, 1, 2, 3, 4, 5, 6 under Sonogashira coupling condition (e.g., in the presence of a catalyst, Pd(PPh₃)₂Cl₂, CuI, and a base, such as TEA and DIPEA) can yield the tricyclic derivatives of formula 6-8. In a similar manner, Heck coupling of compound 6-2 with olefin derivatives 6-10 where n is 0, 1, 2, 3, 4, 5, 6 can produce the tricyclic vinyl compounds 6-11 under standard Heck conditions (e.g., in the presence of a palladium catalyst, such as Pd(OAc)₂, a base, such as TEA and a ligand such as tris(2-methoxyphenyl)phosphine). Alternatively, reactions of the tricyclic chloride derivatives 6-2 with vinyl boronic acid or boronate esters 6-9 where n is 0, 1, 2, 3, 4, 5, 6 can also afford the tricyclic vinyl compounds 6-11 under standard Suzuki conditions (e.g., in the presence of a palladium catalyst, such as Pd(dppf)Cl₂ and a base, such as K₂CO₃).

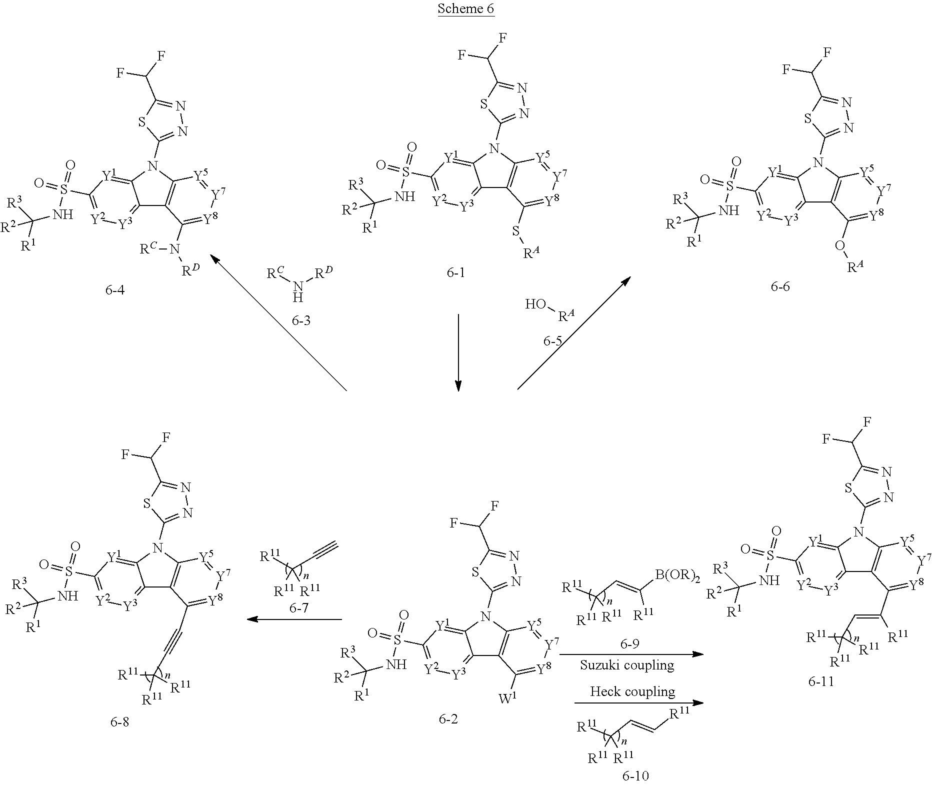

A series of tricyclic intermediates of formula 7-7 can be prepared by the methods outlined in Scheme 6. Sulfonamides 7-3 can be prepared by reaction of the sulfonyl chlorides 7-1 with an amine 7-2 in the presence of a base such as Hunig's base. Coupling of the sulfonamides 7-3 with 2-cyanoacetamide in the presence of a base, such as NaH, t-BuONa, or t-BuOK can afford compounds 7-4 which can be transformed into indole derivatives 7-5 by the nitro group with a reductive reagent such as Zn/FeCl₃ in acid media or Fe/NH₄C₁ followed the ring closure under the reaction conditions. Treatment of the indole derivatives 7-5 with trialkyl orthoformates 7-6 in the presence of an acid such as p-TsOH, or HCl can form the desired product indole-pyrimidones 7-7 which can be transformed into the intermediates 7-8 where W¹ is halogen (e.g., Cl, or Br) or pseudohalogen (e.g., OTf or OMs) either by reaction with a halogenation reagent such as SOCl₂, POCl₃ or POBr₃ with or without the catalytic of DMF (where W¹ is Cl or Br) or reaction with TfCl or MsCl (where W¹ is OTf or OMs) in the presence of a base such as Hunig's base.

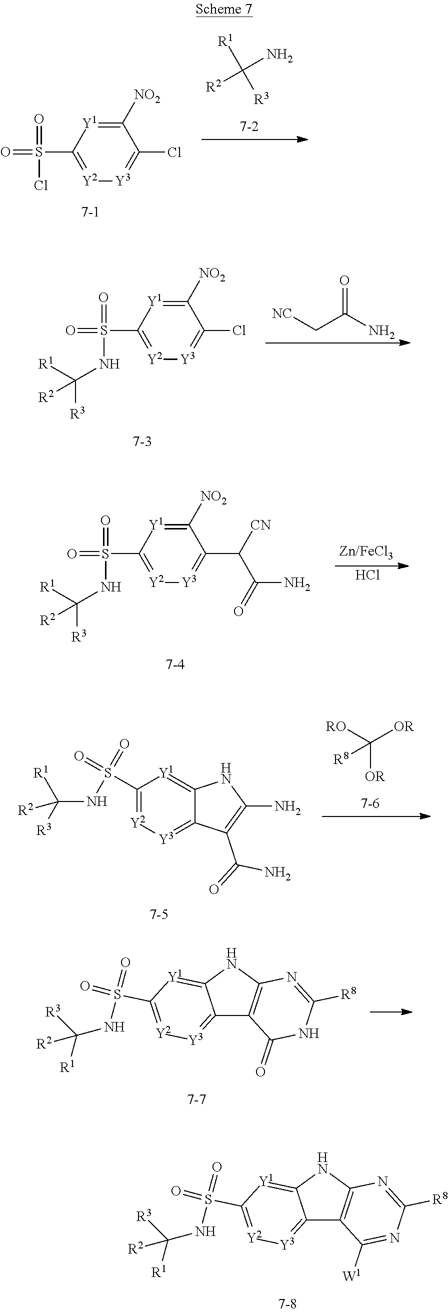

A series of tricyclic intermediates of formula 8-8 can be prepared by the methods outlined in Scheme 8. Coupling of compounds 8-1 where W² is halogen (e.g., Cl, Br, or I) or pseudohalogen (e.g., OTf or OMs), and W³ is halogen (e.g., Br, or I) or pseudohalogen (e.g., OTf) with compounds 8-2 under Buchwald coupling conditions (e.g., in the presence of a palladium catalyst, such as BrettPhos Pd G3, t-BuXphos Pd G3, RuPhos Pd G3 or XantPhos Pd G3 and a base, such as t-BuOK, t-BuONa, C⁵₂CO₃, or K₂CO₃), followed by ring closure by intra-molecular Heck reaction under the standard Heck reaction conditions (e.g., in the presence of a palladium catalyst, such as dichlorobis(triphenylphosphine)palladium, palladium diacetate, or tetrakis(triphenylphosphine)palladium and a base, such as Na₂CO₃, K₂CO₃, or NaOAc) can afford tricyclic compounds 8-3, which can be transformed into the corresponding sulfonyl chlorides 8-4 by oxidation reagents, such as N-chlorosuccinimide, sodium hypochlorite or treatment with a suitable reagent such as 1,3-dichloro-5,5-dimethylimidazolidine-2,4-dione. Reaction of the sulfonyl chlorides 8-4 with an amine 8-5 in the presence of a base such as Hunig's base can produce the sulfonamides 8-6 which can be transformed into compounds 8-7 by oxidative reagents such as hydrogen peroxide, oxone, and m-chloroperbenzoic acid. The compounds 8-7 can be converted into the intermediates 8-8 where W¹ is halogen (e.g., Cl, or Br) or pseudohalogen (e.g., OTf or OMs) by reaction with a halogenation reagent such as SOCl₂, POCl₃ or POBr₃ or reaction with TfCl or MsCl in the presence of a base such as Hunig's base.

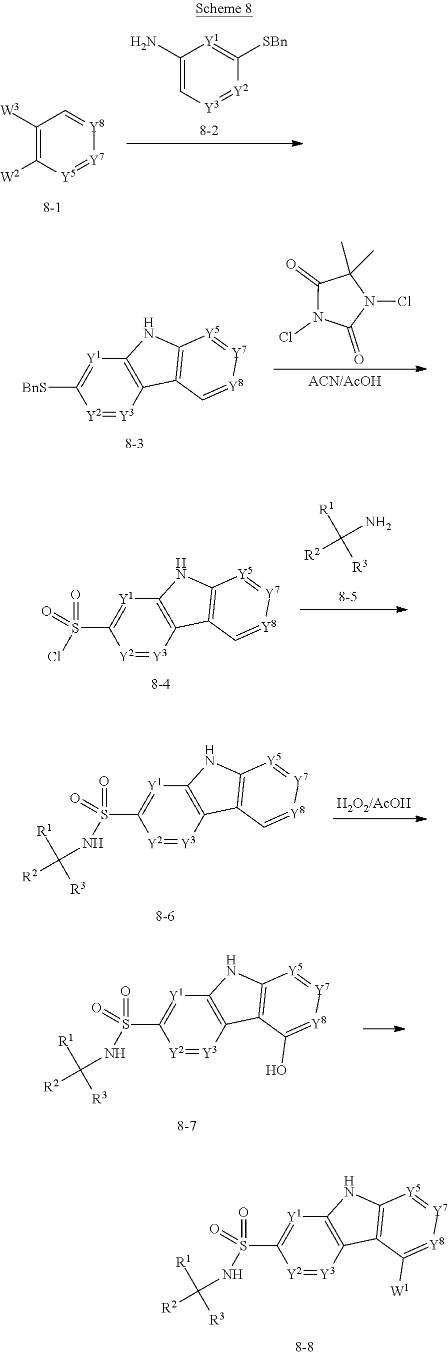

Alternatively, a series of tricyclic intermediates of formula 9-10 can be prepared by the methods outlined in Scheme 9. Tricyclic compounds 9-3 can be obtained in the similar way as describes in scheme 5 for the tricyclic compounds 9-3 by reaction with a suitable aniline 9-2. The removal of benzyl group in compounds 9-3 to the corresponding OH compounds 9-4 can be achieved by hydrogenation in the presence of a catalyst, such as Pd/C or Pd(OH)₂/C. Treatment the compounds 9-4 with trifluoromethanesulfonic anhydride afford the compounds 9-5 which can be transformed into 9-6 by reaction with phenylmethanethiol or sodium phenylmethanethiolate in the presence of a base such as Hunig's base, C⁵₂CO₃, t-BuOK, t-BuONa. The oxidation of compounds 9-6 with oxidation reagents, such as N-chlorosuccinimide, sodium hypochlorite can form the sulfonyl chlorides 9-7 which then can be transformed into the desired intermediates 9-10 by reaction with a suitable amine 9-8 in the presence of a base, such as Hunig's base, Na₂CO₃, or K₂CO₃, followed by removal of the protecting group Tf in the products 9-9 under basic conditions such as NaOH, or KOH.

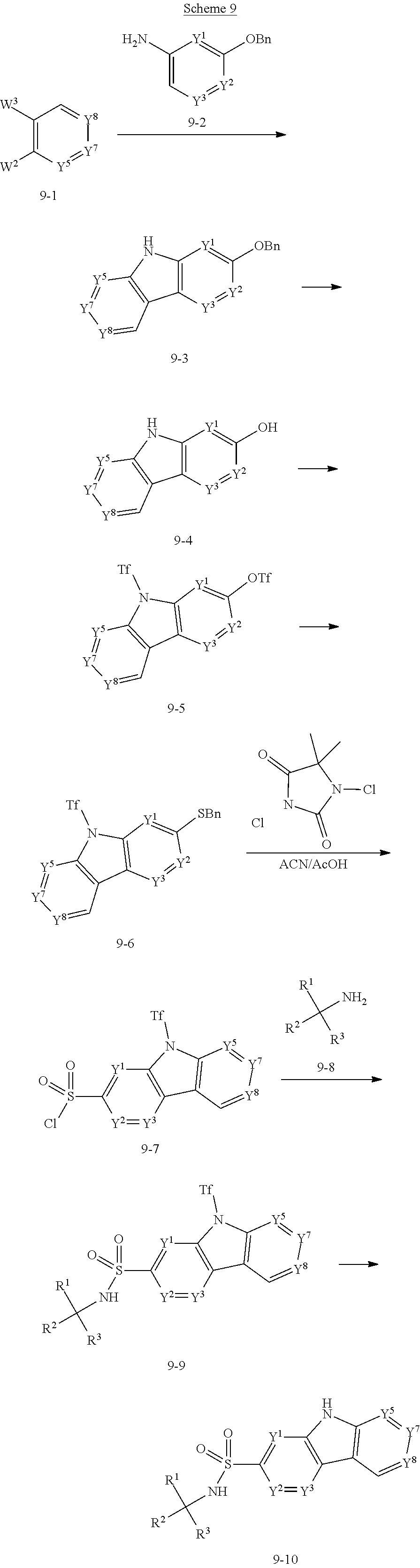

Alternatively, a series of tricyclic intermediates of formula 10-3 and 10-5 can be prepared by the methods outlined in the scheme 10. The compounds 10-3 and 10-5 can be prepared by Buchwald coupling compounds 10-1 where W² is halogen (e.g., Cl, Br, or I) or pseudohalogen (e.g., OTf or OMs), with compounds 10-2 and 10-4, respectively under standard conditions (e.g., in the presence of a palladium catalyst, such as BrettPhos Pd G3, t-BuXphos Pd G3, RuPhos Pd G3 or XantPhos Pd G3 and a base, such as t-BuOK, t-BuONa, C⁵₂CO₃, or K₂CO₃), followed by intramolecular ring closure in the presence of a Lewis acid, such as AlCl₃, ZnCl₂ or other acidic media such as polyphosphoric acid, POCl₃.

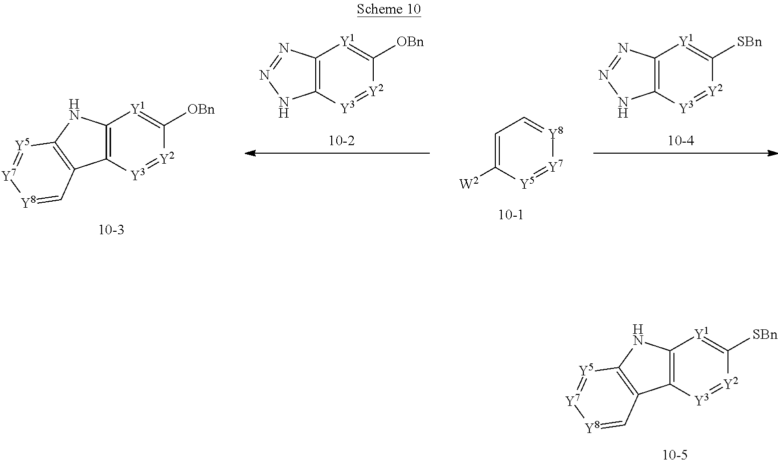

In a similar manner, a series of tricyclic intermediates of formula 11-3 and 11-5 can be prepared by the methods outlined in the scheme 11. The compounds 11-3 and 11-5 can be prepared by Buchwald coupling compounds 11-1 where W³ is halogen (e.g., Cl, Br, or I) or pseudohalogen (e.g., OTf or OMs), with compounds 11-2 and 11-5, respectively under standard conditions (e.g., in the presence of a palladium catalyst, such as BrettPhos Pd G3, t-BuXphos Pd G3, RuPhos Pd G3 or XantPhos Pd G3 and a base, such as t-BuOK, t-BuONa, C⁵₂CO₃, or K₂CO₃), followed by ring closure by intramolecular Heck reaction under the standard reaction condition (e.g., in the presence of a palladium catalyst, such as dichlorobis(triphenylphosphine)palladium, palladium diacetate, tetrakis(triphenylphosphine)palladium and a base, such as Na₂CO₃, K₂CO₃, or NaOAc).

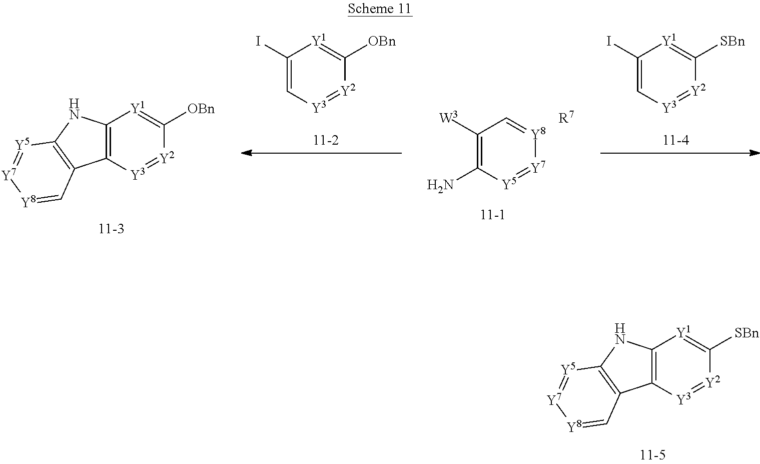

A series of tricyclic intermediates of formula 12-7 where A is O or S can be prepared by the methods outlined in the scheme 12. The compounds 12-3 can be prepared by nucleophile alkylation of compounds 12-1 where W² is halogen (e.g., F, Cl, Br, or I) or pseudohalogen (e.g., OTf or OMs), with alkyl 2-cyanoacetates 12-2 where R is alkyl (e.g., Me, Et or t-Bu) in the presence of a strong base, such as t-BuOK, t-BuONa, NaH). Reduction of the nitro group in 12-3 can be achieved by treatment with a reductive reagent such as Zn dust, or Fe powder in acidic conditions (such as acetic acid or HCl), followed by intramolecular ring closure to produce compounds 12-4. Heating the mixture of compounds 12-4 with an acetal 12-5 bearing alpha-H in the presence of a base such as NaOMe or NaOEt can yield tricyclic compounds 12-6. Halogenation of compounds 12-6 can provide the desired intermediates 12-7 (where W¹ is Cl or Br) with a halogenation reagent such as SOCl₂, POCl₃ or POBr₃ or 12-7 (where W¹ is OTf or OMs) with TfCl or MsCl in the presence of a base such as Hunig's base.

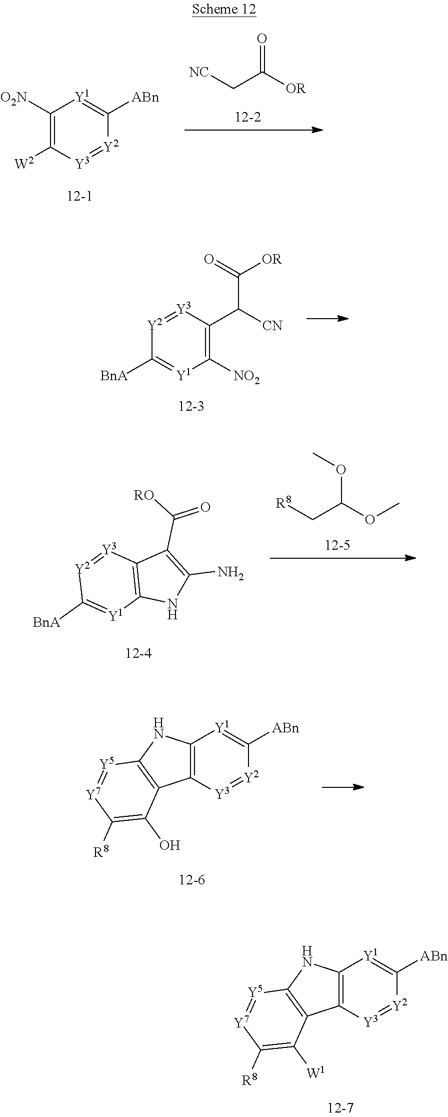

### Example 1: N-(1-Cyanocyclopropyl)-9-(5-(difluoromethyl)-1,3,4-thiadiazol-2-yl)-4-(2-morpholinoethoxy)-9H-pyrimido[4,5-b]indole-7-sulfonamide

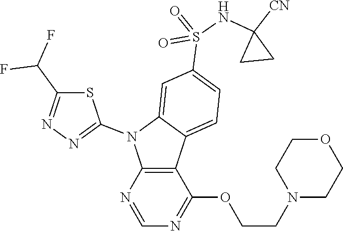 [SMILES: N#CC1(NS(=O)(=O)c2ccc3c4c(OCCN5CCOCC5)ncnc4n(-c4nnc(C(F)F)s4)c3c2)CC1]

### Step 1: N-(1-cyanocyclopropyl)-4-(2-morpholinoethoxy)-9H-pyrimido[4,5-b]indole-7-sulfonamide

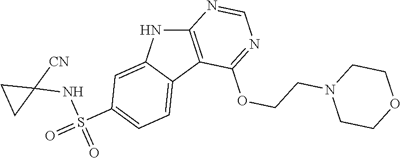 [SMILES: N#CC1(NS(=O)(=O)c2ccc3c(c2)[nH]c2ncnc(OCCN4CCOCC4)c23)CC1]

To a solution of 2-morpholinoethan-1-ol (567 mg, 4.3 mmol) in THF (15 mL) was added NaH (346 mg, 8.6 mmol, 60% dispersion in mineral oil) at 0° C. The reaction mixture was heated under reflux for 2 h. After cooled to r.t., 4-chloro-N-(1-cyanocyclopropyl)-9H-pyrimido[4,5-b]indole-7-sulfonamide (1.0 g, 2.9 mmol, Int A) was added to the reaction mixture. The resulting mixture was stirred at 60° C. for 30 min., cooled to r.t., quenched by saturated aq. NH₄Cl (1 mL), and then concentrated under reduced pressure. The residue was purified by flash chromatography on a C18 column eluting with MeCN/H₂O (5-29%) to afford the title compound (400 mg) as a yellow solid. LCMS calculated for C₂₀H₂₃N₆O₄S [M+H]+: m/z=443.1; Found: 443.2.

### Step 2: N-(1-cyanocyclopropyl)-9-(5-(difluoromethyl)-1,3,4-thiadiazol-2-yl)-4-(2-morpholinoethoxy)-9H-pyrimido[4,5-b]indole-7-sulfonamide

### Method A:

A mixture of N-(1-cyanocyclopropyl)-4-(2-morpholinoethoxy)-9H-pyrimido[4,5-b]indole-7-sulfonamide (400 mg, 0.9 mmol), 2-bromo-5-(difluoromethyl)-1,3,4-thiadiazole (484.1 mg, 2.3 mmol), CuI (138 mg, 0.7 mmol), (1R, 2R)—N¹,N²-dimethylcyclohexane-1,2-diamine (205.5 mg, 1.45 mmol) and CsF (550 mg, 3.62 mmol) in 1,4 dioxane (4 mL) was degassed and recharged with N₂ for 3 cycles. The mixture was stirred at 110° C. for 5 h.. After cooled to r.t., the reaction mixture was diluted with H₂O (10 mL), extracted with EA (10 mL×3). The combined organic layers were washed with brine, dried over Na₂SO₄, filtered and then concentrated under reduced pressure. The residue was purified by flash chromatography on a silica gel column eluting with MeOH/DCM (0-5%) to afford the crude product (210 mg) which was purified by prep-HPLC on a C18 column eluting with MeCN/H₂O (50-60% with 0.5% TFA) to afford the title compound (158.2 mg) as white solid. LCMS calc. for C₂₃H₂₃F₂N₈O₄S₂ [M+H]⁺: m/z=577.1; Found:577.1.

### Method B:

To a mixture of N-(1-cyanocyclopropyl)-4-(2-morpholinoethoxy)-9H-pyrimido[4,5-b]indole-7-sulfonamide (89 mg, 0.2 mmol) and 2-bromo-5-(difluoromethyl)-1,3,4-thiadiazole (130 mg, 0.6 mmol) in DMF (3 mL) was added Cs₂CO₃ (196 mg, 0.6 mmol). The mixture was stirred at r.t. for 16 h., and then concentrated under reduced pressure. The residue was purified by flash chromatography on a C18 column eluting with MeCN/H₂O (40-55% with 0.5% TFA) to afford the title compound (43 mg) as white solid. LCMS calc. for C₂₃H₂₃F₂N₈O₄S₂ [M+H]⁺: m/z=577.1; Found: 577.1.

The compounds listed in Table 1 below were prepared by using an lnt A and an appropriate It X (alcohol derivative) or commercially available material (CAM, alcohol derivative) as the methods substantially analogous to those described for preparing Example 1.

TABLE 1
Preparations of Examples (Ex)

| | Int A | | | LCMS |
|---|---|---|---|---|
| Ex | Int X | | | Cacl./ |
| # | Method | Structure | Name | Found |
| 2 | Int A CAM Method A | 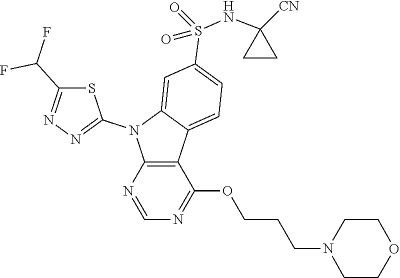 [SMILES: N#CC1(NS(=O)(=O)c2ccc3c4c(OCCCN5CCOCC5)ncnc4n(-c4nnc(C(F)F)s4)c3c2)CC1] | N-(1-Cyanocyclopropyl)-9-(5- (difluoromethyl)-1,3,4-thiadiazol-2- yl)-4-(3-morpholinopropoxy)-9H- pyrimido[4,5-b]indole-7- sulfonamide | [M + H]⁺ 591.1 591.1 |
| 3 | Int A CAM Method B | 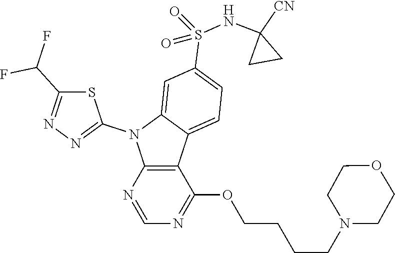 [SMILES: N#CC1(NS(=O)(=O)c2ccc3c4c(OCCCCN5CCOCC5)ncnc4n(-c4nnc(C(F)F)s4)c3c2)CC1] | N-(1-Cyanocyclopropyl)-9-(5- (difluoromethyl)-1,3,4-thiadiazol-2- yl)-4-(4-morpholinobutoxy)-9H- pyrimido[4,5-b]indole-7- sulfonamide | [M + H]⁺ 605.2 605.2 |
| 4 | Int A Int 1 Method B | 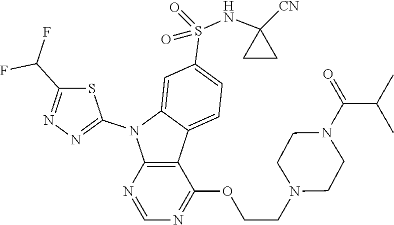 [SMILES: CC(C)C(=O)N1CCN(CCOc2ncnc3c2c2ccc(S(=O)(=O)NC4(C#N)CC4)cc2n3-c2nnc(C(F)F)s2)CC1] | N-(1-Cyanocyclopropyl)-9-(5- (difluoromethyl)-1,3,4-thiadiazol-2- yl)-4-(2-(4-isobutyrylpiperazin-1- yl)ethoxy)-9H-pyrimido[4,5- b]indole-7-sulfonamide | [M + H]⁺ 646.2 646.2 |
| 5 | Int A Int 2 Method B | 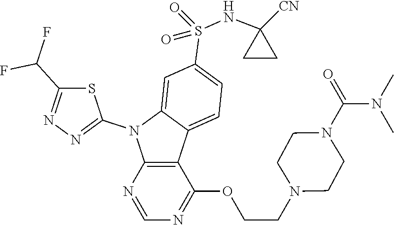 [SMILES: CN(C)C(=O)N1CCN(CCOc2ncnc3c2c2ccc(S(=O)(=O)NC4(C#N)CC4)cc2n3-c2nnc(C(F)F)s2)CC1] | 4-(2-((7-(N-(1- Cyanocyclopropyl)sulfamoyl)-9-(5- (difluoromethyl)-1,3,4-thiadiazol-2- yl)-9H-pyrimido[4,5-b]indol-4- yl)oxy)ethyl)-N,N- dimethylpiperazine-1-carboxamide | [M + H]⁺ 647.2 647.2 |
| 6 | Int A CAM Method B | 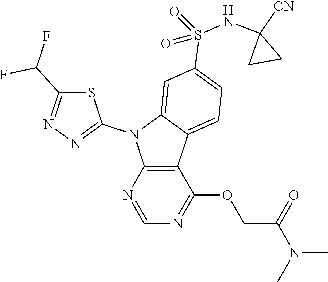 [SMILES: CN(C)C(=O)COc1ncnc2c1c1ccc(S(=O)(=O)NC3(C#N)CC3)cc1n2-c1nnc(C(F)F)s1] | 2-((7-(N-(1- Cyanocyclopropyl)sulfamoyl)-9-(5- (difluoromethyl)-1,3,4-thiadiazol-2- yl)-9H-pyrimido[4,5-b]indol-4- yl)oxy)-N,N-dimethylacetamide | [M + H]⁺ 549.1 549.1 |
| 7 | Int A CAM Method A | 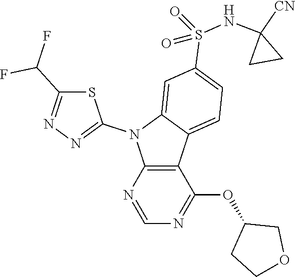 [SMILES: N#CC1(NS(=O)(=O)c2ccc3c4c(O[C@H]5CCOC5)ncnc4n(-c4nnc(C(F)F)s4)c3c2)CC1] | (S)-N-(1-Cyanocyclopropyl)-9-(5- (difluoromethyl)-1,3,4-thiadiazol-2- yl)-4-((tetrahydrofuran-3-yl)oxy)- 9H-pyrimido[4,5-b]indole-7- sulfonamide | [M + H]⁺ 534.1 534.0 |
| 8 | Int A CAM Method A | 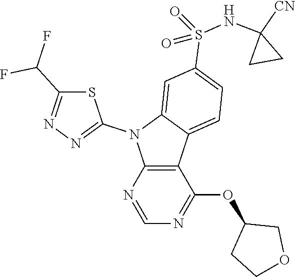 [SMILES: N#CC1(NS(=O)(=O)c2ccc3c4c(O[C@@H]5CCOC5)ncnc4n(-c4nnc(C(F)F)s4)c3c2)CC1] | (R)-N-(1-Cyanocyclopropyl)-9-(5- (difluoromethyl)-1,3,4-thiadiazol-2- yl)-4-((tetrahydrofuran-3-yl)oxy)- 9H-pyrimido[4,5-b]indole-7- sulfonamide | [M + H]⁺ 534.1 534.0 |
| 9 | Int A CAM Method A | 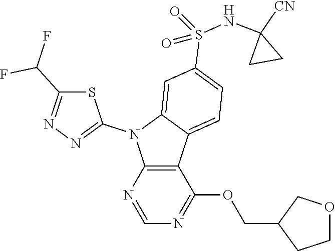 [SMILES: N#CC1(NS(=O)(=O)c2ccc3c4c(OCC5CCOC5)ncnc4n(-c4nnc(C(F)F)s4)c3c2)CC1] | N-(1-Cyanocyclopropyl)-9-(5- (difluoromethyl)-1,3,4-thiadiazol-2- yl)-4-((tetrahydrofuran-3- yl)methoxy)-9H-pyrimido[4,5- b]indole-7-sulfonamide | [M + H]⁺ 548.1 548.1 |
| 10 | Int A CAM Method A | 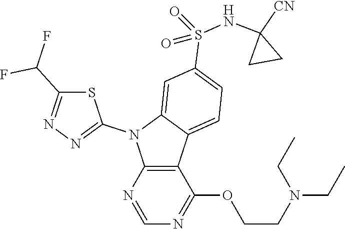 [SMILES: CCN(CC)CCOc1ncnc2c1c1ccc(S(=O)(=O)NC3(C#N)CC3)cc1n2-c1nnc(C(F)F)s1] | N-(1-Cyanocyclopropyl)-4-(2- (diethylamino)ethoxy)-9-(5- (difluoromethyl)-1,3,4-thiadiazol-2- yl)-9H-pyrimido[4,5-b]indole-7- sulfonamide | [M + H]⁺ 563.1 563.1 |
| 11 | Int A CAM Method B | 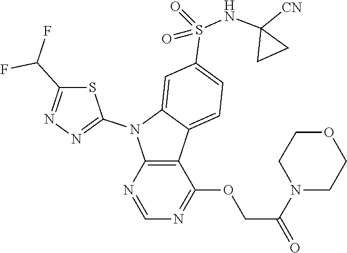 [SMILES: N#CC1(NS(=O)(=O)c2ccc3c4c(OCC(=O)N5CCOCC5)ncnc4n(-c4nnc(C(F)F)s4)c3c2)CC1] | N-(1-Cyanocyclopropyl)-9-(5- (difluoromethyl)-1,3,4-thiadiazol-2- yl)-4-(2-morpholino-2-oxoethoxy)- 9H-pyrimido[4,5-b]indole-7- sulfonamide | [M + H]⁺ 591.1 591.1 |
| 12 | Int A CAM Method B | 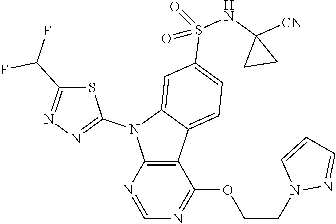 [SMILES: N#CC1(NS(=O)(=O)c2ccc3c4c(OCCn5cccn5)ncnc4n(-c4nnc(C(F)F)s4)c3c2)CC1] | 4-(2-(1H-Pyrazol-1-yl)ethoxy)-N- (1-cyanocyclopropyl)-9-(5- (difluoromethyl)-1,3,4-thiadiazol-2- yl)-9H-pyrimido[4,5-b]indole-7- sulfonamide | [M + H]⁺ 558.1 558.1 |

### Example 13 I-1-Cyanocyclopropyl)-9-(5-(difluoromethyl)-1,3,4-thiadiazol-2-yl)-4-(methyl(2-morpholinoethyl)amino)-9H-pyrimido[4,5-b]indole-7-sulfonamide

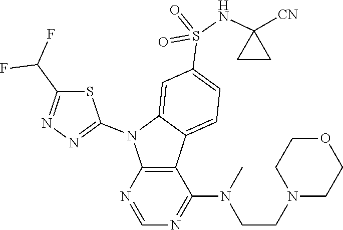 [SMILES: CN(CCN1CCOCC1)c1ncnc2c1c1ccc(S(=O)(=O)NC3(C#N)CC3)cc1n2-c1nnc(C(F)F)s1]

### Step 1: N-(1-cyanocyclopropyl)-4-(methyl(2-morpholinoethyl)amino)-9H-pyrimido[4,5-b]indole-7-sulfonamide

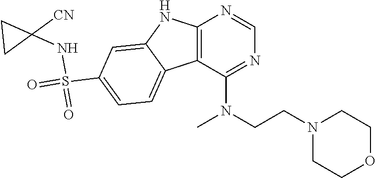 [SMILES: CN(CCN1CCOCC1)c1ncnc2[nH]c3cc(S(=O)(=O)NC4(C#N)CC4)ccc3c12]

To a solution of 4-chloro-N-(1-cyanocyclopropyl)-9H-pyrimido[4,5-b]indole-7-sulfonamide (1.76 g, 5.0 mmol, Int A) in MeCN (20 mL) was added N-methyl-2-morpholinoethan-1-amine (1.0 g, 6.6 mmol) and Et₃N (1.54 g, 15.2 mmol). The mixture was stirred at 80° C. for 1 h., and then cooled to r.t. The reaction mixture was filtered and the filter cake was washed with water, dried under reduced pressure to afford the title compound (1.5 g) as brown solid. LCMS calculated for C₂₁H₂₆N₇O₃S [M+H]⁺: m/z=456.2; Found: 456.2.

### Step 2: N-(1-cyanocyclopropyl)-9-(5-(difluoromethyl)-1,3,4-thiadiazol-2-yl)-4-(methyl(2-morpholinoethyl)amino)-9H-pyrimido[4,5-b]indole-7-sulfonamide

### Method C:

To a solution of N-(1-cyanocyclopropyl)-4-(methyl(2-morpholinoethyl)amino)-9H-pyrimido[4,5-b]indole-7-sulfonamide (1.5 g, 3.3 mmol), 2-bromo-5-(difluoromethyl)-1,3,4-thiadiazole (2.8 g, 13.2 mmol), CuI (0.5 g, 2.6 mmol), (1R, 2R)—N¹,N²-dimethylcyclohexane-1,2-diamine (0.75 g, 5.3 mmol) and CsF (2.0 g, 13.2 mmol) in 1,4 dioxane (15 mL) was degassed and recharged with N₂ for 3 cycles. The mixture was stirred at 110° C. overnight. After cooled to r.t., the reaction mixture was concentrated under reduced pressure. The residue was purified by flash chromatography on a silica gel column eluting with MeOH/DCM (0-6%) to afford the crude product (500 mg) which was re-purified by prep-HPLC on a C18 column eluting with MeCN/H₂O (50-60%) to afford the title product (325 mg) as a white solid. ¹H NMR (400 MHz, DMSO-d⁶) δ 9.49 (d, J=1.6 Hz, 1H), 9.36 (s, 1H), 8.68 (s, 1H), 8.26 (d, J=8.4 Hz, 1H), 8.00 (dd, J=8.4, 1.8 Hz, 1H), 7.7 (t, J_{F}-H=53.6 Hz, 1H), 3.95 (t, J=6.4 Hz, 2H), 3.41 (s, 3H), 3.35 (t, J=4.6 Hz, 4H), 2.65 (t, J=6.4 Hz, 2H), 2.30 (t, J=4.4 Hz, 4H), 1.46-1.39 (m, 2H), 1.34-1.25 (m, 2H). LCMS calc. for C₂₄H₂₆F₂N₉03S₂ [M+H]⁺: m/z=590.2; Found: 590.2.

### Method D:

To a mixture of N-(1-cyanocyclopropyl)-4-(methyl(2-morpholinoethyl)amino)-9H-pyrimido[4,5-b]indole-7-sulfonamide(91 mg, 0.2 mmol) and 2-bromo-5-(difluoromethyl)-1,3,4-thiadiazole (173 mg, 0.8 mmol) in DMF (3 mL) was added Cs₂CO₃ (196 mg, 0.6 mmol). The mixture was stirred at r.t. for 16 h., and then concentrated under reduced pressure. The residue was purified by flash chromatography on a C18 column eluting with MeCN/H₂O (50-55% with 0.5% TFA) to afford the title compound (53 mg) as white solid. LCMS calc. for C₂₄H₂₆F₂N₉03S₂ [M+H]⁺: m/z=590.2; Found: 590.2.

The compounds listed in Table 2 below were prepared by using an IntA (sulfonamide derivative) and an appropriate IntX (amine derivative) or commercially available material (CAM, amine derivative) as the methods substantially analogous to those described for preparing Example 13.

TABLE 2
Preparations of Examples (Ex)

| | Int A | | | LCMS |
|---|---|---|---|---|
| Ex | Int X | | | Cacl./ |
| # | Method | Structure | Name | Found |
| 14 | Int A CAM Method C | 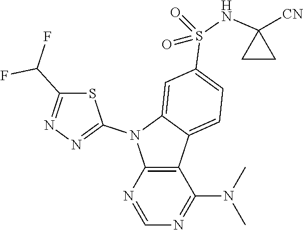 [SMILES: CN(C)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(dimethylamino)-9H- pyrimido[4,5-b]indole-7-sulfonamide | [M + H]⁺ 491.1 491.1 |
| 15 | Int A CAM Method C | 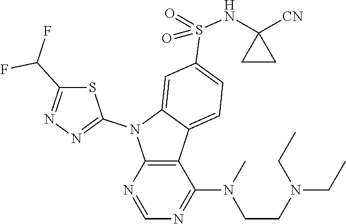 [SMILES: CCN(CC)CCN(C)c1ncnc2c1c1ccc(S(=O)(=O)NC3(C#N)CC3)cc1n2-c1nnc(C(F)F)s1] | N-(1-Cyanocyclopropyl)-4-((2- (diethylamino)ethyl)(methyl)amino)- 9-(5-(difluoromethyl)-1,3,4- thiadiazol-2-yl)-9H-pyrimido[4,5- b]indole-7-sulfonamide | [M + H]⁺ 576.2 576.2 |
| 16 | Int A CAM Method C | 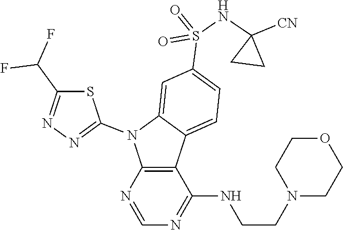 [SMILES: N#CC1(NS(=O)(=O)c2ccc3c4c(NCCN5CCOCC5)ncnc4n(-c4nnc(C(F)F)s4)c3c2)CC1] | N-(1-Cyanocyclopropyl)-9-(5- (difluoromethyl)-1,3,4-thiadiazol-2- yl)-4-((2-morpholinoethyl)amino)- 9H-pyrimido[4,5-b]indole-7- sulfonamide | [M + H]⁺ 576.1 576.1 |
| 17 | Int A CAM Method D | 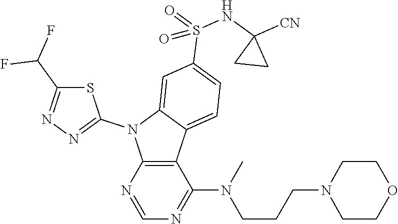 [SMILES: CN(CCCN1CCOCC1)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(methyl(3- morpholinopropyl)amino)-9H- pyrimido[4,5-b]indole-7-sulfonamide | [M + H]⁺ 604.2 604.2 |
| 18 | Int A CAM Method C | 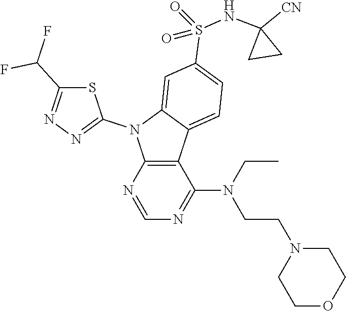 [SMILES: CCN(CCN1CCOCC1)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(ethyl(2- morpholinoethyl)amino)-9H- pyrimido [4,5-b]indole-7-sulfonamide | [M + H]⁺ 604.2 604.2 |
| 19 | Int A CAM Method D | 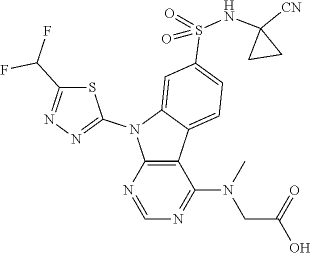 [SMILES: CN(CC(=O)O)c1ncnc2c1c1ccc(S(=O)(=O)NC3(C#N)CC3)cc1n2-c1nnc(C(F)F)s1] | N-(7-(N-(1- Cyanocyclopropyl)sulfamoyl)-9-(5- (difluoromethyl)-1,3,4-thiadiazol-2- yl)-9H-pyrimido[4,5-b]indol-4-yl)-N- methylglycine | [M + H]⁺ 535.1 535.1 |
| 20 | Int A CAM Method D | 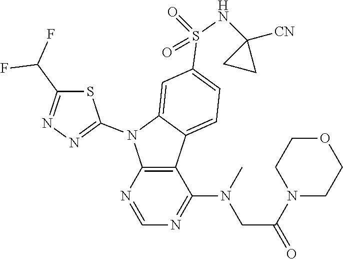 [SMILES: CN(CC(=O)N1CCOCC1)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(methyl(2-morpholino-2- oxoethyl)amino)-9H-pyrimido[4,5- b]indole-7-sulfonamide | [M + H]⁺ 604.1 604.0 |
| 21 | Int A CAM Method D | 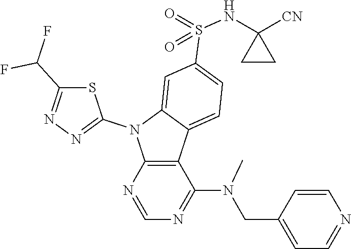 [SMILES: CN(Cc1ccncc1)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(methyl(pyridin-4- ylmethyl)amino)-9H-pyrimido[4,5- b]indole-7-sulfonamide | [M + H]⁺ 568.1 568.1 |
| 22 | Int A CAM Method D | 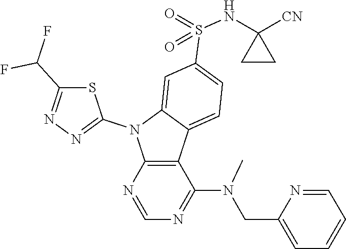 [SMILES: CN(Cc1ccccn1)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(methyl(pyridin-2- ylmethyl)amino)-9H-pyrimido[4,5- b]indole-7-sulfonamide | [M + H]⁺ 568.1 568.1 |
| 23 | Int A CAM Method D | 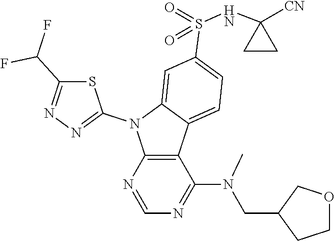 [SMILES: CN(CC1CCOC1)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(methyl((tetrahydrofuran-3- yl)methyl)amino)-9H-pyrimido[4,5- b]indole-7-sulfonamide | [M + H]⁺ 561.1 561.1 |
| 24 | Int A CAM Method D | 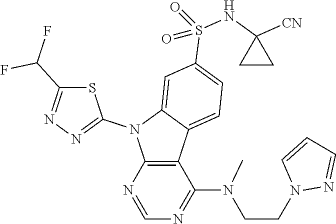 [SMILES: CN(CCn1cccn1)c1ncnc2c1c1ccc(S(=O)(=O)NC3(C#N)CC3)cc1n2-c1nnc(C(F)F)s1] | 4-((2-(1H-Pyrazol-1- yl)ethyl)(methyl)amino)-N-(1- cyanocyclopropyl)-9-(5- (difluoromethyl)-1,3,4-thiadiazol-2- yl)-9H-pyrimido[4,5-b]indole-7- sulfonamide | [M + H]⁺ 571.1 571.1 |

### Example 25: N-(1-Cyanocyclopropyl)-9-(5-(difluoromethyl)-1,3,4-thiadiazol-2-yl)-4-(methylthio)-911-pyrimido[4,5-b]indole-7-sulfonamide

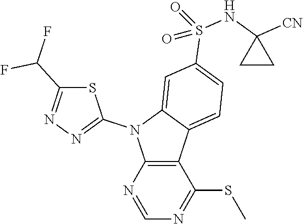 [SMILES: CSc1ncnc2c1c1ccc(S(=O)(=O)NC3(C#N)CC3)cc1n2-c1nnc(C(F)F)s1]

### Step 1: N-(1-cyanocyclopropyl)-4-(methylthio)-9H-pyrimido[4,5-b]indole-7-sulfonamide

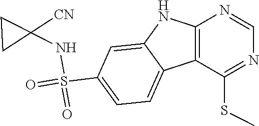 [SMILES: CSc1ncnc2[nH]c3cc(S(=O)(=O)NC4(C#N)CC4)ccc3c12]

To a solution of 4-chloro-N-(1-cyanocyclopropyl)-9H-pyrimido[4,5-b]indole-7-sulfonamide (100 mg, 0.3 mmol, Int A) in DMF (2 mL) was added NaSMe (35 mg, 0.5 mmol). The reaction mixture was stirred at 100° C. for 2 h. and then cooled to r.t., diluted with water (4 mL) and filtered. The filter cake was dried under reduced pressure to afford the title compound (90 mg) as a yellow solid. LCMS calculated for C₁₅H₁₄N₅O₂S₂[M+H]⁺: m/z=360.1; Found: 360.0.

Step 2: N-(1-cyanocyclopropyl)-9-(5-(difluoromethyl)-1,3,4-thiadiazol-2-yl)-4-(methylthio)-9H-pyrimido[4,5-b]indole-7-sulfonamide A mixture of N-(1-cyanocyclopropyl)-4-(methylthio)-9H-pyrimido[4,5-b]indole-7-sulfonamide (36 mg, 0.1 mmol), 2-bromo-5-(difluoromethyl)-1,3,4-thiadiazole (43 mg, 0.2 mmol) and Cs₂CO₃ (65 mg, 0.2 mmol) in DMF (1 mL) was stirred at r.t. overnight. The reaction mixture was filtered, and concentrated under reduced pressure. The residue was purified by prep-HPLC on a C18 column eluting with MeCN/H₂O (30-75%, with 0.5% TFA) to afford the title product (27.5 mg) as a white solid. ¹H NMR (400 MHz, DMSO-d₆) δ 9.49 (s, 2H), 9.18 (s, 1H), 8.43 (d, J=8.4 Hz, 1H), 8.21 (d, J=8.4 Hz, 1H), 7.70 (t, J_{F}-H=53.4 Hz, 1H), 2.67 (s, 3H), 1.49-1.39 (m, 2H), 1.34-1.22 (m, 2H). LCMS calc. for C₁₈H₁₄F₂N₇O₂S₃ [M+H]⁺: m/z=494.0; Found: 494.0.

The compounds listed in Table 3 below were prepared by using an Int A and an appropriate Int X (mercaptan derivative) or commercially available material (CAM, mercaptan derivative) as the methods substantially analogous to those described for preparing Example 25.

TABLE 3
Preparations of Examples (Ex)

| | | | | LCMS |
|---|---|---|---|---|
| Ex | Int A | | | Cacl./ |
| # | Int X | Structure | Name | Found |
| 26 | Int A | 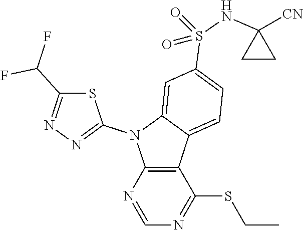 [SMILES: CCSc1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(ethylthio)-9H-pyrimido[4,5- b]indole-7-sulfonamide | [M + H]⁺ 508.0 508.0 |
| 27 | CAM | 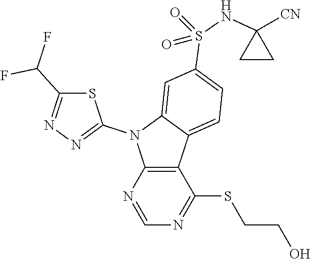 [SMILES: N#CC1(NS(=O)(=O)c2ccc3c4c(SCCO)ncnc4n(-c4nnc(C(F)F)s4)c3c2)CC1] | N-(1-Cyanocyclopropyl)-9-(5- (difluoromethyl)-1,3,4-thiadiazol-2- yl)-4-((2-hydroxyethyl)thio)-9H- pyrimido[4,5-b]indole-7-sulfonamide | [M + H]⁺ 524.0 524.0 |

### Example 28: N-(1-Cyanocyclopropyl)-4-(4-(diethylamino)piperidin-1-yl)-9-(5-(difluoromethyl)-1,3,4-thiadiazol-2-yl)-9H-pyrimido[4,5-b]indole-7-sulfonamide

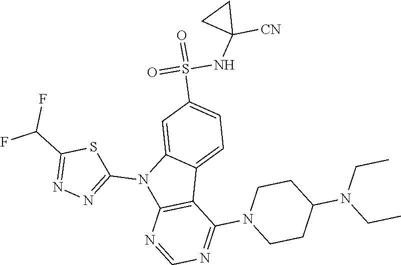 [SMILES: CCN(CC)C1CCN(c2ncnc3c2c2ccc(S(=O)(=O)NC4(C#N)CC4)cc2n3-c2nnc(C(F)F)s2)CC1]

To a mixture of 4-chloro-N-(1-cyanocyclopropyl)-9-(5-(difluoromethyl)-1,3,4-thiadiazol-2-yl)-9H-pyrimido[4,5-b]indole-7-sulfonamide (144 mg, 0.3 mmol, ItB), DIEA (78 mg, 0.6 mmol) in MeCN (2 mL) was added N,N-diethylpiperidin-4-amine (40 mg, 0.33 mmol). The reaction mixture was stirred at 60° C. for 3 h.. The resulting mixture was concentrated under reduced pressure. The residue was purified by prep-HPLC on a C18 column eluting with MeCN/H₂O (30-4000, with 0.500 TFA) to afford the title compound (130 mg) as a white solid. LCMS calc. for C₂₆H₃₀F₂N₉O₂S₂[M+H]⁺: m/z=602.2; Found: 602.2.

The compounds listed in Table 4 below were prepared by using an IntB and an appropriate ItX(amine derivative) or commercially available material (CAM, amine derivative) as the methods substantially analogous to those described for preparing Example 28.

TABLE 4
Preparations of Examples (Ex)

| | | | | LCMS |
|---|---|---|---|---|
| Ex | Int B | | | Cacl./ |
| # | Int X | Structure | Name | Found |
| 29 | Int B CAM | 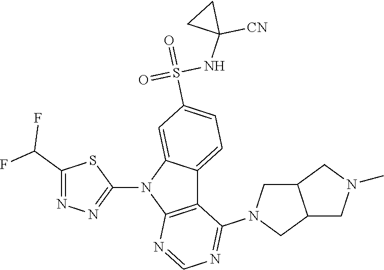 [SMILES: CN1CC2CN(c3ncnc4c3c3ccc(S(=O)(=O)NC5(C#N)CC5)cc3n4-c3nnc(C(F)F)s3)CC2C1] | N-(1-Cyanocyclopropyl)-9-(5- (difluoromethyl)-1,3,4-thiadiazol-2- yl)-4-(5- methylhexahydropyrrolo[3,4- c]pyrrol-2(1H)-yl)-9H-pyrimido[4,5- b]indole-7-sulfonamide | [M + H]⁺ 572.1 572.1 |
| 30 | Int B CAM | 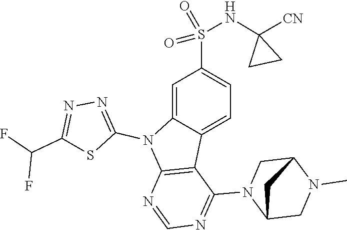 [SMILES: CN1C[C@H]2C[C@@H]1CN2c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-((1R,4R)-5-methyl-2,5- diazabicyclo[2.2.1]heptan-2-yl)-9H- pyrimido[4,5-b]indole-7-sulfonamide | [M + H]⁺ 558.1 558.2 |
| 31 | Int B CAM | 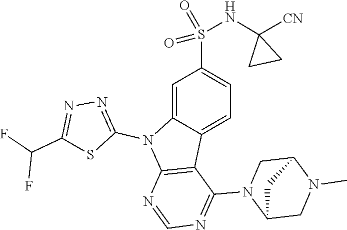 [SMILES: CN1C[C@@H]2C[C@H]1CN2c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-((1S,4S)-5-methyl-2,5- diazabicyclo[2.2.1]heptan-2-yl)-9H- pyrimido[4,5-b]indole-7-sulfonamide | [M + H]⁺ 558.1 558.1 |
| 32 | Int B Int 3 | 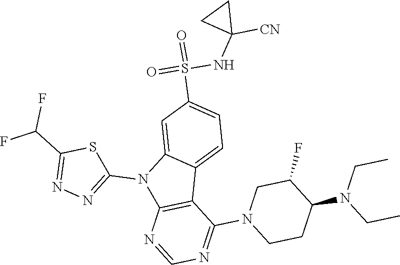 [SMILES: CCN(CC)[C@H]1CCN(c2ncnc3c2c2ccc(S(=O)(=O)NC4(C#N)CC4)cc2n3-c2nnc(C(F)F)s2)C[C@@H]1F] | N-(1-Cyanocyclopropyl)-4-((3S,4S)- 4-(diethylamino)-3-fluoropiperidin-1- yl)-9-(5-(difluoromethyl)-1,3,4- thiadiazol-2-yl)-9H-pyrimido[4,5- b]indole-7-sulfonamide | [M + H]⁺ 620.2 620.2 |
| 33 | Int B Int 4 | 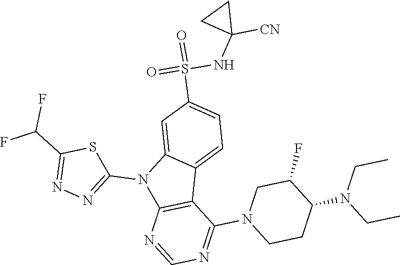 [SMILES: CCN(CC)[C@@H]1CCN(c2ncnc3c2c2ccc(S(=O)(=O)NC4(C#N)CC4)cc2n3-c2nnc(C(F)F)s2)C[C@@H]1F] | N-(1-Cyanocyclopropyl)-4-((3S,4R)- 4-(diethylamino)-3-fluoropiperidin-1- yl)-9-(5-(difluoromethyl)-1,3,4- thiadiazol-2-yl)-9H-pyrimido[4,5- b]indole-7-sulfonamide | [M + H]⁺ 620.2 620.2 |
| 34 | Int B Int 5 | 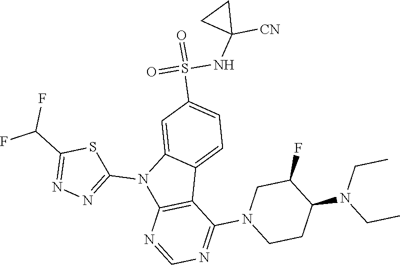 [SMILES: CCN(CC)[C@H]1CCN(c2ncnc3c2c2ccc(S(=O)(=O)NC4(C#N)CC4)cc2n3-c2nnc(C(F)F)s2)C[C@H]1F] | N-(1-Cyanocyclopropyl)-4-((3R,4S)- 4-(diethylamino)-3-fluoropiperidin-1- yl)-9-(5-(difluoromethyl)-1,3,4- thiadiazol-2-yl)-9H-pyrimido[4,5- b]indole-7-sulfonamide | [M + H]⁺ 620.2 620.2 |
| 35 | Int B Int 6 | 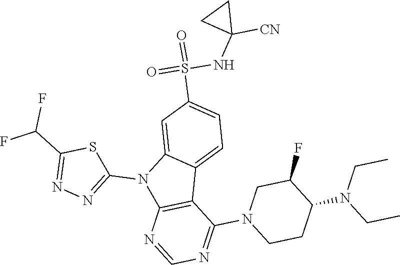 [SMILES: CCN(CC)[C@@H]1CCN(c2ncnc3c2c2ccc(S(=O)(=O)NC4(C#N)CC4)cc2n3-c2nnc(C(F)F)s2)C[C@H]1F] | N-(1-Cyanocyclopropyl)-4-((3R,4R)- 4-(diethylamino)-3-fluoropiperidin-1- yl)-9-(5-(difluoromethyl)-1,3,4- thiadiazol-2-yl)-9H-pyrimido[4,5- b]indole-7-sulfonamide | [M + H]⁺ 620.2 620.2 |
| 36 | Int B Int 7 | 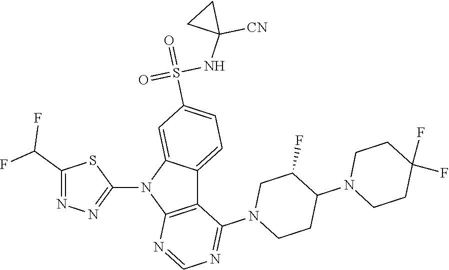 [SMILES: N#CC1(NS(=O)(=O)c2ccc3c4c(N5CCC(N6CCC(F)(F)CC6)[C@@H](F)C5)ncnc4n(-c4nnc(C(F)F)s4)c3c2)CC1] | N-(1-Cyanocyclopropyl)-9-(5- (difluoromethyl)-1,3,4-thiadiazol-2- yl)-4-((3′S)-3′,4,4-trifluoro-[1,4′- bipiperidin]-1′-yl)-9H-pyrimido[4,5- b]indole-7-sulfonamide | [M + H]⁺ 668.2 668.2 |
| 37 | Int B CAM | 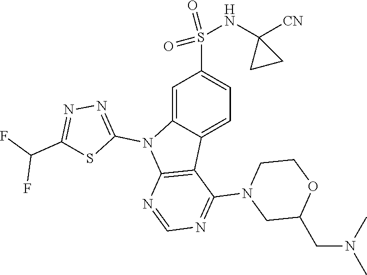 [SMILES: CN(C)CC1CN(c2ncnc3c2c2ccc(S(=O)(=O)NC4(C#N)CC4)cc2n3-c2nnc(C(F)F)s2)CCO1] | N-(1-Cyanocyclopropyl)-9-(5- (difluoromethyl)-1,3,4-thiadiazol-2- yl)-4-(2- ((dimethylamino)methyl)morpholino)- 9H-pyrimido[4,5-b]indole-7- sulfonamide | [M + H]⁺ 590.2 590.2 |
| 38 | Int B Int 8 | 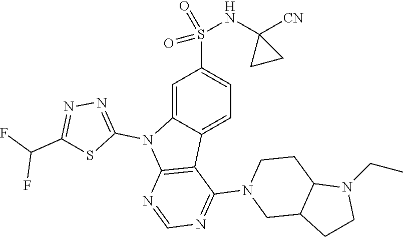 [SMILES: CCN1CCC2CN(c3ncnc4c3c3ccc(S(=O)(=O)NC5(C#N)CC5)cc3n4-c3nnc(C(F)F)s3)CCC21] | N-(1-Cyanocyclopropyl)-9-(5- (difluoromethyl)-1,3,4-thiadiazol-2- yl)-4-(1-ethyloctahydro-5H- pyrrolo[3,2-c]pyridin-5-yl)-9H- pyrimido[4,5-b]indole-7-sulfonamide | [M + H]⁺ 600.2 600.2 |
| 39 | Int B Int 9 | 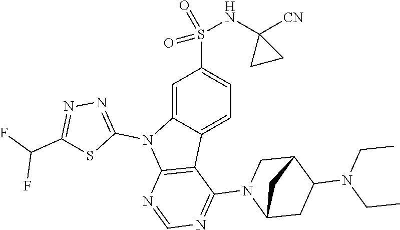 [SMILES: CCN(CC)C1C[C@H]2C[C@@H]1CN2c1ncnc2c1c1ccc(S(=O)(=O)NC3(C#N)CC3)cc1n2-c1nnc(C(F)F)s1] | N-(1-Cyanocyclopropyl)-4-((1R,4R)- 5-(diethylamino)-2- azabicyclo[2.2.1]heptan-2-yl)-9-(5- (difluoromethyl)-1,3,4-thiadiazol-2- yl)-9H-pyrimido[4,5-b]indole-7- sulfonamide | [M + H]⁺ 614.2 614.2 |
| 40 | Int B Int 10 | 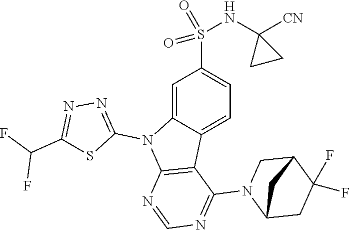 [SMILES: N#CC1(NS(=O)(=O)c2ccc3c4c(N5C[C@H]6C[C@@H]5CC6(F)F)ncnc4n(-c4nnc(C(F)F)s4)c3c2)CC1] | N-(1-Cyanocyclopropyl)-4-((1R,4R)- 5,5-difluoro-2- azabicyclo [2.2.1]heptan-2-yl)-9-(5- (difluoromethyl)-1,3,4-thiadiazol-2- yl)-9H-pyrimido[4,5-b]indole-7- sulfonamide | [M + H]⁺ 579.1 579.2 |
| 41 | Int B CAM | 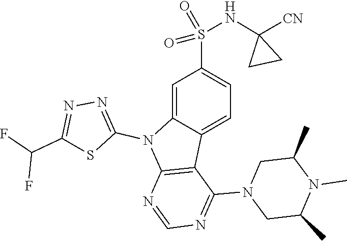 [SMILES: C[C@@H]1CN(c2ncnc3c2c2ccc(S(=O)(=O)NC4(C#N)CC4)cc2n3-c2nnc(C(F)F)s2)C[C@H](C)N1C] | N-(1-Cyanocyclopropyl)-9-(5- (difluoromethyl)-1,3,4-thiadiazol-2- yl)-4-((3R,5S)-3,4,5- trimethylpiperazin-1-yl)-9H- pyrimido[4,5-b]indole-7-sulfonamide | [M + H]⁺ 574.2 574.2 |
| 42 | Int B CAM | 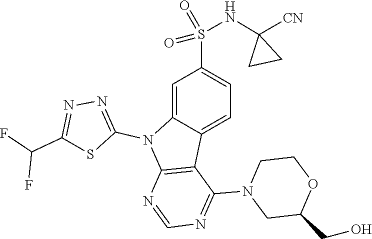 [SMILES: N#CC1(NS(=O)(=O)c2ccc3c4c(N5CCO[C@@H](CO)C5)ncnc4n(-c4nnc(C(F)F)s4)c3c2)CC1] | (R)-N-(1-Cyanocyclopropyl)-9-(5- (difluoromethyl)-1,3,4-thiadiazol-2- yl)-4-(2- (hydroxymethyl)morpholino)-9H- pyrimido[4,5-b]indole-7-sulfonamide | [M + H]⁺ 563.1 563.1 |
| 43 | Int B Int 11 | 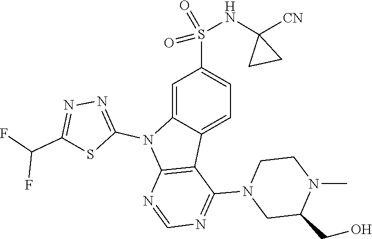 [SMILES: CN1CCN(c2ncnc3c2c2ccc(S(=O)(=O)NC4(C#N)CC4)cc2n3-c2nnc(C(F)F)s2)C[C@@H]1CO] | (R)-N-(1-Cyanocyclopropyl)-9-(5- (difluoromethyl)-1,3,4-thiadiazol-2- yl)-4-(3-(hydroxymethyl)-4- methylpiperazin-1-yl)-9H- pyrimido[4,5-b]indole-7-sulfonamide | [M + H]⁺ 576.1 576.1 |
| 44 | Int B CAM | 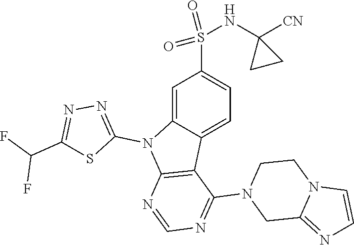 [SMILES: N#CC1(NS(=O)(=O)c2ccc3c4c(N5CCn6ccnc6C5)ncnc4n(-c4nnc(C(F)F)s4)c3c2)CC1] | N-(1-Cyanocyclopropyl)-9-(5- (difluoromethyl)-1,3,4-thiadiazol-2- yl)-4-(5,6-dihydroimidazo[1,2- a]pyrazin-7(8H)-yl)-9H- pyrimido[4,5-b]indole-7-sulfonamide | [M + H]⁺ 569.2 569.2 |
| 45 | Int B CAM | 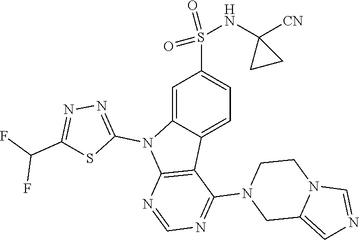 [SMILES: N#CC1(NS(=O)(=O)c2ccc3c4c(N5CCn6cncc6C5)ncnc4n(-c4nnc(C(F)F)s4)c3c2)CC1] | N-(1-Cyanocyclopropyl)-9-(5- (difluoromethyl)-1,3,4-thiadiazol-2- yl)-4-(5,6-dihydroimidazo[1,5- a]pyrazin-7(8H)-yl)-9H- pyrimido[4,5-b]indole-7-sulfonamide | [M + H]⁺ 569.2 569.2 |
| 46 | Int B CAM | 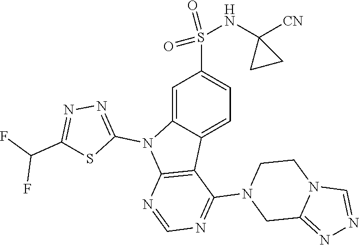 [SMILES: N#CC1(NS(=O)(=O)c2ccc3c4c(N5CCn6cnnc6C5)ncnc4n(-c4nnc(C(F)F)s4)c3c2)CC1] | N-(1-Cyanocyclopropyl)-9-(5- (difluoromethyl)-1,3,4-thiadiazol-2- yl)-4-(5,6-dihydro- [1,2,4]triazolo[4,3-a]pyrazin-7(8H)- yl)-9H-pyrimido[4,5-b]indole-7- sulfonamide | [M + H]⁺ 570.1 570.1 |
| 47 | Int B CAM | 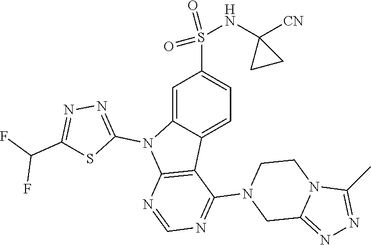 [SMILES: Cc1nnc2n1CCN(c1ncnc3c1c1ccc(S(=O)(=O)NC4(C#N)CC4)cc1n3-c1nnc(C(F)F)s1)C2] | N-(1-Cyanocyclopropyl)-9-(5- (difluoromethyl)-1,3,4-thiadiazol-2- yl)-4-(3-methyl-5,6-dihydro- [1,2,4]triazolo[4,3-a]pyrazin-7(8H)- yl)-9H-pyrimido[4,5-b]indole-7- sulfonamide | [M + H]⁺ 584.1 584.1 |
| 48 | Int B CAM | 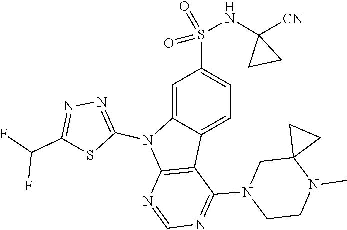 [SMILES: CN1CCN(c2ncnc3c2c2ccc(S(=O)(=O)NC4(C#N)CC4)cc2n3-c2nnc(C(F)F)s2)CC12CC2] | N-(1-Cyanocyclopropyl)-9-(5- (difluoromethyl)-1,3,4-thiadiazol-2- yl)-4-(4-methyl-4,7- diazaspiro[2.5]octan-7-yl)-9H- pyrimido[4,5-b]indole-7-sulfonamide | [M + H]⁺ 572.1 572.1 |
| 49 | Int B CAM | 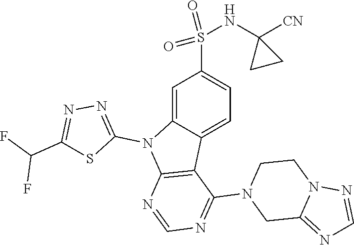 [SMILES: N#CC1(NS(=O)(=O)c2ccc3c4c(N5CCn6ncnc6C5)ncnc4n(-c4nnc(C(F)F)s4)c3c2)CC1] | N-(1-Cyanocyclopropyl)-9-(5- (difluoromethyl)-1,3,4-thiadiazol-2- yl)-4-(5,6-dihydro- [1,2,4]triazolo[1,5-a]pyrazin-7(8H)- yl)-9H-pyrimido[4,5-b]indole-7- sulfonamide | [M + H]⁺ 570.1 570.1 |
| 50 | Int B Int 12 | 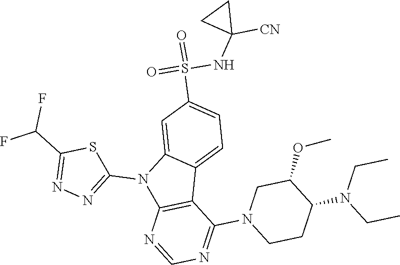 [SMILES: CCN(CC)[C@@H]1CCN(c2ncnc3c2c2ccc(S(=O)(=O)NC4(C#N)CC4)cc2n3-c2nnc(C(F)F)s2)C[C@@H]1OC] | N-(1-Cyanocyclopropyl)-4-((3S,4R)- 4-(diethylamino)-3- methoxypiperidin-1-yl)-9-(5- (difluoromethyl)-1,3,4-thiadiazol-2- yl)-9H-pyrimido[4,5-b]indole-7- sulfonamide | [M + H]⁺ 632.2 632.2 |
| 51 | Int B Int 13 | 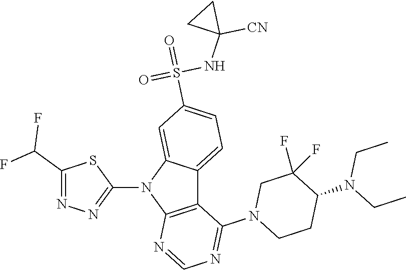 [SMILES: CCN(CC)[C@@H]1CCN(c2ncnc3c2c2ccc(S(=O)(=O)NC4(C#N)CC4)cc2n3-c2nnc(C(F)F)s2)CC1(F)F] | (R)-N-(1-Cyanocyclopropyl)-4-(4- (diethylamino)-3,3-difluoropiperidin- 1-yl)-9-(5-(difluoromethyl)-1,3,4- thiadiazol-2-yl)-9H-pyrimido[4,5- b]indole-7-sulfonamide | [M + H]⁺ 638.2 638.2 |
| 52 | Int B Int 14 | 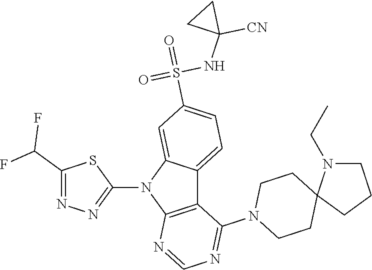 [SMILES: CCN1CCCC12CCN(c1ncnc3c1c1ccc(S(=O)(=O)NC4(C#N)CC4)cc1n3-c1nnc(C(F)F)s1)CC2] | N-(1-Cyanocyclopropyl)-9-(5- (difluoromethyl)-1,3,4-thiadiazol-2- yl)-4-(1-ethyl-1,8- diazaspiro[4.5]decan-8-yl)-9H- pyrimido[4,5-b]indole-7-sulfonamide | [M + H]⁺ 614.2 614.2 |
| 53 | Int B CAM | 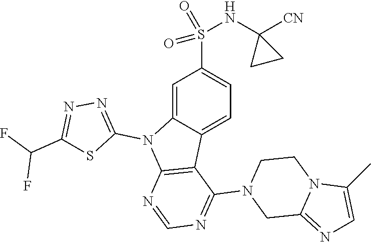 [SMILES: Cc1cnc2n1CCN(c1ncnc3c1c1ccc(S(=O)(=O)NC4(C#N)CC4)cc1n3-c1nnc(C(F)F)s1)C2] | N-(1-Cyanocyclopropyl)-9-(5- (difluoromethyl)-1,3,4-thiadiazol-2- yl)-4-(3-methyl-5,6- dihydroimidazo[1,2-a]pyrazin-7(8H)- yl)-9H-pyrimido[4,5-b]indole-7- sulfonamide | [M + H]⁺ 583.1 583.1 |
| 54 | Int B CAM | 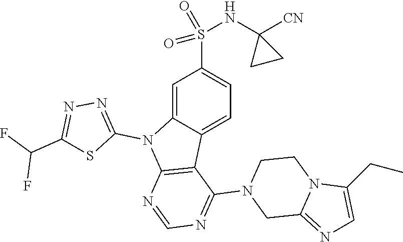 [SMILES: CCc1cnc2n1CCN(c1ncnc3c1c1ccc(S(=O)(=O)NC4(C#N)CC4)cc1n3-c1nnc(C(F)F)s1)C2] | N-(1-Cyanocyclopropyl)-9-(5- (difluoromethyl)-1,3,4-thiadiazol-2- yl)-4-(3-ethyl-5,6- dihydroimidazo[1,2-a]pyrazin-7(8H)- yl)-9H-pyrimido[4,5-b]indole-7- sulfonamide | [M + H]⁺ 597.1 597.1 |
| 55 | Int B CAM | 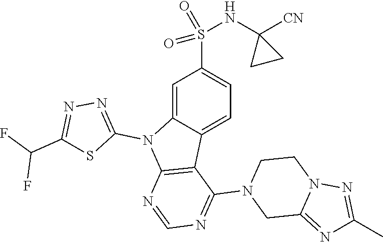 [SMILES: Cc1nc2n(n1)CCN(c1ncnc3c1c1ccc(S(=O)(=O)NC4(C#N)CC4)cc1n3-c1nnc(C(F)F)s1)C2] | N-(1-Cyanocyclopropyl)-9-(5- (difluoromethyl)-1,3,4-thiadiazol-2- yl)-4-(2-methyl-5,6-dihydro- [1,2,4]triazolo[1,5-a]pyrazin-7(8H)- yl)-9H-pyrimido[4,5-b]indole-7- sulfonamide | [M + H]⁺ 584.1 584.1 |
| 56 | Int B Int 15 | 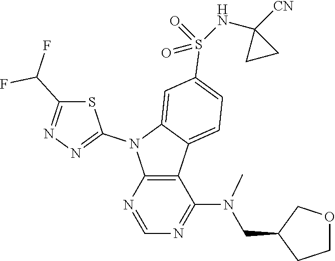 [SMILES: CN(C[C@@H]1CCOC1)c1ncnc2c1c1ccc(S(=O)(=O)NC3(C#N)CC3)cc1n2-c1nnc(C(F)F)s1] | (S)-N-(1-Cyanocyclopropyl)-9-(5- (difluoromethyl)-1,3,4-thiadiazol-2- yl)-4-(methyl((tetrahydrofuran-3- yl)methyl)amino)-9H-pyrimido[4,5- b]indole-7-sulfonamide | [M + H]⁺ 561.1 561.1 |
| 57 | Int B Int 16 | 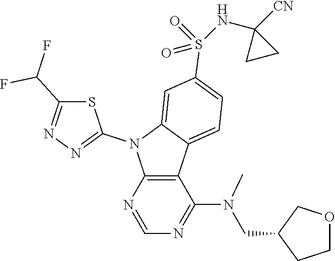 [SMILES: CN(C[C@H]1CCOC1)c1ncnc2c1c1ccc(S(=O)(=O)NC3(C#N)CC3)cc1n2-c1nnc(C(F)F)s1] | (R)-N-(1-Cyanocyclopropyl)-9-(5- (difluoromethyl)-1,3,4-thiadiazol-2- yl)-4-(methyl((tetrahydrofuran-3- yl)methyl)amino)-9H-pyrimido[4,5- b]indole-7-sulfonamide | [M + H]⁺ 561.1 561.1 |
| 58 | Int B CAM | 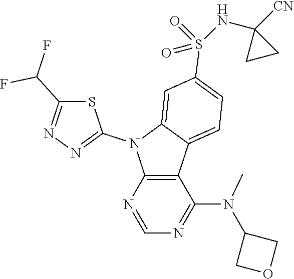 [SMILES: CN(c1ncnc2c1c1ccc(S(=O)(=O)NC3(C#N)CC3)cc1n2-c1nnc(C(F)F)s1)C1COC1] | N-(1-Cyanocyclopropyl)-9-(5- (difluoromethyl)-1,3,4-thiadiazol-2- yl)-4-(methyl(oxetan-3-yl)amino)- 9H-pyrimido[4,5-b]indole-7- sulfonamide | [M + H]⁺ 533.1 533.2 |
| 59 | Int B Int 17 | 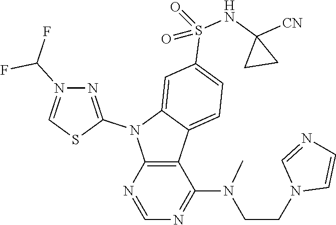 | 4-((2-(1H-Imidazol-1- yl)ethyl)(methyl)amino)-N-(1- cyanocyclopropyl)-9-(5- (difluoromethyl)-1,3,4-thiadiazol-2- yl)-9H-pyrimido[4,5-b]indole-7- sulfonamide | [M + H]⁺ 571.1 571.2 |
| 60 | Int B Int 18 | 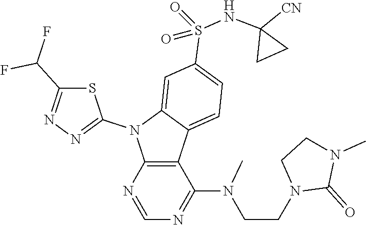 [SMILES: CN1CCN(CCN(C)c2ncnc3c2c2ccc(S(=O)(=O)NC4(C#N)CC4)cc2n3-c2nnc(C(F)F)s2)C1=O] | N-(1-Cyanocyclopropyl)-9-(5- (difluoromethyl)-1,3,4-thiadiazol-2- yl)-4-(methyl(2-(3-methyl-2- oxoimidazolidin-1-yl)ethyl)amino)- 9H-pyrimido[4,5-b]indole-7- sulfonamide | [M + H]⁺ 603.1 603.1 |
| 61 | Int B Int 19 | 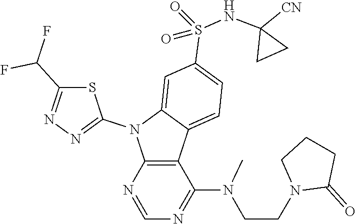 [SMILES: CN(CCN1CCCC1=O)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(methyl(2-(2-oxopyrrolidin-1- yl)ethyl)amino)-9H-pyrimido[4,5- b]indole-7-sulfonamide | [M + H]⁺ 588.1 588.1 |
| 62 | Int B CAM | 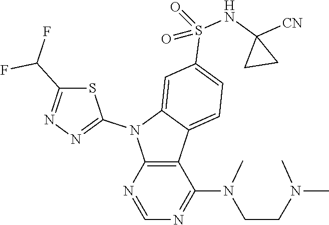 [SMILES: CN(C)CCN(C)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-((2- (dimethylamino)ethyl)(methyl)amino)- 9H-pyrimido[4,5-b]indole-7- sulfonamide | [M + H]⁺ 548.1 548.1 |
| 63 | Int B CAM | 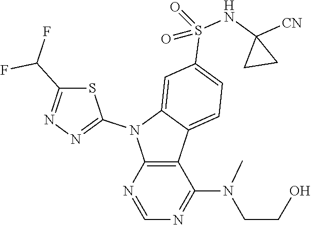 [SMILES: CN(CCO)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-((2- hydroxyethyl)(methyl)amino)-9H- pyrimido[4,5-b]indole-7-sulfonamide | [M + H]⁺ 521.1 521.1 |
| 64 | Int B Int 20 | 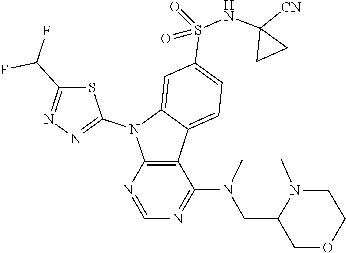 [SMILES: CN(CC1COCCN1C)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(methyl((4-methylmorpholin-3- yl)methyl)amino)-9H-pyrimido[4,5- b]indole-7-sulfonamide | [M + H]⁺ 590.2 590.2 |
| 65 | Int B Int 21 | 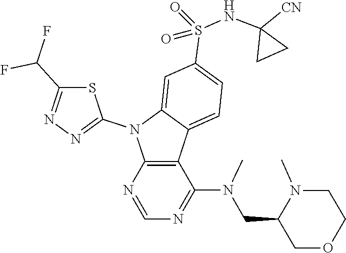 [SMILES: CN(C[C@@H]1COCCN1C)c1ncnc2c1c1ccc(S(=O)(=O)NC3(C#N)CC3)cc1n2-c1nnc(C(F)F)s1] | (R)-N-(1-Cyanocyclopropyl)-9-(5- (difluoromethyl)-1,3,4-thiadiazol-2- yl)-4-(methyl((4-methylmorpholin-3- yl)methyl)amino)-9H-pyrimido[4,5- b]indole-7-sulfonamide | [M + H]⁺ 590.2 590.2 |
| 66 | Int B Int 22 | 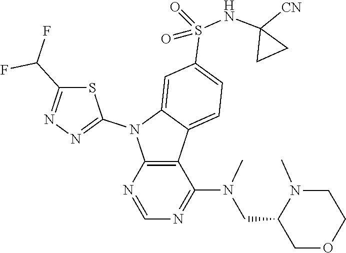 [SMILES: CN(C[C@H]1COCCN1C)c1ncnc2c1c1ccc(S(=O)(=O)NC3(C#N)CC3)cc1n2-c1nnc(C(F)F)s1] | (S)-N-(1-Cyanocyclopropyl)-9-(5- (difluoromethyl)-1,3,4-thiadiazol-2- yl)-4-(methyl((4-methylmorpholin-3- yl)methyl)amino)-9H-pyrimido[4,5- b]indole-7-sulfonamide | [M + H]⁺ 590.2 590.2 |
| 67 | Int B CAM | 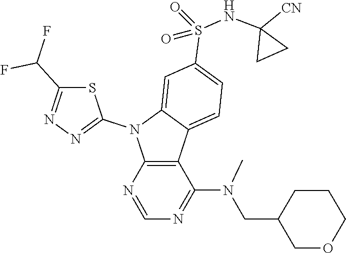 [SMILES: CN(CC1CCCOC1)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(methyl((tetrahydro-2H-pyran- 4-yl)methyl)amino)-9H- pyrimido[4,5-b]indole-7-sulfonamide | [M + H]⁺ 575.1 575.1 |
| 68 | Int B CAM | 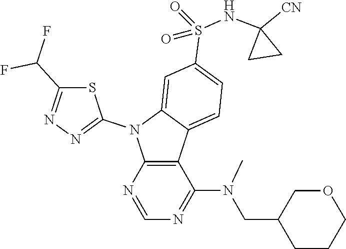 [SMILES: CN(CC1CCCOC1)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(methyl((tetrahydro-2H-pyran- 3-yl)methyl)amino)-9H- pyrimido[4,5-b]indole-7-sulfonamide | [M + H]⁺ 575.1 575.1 |
| 69 | Int B CAM | 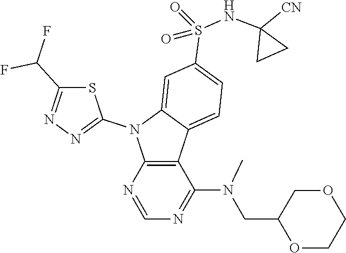 [SMILES: CN(CC1COCCO1)c1ncnc2c1c1ccc(S(=O)(=O)NC3(C#N)CC3)cc1n2-c1nnc(C(F)F)s1] | 4-(((1,4-Dioxan-2- yl)methyl)(methyl)amino)-N-(1- cyanocyclopropyl)-9-(5- (difluoromethyl)-1,3,4-thiadiazol-2- yl)-9H-pyrimido[4,5-b]indole-7- sulfonamide | [M + H]⁺ 577.1 577.1 |
| 70 | Int B CAM | 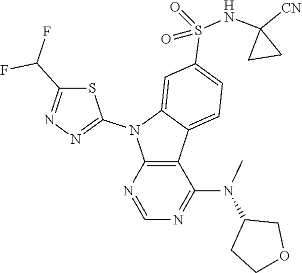 | (S)-N-(1-Cyanocyclopropyl)-9-(5- (difluoromethyl)-1,3,4-thiadiazol-2- yl)-4-(methyl(tetrahydrofuran-3- yl)amino)-9H-pyrimido[4,5-b]indole- 7-sulfonamide | [M + H]⁺ 547.1 547.1 |
| 71 | Int B Int 23 | 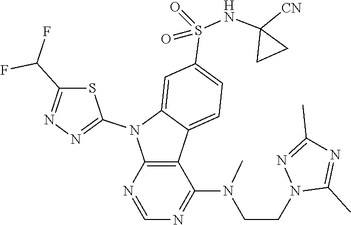 [SMILES: Cc1nc(C)n(CCN(C)c2ncnc3c2c2ccc(S(=O)(=O)NC4(C#N)CC4)cc2n3-c2nnc(C(F)F)s2)n1] | N-(1-Cyanocyclopropyl)-9-(5- (difluoromethyl)-1,3,4-thiadiazol-2- yl)-4-((2-(3,5-dimethyl-1H-1,2,4- triazol-1-yl)ethyl)(methyl)amino)- 9H-pyrimido[4,5-b]indole-7- sulfonamide | [M + H]⁺ 600.1 600.1 |
| 72 | Int B Int 24 | 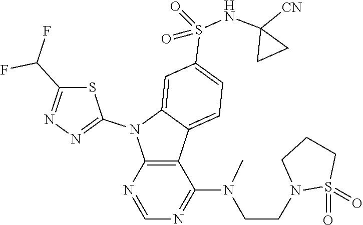 [SMILES: CN(CCN1CCCS1(=O)=O)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-((2-(1,1-dioxidoisothiazolidin- 2-yl)ethyl)(methyl)amino)-9H- pyrimido [4,5-b]indole-7-sulfonamide | [M + H]⁺ 624.1 624.1 |
| 73 | Int B Int 25 | 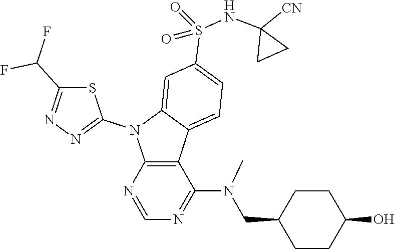 [SMILES: CN(C[C@H]1CC[C@@H](O)CC1)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-((((1s,4s)-4- hydroxycyclohexyl)methyl)(methyl) amino)-9H-pyrimido[4,5-b]indole-7- sulfonamide | [M + H]⁺ 589.2 589.2 |
| 74 | Int B CAM | 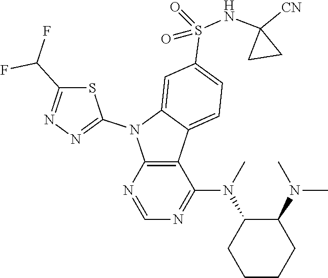 [SMILES: CN(C)[C@H]1CCCC[C@@H]1N(C)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(((1S,2S)-2- (dimethylamino)cyclohexyl)(methyl) amino)-9H-pyrimido[4,5-b]indole-7- sulfonamide | [M + H]⁺ 602.2 602.3 |
| 75 | Int B CAM | 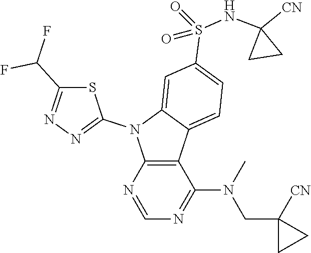 [SMILES: CN(CC1(C#N)CC1)c1ncnc2c1c1ccc(S(=O)(=O)NC3(C#N)CC3)cc1n2-c1nnc(C(F)F)s1] | N-(1-Cyanocyclopropyl)-4-(((1- cyanocyclopropyl)methyl)(methyl) amino)-9-(5-(difluoromethyl)-1,3,4- thiadiazol-2-yl)-9H-pyrimido[4,5- b]indole-7-sulfonamide | [M + H]⁺ 556.1 556.1 |
| 76 | Int B CAM | 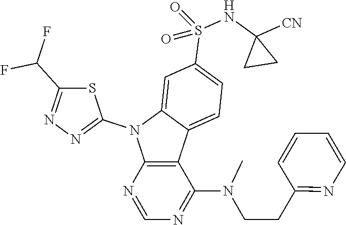 [SMILES: CN(CCc1ccccn1)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(methyl(2-(pyridin-2- yl)ethyl)amino)-9H-pyrimido[4,5- b]indole-7-sulfonamide | [M + H]⁺ 582.1 582.1 |
| 77 | Int B Int 26 | 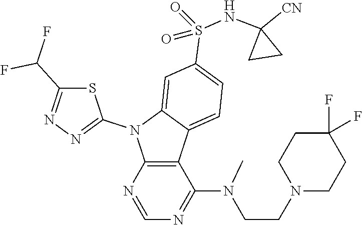 [SMILES: CN(CCN1CCC(F)(F)CC1)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-((2-(4,4-difluoropiperidin-1- yl)ethyl)(methyl)amino)-9H- pyrimido[4,5-b]indole-7-sulfonamide | [M + H]⁺ 624.2 624.2 |
| 78 | Int B Int 27 | 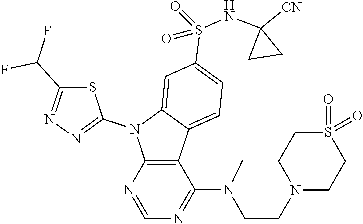 [SMILES: CN(CCN1CCS(=O)(=O)CC1)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-((2-(1,1- dioxidothiomorpholino)ethyl)(methyl) amino)-9H-pyrimido[4,5-b]indole-7- sulfonamide | [M + H]⁺ 638.1 638.1 |
| 79 | Int B CAM | 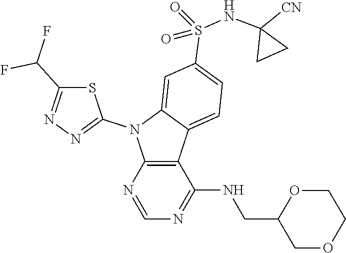 [SMILES: N#CC1(NS(=O)(=O)c2ccc3c4c(NCC5COCCO5)ncnc4n(-c4nnc(C(F)F)s4)c3c2)CC1] | 4-(((1,4-Dioxan-2-yl)methyl)amino)- N-(1-cyanocyclopropyl)-9-(5- (difluoromethyl)-1,3,4-thiadiazol-2- yl)-9H-pyrimido[4,5-b]indole-7- sulfonamide | [M + H]⁺ 563.1 563.2 |
| 80 | Int B Int 28 | 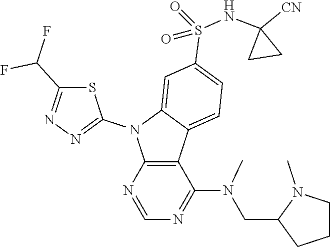 [SMILES: CN(CC1CCCN1C)c1ncnc2c1c1ccc(S(=O)(=O)NC3(C#N)CC3)cc1n2-c1nnc(C(F)F)s1] | N-(1-Cyanocyclopropyl)-9-(5- (difluoromethyl)-1,3,4-thiadiazol-2- yl)-4-(methyl((1-methylpyrrolidin-2- yl)methyl)amino)-9H-pyrimido[4,5- b]indole-7-sulfonamide | [M + H]⁺ 574.2 574.2 |
| 81 | Int B CAM | 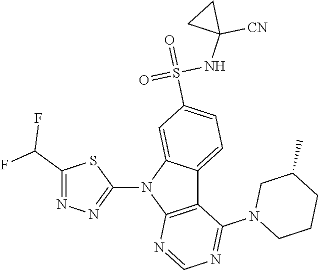 [SMILES: C[C@@H]1CCCN(c2ncnc3c2c2ccc(S(=O)(=O)NC4(C#N)CC4)cc2n3-c2nnc(C(F)F)s2)C1] | (R)-N-(1-Cyanocyclopropyl)-9-(5- (difluoromethyl)-1,3,4-thiadiazol-2- yl)-4-(3-methylpiperidin-1-yl)-9H- pyrimido[4,5-b]indole-7-sulfonamide | [M + H]⁺ 545.1 545.1 |

### Example 82: N-(7-(N-(1-Cyanocyclopropyl)sulfamoyl)-9-(5-(difluoromethyl)-1,3,4-thiadiazol-2-yl)-9H-pyrimido[4,5-b]indol-4-yl)-2-morpholinoacetamide

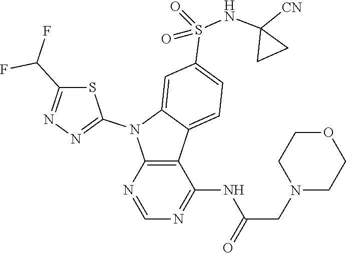 [SMILES: N#CC1(NS(=O)(=O)c2ccc3c4c(NC(=O)CN5CCOCC5)ncnc4n(-c4nnc(C(F)F)s4)c3c2)CC1]

### Step 1: 2-morpholinoacetamide

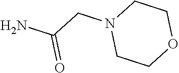

To a mixture of 2-chloroacetamide (2.8 g, 30 mmol) in EtOH (30 mL), NaI (6.0 g, 40 mmol) and K₂CO₃ (8.28 g, 60 mmol) was added morpholine (2.0 g, 20 mmol). The mixture was stirred at 85° C. for 6 h.. The reaction mixture was diluted with H₂O and extracted with EA (30 mL×3). The combined organic layers were washed with brine, dried over Na₂SO₄, filtered and concentrated under reduced pressure. The residue was purified by flash chromatography on a silica gel column eluting with MeOH/DCM (2-10%) to afford the title compound (1.1 g) as yellow solid. LCMS calc for C₆H₁₃N₂O₂ [M+H]⁺: m/z=145.1; Found: 145.2.

### Step 2: N-(7-(N-(1-cyanocyclopropyl)sulfamoyl)-9H-pyrimido[4,5-b]indol-4-yl)-2-morpholinoacetamide

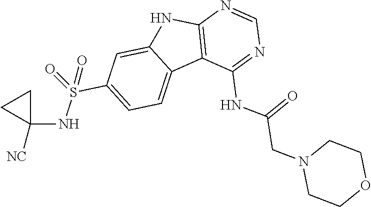 [SMILES: N#CC1(NS(=O)(=O)c2ccc3c(c2)[nH]c2ncnc(NC(=O)CN4CCOCC4)c23)CC1]

A mixture of 4-chloro-N-(1-cyanocyclopropyl)-9H-pyrimido[4,5-b]indole-7-sulfonamide (250 mg, 0.72 mmol, Int A), Pd(OAc)₂ (50 mg, 0.22 mmol), C⁵₂CO₃ (705 mg, 2.16 mmol), xantphos (209 mg, 0.36 mmol) and 2-morpholinoacetamide (156 mg, 1.08 mmol) in 1,4-dioxane (5 mL) was degassed and recharged with N₂ for 3 cycles. The mixture was stirred at 90° C. for 16 h.. The reaction mixture was diluted with H₂O (20 mL) and extracted with DCM (20 mL×3). The combined organic layers were washed with brine, dried over Na₂SO₄, filtered and concentrated under reduced pressure. The residue was purified by prep-TLC on a silica gel layer eluting with MeOH/DCM (0-10%) to afford the title compound (60 mg) as yellow solid. LCMS calc for C₂₀H₂₂N₇O₄S [M+H]⁺: m/z=456.1; Found: 456.1.

### Step 3: N-(7-(N-(1-cyanocyclopropyl)sulfamoyl)-9-(5-(difluoromethyl)-1,3,4-thiadiazol-2-yl)-9H-pyrimido[4,5-b]indol-4-yl)-2-morpholinoacetamide

A mixture of N-(7-(N-(1-cyanocyclopropyl)sulfamoyl)-9H-pyrimido[4,5-b]indol-4-yl)-2-morpholinoacetamide (55 mg, 0.12 mmol), 2-bromo-5-(difluoromethyl)-1,3,4-thiadiazole (103 mg, 0.48 mmol) and Cs₂CO₃ (117 mg, 0.36 mmol) in DMF (2 mL) was stirred at r.t. overnight. The reaction mixture was filtered, and concentrated under reduced pressure. The residue was purified by prep-HPLC on a C18 column eluting with MeCN/H₂O (15-75%, with 0.1% TFA) to afford the title product (13.6 mg) as white solid. LCMS calc. for C₂₃H₂₂F₂N₉O₄S₂ [M+H]⁺: m/z=590.1; Found: 590.1.

### Example 83: N-(1-Cyanocyclopropyl)-9-(5-(difluoromethyl)-1,3,4-thiadiazol-2-yl)-4-((2-morpholinoethyl)thio)-9H-pyrimido[4,5-b]indole-7-sulfonamide

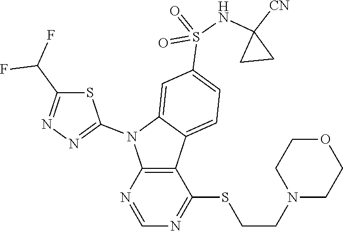 [SMILES: N#CC1(NS(=O)(=O)c2ccc3c4c(SCCN5CCOCC5)ncnc4n(-c4nnc(C(F)F)s4)c3c2)CC1]

### Step 1: N-(1-cyanocyclopropyl)-9-(5-(difluoromethyl)-1,3,4-thiadiazol-2-yl)-4-mercapto-9H-pyrimido[4,5-b]indole-7-sulfonamide

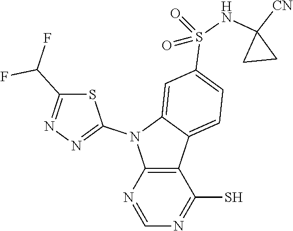 [SMILES: N#CC1(NS(=O)(=O)c2ccc3c4c(S)ncnc4n(-c4nnc(C(F)F)s4)c3c2)CC1]

To a solution of4-chloro-N-(1-cyanocyclopropyl)-9-[5-(difluoromethyl)-1,3,4-thiadiazol-2-yl]pyrimido[4,5-b]indole-7-sulfonamide (60 mg, 0.13 mmol, Int B) in DMAc (6 mL) was added NaHS (20.9 mg, 0.37 mmol). The mixture was stirred at 100° C. for 3 h., and then poured into water and extracted with EA (20 mL×3). The organic layers were washed with brine, dried over Na₂SO₄ and concentrated to afford the title crude compound (52 mg) as yellow solid. LCMS calc. for C₁₇H₁₂F₂N₇O₂S₃ [M+H]⁺: m/z=480.0; Found: 479.8.

### Step 2: N-(1-cyanocyclopropyl)-9-(5-(difluoromethyl)-1,3,4-thiadiazol-2-yl)-4-((2-morpholinoethyl)thio)-9H-pyrimido[4,5-b]indole-7-sulfonamide

To a mixture of N-(1-cyanocyclopropyl)-9-(5-(difluoromethyl)-1,3,4-thiadiazol-2-yl)-4-mercapto-9H-pyrimido[4,5-b]indole-7-sulfonamide (50 mg, 0.1 mmol) and 4-(2-chloroethyl)morpholine (19.4 mg, 0.1 mmol) in MeCN (2 mL) was added K₂CO₃ (43.2 mg, 0.31 mmol). The mixture was stirred at 60° C. for 2 h.. The reaction mixture was quenched with water and extracted with EA (20 mL×3). The organic layers were washed with brine, dried over Na₂SO₄ and concentrated under reduced pressure. The residue was purified by prep-HPLC on a C18 column eluting with MeCN/H₂O (25-75%, with 0.1% TFA) to afford the title compound (31.3 mg) as white solid. LCMS calc. for C₂₃H₂₃F₂N₈, O₃S₃ [M+H]⁺: m/z=593.1; Found: 593.0.

### Example 84: N-(1-Cyanocyclopropyl)-9-(5-(difluoromethyl)-1,3,4-thiadiazol-2-yl)-4-((2-methoxyethyl)thio)-9H-pyrimido[4,5-b]indole-7-sulfonamide

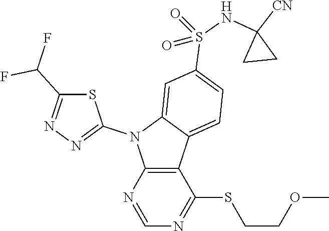 [SMILES: COCCSc1ncnc2c1c1ccc(S(=O)(=O)NC3(C#N)CC3)cc1n2-c1nnc(C(F)F)s1]

This compound was prepared by procedures analogous to those described for Example 83 Step 2 using 2-methoxyethylchloride to replace 4-(2-chloroethyl)morpholine. LCMS calc. for C₂₀H₁₈F₂N₇03S₃ [M+H]⁺: m/z=538.1; Found: 538.0.

### Example 85: (E)-N-(1-Cyanocyclopropyl)-9-(5-(difluoromethyl)-1,3,4-thiadiazol-2-yl)-4-(3-morpholinoprop-1-en-1-yl)-9H-pyrimido[4,5-b]indole-7-sulfonamide

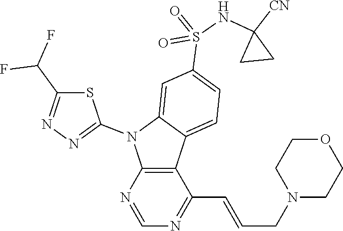 [SMILES: N#CC1(NS(=O)(=O)c2ccc3c4c(/C=C/CN5CCOCC5)ncnc4n(-c4nnc(C(F)F)s4)c3c2)CC1]

### Step 1: (E)-4-(3-(4,4,5,5-tetramethyl-1, 3,2-dioxaborolan-2-yl)allyl)morpholine

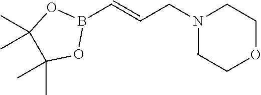 [SMILES: CC1(C)OB(/C=C/CN2CCOCC2)OC1(C)C]

A mixture of (E)-2-(3-chloroprop-1-en-1-yl)-4,4,5,5-tetramethyl-1,3,2-dioxaborolane (500 mg, 2.5 mmol), morpholine (323 mg, 3.7 mmol) and K₂CO₃ (683 mg, 5.0 mmol) in ACN (6 mL) was degassed and recharged with N₂ for 3 cycles. The mixture was stirred at 25° C. for 12 h., diluted with H₂O and extracted with EA (20 mL×3). The combined organic layers were washed with brine, dried over Na₂SO₄, filtered and concentrated under reduced pressure. The residue was purified by prep-HPLC on a C18 column eluting with MeCN/H₂O (25-75%) to afford the title compound (100 mg) as white solid. LCMS calc. for C₁₃H₂₅BNO₃ [M+H]⁺: m/z=254.2; Found: 254.1.

### Step 2: (E)-N-(1-cyanocyclopropyl)-9-(5-(difluoromethyl)-1,3,4-thiadiazol-2-yl)-4-(3-morpholinoprop-1-en-1-yl)-9H-pyrimido[4,5-b]indole-7- sulfonamide

A mixture of 4-chloro-N-(1-cyanocyclopropyl)-9-(5-(difluoromethyl)-1,3,4-thiadiazol-2-yl)-9H-pyrimido[4,5-b]indole-7-sulfonamide (80 mg, 0.17 mmol, Int B), (E)-4-(3-(4,4,5,5-tetramethyl-1,3,2-dioxaborolan-2-yl)allyl)morpholine (63 mg, 0.25 mmol), NaHCO₃ (42 mg, 0.5 mmol) and Pd(dppf)Cl₂ (12 mg, 0.017 mmol) in 1,4-dioxane (10 mL) was degassed and recharged with N₂ for 3 cycles and then stirred at 70° C. for 12 h.. The reaction mixture was diluted with H₂O and extracted with EA (20 mL×3). The combined organic layers were washed with brine, dried over Na₂SO₄, filtered and concentrated under reduced pressure. The residue was purified by prep-HPLC on a C18 column eluting with MeCN/H₂O (20-55% with 0.1% TFA) to afford the title compound (5.2 mg) as white solid. LCMS calc. for C₂₄H₂₃F₂N₈, O₃S₂ [M+H]⁺: m/z=573.1; Found: 573.0.

### Example 86: N-(1-Cyanocyclopropyl)-9-(5-(difluoromethyl)-1,3,4-thiadiazol-2-yl)-4-(3-morpholinoprop-1-yn-1-yl)-9H-pyrimido[4,5-b]indole-7-sulfonamide

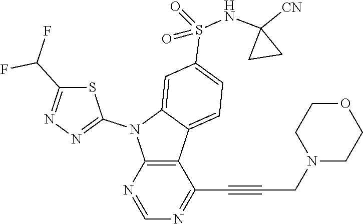 [SMILES: N#CC1(NS(=O)(=O)c2ccc3c4c(C#CCN5CCOCC5)ncnc4n(-c4nnc(C(F)F)s4)c3c2)CC1]

To a solution of 4-chloro-N-(1-cyanocyclopropyl)-9-(5-(difluoromethyl)-1,3,4-thiadiazol-2-yl)-9H-pyrimido[4,5-b]indole-7-sulfonamide (60.0 mg, 0.12 mmol, Int B) in DMF (6 mL) was added TEA (38 mg, 0.38 mmol), and then stirred at 25° C. for 45 min. To the above mixture Pd(PPh₃)₂Cl₂ (9 mg, 0.013 mmol), CuI (12 mg, 0.06 mmol) and 4-(prop-2-yn-1-yl)morpholine (46.7 mg, 0.374 mmol) was added. The reaction mixture was stirred at 45° C. for 4 h., and then diluted with H₂O, extracted with EA (20 mL×3). The combined organic layers were washed with brine, dried over Na₂SO₄, filtered and concentrated under reduced pressure. The residue was purified by prep-TLC on a silica gel layer eluting with MeOH/DCM (5%) to afford the title compound (12.4 mg) as yellow solid. LCMS calc. for C₂₄H₂₁F₂N₈, O₃S₂ [M+H]⁺: m/z=571.1; Found: 571.0.

### Example 87: N-(1-Cyanocyclopropyl)-9-(5-(difluoromethyl)-1,3,4-thiadiazol-2-yl)-4-(3-morpholinopropyl)-9H-pyrimido[4,5-b]indole-7-sulfonamide

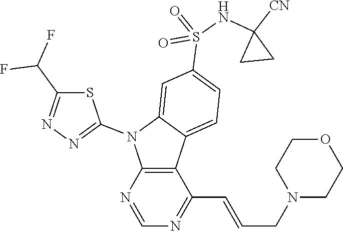 [SMILES: N#CC1(NS(=O)(=O)c2ccc3c4c(/C=C/CN5CCOCC5)ncnc4n(-c4nnc(C(F)F)s4)c3c2)CC1]

A mixture of N-(1-cyanocyclopropyl)-9-[5-(difluoromethyl)-1,3,4-thiadiazol-2-yl]-4-[3-(morpholin-4-yl)prop-1-yn-1-yl]pyrimido[4,5-b]indole-7-sulfonamide (25 mg, 0.044 mmol, Example 86) in EtOAc (10 mL) was added Pd/C (10 mg, 10% on carbon) and Pd(OH)₂/C (10 mg, 10% on carbon) was degassed and recharged with H₂ for 3 cycles. After stirred at r.t. overnight, the reaction mixture was filtered. The filtrate was evaporated under reduced pressure. The residue was purified by prep-TLC on a silica gel layer eluting with MeOH/DCM (10%) to afford the title compound (15.4 mg) as white solid. LCMS calc. for C₂₄H₂₅F₂N₈, O₃S₂ [M+H]⁺: m/z=575.1; Found: 575.1.

### Example 88: N-(1-Cyanocyclopropyl)-9-(5-(difluoromethyl)-1,3,4-thiadiazol-2-yl)-4-(3-hydroxy-3-methylbut-1-yn-1-yl)-9H-pyrimido[4,5-b]indole-7-sulfonamide

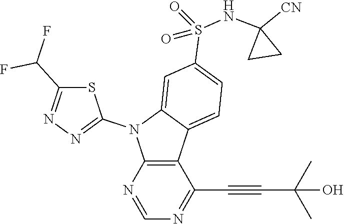 [SMILES: CC(C)(O)C#Cc1ncnc2c1c1ccc(S(=O)(=O)NC3(C#N)CC3)cc1n2-c1nnc(C(F)F)s1]

This compound was prepared by procedures analogous to those described for Example 86 using 2-methyl-3-butyn-2-ol and Int B as starting material. LCMS calc. for C₂₂H₁₈F₂N₇03S₂ [M+H]⁺: m/z=530.1; Found: 530.0.

### Example 89: N-(1-Cyanocyclopropyl)-9-(5-(difluoromethyl)-1,3,4-thiadiazol-2-yl)-4-(3-hydroxy-3-methylbutyl)-9H-pyrimido[4,5-b]indole-7-sulfonamide

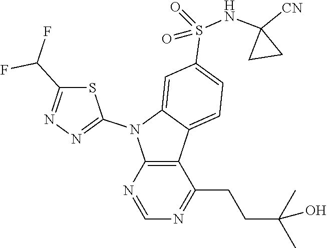 [SMILES: CC(C)(O)CCc1ncnc2c1c1ccc(S(=O)(=O)NC3(C#N)CC3)cc1n2-c1nnc(C(F)F)s1]

This compound was prepared by procedures analogous to those described for Example 87 using N-(1-cyanocyclopropyl)-9-(5-(difluoromethyl)-1,3,4-thiadiazol-2-yl)-4-(3-hydroxy-3-methylbut-1-yn-1-yl)-9H-pyrimido[4,5-b]indole-7-sulfonamide (Example 88). LCMS calc. for C₂₂H₂₂F₂N₇03S₂ [M+H]⁺: m/z=534.1; Found: 534.1.

### Example 90: N-(1-Cyanocyclopropyl)-9-(5-(difluoromethyl)-1,3,4-thiadiazol-2-yl)-4-((4-methylmorpholin-3-yl)methoxy)-9H-pyrimido[4,5-b]indole-7-sulfonamide

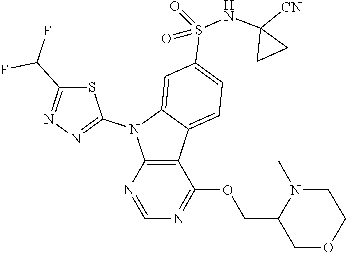 [SMILES: CN1CCOCC1COc1ncnc2c1c1ccc(S(=O)(=O)NC3(C#N)CC3)cc1n2-c1nnc(C(F)F)s1]

To a mixture of 4-chloro-N-(1-cyanocyclopropyl)-9-[5-(difluoromethyl)-1,3,4-thiadiazol-2-yl]pyrimido[4,5-b]indole-7-sulfonamide (50 mg, 0.1 mmol, Int B) and (4-methylmorpholin-3-Y¹)methanol (20 mg, 0.16 mmol) in THF (2 mL) was added sodium tert-butoxide (20 mg, 0.21 mmol) at 0° C., and then stirred at 25° C. for 2 h.. The reaction mixture was quenched with saturated aq. NH₄Cl (10 mL) and extracted with EA (10 mL×3). The organic layers were washed with brine, dried over Na₂SO₄ and concentrated under reduced pressure. The residue was purified by prep-TLC on a silica gel layer eluting with MeOH/DCM (5%) to give the title compound (19.2 mg) as a white solid. LCMS calc. for C₂₃H₂₃F₂N₈04S₂ [M+H]⁺: m/z=577.1; Found: 577.1.

### Example 91: N-(1-Cyanocyclopropyl)-9-(5-(difluoromethyl)-1,3,4-thiadiazol-2-yl)-4-(2-(1,1-dioxidoisothiazolidin-2-yl)ethoxy)-9H-pyrimido[4,5-b]indole-7-sulfonamide

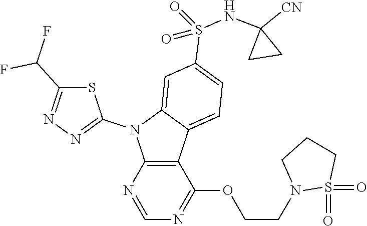 [SMILES: N#CC1(NS(=O)(=O)c2ccc3c4c(OCCN5CCCS5(=O)=O)ncnc4n(-c4nnc(C(F)F)s4)c3c2)CC1]

### Step 1: methyl ((3-chloropropyl)sulfonyl)glycinate

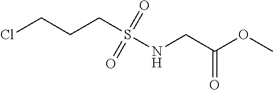

To a mixture of methyl 2-aminoacetate (lg, 11.2 mmol) and DIEA (5.79 g, 44.8 mmol) in THF (20 mL) was added 3-chloropropane-1-sulfonyl chloride (2.18 g, 12.3 mmol) at 0° C., and then stirred at r.t. for 2 h. The reaction mixture was diluted with water and extracted with EA (20 mL×3). The combined organic layers were washed with brine, dried over Na₂SO₄ and concentrated under reduced pressure. The residue was purified by flash chromatography on a silica gel column eluting with EtOAc/PE (30-40%) to afford the title compound (1.2 g) as yellow oil. LCMS calc for C₆H₁₃ClNO₄S [M+H]⁺: m/z=230.0; Found: 230.0

### Step 2: methyl 2-(1,1-dioxidoisothiazolidin-2-yl)acetate

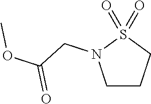

To a solution of methyl 2-[(3-chloropropane)sulfonamido]acetate (500 mg, 2.2 mmol) in DMF (10 mL) was added NaH (105 mg, 2.6 mmol, 60% dispersion in mineral oil) at 0° C., and then stirred at 0° C. for 3 h.. The resulting mixture was quenched with water and extracted with EA (30 mL×3). The combined organic layers were washed with brine, dried over Na₂SO₄, filtered and concentrated under reduced pressure. The residue was purified by prep-TLC on a silica gel layer eluting with EA/PE (0-50%) to afford the title compound (200 mg) as yellow oil. LCMS calc for C₆H₁₂NO₄S [M+H]⁺: m/z=194.0; Found: 194.0.

### Step 3: 2-(2-hydroxyethyl)isothiazolidine 1,1-dioxide

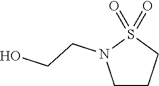

To a solution of methyl 2-[(3-chloropropane)sulfonamido]acetate (200 mg, 1.05 mmol) in THF (10 mL) was added LiBH₄ (114 mg, 5.2 mmol) at 0° C. The mixture was stirred at r.t. overnight. The reaction mixture was quenched with saturated aq. NH₄Cl (10 mL) and extracted with EA (20 mL×3). The combined organic layers were washed with brine, dried over Na₂SO₄, filtered and concentrated under reduced pressure. The residue was purified by prep-TLC on a silica gel layer eluting with MeOH/DCM (0-5%) to afford the title compound (50 mg) as yellow oil. LCMS calc for C₅H₁₂NO₃S [M+H]⁺: m/z=166.0; Found: 166.0.

### Step 4: N-(1-cyanocyclopropyl)-9-(5-(difluoromethyl)-1,3,4-thiadiazol-2-yl)-4-(2-(1,1-dioxidoisothiazolidin-2-yl)ethoxy)-9H-pyrimido[4,5-b]indole-7-sulfonamide

This compound was prepared by procedures analogous to those described for Example 90 using 2-(2-hydroxyethyl)isothiazolidine 1,1-dioxide and Int B as starting material. LCMS calc. for C₂₂H₂₁F₂N₈O₅S₃ [M+H]⁺: m/z=611.1; Found: 611.0.

TABLE 5
¹H NMR data of Examples (Ex)

| Ex # | ¹H NMR: (MHz, Solvent) δ |
|---|---|
| 2 | ¹H NMR: (400 MHz, DMSO-d⁶) δ 9.58-9.38 (m, 2H), 9.04 (s, 1H), 8.41 (d, J = |
| | 8.0 Hz, 1H), 8.10 (d, J = 8.0 Hz, 1H), 7.71 (t, J_{F-H} = 53.2 Hz, 1H), 4.89-4.69 (m, |
| | 2H), 4.19-3.91 (m, 2H), 3.72-3.43 (m, 6H), 3.23-3.09 (m, 2H), 2.41-2.30 (m, |
| | 2H), 1.49-1.38 (m, 2H), 1.35-1.24 (m, 2H). |
| 3 | ¹H NMR: (400 MHz, DMSO-d⁶) δ. 9.70-9.29 (m, 2H), 8.99 (s, 1H), 8.32 (d, J = |
| | 8.0 Hz, 1H), 8.09 (d, J = 8.0 Hz, 1H), 7.71 (t, J_{F-H} = 53.2 Hz, 1H), 4.78-4.65 (m, |
| | 2H), 3.61-3.48 (m, 4H), 2.44-2.24 (m, 6H), 2.02-1.89 (m, 2H), 1.75-1.62 (m, |
| | 2H), 1.48-1.38 (m, 2H), 1.32-1.22 (m, 2H). |
| 4 | ¹H NMR: (400 MHz, DMSO-d⁶) δ 9.58-9.41 (m, 2H), 9.07 (s, 1H), 8.49 (d, J = |
| | 8.0 Hz, 1H), 8.10 (d, J = 8.0 Hz, 1H), 7.71 (t, J_{F-H} = 53.2 Hz, 1H), 5.07 (s, 2H), |
| | 4.60-3.75 (m, 5H), 3.45-3.01 (m, 5H), 2.94-2.87 (m, 1H), 1.49-1.39 (m, 2H), |
| | 1.36-1.26 (m, 2H), 1.01 (d, J = 8.0 Hz, 6H). |
| 5 | ¹H NMR: (400 MHz, DMSO-d⁶) δ 9.49-9.38 (m, 2H), 9.00 (s, 1H), 8.36 (d, J = |
| | 8.0 Hz, 1H), 8.10 (d, J = 8.0 Hz, 1H), 7.71 (t, J_{F-H} = 53.2 Hz, 1H), 4.94-4.72 (m, |
| | 2H), 3.15-3.05 (m, 4H), 3.00-2.90 (m, 2H), 2.71 (s, 6H), 2.60-2.53 (m, 4H), |
| | 1.47-1.40 (m, 2H), 1.31-1.25 (m, 2H). |
| 6 | ¹H NMR: (400 MHz, DMSO-d⁶) δ 9.50-9.39 (m, 2H), 8.97 (s, 1H), 8.37 (d, J = |
| | 8.0 Hz, 1H), 8.12 (d, J = 8.0 Hz, 1H), 7.70 (t, J_{F-H} = 53.2 Hz, 1H), 5.53 (s, 2H), |
| | 3.07 (s, 3H), 2.87 (s, 3H), 1.48-1.39 (m, 2H), 1.32-1.23 (m, 2H). |
| 7 | ¹H NMR: (400 MHz, DMSO-d⁶) δ. 9.46-9.44 (m, 2H), 9.00 (s, 1H), 8.36 (d, J = |
| | 8.0 Hz, 1H), 8.07 (d, J = 8.0 Hz, 1H), 7.71 (t, J_{F-H} = 53.2 Hz, 1H) (m, 1H), |
| | 5.99-5.92 (m, 1H), 4.14-3.98 (m, 3H), 3.92-3.84 (m, 1H), 2.47-2.37 (m, 1H), |
| | 2.37-2.27 (m, 1H), 1.48-1.39 (m, 2H), 1.31-1.24 (m, 2H). |
| 8 | ¹H NMR: (400 MHz, DMSO-d⁶) δ. 9.46-9.44 (m, 2H), 9.00 (s, 1H), 8.36 (d, J = |
| | 8.0 Hz, 1H), 8.07 (d, J = 8.0 Hz, 1H), 7.71 (t, J_{F-H} = 53.2 Hz, 1H), 5.99-5.92 (m, |
| | 1H), 4.13-3.98 (m, 3H), 3.95-3.82 (m, 1H), 2.46-2.38 (m, 1H), 2.36-2.28 (m, |
| | 1H), 1.48-1.38 (m, 2H), 1.31-1.24 (m, 2H). |
| 9 | ¹H NMR: (400 MHz, DMSO-d⁶) δ 9.45-9.44 (m, 2H), 9.00 (s, 1H), 8.35-8.33 |
| | (d, J = 8.0 Hz, 1H), 8.07 (d, J = 8.0 Hz, 1H), 7.71 (t, J_{F-H} = 53.2 Hz, 1H), 4.78- |
| | 4.56 (m, 2H), 3.96-3.82 (m, 2H), 3.78-3.63 (m, 2H), 2.98-2.80 (m, 1H), 2.20- |
| | 2.11 (m, 1H), 1.85-1.77 (m, 1H), 1.47-1.39 (m, 2H), 1.30-1.25 (m, 2H). |
| 10 | ¹H NMR: (400 MHz, DMSO-d⁶) δ 9.50-9.48 (m, 2H), 9.08 (s, 1H), 8.47 (d, J = |
| | 8.4 Hz, 1H), 8.11 (d, J = 8.4 Hz, 1H), 7.71 (t, J_{F-H} = 53.2 Hz, 1H), 5.06-5.02 (m, |
| | 2H), 3.92-3.76 (m, 2H), 3.37-3.31 (m, 4H), 1.48-1.42 (m, 2H), 1.35-1.23 (m, |
| | 8H |
| 11 | ¹H NMR: (400 MHz, DMSO-d⁶) δ 9.50-9.40 (m, 2H), 8.99 (s, 1H), 8.38 (d, J = |
| | 8.0 Hz, 1H), 8.10 (d, J = 8.4 Hz, 1H), 7.71 (t, J_{F-H} = 53.2 Hz, 1H), 5.58 (s, 2H), |
| | 3.70-3.44 (m, 8H), 1.49-1.40 (m, 2H), 1.32-1.24 (m, 2H). |
| 12 | ¹H NMR: (400 MHz, DMSO-d⁶) δ 9.45-9.39 (m, 2H), 9.00-8.96 (m, 1H), 8.27 |
| | (d, J = 8.0 Hz, 1H), 8.06 (d, J = 8.0 Hz, 1H), 7.98-7.94 (m, 1H), 7.71 (t, J_{F-H} = |
| | 53.2 Hz, 1H), 7.49 (s, 1H), 6.26 (s, 1H), 5.05-4.98 (m, 2H), 4.78-4.70 (m, 2H), |
| | 1.47-1.41 (m, 2H), 1.31-1.25 (m, 2H). |
| 14 | ¹H NMR: (400 MHz, DMSO-d⁶) δ 9.48 (s, 1H), 9.36 (s, 1H), 8.73-8.66 (m, 1H), |
| | 8.28 (d, J = 8.0 Hz, 1H), 7.98 (d, J = 8.0 Hz, 1H), 7.71 (t, J_{F-H} = 53.2 Hz, 1H), |
| | 3.37 (s, 6H), 1.45-1.38 (m, 2H), 1.31-1.23 (m, 2H). |
| 15 | ¹H NMR: (400 MHz, DMSO-d⁶) δ 9.52 (s, 1H), 9.42 (s, 1H), 8.77 (s, 1H), 8.35 |
| | (d, J = 8.4 Hz, 1H), 8.02 (d, J = 8.4 Hz, 1H), 7.71 (t, J_{F-H} = 53.2 Hz, 1H), 4.18- |
| | 4.10 (m, 2H), 3.54-3.47 (m, 5H), 3.34-3.22 (m, 4H), 1.46-1.40 (m, 2H), 1.33- |
| | 1.29 (m, 2H), 1.29-1.22 (m, 6H). |
| 16 | ¹H NMR: (400 MHz, DMSO-d⁶) δ 9.47 (s, 1H), 9.40 (s, 1H), 8.71 (d, J = 8.0 |
| | Hz, 1H), 8.07 (d, J = 8.0 Hz, 1H), 7.71 (t, J_{F-H} = 53.2 Hz, 1H), 4.06-4.03 (m, |
| | 4H), 3.68-3.66 (m, 4H), 3.52-3.50 (m, 2H), 3.25-3.15 (m, 2H), 1.45-1.42 (m, |
| | 2H), 1.33-1.31 (m, 2H). |
| 17 | ¹H NMR: (400 MHz, DMSO-d⁶) δ 9.49 (s, 1H), 9.37 (s, 1H), 8.69 (s, 1H), 8.24 |
| | (d, J = 8.0 Hz, 1H), 8.00 (d, J = 8.0 Hz, 1H), 7.71 (t, J_{F-H} = 53.2 Hz, 1H), 3.89 |
| | (t, J = 4.0 Hz, 2H) 3.36 (s, 3H), 3.23-3.21 (m, 4H), 2.19-2.07 (m, 6H), 1.88- |
| | 1.85 (m, 2H), 1.44-1.41 (m, 2H), 1.29-1.25 (m, 2H). |
| 18 | ¹H NMR: (400 MHz, DMSO-d⁶) δ 9.53 (s, 1H), 9.43 (s, 1H), 8.81 (s, 1H), 8.18 |
| | (d, J = 8.0 Hz, 1H), 8.06 (d, J = 8.0 Hz, 1H), 7.71 (t, J_{F-H} = 53.2 Hz, 1H), 4.15- |
| | 4.05 (m, 2H), 3.89-3.87 (m, 2H), 3.68-3.58 (m, 4H), 3.30-2.80 (m, 4H), 2.00- |
| | 1.97 (m, 1H), 1.79-1.77 (m, 1H), 1.45-1.42 (m, 2H), 1.33-1.30 (m, 5H). |
| 19 | ¹H NMR: (400 MHz, DMSO-d⁶) δ 12.86 (s, 1H), 9.51 (s, 1H), 9.38 (s, 1H), 8.70 |
| | (s, 1H), 8.32 (d, J = 8.0 Hz, 1H), 8.00 (d, J = 8.0 Hz, 1H), 7.71 (t, J_{F-H} = 53.2 |
| | Hz, 1H), 4.46 (s, 2H), 3.54 (s, 3H), 1.44-1.41 (m, 2H), 1.29-1.26 (m, 2H). |
| 20 | ¹H NMR: (400 MHz, DMSO-d⁶) δ 9.51 (s, 1H), 9.37 (s, 1H), 8.67 (s, 1H), 8.27 |
| | (d, J = 8.0 Hz, 1H), 7.99 (d, J = 8.0 Hz, 1H), 7.71 (t, J_{F-H} = 53.2 Hz, 1H), 4.71 |
| | (s, 2H), 3.65-3.48 (m, 11H), 1.41-1.40 (m, 2H), 1.29-1.25 (m, 2H). |
| 21 | ¹H NMR: (400 MHz, DMSO-d⁶) δ 9.55-9.51 (m, 1H), 9.40 (s, 1H), 8.70 (s, 1H), |
| | 8.36 (d, J = 8.4 Hz, 1H), 7.98 (d, J = 8.4 Hz, 1H), 7.69 (t, J_{F-H} = 53.2 Hz, 1H), |
| | 7.67-7.55 (m, 4H), 5.19 (s, 2H), 3.97 (s, 3H), 1.46-1.40 (m, 2H), 1.32-1.26 (m, |
| | 2H). |
| 22 | ¹H NMR: (400 MHz, DMSO-d⁶) δ 9.52 (s, 1H), 9.38 (s, 1H), 8.68 (s, 1H), 8.60 |
| | (d, J = 4.0 Hz, 1H), 8.35 (d, J = 8.0 Hz, 1H), 7.98-7.90 (m, 2H), 7.81-7.55 (m, |
| | 1H), 7.50-7.40 (m, 2H), 5.18 (s, 2H), 3.47 (s, 3H), 1.44-1.41 (m, 2H), 1.29-1.26 |
| | (m, 2H). |
| 23 | ¹H NMR: (400 MHz, DMSO-d⁶) δ 9.49 (s, 1H), 9.38 (s, 1H), 8.75-8.66 (m, 1H), |
| | 8.28 (d, J = 8.0 Hz, 1H), 7.98 (d, J = 8.0 Hz, 1H), 7.71 (t, JF-H = 53.2 Hz, 1H), |
| | 3.93-3.82 (m, 2H), 3.76-3.66 (m, 2H), 3.63-3.56 (m, 1H), 3.46-3.40 (m, 4H), |
| | 2.89-2.75 (m, 1H), 2.05-1.93 (m, 1H), 1.60-1.49 (m, 1H), 1.47-1.37 (m, 2H), |
| | 1.32-1.23 (m, 2H). |
| 24 | ¹H NMR: (400 MHz, DMSO-d⁶) δ 9.47 (s, 1H), 9.37 (s, 1H), 8.65 (s, 1H), 8.15 |
| | (d, J = 8.0 Hz, 1H), 7.98 (d, J = 8.0 Hz, 1H), 7.71 (t, J_{F-H} = 53.2 Hz, 1H), 7.29 |
| | (s, 1H), 6.08 (t, J = 2.0 Hz, 1H), 4.51 (t, J = 6.0 Hz, 2H), 4.24 (t, J = 6.0 Hz, |
| | 2H), 3.29 (s, 3H), 1.45-1.41 (m, 2H), 1.29-1.26 (m, 2H). |
| 26 | ¹H NMR: (400 MHz, DMSO-d⁶) δ 9.55-9.45 (m, 2H), 9.18 (s, 1H), 8.44 (d, J = |
| | 8.4 Hz, 1H), 8.16 (d, J = 8.4 Hz, 1H), 7.71 (t, J_{F-H} = 53.2 Hz, 1H), 3.59-3.54 (m, |
| | 2H), 1.49-1.43 (m, 5H), 1.28-1.23 (m, 2H). |
| 27 | ¹H NMR: (400 MHz, DMSO-d⁶) δ 9.52-9.47 (m, 2H), 9.16 (s, 1H), 8.48 (d, J = |
| | 8.0 Hz, 1H), 8.16 (d, J = 8.0 Hz, 1H), 7.71 (t, J_{F-H} = 53.2 Hz, 1H), 5.20 (s, 1H), |
| | 3.82-3.76 (m, 2H), 3.69-3.64 (m, 2H), 1.47-1.42 (m, 2H), 1.31-1.25 (m, 2H). |
| 33 | ¹H NMR: (600 MHz, DMSO-d⁶) δ 9.50 (s, 1H), 8.78 (s, 1H), 8.15 (d, J = 8.4 |
| | Hz, 1H), 8.05 (d, J = 8.4 Hz, 1H), 7.68 (t, J_{F-H} = 53.2 Hz, 1H), 5.08 (d, J_{F-H} = |
| | 48.6 Hz, 1H), 4.56-4.48 (m, 2H), 3.72-3.60 (m, 1H), 3.31-3.22 (m, 1H), 3.05- |
| | 2.90 (m, 1H), 2.76-2.60 (m, 4H), 2.20-2.10 (m, 1H), 1.89-1.78 (m, 1H), 1.50- |
| | 1.40 (m, 2H), 1.31-1.25 (m, 2H), 0.99 (t, J = 7.2 Hz, 6H). |
| 56 | ¹H NMR: (400 MHz, DMSO-d⁶) δ 9.52-9.46 (m, 1H), 9.38 (s, 1H), 8.72 (s, 1H), |
| | 8.28 (d, J = 8.4 Hz, 1H), 8.00 (d, J = 8.4 Hz,, 1H), 7.70 (t, J_{F-H} = 53.2 Hz, 1H), |
| | 3.94-3.81 (m, 2H), 3.78-3.66 (m, 2H), 3.64-3.56 (m, 1H), 3.47-3.39 (m, 4H), |
| | 2.87-2.77 (m, 1H), 2.03-1.95 (m, 1H), 1.58-1.50 (m, 1H), 1.46-1.39 (m, 2H), |
| | 1.31-1.26 (m, 2H) |
| 57 | ¹H NMR: (400 MHz, DMSO-d⁶) δ 9.52-9.46 (m, 1H), 9.38 (s, 1H), 8.72 (s, 1H), |
| | 8.28 (d, J = 8.4 Hz, 1H), 8.00 (d, J = 8.4 Hz, 1H), 7.70 (t, J_{F-H} = 53.2 Hz, 1H), |
| | 3.94-3.81 (m, 2H), 3.78-3.66 (m, 2H), 3.64-3.56 (m, 1H), 3.47-3.39 (m, 4H), |
| | 2.87-2.77 (m, 1H), 2.03-1.95 (m, 1H), 1.58-1.50 (m, 1H), 1.46-1.39 (m, 2H), |
| | 1.31-1.26 (m, 2H) |
| 58 | ¹H NMR: (400 MHz, DMSO-d⁶) δ 9.56-9.54 (m, 1H), 9.22 (s, 1H), 8.67 (d, J = |
| | 8.8 Hz, 1H), 8.05 (d, J = 8.8 Hz, 1H), 7.70 (t, J_{F-H} = 53.2 Hz, 1H), 5.42-5.39 (m, |
| | 1H), 4.94-4.88 (m, 1H), 4.69-4.66 (m, 1H), 4.56 (s, 1H), 3.99-3.93 (m, 1H), |
| | 3.88 (s, 3H), 3.68-3.64 (m, 1H), 1.49-1.43 (m, 2H), 1.33-1.28 (m, 2H). |
| 59 | ¹H NMR: (400 MHz, DMSO-d⁶) δ 9.49 (s, 1H), 9.42 (s, 1H), 9.03 (s, 1H), 8.54 |
| | (s, 1H), 8.25 (d, J = 8.0 Hz, 1H), 8.00 (d, J = 8.0 Hz, 1H), 7.71 (t, J_{F-H} = 53.2 |
| | Hz, 1H), 7.55 (s, 0.26 H), 7.48 (s, 1H), 4.62-4.59 (m, 2H), 4.32-4.30 (m, 2H), |
| | 3.46 (s, 3H), 1.44-1.42 (m, 2H), 1.31-1.28 (m, 2H). |
| 60 | ¹H NMR: (400 MHz, DMSO-d⁶) δ 9.48 (s, 1H), 9.37 (s, 1H), 8.69 (s, 1H), 8.22 |
| | (d, J = 8.0 Hz, 1H), 7.98 (d, J = 8.0 Hz, 1H), 7.68 (t, J_{F-H} = 53.2 Hz, 1H), 4.01- |
| | 3.98 (m, 2H), 3.48-3.45 (m, 2H), 3.35 (s, 3H), 3.14-3.10 (m, 2H), 2.97-2.93 (m, |
| | 2H), 2.44 (s, 3H), 1.44-1.41 (m, 2H), 1.29-1.26 (m, 2H). |
| 61 | ¹H NMR: (400 MHz, DMSO-d⁶) δ 9.49 (s, 1H), 9.37 (s, 1H), 8.71 (s, 1H), 8.23 |
| | (d, J = 8.0 Hz, 1H), 7.99 (d, J = 8.0 Hz, 1H), 7.68 (t, J_{F-H} = 53.2 Hz, 1H), 4.05- |
| | 4.02 (m, 2H), 3.61-3.55 (m, 2H), 3.43 (s, 3H), 3.24-3.21 (m, 2H), 1.99-1.95 (m, |
| | 2H), 1.68-1.65 (m, 2H), 1.44-1.41 (m, 2H), 1.29-1.26 (m, 2H). |
| 62 | ¹H NMR: (400 MHz, DMSO-d⁶) δ 9.52 (s, 1H), 9.42 (s, 1H), 8.76 (s, 1H), 8.35 |
| | (d, J = 8.4 Hz, 1H), 8.03 (d, J = 8.4 Hz, 1H), 7.69 (t, J_{F-H} = 53.2 Hz, 1H), 4.18- |
| | 4.15 (m, 2H), 3.55-3.45 (m, 5H), 2.91 (s, 6H), 1.45-1.36 (m, 2H), 1.31-1.23 (m, |
| | 2H). |
| 63 | ¹H NMR: (400 MHz, DMSO-d⁶) δ 9.51-9.48 (m, 1H), 9.37 (s, 1H), 8.69 (s, 1H), |
| | 8.31 (d, J = 8.4 Hz, 1H), 7.99 (d, J = 8.4 Hz, 1H), 7.69 (t, J_{F-H} = 53.2 Hz, 1H), |
| | 4.88-4.83 (m, 1H), 3.95-3.88 (m, 2H), 3.80-3.73 (m, 2H), 3.44 (s, 3H), 1.46- |
| | 1.39 (m, 2H), 1.31-1.25 (m, 2H). |
| 64 | ¹H NMR: (400 MHz, DMSO-d⁶) δ 9.52 (s, 1H), 9.44 (s, 1H), 8.80 (s, 1H), 8.37 |
| | (d, J = 8.0 Hz, 1H), 8.01 (d, J = 8.0 Hz, 1H), 7.69 (t, J_{F-H} = 53.2 Hz, 1H), 4.26- |
| | 3.98 (m, 9H), 3.39-3.02 (m, 6H), 1.44-1.42 (m, 2H), 1.31-1.28 (m, 2H). |
| 65 | ¹H NMR: (400 MHz, DMSO-d⁶) δ 9.52 (s, 1H), 9.44 (s, 1H), 8.80 (s, 1H), 8.37 |
| | (d, J = 8.0 Hz, 1H), 8.01 (d, J = 8.0 Hz, 1H), 7.69 (t, J_{F-H} = 53.2 Hz, 1H), 4.26- |
| | 3.98 (m, 9H), 3.39-3.12 (m, 3H), 3.03 (s, 3H), 1.44-1.42 (m, 2H), 1.31-1.28 (m, |
| | 2H). |
| 66 | ¹H NMR: (400 MHz, DMSO-d⁶) δ 9.52 (s, 1H), 9.44 (s, 1H), 8.80 (s, 1H), 8.37 |
| | (d, J = 8.0 Hz, 1H), 8.01 (d, J = 8.0 Hz, 1H), 7.69 (t, J_{F-H} = 53.2 Hz, 1H), 4.26- |
| | 3.98 (m, 9H), 3.39-3.12 (m, 3H), 3.03 (s, 3H), 1.44-1.42 (m, 2H), 1.31-1.28 (m, |
| | 2H). |
| 67 | ¹H NMR: (400 MHz, DMSO-d⁶) δ 9.49 (s, 1H), 9.38 (s, 1H), 8.69 (s, 1H), 8.26 |
| | (d, J = 8.0 Hz, 1H), 7.99 (d, J = 8.0 Hz, 1H), 7.68 (t, J_{F-H} = 53.2 Hz, 1H), 3.80- |
| | 3.78 (m, 4H), 3.49 (s, 3H), 3.27-3.24 (m, 2H), 2.25-2.18 (m, 1H), 1.60-1.51 (m, |
| | 2H), 1.47-1.42 (m, 2H), 1.29-1.26 (m, 2H), 1.15-1.11 (m, 2H). |
| 68 | ¹H NMR: (400 MHz, DMSO-d⁶) δ 9.49 (s, 1H), 9.37 (s, 1H), 8.70 (s, 1H), 8.28 |
| | (d, J = 8.0 Hz, 1H), 7.99 (d, J = 8.0 Hz, 1H), 7.68 (t, J_{F-H} = 53.2 Hz, 1H), 3.80- |
| | 3.65 (m, 4H), 3.40 (s, 3H), 3.28-3.26 (m, 1H), 3.16-3.10 (m, 1H), 2.18-2.16 (m, |
| | 1H), 1.77-1.75 (m, 1H), 1.50-1.41 (m, 4H), 1.30-1.20 (m, 3H). |
| 69 | ¹H NMR: (400 MHz, DMSO-d⁶) δ 9.54-9.47 (m, 1H), 9.37 (s, 1H), 8.72 (s, 1H), |
| | 8.30 (d, J = 8.8 Hz, 1H), 8.00 (d, J = 8.8 Hz, 1H), 7.68 (t, J_{F-H} = 53.2 Hz, 1H), |
| | 4.05-3.98 (m, 1H), 3.95-3.86 (m, 2H), 3.82-3.77 (m, 1H), 3.73-3.68 (m, 1H), |
| | 3.65-3.60 (m, 1H), 3.59-3.53(m, 1H), 3.51-3.41 (m, 4H), 3.27-3.24 (m, 1H), |
| | 1.48-1.38 (m, 2H), 1.33-1.24 (m, 2H). |
| 70 | ¹H NMR: (400 MHz, DMSO-d⁶) δ 9.49 (s, 1H), 9.38 (s, 1H), 8.75 (s, 1H), 8.25 |
| | (d, J = 8.0 Hz, 1H), 8.01 (d, J = 8.0 Hz, 1H), 7.68 (t, J_{F-H} = 53.2 Hz, 1H), 5.24- |
| | 5.22 (m, 1H), 4.06-4.04 (m, 1H), 3.97-3.94 (m, 2H), 3.70-3.66 (m, 1H), 3.31 (s, |
| | 3H), 2.41-2.40 (m, 1H), 2.17-2.15 (m, 1H), 1.42-1.41 (m, 2H), 1.29-1.26 (m, |
| | 2H). |
| 71 | ¹H NMR: (400 MHz, DMSO-d⁶) δ 9.48 (s, 1H), 9.37 (s, 1H), 8.65 (s, 1H), 8.20 |
| | (d, J = 8.0 Hz, 1H), 8.00 (d, J = 8.0 Hz, 1H), 7.69 (t, J_{F-H} = 53.2 Hz, 1H), 4.41- |
| | 4.34 (m, 2H), 4.22-4.19 (m, 2H), 3.38 (s, 3H), 2.19 (s, 3H), 1.90 (s, 3H), 1.44- |
| | 1.41 (m, 2H), 1.29-1.23 (m, 2H). |
| 72 | ¹H NMR: (400 MHz, DMSO-d⁶) δ 9.50-9.47 (m, 1H), 9.37 (s, 1H), 8.70 (s, 1H), |
| | 8.27 (d, J = 8.0 Hz, 1H), 7.98 (d, J = 8.0 Hz, 1H), 7.71 (t, J_{F-H} = 53.2 Hz, 1H), |
| | 4.07-4.00 (m, 2H), 3.47 (s, 3H), 3.37-3.33 (m, 2H), 3.26-3.20 (m, 2H), 3.14- |
| | 3.08 (m, 2H), 2.17-2.08 (m, 2H), 1.46-1.40 (m, 2H), 1.30-1.25 (m, 2H). |
| 74 | ¹H NMR: (400 MHz, DMSO-d⁶) δ 9.55-9.42 (m, 2H), 8.73 (s, 1H), 8.45 (d, J = |
| | 8.0 Hz, 1H), 7.99 (d, J = 8.0 Hz, 1H), 7.70 (t, J_{F-H} = 53.2 Hz, 1H), 5.26 (s, 1H), |
| | 3.86 (s, 1H), 3.41 (s, 3H), 2.83 (s, 6H), 2.26-2.17 (m, 1H), 2.02-1.94 (m, 1H), |
| | 1.87-1.79 (m, 3H), 1.69-1.64 (m, 1H), 1.50-1.35 (m, 4H), 1.34-1.29 (m, 2H). |
| 75 | ¹H NMR: (400 MHz, DMSO-d⁶) δ 9.51 (s, 1H), 9.40 (s, 1H), 8.76 (s, 1H), 8.28 |
| | (d, J = 8.0 Hz, 1H), 8.03 (d, J = 8.0 Hz, 1H), 7.69 (t, J_{F-H} = 53.2 Hz, 1H), 4.08 |
| | (s, 2H), 3.55 (s, 3H), 1.45-1.37 (m, 4H), 1.30-1.23 (m, 4H). |
| 76 | ¹H NMR: (400 MHz, DMSO-d⁶) δ 9.48 (s, 1H), 9.38 (s, 1H), 8.56-9.38 (m, 2H), |
| | 8.21 (d, J = 8.0 Hz, 1H), 7.99-7.95 (m, 2H), 7.70 (t, J_{F-H} = 53.2 Hz, 1H), 7.62- |
| | 7.59 (m, 1H), 7.54-7.45 (m, 1H), 4.26-4.22 (m, 2H), 3.45 (s, 3H), 3.33-3.30 (m, |
| | 2H), 1.45-1.42 (m, 2H), 1.30-1.27 (m, 2H). |
| 77 | ¹H NMR: (400 MHz, DMSO-d⁶) δ 9.52 (s, 1H), 9.41 (s, 1H), 8.76 (s, 1H), 8.34 |
| | (d, J = 8.0 Hz, 1H), 8.01 (d, J = 8.0 Hz, 1H), 7.69 (t, J_{F-H} = 53.2 Hz, 1H), 4.20- |
| | 4.08 (m, 2H), 3.96-3.55 (m, 6H), 3.49 (s, 3H), 2.41-2.11 (m, 4H), 1.44-1.41 (m, |
| | 2H), 1.32-1.29 (m, 2H). |
| 78 | ¹H NMR: (400 MHz, DMSO-d⁶) δ 9.50 (s, 1H), 9.39 (s, 1H), 8.72 (s, 1H), 8.30 |
| | (d, J = 8.0 Hz, 1H), 8.00 (d, J = 8.0 Hz, 1H), 7.70 (t, J_{F-H} = 53.2 Hz, 1H), 4.05- |
| | 3.98 (m, 2H), 3.45 (s, 3H), 3.32-2.90 (m, 10H), 1.44-1.41 (m, 2H), 1.31-1.28 |
| | (m, 2H). |
| 79 | ¹H NMR: (400 MHz, DMSO-d⁶) δ 9.44 (s, 1H), 9.35 (s, 1H), 8.75 (m, 2H), |
| | 8.09-8.01 (m, 2H), 7.67 (t, J_{F-H} = 53.2 Hz, 1H), 3.94-3.87 (m, 1H), 3.86-3.80 |
| | (m, 1H), 3.79-3.70 (m, 3H), 3.67-3.62 (m, 1H), 3.61-3.55 (m, 1H), 3.55-3.45 |
| | (m, 2H), 1.45-1.41 (m, 2H), 1.30-1.26 (m, 2H). |
| 82 | ¹H NMR: (400 MHz, DMSO-d⁶) δ11.90 (b s, 1H), 9.53-9.52 (m, 2H), 9.23 (s, |
| | 1H), 8.20 (d, J = 8.0 Hz, 1H), 8.07 (d, J = 8.0 Hz, 1H), 7.70 (t, J_{F-H} = 53.2 Hz, |
| | 1H), 4.52-4.48 (m, 2H), 3.86-3.31 (m, 8H), 1.46-1.44 (m, 2H), 1.31-1.28 (m, |
| | 2H). |
| 83 | ¹H NMR: (400 MHz, DMSO-d⁶) δ 9.56-9.49 (m, 2H), 9.22 (s, 1H), 8.44 (d, J = |
| | 8.4 Hz, 1H), 8.20 (d, J = 8.4 Hz, 1H), 7.71 (t, J_{F-H} = 53.2 Hz, 1H), 7.59 (s, 0.47 |
| | H), 4.11-3.88 (m, 4H), 3.75-3.56 (m, 6H), 3.22 (s, 2H), 1.48-1.41 (m, 2H), 1.34- |
| | 1.25(m, 2H), |
| 84 | ¹H NMR: (400 MHz, DMSO-d⁶) δ 9.54-9.45 (m, 2H), 9.18 (s, 1H), 8.43 (d, J = |
| | 8.4 Hz, 1H), 8.16 (d, J = 8.4 Hz, 1H), 7.57 (t, J_{F-H} = 53.2 Hz, 1H), 3.76-3.73 (m, |
| | 4H), 3.15 (s, 3H), 1.47-1.43 (m, 2H), 1.30-1.26 (m, 2H). |
| 85 | ¹H NMR: (400 MHz, DMSO-d⁶) δ 9.58-9.53 (m, 2H), 9.32 (s, 1H), 8.82 (d, J = |
| | 8.4 Hz, 1H), 8.11 (d, J = 8.4 Hz, 1H), 7.93-7.84 (m, 1H), 7.68 (t, J_{F-H} = 53.2 Hz, |
| | 1H), 7.55-7.48 (m, 1H), 4.08 (s, 2H), 3.81 (s, 4H), 3.14 (s, 4H), 1.49-1.43 (m, |
| | 2H), 1.35-1.29 (m, 2H). |
| 86 | ¹H NMR: (400 MHz, DMSO-d⁶) δ 9.56 (s, 1H), 9.51 (s, 1H), 9.33 (s, 1H), 8.77 |
| | (d, J = 8.0 Hz, 1H), 8.15 (d, J = 8.0 Hz, 1H), 7.72 (t, J_{F-H} = 53.2 Hz, 1H), 3.97 |
| | (s, 2H), 3.69 (s, 4H), 3.59 (s, 2H), 2.74-2.67 (m, 2H), 1.48-1.44 (m, 2H), 1.31- |
| | 1.27 (m, 2H). |
| 87 | ¹H NMR: (400 MHz, DMSO-d⁶) δ 9.52-9.47 (m, 2H), 9.24 (s, 1H), 8.67 (d, J = |
| | 8.0 Hz, 1H), 8.09 (d, J = 8.0 Hz, 1H), 7.68 (t, J_{F-H} = 53.2 Hz, 1H), 3.47-3.40 (m, |
| | 6H), 2.35-2.22 (m, 4H), 2.11-2.03 (m, 2H), 1.44-1.40 (m, 2H), 1.29-1.24 (m, |
| | 2H). |
| 88 | ¹H NMR: (400 MHz, DMSO-d⁶) δ 9.55-9.50 (m, 2H), 9.33 (s, 1H), 8.75 (d, J = |
| | 8.4 Hz, 1H), 8.17 (d, J = 8.4 Hz, 1H), 7.72 (t, J_{F-H} = 53.2 Hz, 1H), 6.09 (s, 1H), |
| | 1.68 (s, 6H), 1.48-1.45 (m, 2H), 1.30-1.23 (m, 2H). |
| 89 | ¹H NMR: (400 MHz, DMSO-d⁶) δ 9.52-9.50 (m, 1H), 9.48 (s, 1H), 9.23 (s, 1H), |
| | 8.58 (d, J = 8.4 Hz, 1H), 8.09 (d, J = 8.4 Hz, 1H), 7.68 (t, J_{F-H} = 53.2 Hz, 1H), |
| | 4.60 (s, 1H), 3.45-3.99 (m, 2H), 1.94-1.88 (m, 2H), 1.45-1.41 (m, 2H), 1.28- |
| | 1.25 (m, 2H), 1.24 (s, 6H). |
| 90 | ¹H NMR: (400 MHz, DMSO-d⁶) δ 9.52-9.40 (m, 1H), 9.02 (s, 1H), 8.36 (d, J = |
| | 8.4 Hz, 1H), 8.12 (d, J = 8.4 Hz, 1H), 7.70 (t, J_{F-H} = 53.2 Hz, 1H), 4.84-4.73 (m, |
| | 2H), 4.02-3.95 (m, 1H), 3.78-3.71 (m, 1H), 3.62-3.52 (m, 2H), 2.76-2.69 (m, |
| | 1H), 2.68-2.62 (m, 1H), 2.39 (s, 3H), 2.33-2.26 (m, 1H), 1.48-1.38 (m, 2H), |
| | 1.31-1.25 (m, 2H). |
| 91 | ¹H NMR: (400 MHz, DMSO-d⁶) δ 9.59-9.36 (m, 2H), 9.03 (s, 1H), 8.51 (d, J = |
| | 8.0 Hz, 1H), 8.06 (d, J = 8.0 Hz, 1H), 7.70 (t, J_{F-H} = 53.2 Hz, 1H), 4.89-4.77 (m, |
| | 2H), 3.61-3.51 (m, 2H), 3.49-3.38 (m, 2H), 3.25-3.13 (m, 2H), 2.30-2.18 (m, |
| | 2H), 1.50-1.38 (m, 2H), 1.33-1.25 (m, 2H). |

### Example 92: N-(1-Cyanocyclopropyl)-9-(5-(difluoromethyl)-1,3,4-thiadiazol-2-yl)-4-(morpholin-3-ylmethoxy)-911-pyrimido[4,5-b]indole-7-sulfonamide

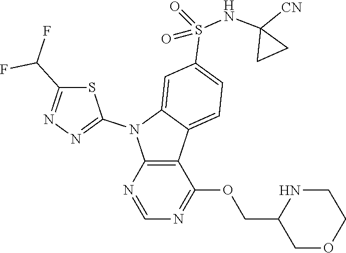 [SMILES: N#CC1(NS(=O)(=O)c2ccc3c4c(OCC5COCCN5)ncnc4n(-c4nnc(C(F)F)s4)c3c2)CC1]

### Step 1: tert-butyl 3-(((7-(N-(1-cyanocyclopropyl)sulfamoyl)-9-(5-(difluoromethyl)-1,3,4-thiadiazol-2-yl)-9H-pyrimido[4,5-b]indol-4-yl)oxy)methyl)morpholine-4-carboxylate

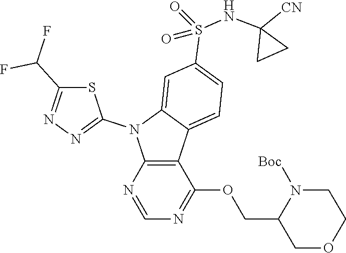 [SMILES: CC(C)(C)OC(=O)N1CCOCC1COc1ncnc2c1c1ccc(S(=O)(=O)NC3(C#N)CC3)cc1n2-c1nnc(C(F)F)s1]

To a solution of 4-chloro-N-(1-cyanocyclopropyl)-9-[5-(difluoromethyl)-1,3,4-thiadiazol-2-yl]pyrimido[4,5-b]indole-7-sulfonamide (60 mg, 0.12 mmol, Int B) and tert-butyl 3-(hydroxymethyl)morpholine-4-carboxylate (41 mg, 0.18 mmol) in THF (4 mL) was added t-BuONa (24 mg, 0.24 mmol) at 0° C. The mixture was stirred at 25° C. for 2 h., quenched with saturated aq. NH₄Cl (10 mL), and extracted with DCM (10 mL×3). The combined organic layers were washed with brine, dried over Na₂SO₄ and concentrated under reduced pressure. The residue was purified by prep-TLC on a silica gel layer eluting with EtOAc/PE (50%) to afford the title compound (63 mg) as white solid. LCMS calc. for C₂₇H₂₇F₂Ns06S₂ [M−H]⁻: m/z=661.2; Found: 661.1.

### Step 2: N-(1-cyanocyclopropyl)-9-(5-(difluoromethyl)-1,3,4-thiadiazol-2-yl)-4-(morpholin-3-ylmethoxy)-9H-pyrimido[4,5-b]indole-7-sulfonamide

A solution of tert-butyl 3-(((7-(N-(1-cyanocyclopropyl)sulfamoyl)-9-(5-(difluoromethyl)-1,3,4-thiadiazol-2-yl)-9H-pyrimido[4,5-b]indol-4-yl)oxy)methyl)morpholine-4-carboxylate (60 mg, 0.1 mmol) in DCM (4 mL) and TFA (2 mL) was stirred at r.t. for 1 h.. The mixture was concentrated under reduced pressure. The residue was purified by prep-HPLC on a C18 column eluting with MeCN/H₂O (35-65%) to afford the title compound (26.2 mg) as white solid. ¹H NMR: (400 MHz, DMSO-d⁶)¹H NMR: (400 MHz, DMSO-d⁶) δ 9.55-9.43 (m, 2H), 9.34-9.22 (m, 1H), 9.07 (s, 1H), 8.57 (d, J=8.0 Hz, 1H), 8.10 (d, J=8.0 Hz, 1H), 7.71 (t, J_{F-H}=53.2 Hz, 1H), 4.99-4.82 (m, 2H), 4.24-4.16 (m, 1H), 4.04-3.93 (m, 2H), 3.84-3.70 (m, 2H), 3.43-3.38 (m, 1H), 3.27-3.19 (m, 1H), 1.48-1.39 (m, 2H), 1.34-1.26 (m, 2H). LCMS calc. for C₂₂H₂₁F₂N₈O₄S₂ [M+H]⁺: m/z=563.1; Found: 563.0.

### Example 93: N-(1-Cyanocyclopropyl)-9-(5-(difluoromethyl)-1,3,4-thiadiazol-2-yl)-4-((3S,4R)-4-(ethylamino)-3-fluoropiperidin-1-yl)-9H-pyrimido[4,5-b]indole-7-sulfonamide

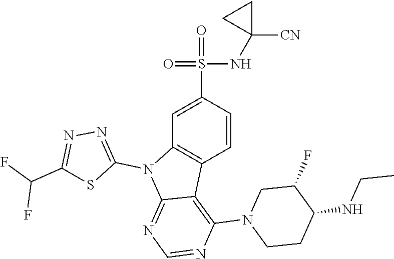 [SMILES: CCN[C@@H]1CCN(c2ncnc3c2c2ccc(S(=O)(=O)NC4(C#N)CC4)cc2n3-c2nnc(C(F)F)s2)C[C@@H]1F]

### Step 1: tert-butyl(3S, 4R)-4-acetamido-3-fluoropiperidine-1-carboxylate

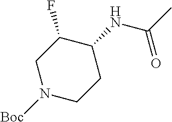 [SMILES: CC(=O)N[C@@H]1CCN(C(=O)OC(C)(C)C)C[C@@H]1F]

To a solution of tert-butyl (3S,4R)-4-amino-3-fluoropiperidine-1-carboxylate (3.0 g, 13.7 mmol) in THFF (30 mL) was added DIEA (2.7 g, 20.6 mmol) at 0˜5° C., followed by dropwise addition of acetyl chloride (1.1 g, 13.7 mmol). The reaction mixture was stirred at 0˜5° C. for 1 h. and then at r.t. for 20 min. The reaction mixture was quenched with water (15 mL), extracted with dichloromethane (30 mL×2). The combined organic layers were washed with brine, dried over Na₂SO₄, filtered and concentrated under reduced pressure to afford the title compound (4.0 g, crude) as white solid. LCMS calc. for C₁₂H₂₁FN₂O₃Na [M+Na]⁺: m/z=283.1; Found: 283.1.

### Step 2: tert-butyl (3S,4R)-4-(ethylamino)-3-fluoropiperidine-1-carboxylate

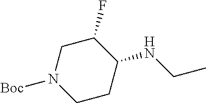 [SMILES: CCN[C@@H]1CCN(C(=O)OC(C)(C)C)C[C@@H]1F]

To a solution of tert-butyl (3S,4R)-4-acetamido-3-fluoropiperidine-1-carboxylate (4.0 g, 15.4 mmol) in THF (15 mL) was added BH₃/THF (40 mL, 160 mmol, 4M) at r.t.. The reaction mixture was stirred at 60° C. overnight, quenched with methanol (50 mL), and concentrated under reduced pressure to afford the title compound (4.0 g, crude) as white solid, which was directly used in the next step without further purification. LCMS calc. for C₁₂H₂₄FN₂O₂[M+H]⁺: m/z=247.2; Found: 247.1.

### Step 3: (3S,4R)—N-ethyl-3-fluoropiperidin-4-amine dihydrochloride

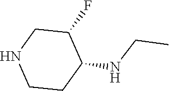

To a solution of tert-butyl (3S,4R)-4-(ethylamino)-3-fluoropiperidine-1-carboxylate (4.0 g, 16.2 mmol) in methanol (8 mL) was added HCl/dioxane (20 mL, 80 mmol, 4M), and stirred at r.t. for 1 h.. The reaction mixture was concentrated under reduced pressure to afford the title compound as HCl salt (3.2 g, crude) as white solid, which was directly used in the next step without further purification. LCMS calc. for C₇H₁₆FN₂ [M+H]⁺: m/z=147.1; Found: 147.1.

### Step 4: N-(1-cyanocyclopropyl)-9-(5-(difluoromethyl)-1,3,4-thiadiazol-2-yl)-4-((3S,4R)-4-(ethylamino)-3-fluoropiperidin-1-yl)-9H-pyrimido[4,5-b]indole-7-sulfonamide

To a mixture of 4-chloro-N-(1-cyanocyclopropyl)-9-(5-(difluoromethyl)-1,3,4-thiadiazol- 2-yl)-9H-pyrimido[4,5-b]indole-7-sulfonamide (5.4 g, 11.2 mmol, Int B) and (3S,4R)—N-ethyl-3-fluoropiperidin-4-amine dihydrochloride (3.2 g, 14.6 mmol) in acetonitrile (100 mL) was added DIEA (5.8 g, 44.9 mmol). The reaction mixture was stirred at 25° C. for 3 h., and concentrated under reduced pressure. To the residue was added acetonitrile (15 mL), then water (80 mL) and Na₂CO₃ aqueous (25 mL). The solid was collected by filtration, dried under vacuum at 40° C. to afford the title compound (4.1 g, 62% yield) as yellow solid. LCMS calc. for C₂₄H₂₅F₃N₉02S₂ [M+H]⁺: m/z=592.2; Found: 592.1.

### Example 94: N-(1-Cyanocyclopropyl)-9-(5-(difluoromethyl)-1,3,4-thiadiazol-2-yl)-4-((3S,5S)-3,5-dimethylpiperazin-1-yl)-9H-pyrimido[4,5-b]indole-7-sulfonamide

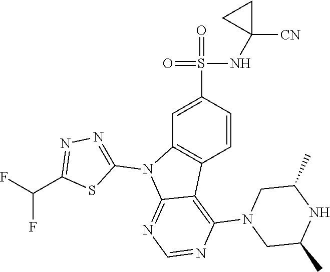 [SMILES: C[C@H]1CN(c2ncnc3c2c2ccc(S(=O)(=O)NC4(C#N)CC4)cc2n3-c2nnc(C(F)F)s2)C[C@H](C)N1]

### Step 1: tert-butyl (2S,6S)-4-(7-(N-(1-cyanocyclopropyl)sulfamoyl)-9-(5-(difluoromethyl)-1,3,4-thiadiazol-2-yl)-9H-pyrimido[4,5-b]indol-4-yl)-2,6-dimethylpiperazine-1-carboxylate

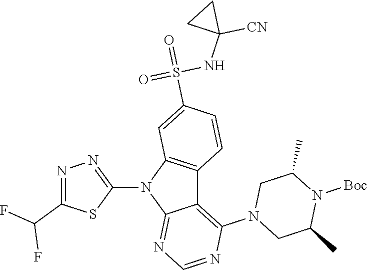 [SMILES: C[C@H]1CN(c2ncnc3c2c2ccc(S(=O)(=O)NC4(C#N)CC4)cc2n3-c2nnc(C(F)F)s2)C[C@H](C)N1C(=O)OC(C)(C)C]

To a mixture of 4-chloro-N-(1-cyanocyclopropyl)-9-(5-(difluoromethyl)-1,3,4-thiadiazol-2-yl)-9H-pyrimido[4,5-b]indole-7-sulfonamide (428 mg, 0.89 mmol, Int B) and tert-butyl (2S,6S)-2,6-dimethylpiperazine-1-carboxylate (200 mg, 0.93 mmol) in acetonitrile (9 mL) was added DIEA (172 mg, 1.33 mmol). The reaction mixture was stirred at 25° C. for 3 h., and concentrated under reduced pressure. To the residue was added acetonitrile (1.5 mL), followed by addition of water (6.5 mL) and Na₂CO₃ aqueous (0.2 mL). The solid was collected by filtration, dried under vacuum at 40° C. to afford the title compound (480 mg, 82% yield) as a yellow solid. LCMS calc. for C₂₈H₃₂F₂N₉O₄S₂ [M+H]⁺: m/z=660.2; Found: 660.1.

### Step 2: N-(1-cyanocyclopropyl)-9-(5-(difluoromethyl)-1,3,4-thiadiazol-2-yl)-4-((3S,5S)-3,5-dimethylpiperazin-1-yl)-9H-pyrimido[4,5-b]indole-7-sulfonamide

To a mixture of tert-butyl (2S,6S)-4-(7-(N-(1-cyanocyclopropyl)sulfamoyl)-9-(5-(difluoromethyl)-1,3,4-thiadiazol-2-yl)-9H-pyrimido[4,5-b]indol-4-yl)-2,6-dimethylpiperazine-1-carboxylate (100 mg, 0.15 mmol) in dichloromethane (1 mL) was added TFA (69 mg, 0.61 mmol). The reaction mixture was stirred at 25° C. for 3 h., and concentrated under reduced pressure. The residue was purified by pre-HPLC on a C18 column eluting with MeCN/H₂O (2-70% with 0.1% NH₄HCO₃) to afford the title compound (40 mg, 47.7% yield) as a white solid.

LCMS calc. for C₂₃H₂₄F₂N₉02S₂ [M+H]⁺: m/z=560.1; Found: 560.1.

### Int A: 4-Chloro-N-(1-cyanocyclopropyl)-9H-pyrimido[4,5-b]indole-7-sulfonamide

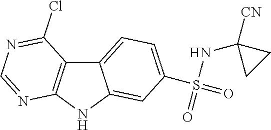 [SMILES: N#CC1(NS(=O)(=O)c2ccc3c(c2)[nH]c2ncnc(Cl)c23)CC1]

### Step 1: 4-chloro-N-(1-cyanocyclopropyl)-3-nitrobenzenesulfonamide

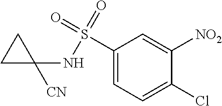 [SMILES: N#CC1(NS(=O)(=O)c2ccc(Cl)c([N+](=O)[O-])c2)CC1]

To a mixture of 1-aminocyclopropane-1-carbonitrile hydrochloride (55.6 g, 469 mmol), pyridine (500 mL) and DMAP (19.1 g, 156 mmol) in MeCN (500 mL) was added 4-chloro-3-nitrobenzenesulfonyl chloride (100 g, 391 mmol) at 0˜5° C. The resulting mixture was then stirred at r.t. for 2 h. The reaction mixture was poured into ice-water (500 mL), and adjusted to pH˜3 with aq. HCl solution (1 N) at 0˜5° C. The aqueous phase was extracted with EtOAc (300 mL×3). The combined organic layers were washed with brine (500 mL), dried over anhydrous Na₂SO₄, filtered and concentrated under reduced pressure. The residue was triturated with MTBE (100 mL) at 20° C. for 2 h. The solid formed was collected by filtration and dried in vacuum to afford the title compound (75 g, 63.6% yield) as a yellow solid. ¹H NMR: (400 MHz, DMSO-d₆) δ 9.54 (s, 1H), 8.51 (s, 1H), 8.09-8.14 (m, 2H), 1.47-1.51 (m, 2H), 1.31-1.35 (m, 2H).

LCMS calc. for C₁₀H₇ClN₃O₄S [M−H]⁻: m/z=300.0. Found: 300.0.

### Step 2: 2-amino-6-(N-(1-cyanocyclopropyl)sulfamoyl)-1H-indole-3-carboxamide

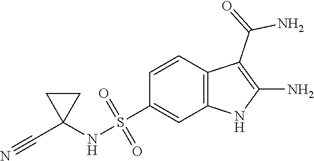 [SMILES: N#CC1(NS(=O)(=O)c2ccc3c(C(N)=O)c(N)[nH]c3c2)CC1]

To a solution of 2-cyanoacetamide (41.8 g, 497 mmol) in DMF (750 mL) was added NaH (39.8 g, 994 mmol, 60% dispersion in mineral oil) at 0° C. The mixture was stirred at 0° C. for 30 min., and 4-chloro-N-(1-cyanocyclopropyl)-3-nitrobenzenesulfonamide (75.0 g, 249 mmol) was added at 0° C. The mixture was stirred at 20° C. for 1 h. The reaction was poured into ice-water (500 mL) in portions, and adjusted to pH˜3 with conc. HCl solution (12 N) at 0˜5° C. To the solution was added DMF (750 mL), followed by addition of FeCl₃ (120 g, 744 mmol) at 20° C. The mixture was heated to 60° C., and Zn (162 g, 2.48 mol) was added in small portions. The mixture was stirred and then heated at 100° C. for 2 h. LCMS showed the reaction was completed. After cooling, the reaction mixture was filtered on celite. The filtrate was extracted with DCM (500 mL×6). The combined organic layers were dried over anhydrous Na₂SO₄, filtered and concentrated in vacuum. The residue was purified by flash chromatography on a silica gel column eluting with EtOAc/PE (0-100%) to afford the title product (30.0 g, 18.9% yield) as yellow foam. LCMS calc. for C₁₃H₁₂N₅O₃S [M−H]⁻: m/z=318.1; Found: 318.0.

### Step 3: N-(1-cyanocyclopropyl)-4-oxo-4,9-dihydro-3H-pyrimido[4,5-b]indole-7-sulfonamide

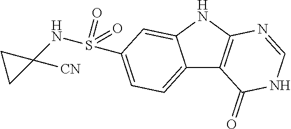 [SMILES: N#CC1(NS(=O)(=O)c2ccc3c(c2)[nH]c2nc[nH]c(=O)c23)CC1]

To a mixture of 2-amino-6-(N-(1-cyanocyclopropyl)sulfamoyl)-1H-indole-3-carboxamide (28.0 g, 87.7 mmol) in trimethoxymethane (560 mL) was added conc. HCl solution (12 N, 161 mL) at 20° C. The mixture was stirred at 60° C. for 1 h. LCMS showed the starting material was consumed completely. The reaction mixture was filtered. The filter cake was dried in vacuum to afford the title product (20.0 g, 69.3% yield) as yellow solid. ¹H NMR: (400 MHz, DMSO-d₆) δ 12.7 (s, 1H), 12.5 (s, 1H), 9.07 (s, 1H), 8.24 (s, 1H), 8.17 (d, J=8.0 Hz, 1H), 7.96 (s, 1H), 7.71 (d, J=10.0 Hz, 1H), 1.37-1.42 (m, 2H), 1.22-1.27 (m, 2H). LCMS calc. for C₁₄H₁₀N₅03S [M−H]⁻: m/z=328.1; Found: 328.1.

### Step 4: 4-chloro-N-(1-cyanocyclopropyl)-9H-pyrimido[4,5-b]indole-7-sulfonamide

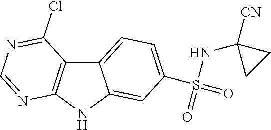 [SMILES: N#CC1(NS(=O)(=O)c2ccc3c(c2)[nH]c2ncnc(Cl)c23)CC1]

A mixture of N-(1-cyanocyclopropyl)-4-oxo-4,9-dihydro-3H-pyrimido[4,5-b]indole-7-sulfonamide (18.0 g, 54.7 mmol) in POCl₃ (720 mL) was stirred at 100° C. for 16 h. LCMS showed the starting material was consumed completely. The mixture was concentrated under reduced pressure. The residue was purified by flash chromatography on a silica gel column eluting with EtOAc/PE (0-100%) to afford the title product (10 g, 47.3% yield) as light yellow solid. LCMS calc. for C₁₄H₉C₁N₅02S [M−H]⁻: m/z=346.0; Found: 345.9.

### Int B: 4-Chloro-N-(1-cyanocyclopropyl)-9-(5-(difluoromethyl)-1,3,4-thiadiazol-2-yl)-9H-pyrimido[4,5-b]indole-7-sulfonamide

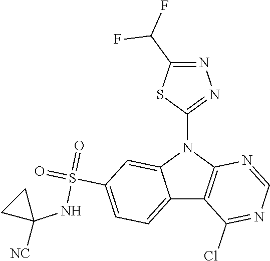 [SMILES: N#CC1(NS(=O)(=O)c2ccc3c4c(Cl)ncnc4n(-c4nnc(C(F)F)s4)c3c2)CC1]

To a solution ofN-(1-cyanocyclopropyl)-9-(5-(difluoromethyl)-1,3,4-thiadiazol-2-yl)-4-(methylthio)-9H-pyrimido[4,5-b]indole-7-sulfonamide (10.0 g, 20.3 mmol, Example 25) in MeCN (100 mL) was added sulfuryl chloride (10.7 g, 101 mmol) at 0˜5° C. The resulting mixture was stirred at r.t. for 5 h.. The precipitate was collected by filtration, washed with MeCN (10 mL×3), and then dried under reduced pressure to afford the title compound (8.2 g) as yellow solid. LCMS calc. for C₁₇H₁₁C₁F₂N₇O₂S₂[M+H]⁺: m/z=482.0. Found: 482.1.

### Int 1: 1-(4-(2-Hydroxyethyl)piperazin-1-yl)-2-methylpropan-1-one

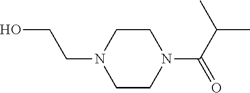

### Step 1: 1-(2-((tert-butyldimethylsilyl)oxy)ethyl)piperazine

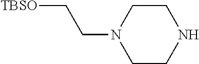 [SMILES: CC(C)(C)[Si](C)(C)OCCN1CCNCC1]

To a mixture of 2-(piperazin-1-yl)ethan-1-ol (5.2 g, 40 mmol) and imidazole (5.5 g, 60 mmol) in DCM (100 mL) was added TBSCI (7.5 g, 50 mmol). The reaction mixture was stirred at r.t. overnight. After concentrated under reduced pressure, the residue was diluted with EtOAc (100 mL) and then washed with water and brine. The organic layers were dried over anhydrous Na₂SO₄, filtered and concentrated under reduced pressure to afford the title compound (9.8 g) as yellow oil. LCMS calc. for Cl₂H₂₉N₂OSi [M+H]⁺: m/z=245.2; Found: 245.2.

### Step 2:1-(4-(2-((tert-butyldimethylsilyl)oxy)ethyl)piperazin-1-yl)-2-methylpropan-1-one

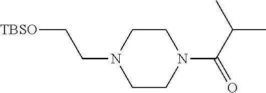 [SMILES: CC(C)C(=O)N1CCN(CCO[Si](C)(C)C(C)(C)C)CC1]

To a mixture of 1-(2-((tert-butyldimethylsilyl)oxy)ethyl)piperazine (2.45 g, 10 mmol),TEA (1.56 g, 15 mmol) in DCM (20 mL) was added isobutyryl chloride (1.28 g, 12 mmol). The reaction mixture was stirred at r.t. for 3 h., and then concentrated under reduced pressure. The residue was diluted with EtOAc (100 mL), and then washed with water and brine. The organic layers were dried over anhydrous Na₂SO₄, filtered and concentrated under reduced pressure. The residue was purified by flash chromatography on a silica gel column eluting with EtOAc/PE (20-55%) to afford the title compound (1.0 g) as colorless oil. LCMS calc. for C₁₆H₃₅N₂O₂Si [M+H]⁺: m/z=315.2; Found: 315.2.

### Step 3: 1-(4-(2-hydroxyethyl)piperazin-1-yl)-2-methylpropan-1-one

A solution of 1-(4-(2-((tert-butyldimethylsilyl)oxy)ethyl)piperazin-1-yl)-2-methylpropan-1-one (1.0 g, 3.3 mmol) in HCl solution (10 mL, 4.0 M in 1,4-dioxane) was stirred under r.t. overnight. The reaction mixture was concentrated under reduced pressure. The resulting solid was washed with PE to afford the title compound (0.45 g) as HCl salt as white solid. LCMS calc. for C₁₀H₂₀N₂O₂ [M+H]⁺: m/z=201.2; Found: 201.1.

### Int 2: 4-(2-Hydroxyethyl)-N,N-dimethylpiperazine-1-carboxamide

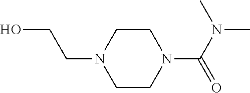

### Step 1: 4-(2-((tert-butyldimethylsilyl)oxy)ethyl)-N,N-dimethylpiperazine-1-carboxamide

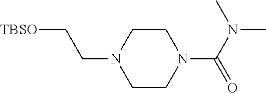 [SMILES: CN(C)C(=O)N1CCN(CCO[Si](C)(C)C(C)(C)C)CC1]

To a mixture of 1-(2-((tert-butyldimethylsilyl)oxy)ethyl)piperazine (2.45 g, 10 mmol, Int 1, Step 1), TEA (1.56 g, 15 mmol) in DCM (20 mL) was added dimethylcarbamoylchloride (1.28 g, 12 mmol). The reaction mixture was stirred at r.t. for 3 h., and then concentrated under reduced pressure. The residue was diluted with EtOAc (100 mL), and then washed with water and brine. The combined layers were dried over anhydrous Na₂SO₄, filtered and concentrated under reduced pressure. The residue was purified by flash chromatography on a silica gel column eluting with EtOAc/PE (60-80%) to afford the title compound (1.5 g) as colorless oil. LCMS calc. for C₁₅H₃₄N₃₀₂Si [M+H]⁺: m/z=316.2; Found: 316.2.

### Step 2: 4-(2-hydroxyethyl)-N,N-dimethylpiperazine-1-carboxamide

A solution of 4-(2-((tert-butyldimethylsilyl)oxy)ethyl)-N,N-dimethylpiperazine-1-carboxamide (1.5 g, 3.9 mmol) in HCl solution (15 mL, 4.0 M in 1,4-dioxane) was stirred under r.t. overnight. The reaction mixture was concentrated under reduced pressure. The resulting solid was washed with PE to afford the title compound (0.9 g) as HCl salt as off-white solid. LCMS calc. for C₉H₂₀N₃₀₂ [M+H]⁺: m/z=202.2; Found: 202.2.

### Int 3: (3S,4S)—N,N-Diethyl-3-fluoropiperidin-4-amine

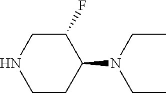

### Step 1: tert-butyl (3S,4S)-4-(diethylamino)-3-fluoropiperidine-1-carboxylate

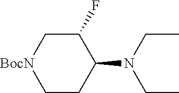 [SMILES: CCN(CC)[C@H]1CCN(C(=O)OC(C)(C)C)C[C@@H]1F]

To a mixture of tert-butyl (3S,4S)-4-amino-3-fluoropiperidine-1-carboxylate (650 mg, 3 mmol) and Cs₂CO₃ (3.91 g, 12 mmol) in MeCN (20 mL) was added iodoethane (1.4 g, 9 mmol). The mixture was stirred at 50° C. overnight. The reaction mixture was concentrated under reduced pressure, diluted with EtOAc (60 mL), and then washed with water and brine. The organic layers were dried over anhydrous Na₂SO₄, filtered and concentrated under reduced pressure. The residue was purified by flash chromatography on a silica gel column eluting with EtOAc/PE (60-80%) to afford the title compound (530 mg) as colorless oil. LCMS calc. for C₁₄H₂₈FN₂O₂[M+H]⁺: m/z=275.2; Found: 275.2.

### Step 2: (3S,4S)—N,N-diethyl-3-fluoropiperidin-4-amine

A solution of tert-butyl (3S,4S)-4-(diethylamino)-3-fluoropiperidine-1-carboxylate (530 mg, 1.93 mmol) in HCl solution (5 mL, 4.0 M in 1,4-dioxane) was stirred under r.t. for 4 h.. The reaction mixture was concentrated under reduced pressure. The resulting solid was washed with PE to afford the title compound (330 mg) as HCl salt as off-white solid. LCMS calc. for C₉H₂₀FN₂ [M+H]⁺: m/z=175.2; Found: 175.2.

The intermediates (Int #, # is integer number) listed in Table 6 below were prepared by using an appropriate commercially available amine and iodoethane as the methods substantially analogous to those described for preparing Int 3.

TABLE 6
Preparations of Examples (Ex)

| | | | LCMS Cacl. |
|---|---|---|---|
| | | | m/z |
| Int # | Structure | Name | Found |
| Int 4 | 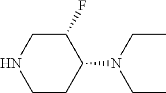 | (3S,4R)-N,N-diethyl-3- fluoropiperidin-4-amine | [M + H]⁺ 175.2 175.2 |
| Int 5 | 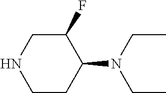 | (3R,4S)-N,N-diethyl-3- fluoropiperidin-4-amine | [M + H]⁺ 175.2 175.2 |
| Int 6 | 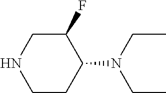 | (3R,4R)-N,N-diethyl-3- fluoropiperidin-4-amine | [M + H]⁺ 175.2 175.2 |
| Int 8 | 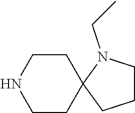 | 1-ethyl-1,8-diazaspiro[4.5]decane | [M + H]⁺ 169.2 169.2 |
| Int 12 | 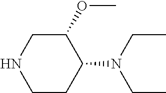 | (3S,4R)-N,N-diethyl-3- methoxypiperidin-4-amine | [M + H]⁺ 187.2 187.2 |
| Int 13 | 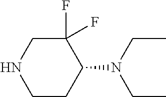 | (R)-N,N-diethyl-3,3- difluoropiperidin-4-amine | [M + H]⁺ 193.2 193.2 |
| Int 14 | 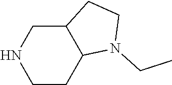 | 1-ethyloctahydro-1H-pyrrolo[3,2- c]pyridine | [M + H]⁺ 155.2 155.2 |

### Int 7: (3′S)-3′,4,4-Trifluoro-1,4′-bipiperidine

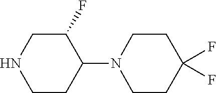 [SMILES: F[C@H]1CNCCC1N1CCC(F)(F)CC1]

### Step 1: tert-butyl (S)-3-fluoro-4-oxopiperidine-1-carboxylate

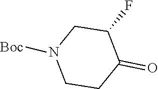 [SMILES: CC(C)(C)OC(=O)N1CCC(=O)[C@@H](F)C1]

To a solution of tert-butyl (3S,4R)-3-fluoro-4-hydroxypiperidine-1-carboxylate (1.0 g, 4.6 mmol) in DCM (10 mL) was added Dess-Martin periodinane (1.16 g, 5.5 mmol). The reaction mixture was stirred at r.t. for 8 h.. After concentrated under reduced pressure, the residue was purified by flash chromatography on a silica gel column eluting with DCM/EA (30-50%) to afford the title compound (500 mg) as white solid. LCMS calc. for C₁₀H₁₇FNO₃ [M+H]⁺: m/z=218.1; Found: 218.1.

### Step 2: tert-butyl (3'S)-3′,4,4-trifluoro-[ ],4′-bipiperidine]-1-1′-carboxylate

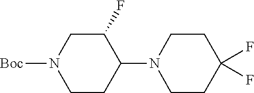 [SMILES: CC(C)(C)OC(=O)N1CCC(N2CCC(F)(F)CC2)[C@@H](F)C1]

To a solution of tert-butyl (S)-3-fluoro-4-oxopiperidine-1-carboxylate (500 mg, 2.30 mmol) in DCM (10 mL) was added 4,4-difluoropiperidine (560 mg, 4.6 mmol). The reaction mixture was stirred at r.t. for 30 min, and then NaBH(OAc)₃ (976 mg, 4.60 mmol) was added. The mixture was stirred at r.t. for 2 h., quenched with water, and then extracted with ethyl acetate (10 mL×3). The combined organic layer were washed with brine, dried over Na₂SO₄, filtered and then concentrated under reduced pressure to afford the title compound (240 mg) as yellow oil.

LCMS calc. for C₁₅H₂₆F₃N₂O₂[M+H]⁺: m/z=323.2; Found: 323.2.

### Step 3: (3'S)-3′,4,4-trifluoro-1,4′-bipiperidine

To a solution of tert-butyl (3'S)-3′,4,4-trifluoro-[1,4′-bipiperidine]-1′-carboxylate (240 mg, 0.77 mmol) in DCM (1 mL) was added HCl solution (1 mL, 4 M in 1,4-dioxane). The reaction mixture was stirred at r.t. for 3 h., and concentrated under reduced pressure to give the title compound (140 mg) as HCl salt as yellow solid. LCMS calc. for C₁₀H₁₈FN₂ [M+H]⁺: m/z=223.1; Found: 223.1.

### Int 9: (1R,4R)—N,N-Diethyl-2-azabicyclo[2.2.1]heptan-5-amine

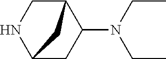

### Step 1: tert-butyl (JR,4R)-5-(diethylamino)-2-azabicyclo[2.2.1]heptane-2-carboxylate

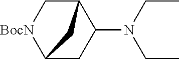 [SMILES: CCN(CC)C1C[C@H]2C[C@@H]1CN2C(=O)OC(C)(C)C]

To a solution of tert-butyl (1R,4R)-5-oxo-2-azabicyclo[2.2.1]heptane-2-carboxylate (420 mg, 2.0 mmol) in DCM (10 mL) was added diethylamine solution (366 mg, 5.0 mmol, 2.0 M in THF). The reaction mixture was stirred at r.t. for 30 min., and then NaBH(OAc)₃ (850 mg, 4.0 mmol) was added. The mixture was stirred at r.t. for additional 2 h., quenched with water, and then extracted with ethyl acetate (10 mL×3). The combined organic layer were washed with brine, dried over Na₂SO₄, filtered and then concentrated under reduced pressure to afford the title compound (440 mg) as yellow oil. LCMS calc. for C₁₅H₂₉N₂O₂ [M+H]⁺: m/z=269.2; Found: 269.2.

### Step 2: (JR,4R)—N,N-diethyl-2-azabicyclo[2.2.1]heptan-5-amine

To a solution of tert-butyl (1R,4R)-5-(diethylamino)-2-azabicyclo[2.2.1]heptane-2-carboxylate (270 mg, 1.0 mmol) in DCM (2 mL) was added HCl solution (2 mL, 4 M in 1,4-dioxane). The reaction mixture was stirred at r.t. for 5 h., and concentrated under reduced pressure to give the title compound (170 mg) as HCl salt as yellow solid. LCMS calc. for C₁₀H₂₁N₂[M+H]⁺: m/z=169.2; Found: 169.2.

### Int 10: (1R,4R)—N,N-Diethyl-2-azabicyclo[2.2.1]heptan-5-amine

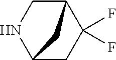

### Step 1: tert-butyl (JR,4R)-5,5-difluoro-2-azabicyclo[2.2.1]heptane-2-carboxylate

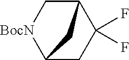 [SMILES: CC(C)(C)OC(=O)N1C[C@H]2C[C@@H]1CC2(F)F]

To a solution of tert-butyl (1R,4R)-5-oxo-2-azabicyclo[2.2.1]heptane-2-carboxylate (420 mg, 2.0 mmol) in DCM (40 mL) was added DAST (1.3 g, 8.0 mmol). The mixture was stirred at r.t. overnight, quenched with aq. NaHCO₃ solution, and then extracted with DCM (10 mL×3). The combined organic layer were washed with brine, dried over Na₂SO₄, filtered and then concentrated under reduced pressure. The residue was purified by flash chromatography on a silica gel column eluting with EtOAc/PE (20-40%) to afford the title compound (120 mg) as yellow oil. LCMS calc. for C₁₁H₁₈F₂NO₂ [M+H]⁺: m/z=234.2; Found: 234.1.

### Step 2: (JR,4R)—N,N-diethyl-2-azabicyclo[2.2.1]heptan-5-amine

To a solution of tert-butyl (1R,4R)-5,5-difluoro-2-azabicyclo[2.2.1]heptane-2-carboxylate (120 mg, 1.0 mmol) in DCM (4 mL) was added TFA (1 mL). The reaction mixture was stirred at r.t. for 3 h.. The reaction mixture was concentrated under reduced pressure to give the title compound (70 mg) as HCl salt as yellow solid. LCMS calc. for C₆H₁₀F₂N [M+H]⁺: m/z=134.1; Found: 134.1.

### Int 11: (R)-(1-Methylpiperazin-2-yl)methanol

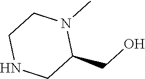

### Step 1: tert-butyl (R)-3-(hydroxymethyl)-4-methylpiperazine-1-carboxylate

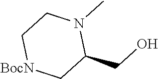 [SMILES: CN1CCN(C(=O)OC(C)(C)C)C[C@@H]1CO]

To a solution of tert-butyl (R)-3-(hydroxymethyl)piperazine-1-carboxylate (500 mg, 2.3 mmol) in MeOH (12 mL) were added sodium acetate (303 mg, 3.7 mmol), paraformaldehyde (375 mg, 4.2 mmol) and sodium cyanoborohydride (218 mg, 3.5 mmol). The mixture was stirred at r.t. for 1 h., quenched with sat. aq. NaHCO₃ (20 mL), and then extracted with DCM (50 mL). The organic phase was dried over anhydrous Na₂SO₄, filtered, and concentrated under reduced pressure. The residue was purified by flash chromatography on a silica gel column eluting with MeOH/DCM (0-5%) to afford the title product (421 mg) as yellow oil. LCMS calc. for C₁₁H₂₃N₂O₃ [M+H]⁺: m/z=231.2; Found: 231.0.

### Step 2: (R)-(1-methylpiperazin-2-yl)methanol

To a solution of tert-butyl (R)-3-(hydroxymethyl)-4-methylpiperazine-1-carboxylate (421 mg, 1.8 mmol) in DCM (3 mL) was added HCl solution (3 mL, 4 M in 1,4-dioxane). The reaction mixture was stirred at r.t. for 5 h.. The reaction mixture was concentrated under reduced pressure to give the title compound (230 mg) as HCl salt as yellow oil. LCMS calc. for C₆H₁₄N₂₀ [M+H]⁺: m/z=131.1; Found: 131.1.

### Int 15: S—N-Methyl-1-(tetrahydrofuran-3-yl)methanamine

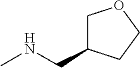

### Step 1: (R)-(tetrahydrofuran-3-yl)methyl methanesulfonate

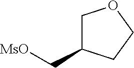 [SMILES: CS(=O)(=O)OC[C@@H]1CCOC1]

To a solution of (S)-(tetrahydrofuran-3-yl)methanol (406 mg, 4.0 mmol) and triethylamine (610 mg, 6.0 mmol) in DCM (4 mL) was added methanesulfonyl chloride (570 mg, 5.0 mmol) at 0° C. The mixture was stirred at r.t. for 2 h., diluted with H₂O, and then extracted with DCM (10 mL×3). The combined organic layers were washed with brine, dried over Na₂SO₄, filtered and concentrated under reduced pressure to afford the title compound (710 mg) as yellow oil. LCMS calc. for C₆H₁₃04S [M+H]⁺: m/z=181.1; Found: 181.1.

### Step 2: (S)—N-methyl-1-(tetrahydrofuran-3-yl)methanamine

A mixture of (R)-(tetrahydrofuran-3-yl)methyl methanesulfonate (710 mg, 4.0 mmol) and MeNH₂ solution (30% in EtOH, 20 mL) in a sealed tube was stirred at 70° C. for 16 h.. The reaction mixture was cooled and concentrated under reduced pressure. The residue was purified by prep-TLC on a silica gel layer eluting with MeOH/DCM (10%) to afford the title compound (120 mg) as colorless oil. LCMS calc. for C₆H₁₃NO [M+H]⁺: m/z=116.1; Found: 116.1.

### Int 16: (R)—N-Methyl-1-(tetrahydrofuran-3-yl)methanamine

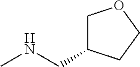

This compound was prepared by procedures analogous to those described for Int 15 Step 1-2 using (R)-(tetrahydrofuran-3-yl)methanol. LCMS calc. for C₆H₁₃NO [M+H]⁺: m/z=116.1; Found: 116.1.

### Int 17: 2-(1H-Imidazol-1-yl)-N-methylethan-1-amine

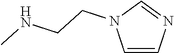

### Step 1: tert-butyl (2-(1H-imidazol-1-yl)ethyl)(methyl)carbamate

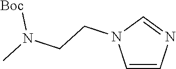 [SMILES: CN(CCn1ccnc1)C(=O)OC(C)(C)C]

A mixture of tert-butyl (2-chloroethyl)(methyl)carbamate (400 mg, 2.05 mmol), 1H-imidazole (836 mg, 12.3 mmol) and K₂CO₃ (849 mg, 6.15 mmol) in DMF (5 mL) was stirred at 80° C. for 3 h.. The reaction mixture was diluted with H₂O, and then extracted with EA (20 mL×3). The combined organic layers were washed with brine, dried over Na₂SO₄, filtered and concentrated under reduced pressure. The residue was purified by prep-TLC on a silica gel layer eluting with MeOH/DCM (10%) to afford the title compound (280 mg) as yellow oil. LCMS calc. for C₁₁H₂₀N₃₀₂ [M+H]⁺: m/z=226.2; Found: 226.1.

### Step 2: 2—(1H-imidazol-1-yl)-N-methylethan-1-amine

To a solution of tert-butyl tert-butyl (2-(1H-imidazol-1-yl)ethyl)(methyl)carbamate (280 mg, 1.8 mmol) in DCM (2 mL) was added HCl solution (2 mL, 8 mmol, 4 M in 1,4-dioxane). The reaction mixture was stirred at r.t. for 2 h., and concentrated under reduced pressure to afford the title compound (130 mg) as HCl salt as yellow oil. LCMS calc. for C₆H₁₂N₃[M+H]⁺: m/z=126.1; Found: 126.1.

### Int 18: 1-Methyl-3-(2-(methylamino)ethyl)imidazolidin-2-one

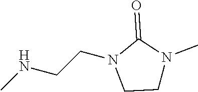

### Step 1: tert-butyl methyl(2-(3-methyl-2-oxoimidazolidin-1-yl)ethyl)carbamate

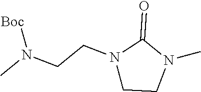

To a mixture of tert-butyl (2-chloroethyl)(methyl)carbamate (200 mg, 1 mmol) and 1-methylimidazolidin-2-one (618 mg, 6.2 mmol) in DMF (5 mL) was added Cs₂CO₃ (1.0 g, 3.1 mmol). The mixture was stirred at 80° C. for 3 h., diluted with H₂O and extracted with EA (20 mL×3). The combined organic layers were washed with brine, dried over Na₂SO₄, filtered and concentrated under reduced pressure. The residue was purified by prep-TLC on a silica gel layer eluting with EA/PE (20%) to afford the title compound (50 mg) as yellow solid. LCMS calc for C₁₂H₂₄N₃₀₃, [M+H]⁺: m/z=258.2; Found: 258.3.

### Step 2: 1-methyl-3-(2-(methylamino)ethyl)imidazolidin-2-one

To a solution of tert-butyl (2-(1H-imidazol-1-yl)ethyl)(methyl)carbamate (50 mg, 0.2 mmol) in DCM (1 mL) was added HCl solution (1 mL, 4 M in 1,4-dioxane). The reaction mixture was stirred at r.t. for 2 h., and concentrated under reduced pressure to afford the title compound (40 mg) as HCl salt as yellow oil. LCMS calc. for C₇H₁₆N₃₀ [M+H]⁺: m/z=158.1; Found: 158.3.

### Int 19: 1-(2-(Methylamino)ethyl)pyrrolidin-2-one

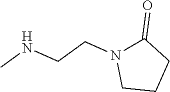

### Step 1: tert-butyl (2-(4-chlorobutanamido)ethyl)(methyl)carbamate

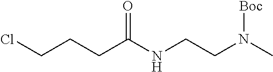 [SMILES: CN(CCNC(=O)CCCCl)C(=O)OC(C)(C)C]

To a mixture of tert-butyl (2-aminoethyl)(methyl)carbamate (1.0 g, 5.7 mmol) and TEA (1.74 g, 17.2 mmol) in DCM (20 mL) was added 4-chlorobutanoyl chloride (1.20 g, 8.6 mmol) at 0° C. The mixture was stirred at 0° C. for 1 h., diluted with H₂O, and then extracted with DCM (10 mL×3). The combined organic layers were washed with brine, dried over Na₂SO₄, filtered and concentrated under reduced pressure. The residue was purified by prep-TLC on a silica gel layer eluting with EA/PE (10%) to afford the title compound (500 mg) as yellow solid. LCMS calc for C₁₂H₂₄C₁N₂03, [M+H]⁺: m/z=279.1; Found: 279.1.

### Step 2: tert-butyl methyl(2-(2-oxopyrrolidin-1-yl)ethyl)carbamate

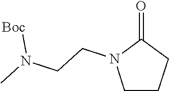 [SMILES: CN(CCN1CCCC1=O)C(=O)OC(C)(C)C]

To a solution of tert-butyl (2-(4-chlorobutanamido)ethyl)(methyl)carbamate (500 mg, 1.8 mmol) in DMF (10 mL) was added NaH (215 mg, 5.4 mmol, 60% dispersion in mineral oil) at 0° C. The mixture was stirred at 0° C. for 1 h.. The reaction mixture was diluted with H₂O and extracted with EtOAc (20 mL×3). The combined organic layers were washed with brine, dried over Na₂SO₄, filtered and concentrated under reduced pressure. The residue was purified by prep-TLC on a silica gel layer eluting with EA/PE (20%) to afford the title compound (300 mg) as yellow solid. LCMS calc for C₁₂H₂₃N₂O₃, [M+H]⁺: m/z=243.1; Found: 243.2.

### Step 3: 1-(2-(methylamino)ethyl)pyrrolidin-2-one

To a solution of tert-butyl methyl(2-(2-oxopyrrolidin-1-yl)ethyl)carbamate (250 mg, 1.0 mmol) in DCM (2 mL) was added HCl solution (2 mL, 4 M in 1,4-dioxane). The reaction mixture was stirred at r.t. for 2 h.. The reaction mixture was concentrated under reduced pressure to afford the title compound (200 mg) as HCl salt as yellow solid. LCMS calc. for C₇H₁₅N₂₀ [M+H]⁺: m/z=143.1; Found: 143.2.

### Int 20: Methyl[(4-methylmorpholin-3-yl)methyl]amine

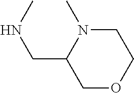

### Step 1: (4-methylmorpholin-3-yl)methyl methanesulfonate

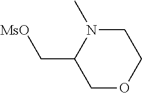

To a mixture of (4-methylmorpholin-3-yl)methanol (400 mg, 3.1 mmol) and triethylamine (617 mg, 6.1 mmol) in DCM (10 mL) was added methanesulfonyl chloride (420 mg, 3.7 mmol) at 0° C. The mixture was stirred at 25° C. for 2 h.. The reaction mixture was diluted with H₂O and extracted with DCM (10 mL×3). The combined organic layers were washed with brine, dried over Na₂SO₄, filtered and concentrated under reduced pressure to give the title compound (600 mg) as yellow oil. LCMS calc. for C₇H₁₆NO₄S [M+H]⁺: m/z=210.1; Found: 210.1.

### Step 2: methyl[(4-methylmorpholin-3-yl)methyl]amine

A solution of 4-methylmorpholine-3-carbaldehyde (600 mg, 4.65 mmol) in 30% MeNH₂ of EtOH (30 mL) was stirred at 70° C. for 16 h. in a sealed tube. The reaction mixture was cooled and concentrated under reduced pressure. The residue was purified by prep-TLC on a silica gel layer eluting with MeOH/DCM (10%) to afford the title compound (80 mg) as colorless oil. ¹H NMR: (400 MHz, DMSO-d⁶) δ 3.89-3.64 (m, 3H), 3.48-3.36 (m, 3H), 3.07-2.94 (m, 2H), 2.74-2.68 (m, 1H), 2.51 (s, 3H), 2.27 (s, 3H). LCMS calc. for C₇H₁₇N₂₀ [M+H]⁺: m/z=145.1; Found: 145.2.

### Int 21: (R)—N-Methyl-1-(4-methylmorpholin-3-yl)methanamine

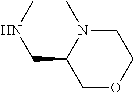

This compound was prepared by procedures analogous to those described for Int 20 Step 1-2 using (R)-(4-methylmorpholin-3-yl)methanol. LCMS calc. for C₇H₁₇N₂₀ [M+H]⁺: m/z=145.1; Found: 145.2.

### Int 22: (S)—N-Methyl-1-(4-methylmorpholin-3-yl)methanamine

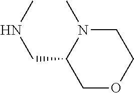

This compound was prepared by procedures analogous to those described for Int 20 Step 1-2 using (S)-(4-methylmorpholin-3-yl)methanol. LCMS calc. for C₇H₁₇N₂₀ [M+H]⁺: m/z=145.1; Found: 145.2.

### Int 23: 2-(3,5-Dimethyl-1H-1,2,4-triazol-1-yl)-N-methylethan-1-amine

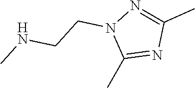

### Step 1: tert-butyl (2-(3,5-dimethyl-IH-1,2,4-triazol-1-yl)ethyl)(methyl)carbamate

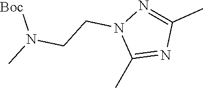

To a mixture of tert-butyl (2-chloroethyl)(methyl)carbamate (100 mg, 0.5 mmol) and 3,5-dimethyl-1H-1,2,4-triazole (297 mg, 3.1 mmol) in DMF (5 mL) was added K₂CO₃ (211 mg, 1.5 mmol). The mixture was stirred at 80° C. for 3 h., diluted with H₂O, and extracted with EA (15 mL×3). The combined organic layers were washed with brine, dried over Na₂SO₄, filtered and concentrated under reduced pressure. The residue was purified by prep-TLC on a silica gel layer eluting with PE/EA=5:1 to afford the title compound (100 mg) as yellow solid. LCMS calc for C₁₂H₂₃N₄₀₂, [M+H]⁺: m/z=255.2; Found: 255.2.

### Step 2: 2-(3,5-dimethyl-IH-1,2,4-triazol-1-yl)-N-methylethan-1-amine

To a solution of tert-butyl (2-(3,5-dimethyl-1H-1,2,4-triazol-1-yl)ethyl)(methyl)carbamate (80 mg, 0.31 mmol) in DCM (1 mL) was added HCl solution (1 mL, 4 M in 1,4-dioxane). The reaction mixture was stirred at r.t. for 2 h.. The reaction mixture was concentrated under reduced pressure to afford the title compound (60 mg) as HCl salt as yellow solid. LCMS calc. for C₇H₁₅N₄[M+H]⁺: m/z=155.1; Found: 155.2.

### Int 24: 2-(2-(Methylamino)ethyl)isothiazolidine 1,1-dioxide

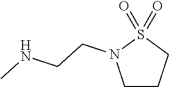

### Step 1: tert-butyl (2-((3-chloropropyl)sulfonamido)ethyl)(methyl)carbamate

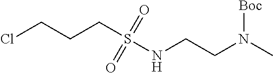 [SMILES: CN(CCNS(=O)(=O)CCCCl)C(=O)OC(C)(C)C]

To a mixture of tert-butyl (2-aminoethyl)(methyl)carbamate (1.0 g, 5.7 mmol) and DIEA (1.0 g, 7.4 mmol) in THF (60 mL) was added 3-chloropropane-1-sulfonyl chloride (1.11 g, 6.2 mmol) at 0° C. The reaction mixture was stirred at r.t. for 2 h., concentrated, diluted with H₂O, and extracted with DCM (30 mL×3). The combined organic layers were washed with brine, dried over Na₂SO₄, filtered and concentrated. The residue was purified by flash chromatography on a silica gel column eluting with EA/PE (0-33%) to afford the title compound (1.1 g) as yellow solid. TLC: R^{f}=0.5 (PE/EA=1:1). ¹H NMR: (400 MHz, DMSO-d⁶) δ 7.27 (s, 1H), 3.79-3.69 (m, 2H), 3.26-3.19 (m, 2H), 3.16-2.99 (m, 4H), 2.85-2.74 (m, 3H), 2.14-2.03 (m, 2H), 1.43-1.33 (m, 9H).

### Step 2: tert-butyl (2-(1,1-dioxidoisothiazolidin-2-yl)ethyl)(methyl)carbamate

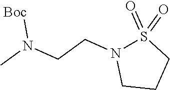 [SMILES: CN(CCN1CCCS1(=O)=O)C(=O)OC(C)(C)C]

To a solution of tert-butyl (2-((3-chloropropyl)sulfonamido)ethyl)(methyl)carbamate (500 mg, 1.6 mmol) in DMF (20 mL) was added NaH (190 mg, 4.7 mmol, 60% dispersion in mineral oil) at 0° C. The mixture was stirred at 0° C. for 2 h., quenched with water and extracted with EA (30 mL×3). The organic phase was washed with brine, dried over Na₂SO₄, filtered and concentrated under reduced pressure. The residue was purified by prep-TLC on a silica gel layer eluting with EA/PE (0-50%) to afford the title compound (320 mg) as yellow solid. TLC: R^{f}=0.6 (PE/EA=1:1). ¹H NMR: (400 MHz, DMSO-d⁶) 6.3.33-3.28 (m, 2H), 3.26-3.11 (m, 4H), 3.05-2.95 (m, 2H), 2.84-2.74 (m, 3H), 2.20 (s, 2H), 1.43-1.33 (m, 9H).

### Step 3: 2-(2-(methylamino)ethyl)isothiazolidine 1,1-dioxide

To a solution of tert-butyl (2-(1,1-dioxidoisothiazolidin-2-yl)ethyl)(methyl)carbamate (300 mg, 1.1 mmol) in DCM (2 mL) was added HCl solution (2 mL, 4 M in 1,4-dioxane). The reaction mixture was stirred at r.t. for 3 h. The reaction mixture was concentrated under reduced pressure to afford the title compound (60 mg) as HCl salt as yellow solid. ¹H NMR: (400 MHz, DMSO-d⁶) δ 3.29-3.18 (m, 6H), 3.12-3.03 (m, 2H), 2.63-2.52 (m, 4H), 2.30-2.19 (m, 2H). LCMS calc for C₆H₁₅N₂₀₂S [M+H]⁺: m/z=179.1; Found: 179.1.

### Int 25: cis-4-((methylamino)methyl)cyclohexan-1-ol

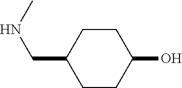

### Step 1: cis-4-((tert-butyldimetylsilyl)oxy)cyclohexane-1-carboxylic acid

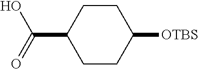 [SMILES: CC(C)(C)[Si](C)(C)O[C@H]1CC[C@@H](C(=O)O)CC1]

To a mixture of cis-4-hydroxycyclohexane-1-carboxylic acid (1 g, 6.9 mmol) and imidazole (1.88 g, 27.8 mmol) in DMF (20 mL) was added TBSCI (2.1 g, 13.9 mmol). The reaction mixture was stirred at 25° C. overnight. The reaction mixture was diluted with H₂O (40 mL) and extracted with EtOAc (30 mL×3). The combined organic layers were washed with brine, dried over Na₂SO₄, filtered and concentrated under reduced pressure. The residue was purified by column chromatography on a silica gel column eluting with EtOAc/PE (5-20%) to afford the title compound (0.85 g) as a colorless oil. LCMS calc. for C₁₃H₂₇03Si [M+H]⁺: m/z=259.2; Found: 259.2.

### Step 2: cis-4-((tert-butyldimethylsilyl)oxy)-N-methylcyclohexane-1-carboxamide

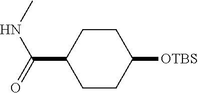 [SMILES: CNC(=O)[C@H]1CC[C@@H](O[Si](C)(C)C(C)(C)C)CC1]

To a mixture of cis-4-((tert-butyldimethylsilyl)oxy)cyclohexane-1-carboxylic acid (0.83 g, 3.2 mmol) and methylamine hydrochloride (0.43 g, 6.4 mmol) in DMF (40 mL) was added HATU (4.88 g, 12.8 mmol) and DIEA (4.14 g, 32.1 mmol) at 0° C. The reaction mixture was stirred at r.t. overnight. The reaction mixture was diluted with H₂O (80 mL) and extracted with EtOAc (50 mL×3). The combined organic layers were washed with brine, dried over Na₂SO₄, filtered and concentrated under reduced pressure. The residue was purified by column chromatography on a silica gel column eluting with EtOAc/PE (10-35%) to afford the title compound (0.6 g) as a colorless oil. LCMS calc. for C₁₄H₂₉NO₂SiNa [M+Na]⁺: m/z=294.2; Found: 294.2.

### Step 3: cis-1-(−4-((tert-butyldimethylsilyl)oxy)cyclohexyl)-N-methylmethanamine

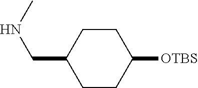 [SMILES: CNC[C@H]1CC[C@@H](O[Si](C)(C)C(C)(C)C)CC1]

To a solution of cis-4-((tert-butyldimethylsilyl)oxy)-N-methylcyclohexane-1-carboxamide (0.5 g, 1.84 mmol) in THF (10 mL) was added BH₃-THF complex solution (10 mL, 1 M in THF) at 0° C. The reaction mixture was stirred at r.t. overnight. The reaction was quenched with MeOH (2 mL) slowly, diluted with water (30 mL) and extracted with EtOAc (30 mL×3). The combined organic layers were washed with brine, dried over Na₂SO₄, filtered and concentrated under reduced pressure. The residue was purified by prep-TLC on a silica gel layer eluting with MeOH/DCM (20%) to afford the title compound (0.4 g) as a colorless oil. LCMS calc. for C₁₄H₃₂ONSi [M+H]⁺: m/z=258.2; Found: 258.2.

### Step 4: cis-4-((methylamino)methyl)cyclohexan-1-ol

A solution of cis-1-((−4-((tert-butyldimethylsilyl)oxy)cyclohexyl)-N-methylmethanamine (0.3 g, 1.16 mmol) in HCl solution (5 mL, 4 M in 1,4-dioxane) was stirred at 25° C. for 2 h.. The reaction mixture was concentrated to afford the title compound (0.2 g) as a yellow solid. LCMS calc. for C₈H₁₈NO [M+H]⁺: m/z=144.1; Found: 144.2.

### Int 26: 2-(4,4-Difluoropiperidin-1-yl)-N-methylethan-1-amine

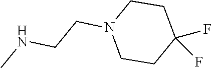

### Step 1: tert-butyl (2-(4,4-difluoropiperidin-1-Y¹)ethyl)(methyl)carbamate

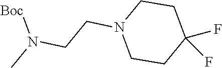 [SMILES: CN(CCN1CCC(F)(F)CC1)C(=O)OC(C)(C)C]

To a mixture of tert-butyl methyl(2-oxoethyl)carbamate (200 mg, 1.2 mmol) and 4,4-difluoropiperidine (167 mg, 1.4 mmol) in THF (10 mL) was added and NaBH₃CN (220 mg, 3.5 mmol) at 0° C. The mixture was stirred at 25° C. for 16 h., diluted with H₂O and extracted with EA (20 mL×3). The combined organic layers were washed with brine, dried over Na₂SO₄, filtered and concentrated under reduced pressure. The residue was purified by prep-TLC on a silica gel layer eluting with EA/PE (20%) to afford the title compound (150 mg) as yellow solid.

LCMS calc for C₁₃H₂₅F₂N₂02, [M+H]⁺: m/z=279.2; Found: 279.2.

### Step 2: 2-(4,4-difluoropiperidin-1-yl)-N-methylethan-1-amine

To a solution of tert-butyl (2-(4,4-difluoropiperidin-1-Y¹)ethyl)(methyl)carbamate (100 mg, 0.36 mmol) in DCM (1 mL) was added HCl solution (1 mL, 4 M in 1,4-dioxane). The reaction mixture was stirred at r.t. for 3 h.. The reaction mixture was concentrated under reduced pressure to afford the title compound (100 mg) as HCl salt as yellow solid. LCMS calc. for C₈H₁₇F₂N₂ [M+H]⁺: m/z=179.1; Found: 179.3.

### Int 27: 4-(2-(Methylamino)ethyl)thiomorpholine 1,1-dioxide

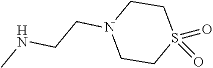

This compound was prepared by procedures analogous to those described for Int 26 Step 1-2 using thiomorpholine 1,1-dioxide. LCMS calc. for C₇H₁₇N₂O₂S [M+H]⁺: m/z=193.1; Found: 193.1.

### Int 28: N-Methyl-1-(1-methylpyrrolidin-2-Y¹)methanamine

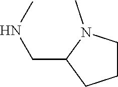

### Step 1: (1-methylpyrrolidin-2-Y¹)methyl methanesulfonate

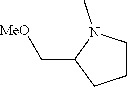

To a mixture of (1-methylpyrrolidin-2-Y¹)methanol (100 mg, 0.86 mmol) and TEA (263 mg, 2.6 mmol) in DCM (4 mL) was added methanesulfonyl chloride (148 mg, 1.3 mmol) dropwise at 0° C. The reaction mixture was stirred at r.t. for 2 h.. The reaction mixture was diluted with H₂O (20 mL) and extracted with DCM (20 mL×3). The combined organic layers were washed with brine, dried over Na₂SO₄, filtered and concentrated under reduced pressure to afford the title compound (130 mg). TLC R^{f}=0.2 (PE/EtOAc=1:1, I₂).

### Step 2: N-methyl-1-(1-methylpyrrolidin-2-Y¹)methanamine

A mixture of (1-methylpyrrolidin-2-Y¹)methyl methanesulfonate (130 mg, 0.67 mmol) in methylamine solution (30 mL, 2.0 M in EtOH) was stirred at 70° C. for 16 h.. The reaction mixture was diluted with H₂O (30 mL) and extracted with DCM (20 mL×3). The organic phase was washed with brine, dried over Na₂SO₄, filtered and concentrated under reduced pressure. The residue was purified by prep-TLC on a silica gel layer eluting with MeOH/DCM (20%) to afford the title compound (60 mg) as a yellow solid. LCMS calc for C₇H₁₇N₂, [M+H]⁺: m/z=129.1; Found: 129.3.

### Example A: Biological Assays

### PARG Enzymatic Activity Assay

HTRF assay was used to measure the ability of compounds to inhibit the activity of PAR in vitro. C-terminal His6—tag PARG expressed in E. coli was purified and stored at −80° C. in aliquots. Assay measurements were performed with 1×buffer comprising 50 mM Tris pH 7.4, 0.1 mg/mL BSA, 3 mM EDTA, 0.4 mM EGTA, 1 mM DTT, 50 mM KCl and 0.01% Tween 20. Compounds dissolved in DMSO were plated into a 384-well assay plate (PerkinElmer, Catalog #: 6008280) in duplicate using a dispenser (Labcyte, Echo 665), and tested on a 10-point 4-fold serial dilution. Add 5 μL Enzyme mix [hPARG (C-terminal His6—tag, 4.2 μM), 65 pM final] to the test wells. The plates were covered and left to incubate for 60 minutes at RT before the addition of 5 μL substrate mix [biotinylated-NAD ribosylated PARP1 (6.1 μM), 8 nM final] to initiate reaction. After incubating 10 minutes at RT, add 2.5 μL Streptavidin-Eu cryptate (Cisbio, Catalog #: 610SAKLA) and 2.5 μL Mab anti 6HIS-XL665 (Cisbio, Catalog #: 61HISXLA) to the plate, and incubate for 60 minutes at RT. Read on a multimode plate reader (PerkinElmer, Envision 2015) in time-resolved fluorescence (TRF) mode, with excitation at 337 nm and emission at both 620 nm and 665 nm. Average HTRF signal of high control (Wells with 1% DMSO) was calculated and as Vehicle Control (VC). Average HTRF signal of low control (no Enzyme) was calculated and as Positive control (PC).

$$\% Inhibition = ( {Signal}_{cmpd} - {Signal}_{Ave\_VC} ) / ( {Signal}_{Ave\_PC} - {Signal}_{Ave\_VC} ) \times 100.$$

IC₅₀ values were determined by fitting the data to the standard 4 parameters with Hill Slope using GraphPad Prism software.

The compounds of the invention were found to be inhibitors of PARG according to the above-described assay. IC₅₀data is proved below in Table 7: a “+” denotes an IC₅₀ value of >1 μM, a “++” denotes an IC₅₀value of 0.1 μM<IC₅₀≤1 μM, and a “+++” denotes an IC₅₀value of ≤0.1 IM.

### Cell Viability Assay

Cell viability studies were conducted in Kuramochi cell line. Cells were maintained in RPMI (Hyclone, Catalog #: SH3080901B) supplemented with 10% v/v FBS (AusGeneX, Catalog #: FBS500-S), 100 v/v Penicillin Streptomycin (Gibco, Catalog #: 15140122). Cells were seeded in 96-well plates (PerkinElmer, Catalog #: 6005680) at a density of 400 cells/well. Compounds dissolved in DMS0 were plated in duplicate using a multichannel pipette, and tested on a 9-point 3-fold serial dilution. Cells were incubated for 7 days in a 37° C. active humidified incubator at 500 CO₂. Cell viability was measured using the Cell Titer-Glo reagent (Promega, Catalog #: G7573) as manufacturer's instructions. Luminescence signal was measured with a multimode plate reader (Perkin Elmer, Envision 2105 or BMG, ClarioStar Plus). Average values of DMS0 treated wells in a plate was calculated and as high control (HC). Average values of only medium in a plate was calculated and as low control (LC).

$$\% Inhibition = ( {Signal}_{Ave\_HC} - {Signal}_{cmpd} ) / ( {Signal}_{Ave\_HC} - {Signal}_{Ave\_LC} ) \times 100.$$

IC₅₀ values were determined by fitting the data to the standard 4 parameters with Hill Slope using GraphPad Prism software. IC₅₀data is proved below: a “+” denotes an IC₅₀value of >1 μM, a “++” denotes an IC₅₀value of 0.1 μM<IC₅₀≤1 μM, and a “+++” denotes an IC₅₀value of ≤0.1 μM.

TABLE 7

| Ex. | PARG enzymatic | Ex. | PARG enzymatic | Ex. | PARG enzymatic |
|---|---|---|---|---|---|
| # | activity (IC₅₀) | # | activity (IC₅₀) | # | activity (IC₅₀) |
| 1 | +++ | 2 | +++ | 3 | +++ |
| 4 | +++ | 5 | +++ | 6 | ++ |
| 7 | +++ | 8 | +++ | 9 | +++ |
| 10 | +++ | 11 | +++ | 12 | +++ |
| 13 | +++ | 14 | +++ | 15 | +++ |
| 16 | +++ | 17 | +++ | 18 | +++ |
| 19 | +++ | 20 | +++ | 21 | +++ |
| 22 | ++ | 23 | +++ | 24 | ++ |
| 25 | +++ | 26 | + | 27 | +++ |
| 28 | +++ | 29 | +++ | 30 | +++ |
| 31 | +++ | 32 | +++ | 33 | +++ |
| 34 | +++ | 35 | +++ | 36 | +++ |
| 37 | +++ | 38 | +++ | 39 | +++ |
| 40 | ++ | 41 | +++ | 42 | +++ |
| 43 | +++ | 44 | +++ | 45 | +++ |
| 46 | +++ | 47 | +++ | 48 | +++ |
| 49 | +++ | 50 | +++ | 51 | +++ |
| 52 | +++ | 53 | +++ | 54 | +++ |
| 55 | +++ | 56 | +++ | 57 | +++ |
| 58 | ++ | 59 | ++ | 60 | +++ |
| 61 | ++ | 62 | +++ | 63 | +++ |
| 64 | +++ | 65 | +++ | 66 | +++ |
| 67 | +++ | 68 | +++ | 69 | +++ |
| 70 | ++ | 71 | + | 72 | ++ |
| 73 | +++ | 74 | +++ | 75 | +++ |
| 76 | +++ | 77 | ++ | 78 | +++ |
| 79 | +++ | 80 | +++ | 81 | +++ |
| 82 | +++ | 83 | ++ | 84 | ++ |
| 85 | +++ | 86 | +++ | 87 | +++ |
| 88 | +++ | 89 | ++ | 90 | ++ |
| 91 | +++ | 92 | ++ | 93 | +++ |
| 94 | +++ | | | | |

Although the present invention has been comprehensively described through its embodiments, it is worth noting that various changes and modifications are obvious to those skilled in the art. Such changes and modifications should be included in the scope of the appended claims of the present invention.

What is claimed:

1. A compound of formula (I), or a pharmaceutically acceptable salt, stereoisomer, solvate, N-oxide, tautomer, isotopic variant, prodrug, or deuterated compound thereof, wherein: 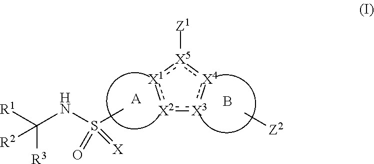 [symbol]
is a single bond or double bond;
X is O or NR⁴;
X is C or N;
X² is N or C;
X³ is N or C;
X⁴ is C or N;
X⁵ is Cor N; F 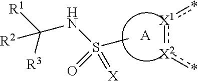
is 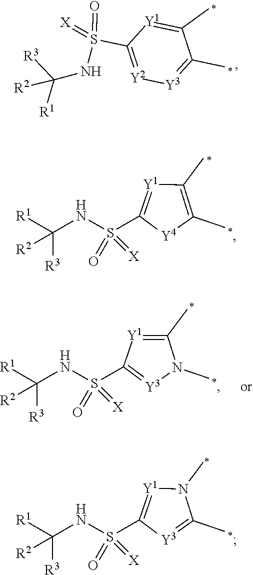 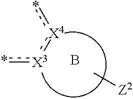
is 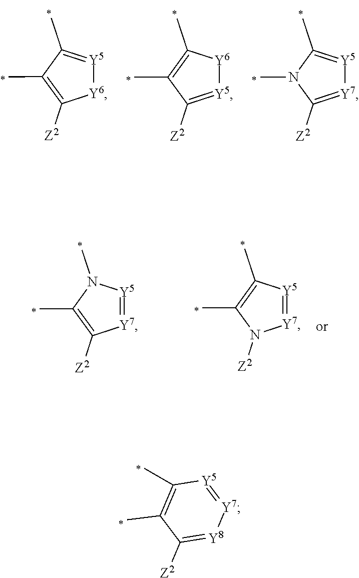
Y¹ and Y³ are each independently N or CR⁵;
Y² is N or CR⁶;
Y⁴ is S, O or NR⁷;
Y⁵, Y⁷ and Y⁸ are each independently N or CR⁸;
Y⁶ is S, O or NR⁹;
Z¹ is 5-10 membered heteroaryl optionally substituted by 1, 2, 3, 4, or 5 substituents independently selected from R¹⁰;
Z² is H, D, halo, CN, NO₂, SF₅, C₁-C₈ alkyl, C₂-C₈ alkenyl, C₂-C₈ alkynyl, C₆-C₁₀ aryl, C₃-C₁₀ cycloalkyl, 5-10 membered heteroaryl, 4-14 membered heterocycloalkyl, NR^{C}R^{D}, OR^{A}, SR^{A}, NHOR^{A}, C(O)R^{B}, C(O)OR^{A}, C(O)NR^{C}R^{D}, OC(O)NR^{C}R^{D}, NR^{C}C(O)R^{B}NR^{C}C(O)NR^{C}R^{D}, NR^{C}C(O)OR^{A}, NR^{C}S(O)₂R^{B}, S(O)R^{B}, S(O)NR^{C}R^{D}, S(O)₂R^{B}, S(O)₂NR^{C}R^{D}, or NR^{C}S(O)₂NR^{C}R^{D}; wherein, the C₁-C₅ alkyl, C₂-C₅ alkenyl, C₂-C₅ alkynyl, C₆-C₁₀ aryl, C₃-C₁₀ cycloalkyl, 5-10 membered heteroaryl, or 4-14 membered heterocycloalkyl is optionally substituted by 1, 2, 3, 4, or 5 substituents independently selected from R¹¹;
R¹, R² and R³ are each independently H, D, CN, C₁-C₃ alkyl, C₂-C₃ alkenyl, C₂-C₃ alkynyl, C₃-C₇ cycloalkyl, or 4-7 membered heterocycloalkyl; wherein, the C₁-C₃ alkyl, C₂-C₃ alkenyl, C₂-C₃ alkynyl, C₃-C₇ cycloalkyl, or 4-7 membered heterocycloalkyl is optionally substituted by 1, 2, 3, 4 or 5 substituents independently selected from D, halo, CN, OH, C₁-C₆ alkyl, C₁-C₆ haloalkyl, —O—C₁-C₆ alkyl, —OC₁-C₆ haloalkyl;
or R² and R³ together with the carbon atom to which they are attached form C₃-C₇ cycloalkyl, or 4-7 membered heterocycloalkyl; wherein, the C₃-C₇ cycloalkyl, 4-7 membered heterocycloalkyl is optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl;
R⁴ is H, D, CN, OR^{B}, or C₁-C₄ alkyl optionally substituted with at least one of R^{4A}; wherein, each R^{4A} is independently D, F, Cl, CN, NH₂, OH, —O—C₁-C₆ alkyl, —OC₁-C₆ haloalkyl, optionally substituted C₃-C₇ cycloalkyl, or optionally substituted 4-7 membered heterocycloalkyl; wherein, optionally substituted is D, halo, CN, OH, C₁-C₄ alkyl, C₁-C₄ haloalkyl, —O—C₁-C₄ alkyl, —OC₁-C₄ haloalkyl;
or R¹ and R⁴ together with the atoms to which they are attached form 5-7 membered partially unsaturated heterocycloalkyl optionally substituted by 1, 2, 3 or 4 substituents independently selected from D, halogen, CN, CF₃, NO₂, oxo, OH, C₁-C₆ alkyl, C₁-C₆ haloalkyl, —O—C₁-C₆ alkyl, —OC₁-C₆ haloalkyl;
R⁵ is H, D, CN, halo, SF₅, OH, NH₂, CHO, COOH, C₁-C₃ alkyl, C₂-C₃ alkenyl, C₂-C₃ alkynyl, C₁-C₃ haloalkyl, C₁-C₃ cyanoalkyl, OR^{A}, NR^{C}R^{D} or C(O)R^{B};
R⁶ is H, D, CN, halo, OH, NH₂, SF₅, C₁-C₃ alkyl, C₁-C₃ haloalkyl, —O—C₁-C₃ alkyl, —OC₁-C₃ haloalkyl, C₁-C₃ cyanoalkyl;
R⁷ and R⁹ are each selected from H, D, C₁-C₆ alkyl, C₁-C₆ haloalkyl, C₂-C₆ alkenyl, C₂-C₆ alkynyl, C₁-C₆ alkyl-OR^{A}, C₁-C₆ alkyl-CN, C₁-C₆ alkyl-NR^{C}R^{D}, C(O)R^{B}, C(O)NR^{C}R^{D};
each R⁸ is independently H, D, halogen, CN, NO₂, C₁-C₆ alkyl, C₁-C₆ haloalkyl, C₂-C₆ alkenyl, C₂-C₆ alkynyl, OR^{A}, SR^{A}, SF₅, NHOR^{A}, C(O)OR^{A}, C(O)R^{B}, C(O)NR^{C}R^{D}, OC(O)NR^{C}R^{D}, NR^{C}R^{D}, NR^{C}C(O)R^{B}, NR^{C}C(O)NR^{C}R^{D}, NR^{C}C(O)OR^{A}, NR^{C}S(O)₂R^{B} B(OR^{C})(OR^{D}), C(═NR^{C})NR^{C}R^{D}, NR^{D}C(═NR^{C})NR^{C}R^{D}, NR^{D}C(═NR^{C})R^{B}, P(O)R^{E}R^{F} P(O)OR^{E}OR^{F}, OP(O)OR^{E}OR^{F}, S(O)(═NR^{B})R^{B}, S(O)R^{B}, S(O)NR^{C}R^{D}, S(O)₂R^{B}, S(O)₂NR^{C}R^{D}, NR^{C}S(O)₂NR^{C}R^{D}, or NR^{C}S(O)(═NR^{B})R^{B}; wherein, the C₁-C₆ alkyl, C₂-C₆ alkenyl, or C₂-C₆ alkynyl is optionally substituted with 1, 2, or 3 substituents independently selected from R¹²;
each R¹⁰ is independently H, D, halo, C₁-C₆ alkyl, C₂-C₆ alkenyl, C₂-C₆ alkynyl, C₁-C₆ haloalkyl, OC₁-C₆ alkyl, OC₁-C₆ haloalkyl, OC₃-₇ cycloalkyl, C₃-C₇ cycloalkyl, CN, NO₂, N₃, or SF₅; wherein, the C₁-C₆ alkyl, C₂-C₆ alkenyl, C₂-C₆ alkynyl or C₃-C₇ cycloalkyl is optionally substituted with 1, 2, or 3 substituents independently selected from R¹²;
two R¹⁰, together with the atom(s) to which they are attached form oxo, C₃-C₁₀ cycloalkyl or 4-10 membered heterocycloalkyl; wherein, the C₃-C₁₀ cycloalkyl or 4-10 membered heterocycloalkyl optionally substituted by 1, 2, or 3 substituents independently selected from D, halo, C₁-C₆ alkyl, C₂-C₆ alkenyl, C₂-C₆ alkynyl, C₁-C₆ haloalkyl, C₁-C₆-cyanoalkyl, CN, NO₂, oxo, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{c}R^{d} NR^{c}C(O)R^{b}, NR^{c}C(O)NR^{c}R^{d} NR^{c}C(O)OR^{a}, B(OR^{c})(OR^{d}), C(═NR^{c})NR^{c}R^{d}, NR^{d}C(═NR^{c})NR^{c}R^{d}, NR^{d}C(═NR^{c})R^{b}, OP(O)OR^{e}OR^{f}, P(O)OR^{e}OR^{f}, S(O)(═NR^{b})R^{b}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d} NR^{c}S(O)(═NR^{b})R^{b}, Cy⁴; wherein, Cy⁴ is C₆-C₁₀ aryl, C₃-C₁₀ cycloalkyl, 5-10 membered heteroaryl, and 4-10 membered heterocycloalkyl; wherein Cy⁴ is optionally substituted by 1, 2, 3 or 4 substituents independently selected from D, halo, CN, NO₂, OH, oxo, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅;
each R¹¹ is independently H, D, halo, CN, NO₂, N₃, oxo, C₁-C₆ alkyl, C₂-C₆ alkenyl, C₂-C₆ alkynyl, OR^{A}, SR^{A}, SF₅, NR^{C}OR^{A}, C(O)R^{B}, C(═S)R^{B}, C(O)NR^{C}R^{D}, C(O)N(R^{c})OR^{A}, C(O)OR^{A}, OC(O)R^{B}, OC(O)NR^{C}R^{D}, NR^{C}R^{D}, NR^{C}C(O)R^{D}, NR^{C}C(O)NR^{C}R^{D} NR^{C}C(O)OR^{A}, B(OR^{C})(OR^{D}), C(═NR^{C})NR^{C}R^{D}, NR^{D}C(═NR^{C})NR^{C}R^{D}, NR^{D}C(═NR^{C})R^{B} SiR^{G}R^{H}R^{I}, P(O)R^{E}R^{F}, P(O)OR^{E}OR^{F}, OP(O)OR^{E}OR^{F}, S(O)(═NR^{B})R^{B}, S(O)R^{B}, S(O)NR^{C}R^{D}, S(O)₂R^{B}, NR^{C}S(O)₂R^{B}, S(O)₂NR^{C}R^{D}, NR^{C}S(O)₂NR^{C}R^{D} NR^{C}S(O)(═NR^{B})R^{B} Cy³, C₁-C₆ alkyl-Cy³, OCy³, or O—C₁-C₆ alkyl-Cy³; wherein, the C₁-C₆ alkyl, C₂-C₆ alkenyl, C₂-C₆ alkynyl is optionally substituted with 1, 2, 3, 4 or 5 substituents independently selected from R¹²;
each R¹² is independently H, D, halo, CN, NO₂, N₃, C₁-C₆ alkyl, C₂-C₆ alkenyl, C₂-C₆ alkynyl, C₁-C₆ haloalkyl, OC₁-C₆ alkylOH, OC₁-C₆ alkyl-O—C₁-C₆ alkyl, OR^{a1}, SR^{a1}, SFs, NHOR^{a1}, C(O)R^{b1}, C(O)NR^{c1}R^{d1}, C(O)OR^{a1}, OC(O)OR^{a1}, OC(O)R^{b1}, OC(O)NR^{c1}R^{d1} NR^{c}R^{d1} NR^{C}C(O)R^{b1}, NR^{C}C(O)NR^{c1}R^{d1} NR^{c1}C(O)OR^{a1}, B(OR^{c1})(OR^{d1}), C(═NR^{c1})NR^{c1}R^{d1} NR^{d1}C(═NR^{c1})NR^{c1}R^{d1} NR^{d1}C(═NR^{c1})R^{b1}, P(O)OR^{e1}OR^{f1} OP(O)OR^{e1}OR, S(O)(═NR^{b1})R^{b1}, S(O)R^{b1}, S(O)NR^{c1}R^{d1}, S(O)₂R^{b}1, NR^{c1} S(O)₂R^{b}1, S(O)₂NR^{cl}R^{dl} NR^{c1}S(O)₂NR^{c1}R^{d1} NR^{c1}S(O)(═NR^{b1})R^{b1} C₆-C₁₀ aryl, C₃-C₁₀ cycloalkyl, 5-membered heteroaryl, and 4-10 membered heterocycloalkyl, wherein, the C₆-C₁₀ aryl, C₃-C₁₀ cycloalkyl, 5-10 membered heteroaryl, 4-10 membered heterocycloalkyl is optionally substituted by 1, 2, 3, 4 or 5 substituents independently selected from D, halo, CN, NO₂, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅;
Cy³ is independently C₆-C₁₀ aryl, C₃-C₁₀ cycloalkyl, 5-10 membered heteroaryl, or 4-membered heterocycloalkyl; wherein Cy³ is optionally substituted with 1, 2, 3, 4 or 5 substituents independently selected from R¹³;
each R¹³ is independently D, halo, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SFS, NHOR^{a1}, C(O)R^{b1}, C(O)NR^{c1}R^{d1}, C(O)OR^{a1}, OC(O)R^{b1}, OC(O)NR^{c1}R^{d1} NR^{c1}R^{d1} NR^{c1}C(O)R^{b1} NR^{c1}C(O)NR^{c1}R^{d1} NR^{c1}C(O)OR^{a1}, B(OR^{c1})(OR^{d1}), C(═NR^{c1})NR^{c1}R^{d1} NR^{d1}C(═NR^{c1})NR^{c1}R^{d1} NR^{d1}C(═NR^{c1})R^{b1}, P(O)R^{e1}R^{f1}, P(O)OR^{e1}OR^{f1}, OP(O)OR^{e1}OR^{f}1, S(O)(═NR^{b1})R^{b1}, S(O)R^{b1}, S(O)NR^{c1}R^{d1}, S(O)₂R^{b1}, NR^{c1}S(O)₂R^{b1}, S(O)₂NR^{c1}R^{d1} NR^{c1}S(O)₂NR^{c1}R^{d1} NR^{c1}S(O)(═NR^{b1})R^{b1}, C₆-C₁₀ aryl, C₃-C₁₀ cycloalkyl, 5-10 membered heteroaryl, and 4-10 membered heterocycloalkyl; wherein, the C₆-C₁₀ aryl, C₃-C₁₀ cycloalkyl, 5-10 membered heteroaryl, 4-10 membered heterocycloalkyl optionally substituted by 1, 2, 3, 4 or 5 substituents independently selected from D, halo, CN, NO₂, NH₂, NHC₁-C₄ alkyl, N(C₁-C₄ alkyl)₂, C₁-C₃ alkyl, C₁-C₃ haloalkyl, OC₁-C₃ alkyl, OC₁-C₃ haloalkyl, OC₂-C₃ alkylOH, OC₂-C₃ alkyl-O—C₁-C₆ alkyl, or SF₅;
each R¹⁴ is independently H, D, NO₂, CN, halo, oxo, SF₅, C₁-C₈ alkyl, C₂-C₈ alkenyl, C₂-C₈ alkynyl, C₆-C₁₄ aryl, C₃-C₁₄ cycloalkyl, 5-14 membered heteroaryl, or 4-14 membered heterocycloalkyl, OR^{a}, SR^{a}, SF₅, NHOR^{a}, C(O)R^{b}, C(O)NR^{c}R^{d}, C(O)OR^{a}, OC(O)R^{b}, OC(O)NR^{c}R^{d}, NR^{c}R^{d} NR^{c}C(O)R^{b}, NR^{c}C(O)NR^{c}R^{d} NR^{c}C(O)OR^{a}, B(OR^{c})(OR^{d}), C(═NR^{c})NR^{c}R^{d}, NR^{d}C(═NR^{c})NR^{c}R^{d}, NR^{d}C(═NR^{c})R^{b}, P(O)R^{e}R^{f}, P(O)OR^{e}OR^{f}, OP(O)OR^{e}OR^{f}, S(O)R^{b}, S(O)NR^{c}R^{d}, S(O)₂R^{b}, NR^{c}S(O)₂R^{b}, S(O)₂NR^{c}R^{d}, NR^{c}S(O)₂NR^{c}R^{d}, or NR^{c}S(O)(═NR^{b})R^{b}; wherein, the C₁-C₈ alkyl, C₂-C₈ alkenyl, C₂-C₈ alkynyl, C₆-C₁₄ aryl, C₃-C₁₄ cycloalkyl, 5-14 membered heteroaryl, or 4-14 membered heterocycloalkyl is optionally substituted by 1, 2, 3, 4 or 5 substituents independently selected from D, NO₂, CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SFs, NHOR^{a1}, C(O)R^{b1}, C(O)NR^{c1}R^{d1}, C(O)OR^{a1}, OC(O)R^{b}1, OC(O)NR^{c1}R^{d1} NR^{c1}R^{d1} NR^{c1}C(O)R^{b}, NR^{c1}C(O)NR^{c1}R^{d1} NR^{c1}C(O)OR^{a1}, B(OR^{c1})(OR^{d1}), C(═NR^{c1})NR^{c1}R^{d1} NR^{d1}C(═NR¹)NR^{c1}R^{d1} NR^{d1}C(═NR^{c1})R^{b}1, P(O)R^{e1}R^{f1}, P(O)OR^{e1}OR^{f}1, OP(O)OR^{e1}OR^{f}1, S(O)(═NR^{b1})R^{b1}, S(O)R^{b1}, S(O)NR^{c1}R^{d1}, S(O)₂R^{b}1, NR^{c1} S(O)₂R^{b}1, S(O)₂NR^{c1}R^{d1} NR^{c1}S(O)₂NR^{c1}R^{d1} NR^{c1}S(O)(═NR^{b1})R^{b1}, C₆-C₁₀ aryl, C₃-C₁₀ cycloalkyl, 5-10 membered heteroaryl, and 4-10 membered heterocycloalkyl;
each R^{A} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocyclalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴;
each R^{B} is independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴;
R^{C} and R^{D} are each independently H, D, C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein the C₁-C₆ alkyl, C₂-C₆ alkenyl, C₂-C₆ alkynyl, C₃-C₁₀ cycloalkyl, 4-10 membered heterocycloalkyl, C₆-C₁₀ aryl, 5-membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴;
or R^{C} and R^{D} together with the N atom to which they are attached form 4-7 membered heterocycloalkyl optionally substituted with 1, 2, 3, 4 or 5 substituents independently selected from D, halo, oxo, CN, C₁-C₄ alkyl, C₁-C₄haloalkyl, C₁-C₄ alkyl-CN, OR^{a}, SR^{a}, C(O)R^{b}, NR^{c}R^{d};
R^{a} and R^{a1}are each independently H, D, C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl, wherein the C₁-C₄ alkyl, C₂-C₄ alkenyl, C₂-C₄ alkynyl, phenyl, C₃-C₇ cycloalkyl, 5-6 membered heteroaryl, or 4-7 membered heterocycloalkyl is optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄haloalkyl, or C₁-C₄haloalkoxy;
R^{b} and R^{b1} are each independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl; wherein the C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, or 4-10 membered heterocycloalkyl-C₁-C₄ alkyl is optionally substituted with 1, 2, 3, 4 or 5 substituents independently selected from D, OH, halo, CN, —NH₂, —NH(C₁-C₄ alkyl), —N(C₁-C₄ alkyl)₂, C₁-C₄ alkyl, C₁-C₄ haloalkyl, —OC₁-C₄ alkyl or —OC₁-C₄haloalkyl, C₆-C₁₀ aryl, C₃-C₁₀ cycloalkyl, 5-10 membered heteroaryl, or 4-membered heterocycloalkyl;
R^{c}, R^{d}, R^{c1} and R^{d1} are each independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, 4-10 membered heterocycloalkyl-C₁-C₄ alkyl, C₆-C₁₀ aryl-C₃-C₁₀ cycloalkyl, C₆-C₁₀ aryl-4-10 membered heterocycloalkyl, C₆-C₁₀ aryl-5-10 membered heteroaryl, bi(C₆-C₁₀ aryl), 5-10 membered heteroaryl-C₃-C₁₀ cycloalkyl, 5-10 membered heteroaryl-4-10 membered heterocycloalkyl, 5-10 membered heteroaryl-C₆-C₁₀ aryl, or bi(5-membered heteroaryl); wherein, the C₁₋₄ alkyl, C₂₋₄ alkenyl, C₂₋₄ alkynyl, C₆-C₁₀ aryl, 5-membered heteroaryl, C₃-C₁₀ cycloalkyl, 4-10 membered heterocycloalkyl, C₆-C₁₀ aryl-C₁-C₄ alkyl, 5-10 membered heteroaryl-C₁-C₄ alkyl, C₃-C₁₀ cycloalkyl-C₁-C₄ alkyl, 4-10 membered heterocycloalkyl-C₁-C₄ alkyl, C₆-C₁₀ aryl-C₃-C₁₀ cycloalkyl, C₆-C₁₀ aryl-4-10 membered heterocycloalkyl, C₆-C₁₀ aryl-5-10 membered heteroaryl, bi(C₆-C₁₀ aryl), 5-10 membered heteroaryl-C₃-C₁₀ cycloalkyl, 5-10 membered heteroaryl-4-10 membered heterocycloalkyl, 5-10 membered heteroaryl-C₆-C₁₀ aryl, or bi(5-10 membered heteroaryl) is optionally substituted with 1, 2, 3, 4 or 5 substituents independently selected from D, halo, OH, CN, —NH₂, —NH(C₁-C₄ alkyl), —N(C₁-C₄ alkyl)₂, C₁-C₄ alkyl, O—C₁-C₄ alkyl, C₁-C₄ haloalkyl, 0-C₁-C₄ haloalkyl, C₁-C₄ alkyl-OH, C₁-C₄ alkyl-CN, C₆-C₁₀ aryl, 5-10 membered heteroaryl, C(O)OR^{a1}, C(O)R^{b1}, S(O)₂R^{b1}, C₁-C₄ alkyl-O—C₁-C₄ alkyl, and C₁-C₄ alkyl-O—C₁-C₄ alkyl-O—;
or R^{c} and R^{d} together with the N atom to which they are attached form a 4-7 membered heterocycloalkyl optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkoxy-C₁-C₄ alkyl, and C₁-C₄ alkoxy-C₁-C₄ alkoxy;
or R^{c} and R^{d1} together with the N atom to which they are attached form a 4-7 membered heterocycloalkyl optionally substituted with 1, 2, or 3 substituents independently selected from D, OH, CN, —NH₂, —NH(C₁-C₄ alkyl), —N(C₁-C₄ alkyl)₂, halo, C₁-C₄ alkyl, C₁-C₄ alkoxy, C₁-C₄ haloalkyl, C₁-C₄ haloalkoxy, C₁-C₄ hydroxyalkyl, C₁-C₄ cyanoalkyl, C₆-C₁₀ aryl, 5-10 membered heteroaryl, C(O)OR^{a1}, C(O)R^{b1}, S(O)₂R^{b1}, C₁-C₄ alkoxy-C₁-C₄ alkyl, and C₁-C₄ alkoxy-C₁-C₄ alkoxy;
R^{E}, R^{e} and R^{e1} are each independently H, D, C₁-C₄ alkyl, C₁-C₄ haloalkyl, C₂-C₄ alkenyl, (C₁-C₄ alkoxy)-C₁-C₄ alkyl, C₂-C₄ alkynyl, C₆-C₁₀ aryl, 5-10 membered heteroaryl, C₃-C₁₀ cycloalkyl, 4-10 membered heterocycloalkyl, C₆-C₁₀ aryl-C₁-C₄ alkyl, C₃-C₁₀ cycloalkyl-C₁-C₄ alkyl, 5-10 membered heteroaryl-C₁-C₄ alkyl, or 4-10 membered heterocycloalkyl-C₁-C₄ alkyl;
R^{F}, R^{f} and R^{f1} are each independently H, D, C₁-C₄ alkyl, C₂-C₄ alkenyl, C₂-C₄ alkynyl, C₆-C₁₀ aryl, 5-10 membered heteroaryl, C₃-C₁₀ cycloalkyl, or 4-10 membered heterocycloalkyl;
R^{G}, R^{H} and R are each independently C₁-C₄ alkyl or phenyl.

2. The compound of claim 1, wherein, the compound having the structure of Formula (Ie): 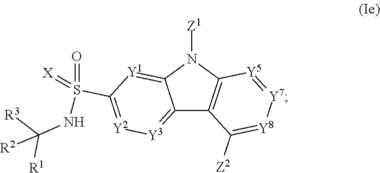
or a pharmaceutically acceptable salt, stereoisomer, solvate, N-oxide, tautomer, isotopic variant, prodrug, or deuterated compound thereof,
wherein, R¹, R², R³, X, Y¹, Y², Y¹, Y⁵, Y⁷, Y⁸, Z¹, and Z² are defined with respect to Formula (I).

3. The compound of claim 1, wherein, Z¹ is 5-6 membered heteroaryl having 1, 2, or 3 heteroatoms independently selected from N, O, S which is optionally substituted by 1, 2, 3, 4 substituents independently selected from R¹⁰; preferably, Z¹ is 5 membered heteroaryl having 1, 2, or 3 heteroatoms independently selected from N, O, S which is optionally substituted by 1, 2, or 3 substituents independently selected from R¹⁰;
preferably, Z¹ is 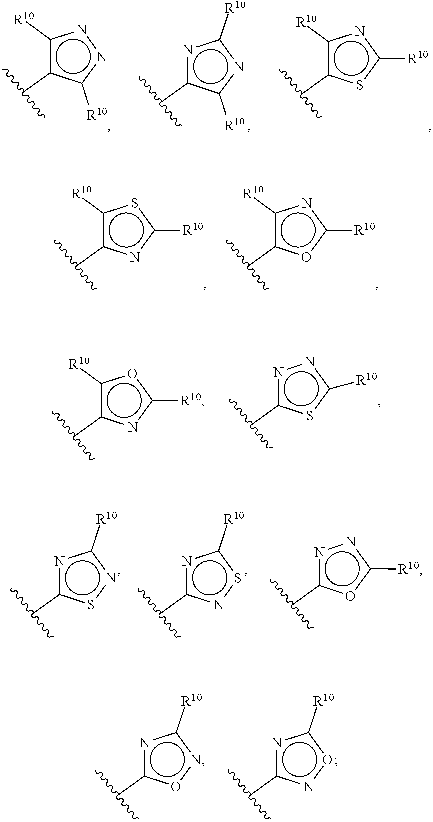
preferably, Z¹ is 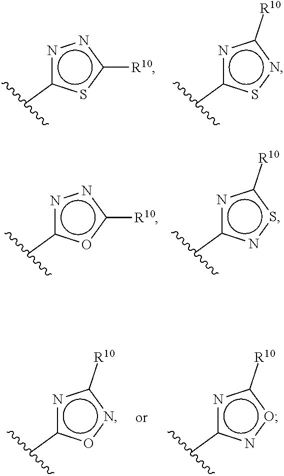
preferably, Z¹ is 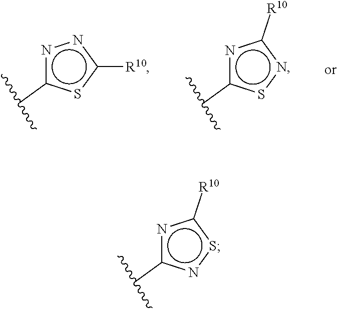
preferably, Z¹ is

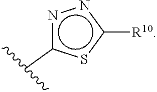

4. The compound of claim 1, wherein, the compound having the structure of Formula (III): 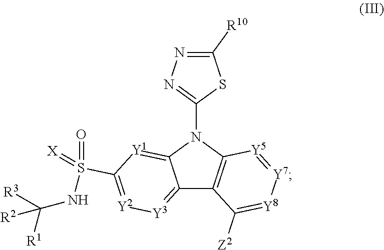
or pharmaceutically acceptable salt, stereoisomer, solvate, N-oxide, tautomer, isotopic variant, prodrug, or deuterated compound thereof; wherein R¹, R², R³, R¹⁰, X, Y, Y², Y³, Y⁵, Y⁷, Y⁸, and Z² are defined with respect to Formula (I);
and/or, R¹ is H, D, CN, C₁-C₃ alkyl optionally substituted by 1, 2, 3, 4 or 5 substituents independently selected from D, halo, CN, OH, Me, CF₃, OMe, OCF₃, OEt;
and/or, R¹ is H, D, CN, CH₃, CD₃, CH₂CH₃, CF₃, CHF₂, CH₂F, CH₂CH₂F, CH₂OH, CH₂OCH₃ or CH₂CN;
and/or, R² and R³ together with the carbon atom to which they are attached form cyclopropyl, cyclobutyl, oxetanyl, or siletanyl; each is optionally substituted by 1, 2, 3 or 4 substituents independently selected from D, halo, CN, NO₂, oxo, OH, C₁-C₆alkyl, C₁-C₆haloalkyl, —O—C₁-C₆ alkyl, —OC₁-C₆haloalkyl;
and/or, the moiety 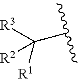
has the structure of

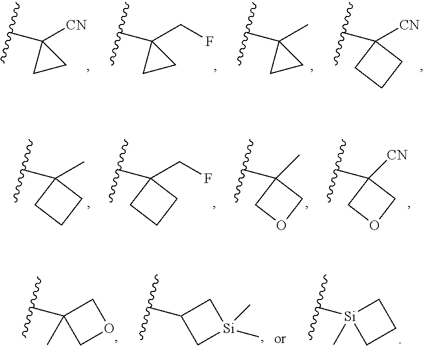

5. The compound of claim 1, wherein, the compound having the structure of Formula (IV): 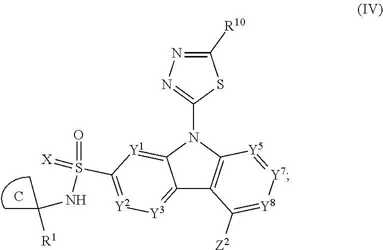
or pharmaceutically acceptable salt, stereoisomer, solvate, N-oxide, tautomer, isotopic variant, prodrug, or deuterated compound thereof; wherein,
ring C is C₃-C₇ cycloalkyl, or 4-7 membered heterocycloalkyl; wherein, the C₃-C₇ cycloalkyl, 4-7 membered heterocycloalkyl is optionally substituted by 1, 2, 3 or 4 substituents independently selected from D, halo, CN, NO₂, oxo, OH, C₁-C₆ alkyl, C₁-C₆haloalkyl, —O—C₁-C₆ alkyl, —OC₁-C₆ haloalkyl;
each R¹, R¹⁰, X, Y¹, Y², Y³, Y⁵, Y⁷, Y⁸, and Z² are defined with respect to Formula (I);
preferably, ring C is cyclopropyl, cyclobutyl, oxetanyl, or siletanyl; each is optionally substituted by 1, 2, 3 or 4 substituents independently selected from D, halo, CN, NO₂, oxo, OH, C₁-C₆ alkyl, C₁-C₆ haloalkyl, —O—C₁-C₆ alkyl, —OC₁-C₆ haloalkyl.

6. The compound of claim 1, wherein, the compound having the structure of Formula (IVa): 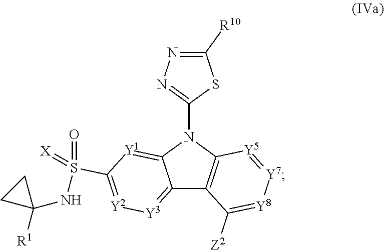
or pharmaceutically acceptable salt, stereoisomer, solvate, N-oxide, tautomer, isotopic variant, prodrug, or deuterated compound thereof; wherein each R¹, R¹⁰, X, Y¹, Y², Y³, Y⁵, Y⁷, Y⁸, and Z² are defined with respect to Formula (I);
preferably, X is O;
preferably, X is NR⁴, and R⁴ is H, D, CN, OR^{B}, C₁-C₄ alkyl optionally substituted with at least one of R^{4A};
preferably, Y¹ is N, Y² is N, and Y³ is CR⁵; Y¹ is N, Y² is N, and Y³ is N; Y¹ is N, Y² is CR⁶, and Y³ is CR⁵; Y¹ is N, Y² is CR⁶, and Y³ is N; Y¹ is CR⁵, Y² is N, and Y³ is CR⁵; Y¹ is CR⁵, Y² is N, and Y³ is N; or Y¹ is CR⁵, Y² is CR⁶, and Y³ is CRs;
preferably, wherein, Y¹ is CH, Y² is CH, and Y³ is CH; or Y¹ is CH, Y² is CF, and Y³ is CH;
preferably, each R⁵ is independently H, D, F, Cl or CH₃;
preferably, each R⁶ is H, D, F, Cl, OH, NH₂, or CN;
preferably, Y⁵ is CR⁸, Y⁷ is CR⁸, and Y⁸ is CR⁸; Y⁵ is CR⁸, Y⁷ is CR⁸, and Y⁸ is N; Y⁵ is CR⁸,
Y⁷ is N, and Y⁸ is CR⁸; Y⁵ is CR⁸, Y⁷ is N, and Y⁸ is N; Y⁵ is N, Y⁷ is CR⁸, and Y⁸ is CR⁸; Y⁵ is N,
Y⁷ is CR, and Y⁸ is N; Y⁵ is N, Y⁷ is N, and Y⁸ is CR⁸; or Y⁵ is N, Y⁷ is N, and Y⁸ is N;
preferably, Y⁵ is N, Y⁷ is CH, and Y⁸ is CH; Y⁵ is N, Y⁷ is CCH₃, and Y⁸ is CH; Y⁵ is N, Y⁷ is CH, and Y⁸ is N; or Y⁵ is N, Y⁷ is CCH₃, and Y⁸ is N;
preferably, wherein, R⁸ is H, D, C₁-C₆ alkyl;
preferably, R⁸ is H, D, CH₃;
preferably, wherein, Rio is H, D, halo, C₁-C₆ alkyl, or C₁-C₆ haloalkyl;
preferably, Rio is H, D, halo, methyl, ethyl, isopropyl, t-butyl, CF₃, CHF₂, CH₂F, or CDF₂;
preferably, wherein, Rio is CHF₂;
preferably, Z² is NR^{C}R^{D}, OR^{A}, SR^{A}, NR^{C}C(O)R^{B}, C_-C₈ alkyl, C₂-C₈ alkenyl, C₂-C₈ alkynyl; wherein, the C₁-C₈ alkyl, C₂-C₈ alkenyl, C₂-C₈ alkynyl is optionally substituted by 1, 2, 3, 4, or 5 substituents independently selected from R¹¹;
preferably, wherein, Z² is NR^{C}R^{D};
preferably, wherein, R^{e} is H, D, C₁-C₆ alkyl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴; preferably, R^{e} is H, D, C₁-C₆ alkyl;
preferably, wherein, R^{e} is H, D, CH₃, CD₃, CH₂CH₃.

7. The compound of claim 1, wherein, R^{D} is H, D, C₁-C₆ alkyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₃-C₁₀ cycloalkyl, 4-10 membered heterocycloalkyl, C₆-C₁₀ aryl, 5-10 membered heteroaryl, C₆-C₁₀ aryl-C₁-C₆ alkyl, 5-10 membered heteroaryl-C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴;
preferably, R^{D} is H, D, —CH₃, —CD₃, —CH₂CH₃, 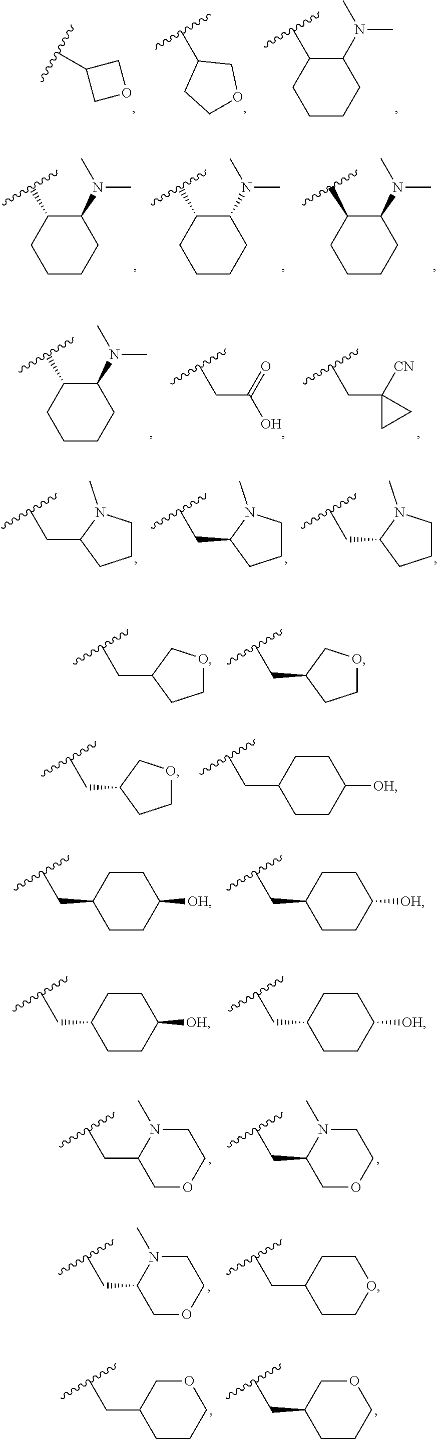 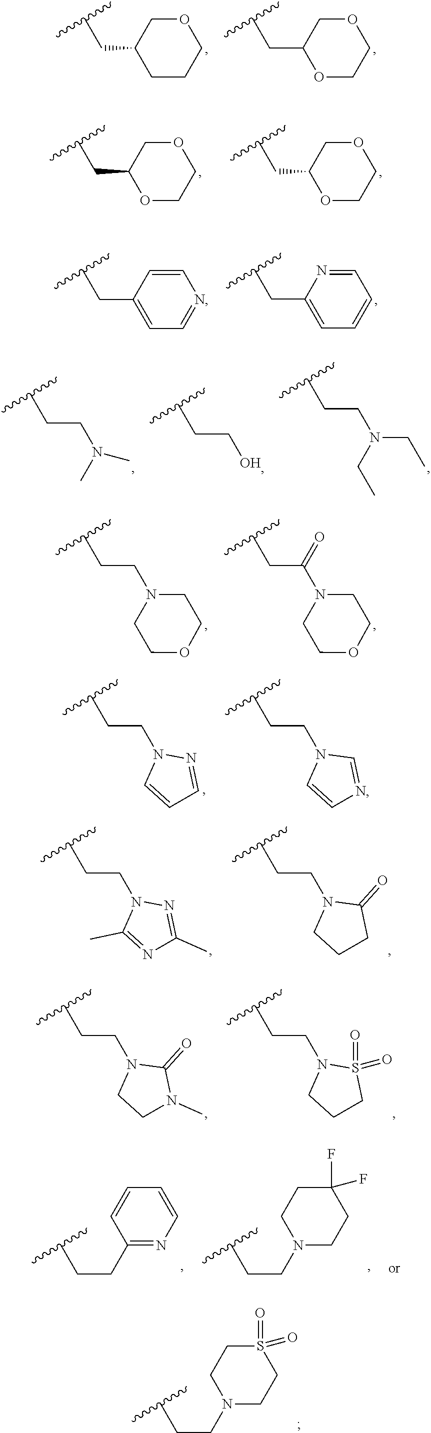
preferably, Z² is OR^{A};
preferably, Z² is SR^{A};
preferably, R is H, D, C₁-C₆ alkyl, C₃-C₁₀ cycloalkyl, 4-10 membered heterocycloalkyl, C₃-IO cycloalkyl-C₁-C₆ alkyl, or 4-10 membered heterocycloalkyl-C₁-C₆ alkyl; wherein, the C₁-C₆ alkyl, C₃-C¹⁰ cycloalkyl, 4-10 membered heterocyclalkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴; preferably, each R^{A} is independently H, D, —CH₃, —CD₃, —CH₂CH₃, —CF₃, —CH₂CH₂OH, —CH₂CH₂OCH₃, tetrahydrofuranyl, 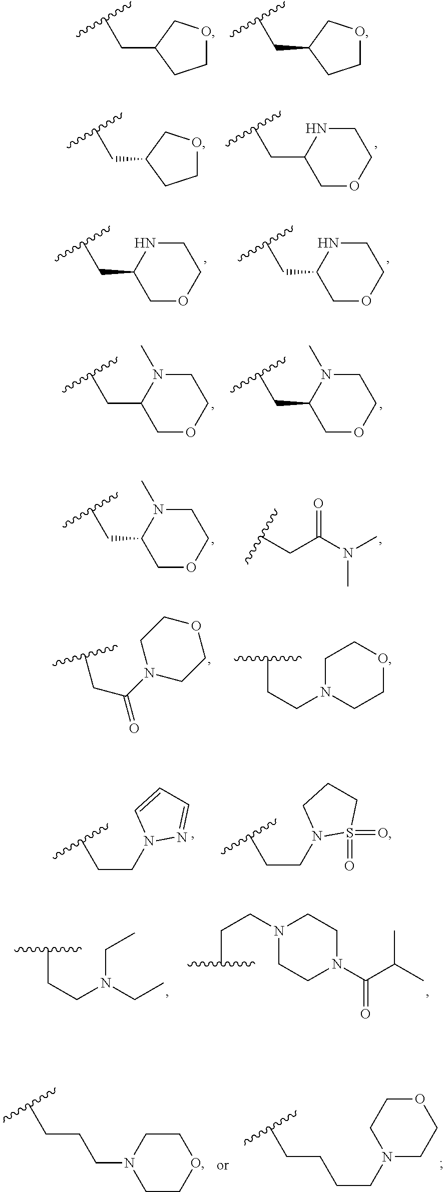
preferably, R^{B} is independently C₁-C₆ alkyl, C₃-C₁₀ cycloalkyl-C₁-C₆ alkyl, or 4-10 membered heterocycloalkyl-C₁-C₆ alkyl; wherein, the C₁-C₆ alkyl, C₃-C₁₀ cycloalkyl-C₁-C₆ alkyl, or 4-10 membered heterocycloalkyl-C₁-C₆ alkyl is optionally substituted with 1, 2, 3, 4 or 5 substituents independently selected from R¹⁴; preferably, R^{B} is 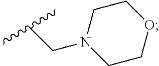 [SMILES: *CN1CCOCC1]
preferably, each R¹⁴ is independently H, D, CN, halo, oxo, SF₅, C₁-C₈ alkyl, C₆-C₁₄ aryl, C₃-C₁₄ cycloalkyl, 5-14 membered heteroaryl, or 4-14 membered heterocycloalkyl, OR^{a}, SR^{a}, SF₅, NHOR^{a}, C(O)R^{b}, C(O)NR^{c}R^{d}, C(O)OR^{a}, NR^{c}R^{d}, or NR^{c}O(O)R^{b}; wherein, the C₁-C₈alkyl, C₆-C₁₄ aryl, C₃-C₁₄ cycloalkyl, 5-14 membered heteroaryl, or 4-14 membered heterocycloalkyl is optionally substituted by 1, 2, 3, 4 or 5 substituents independently selected from D, NO², CN, halo, oxo, SF₅, C₁-C₆ alkyl, C₂-C₆ alkenyl, C₂-C₆ alkynyl, C₁-C₆ haloalkyl, C₁-C₆ alkylOH, C₁-C₆ alkyl-O—C₁-C₆ alkyl, CN, NO₂, N₃, OR^{a1}, SR^{a1}, SF₅, NHOR^{a1}, C(O)R¹, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{d1}; preferably, each R¹⁴ is independently H, D, CN, halo, oxo, SF₅, —CH₃, —CD₃, —CH₂CH₃, —OH, —OCH₃, —OCH₂CH₃, —NH₂, —N(CH₃)₂, —N(CH₂CH₃)₂, —C(O)CH(CH₃)₂, —COOH, —C(O)N(CH₃)₂, or 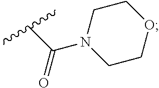 [SMILES: *C(=O)N1CCOCC1]
preferably, Z² is C₁-C₈alkyl, C₂-C₈ alkenyl, C₂-C₈ alkynyl; each is optionally substituted by 1, 2, 3, 4, or 5 substituents independently selected from R¹¹;
preferably, each R¹¹ is independently H, D, halo, CN, N₃, oxo, OR^{A}, SR^{A}, SF₅, C(O)R^{B}, C(O)NR^{C}R^{D}, C(O)OR^{A}, OC(O)R^{B}, OC(O)NR^{C}R^{D}, NR^{C}R^{D}, NR^{C}C(O)R^{D}, Cy³; preferably, each R¹¹ is independently H, D, halo, CN, —OH, —OCH₃, —OCH₂CH₃, —NH₂, —NHCH₃, —N(CH₃)₂, —N(CH₂CH₃)₂, morpholinyl, or pyrazolyl.

8. The compound of claim 1, wherein, the moiety Z² has the structure of SH, OCF₂CF₃, 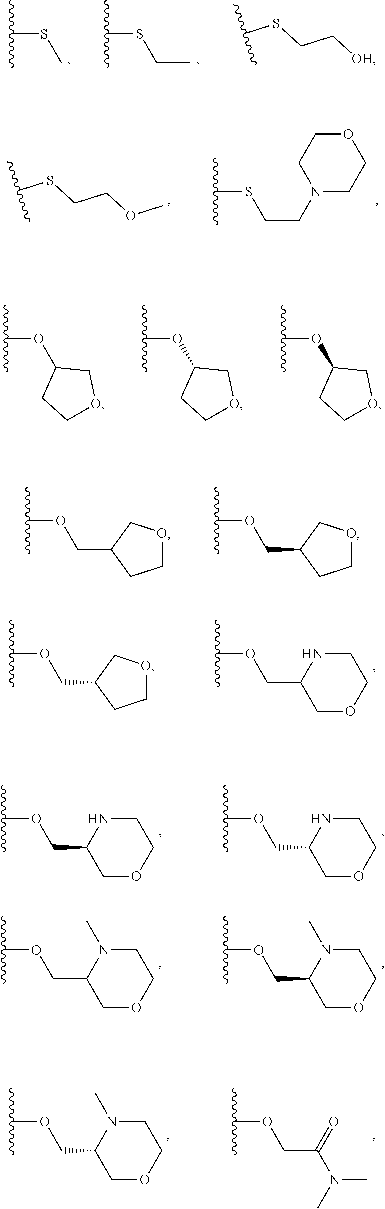 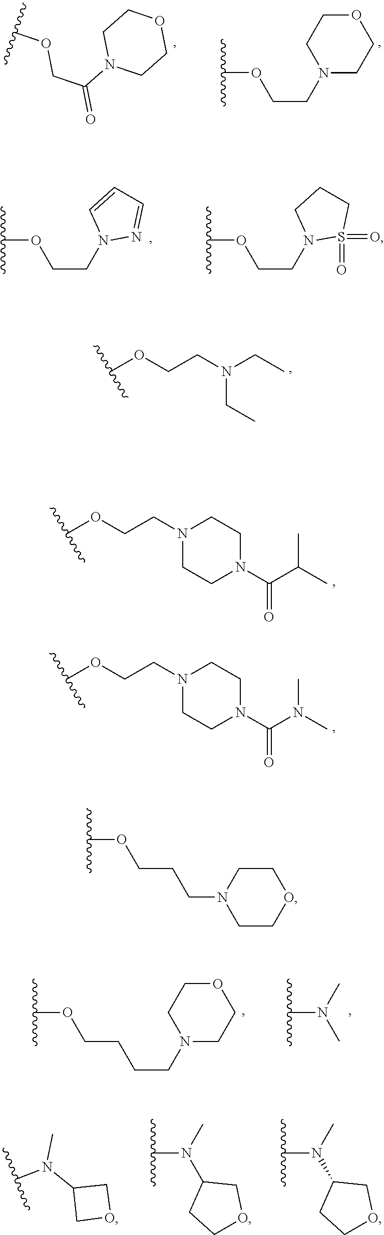 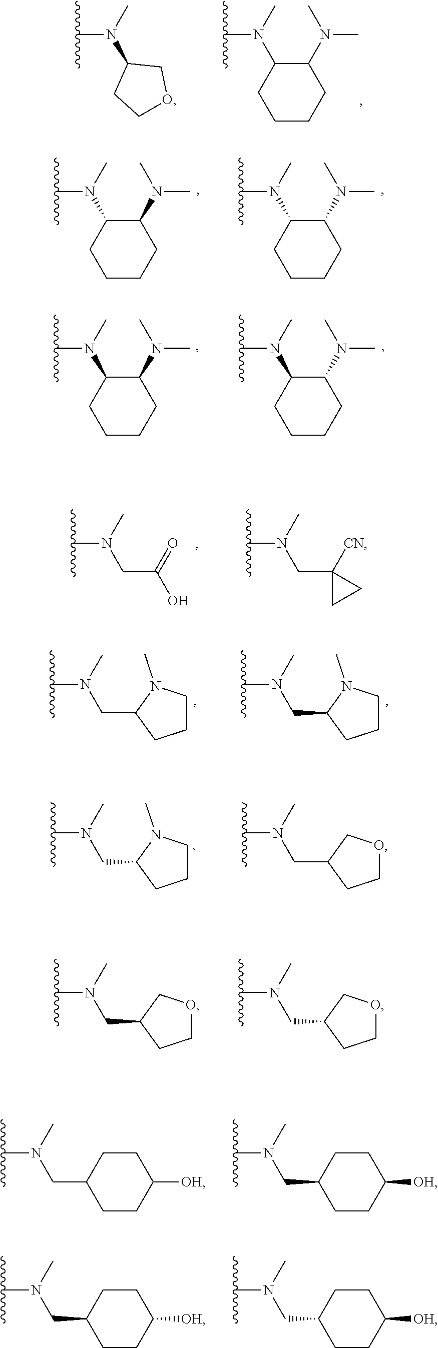 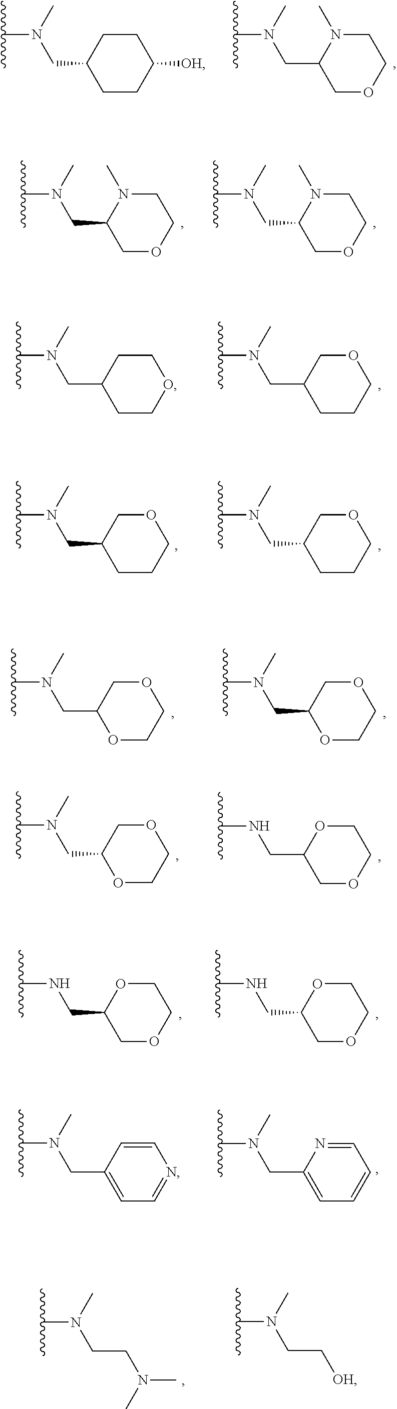 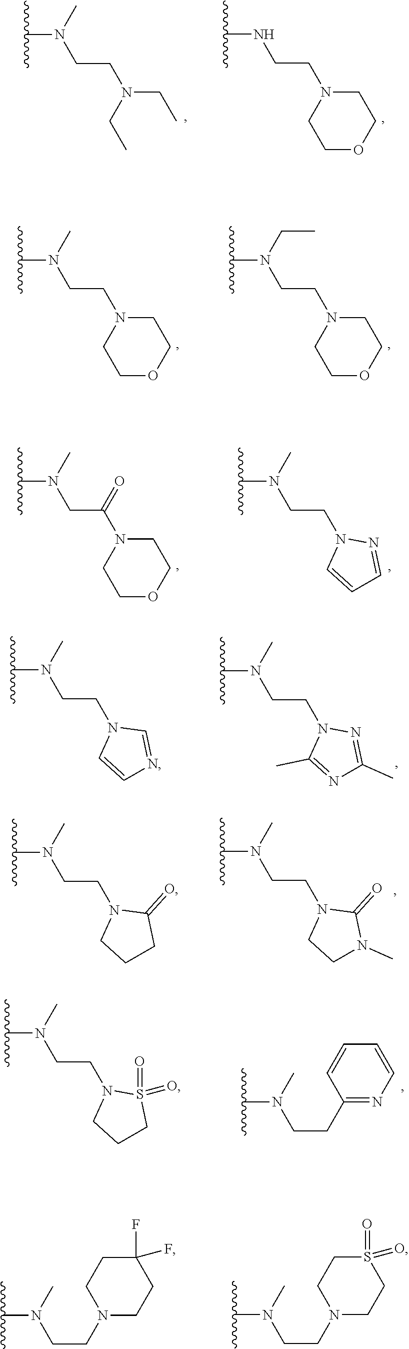 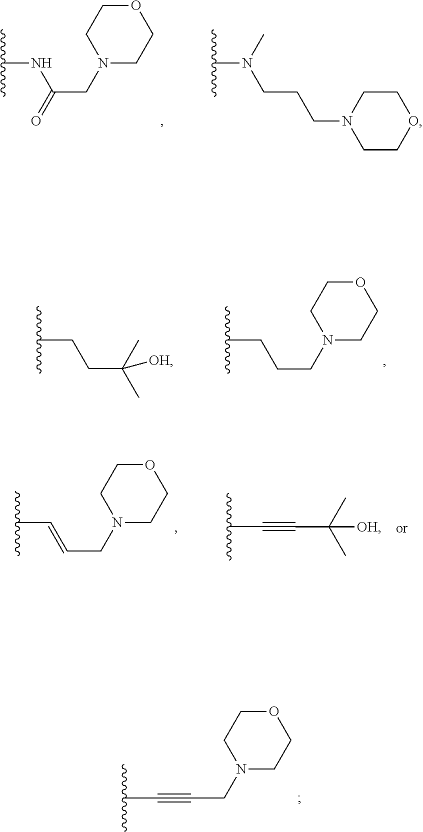
preferably, Z² is 4-14 membered heterocycloalkyl optionally substituted by 1, 2, 3, 4 or substituents independently selected from R¹¹;
preferably, Z² is piperidinyl, piperazinyl, morpholinyl, octahydropyrrolo[3,4-c]pyrrolyl, octahydro-1H-pyrrolo[3,2-c]pyridinyl, 2-azabicyclo[2.2.1]heptanyl, 2,5-diazabicyclo[2.2.1]heptanyl, 5,6,7,8-tetrahydroimidazo[1,5-a]pyrazinyl, 5,6,7,8-tetrahydroimidazo[1,2-a]pyrazinyl, 5,6,7,8-tetrahydro-[1,2,4]triazolo[4,3-a]pyrazinyl, 5,6,7,8-tetrahydro-[1,2,4]triazolo[1,5-a]pyrazinyl, 4,7-diazaspiro[2.5]octanyl, 1,8-diazaspiro[4.5]decanyl; each is optionally substituted by 1, 2, 3, 4 or 5 substituents independently selected from R¹¹;
preferably, each R¹¹ is independently H, D, halo, CN, N₃, oxo, C₁-C₆ alkyl, OR^{A}, SR^{A}, SF₅, NR^{C}OR^{A}, C(O)R^{B}, NR^{C}R^{D} Cy³; wherein, the C₁-C₆ alkyl is optionally substituted 1, 2, 3, 4 or 5 substituents independently selected from R¹²; preferably, each R¹¹ is independently H, D, halo, CN, N₃, oxo, —CH₃, —CD₃, CH₂F, CHF₂, CF₃, —CH₂CH₃, —CH(CH₃)₂, —CH₂OH, —CH₂OCH₃, —CH₂NH₂, —CH₂NHCH₃, —CH₂N(CH₃)₂, —OH, —OCH₃, —OCH₂CH₃, —NH₂, —NHCH₃, —N(CH₃)₂, —NHCH₂CH₃, —N(CH₂CH₃)₂, morpholinyl, pyrazolyl, or 4,4-difluoro-1-piperidinyl;
preferably, wherein, the moiety Z² has the structure of

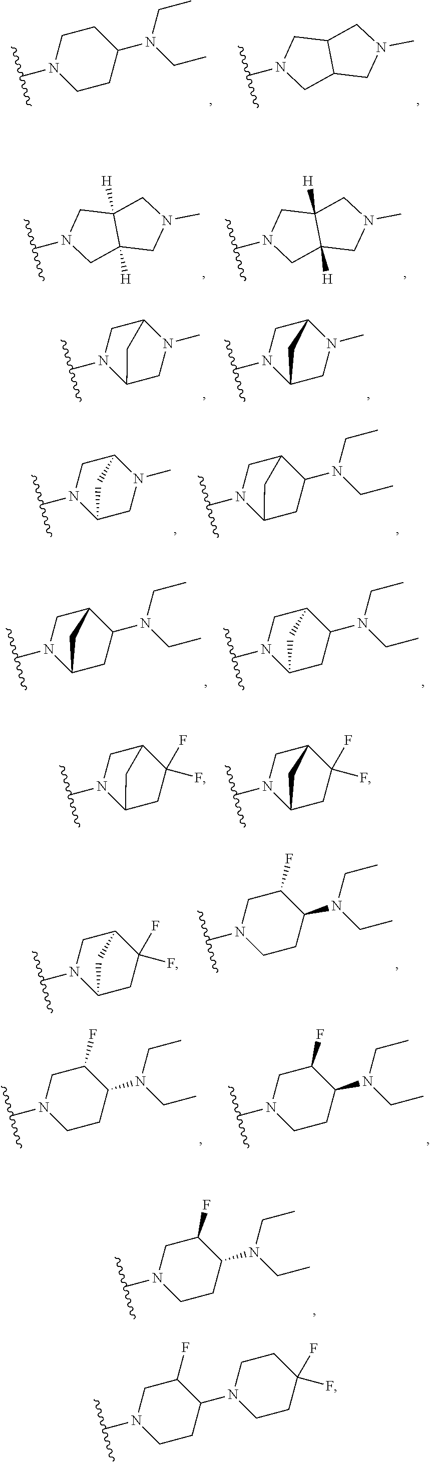

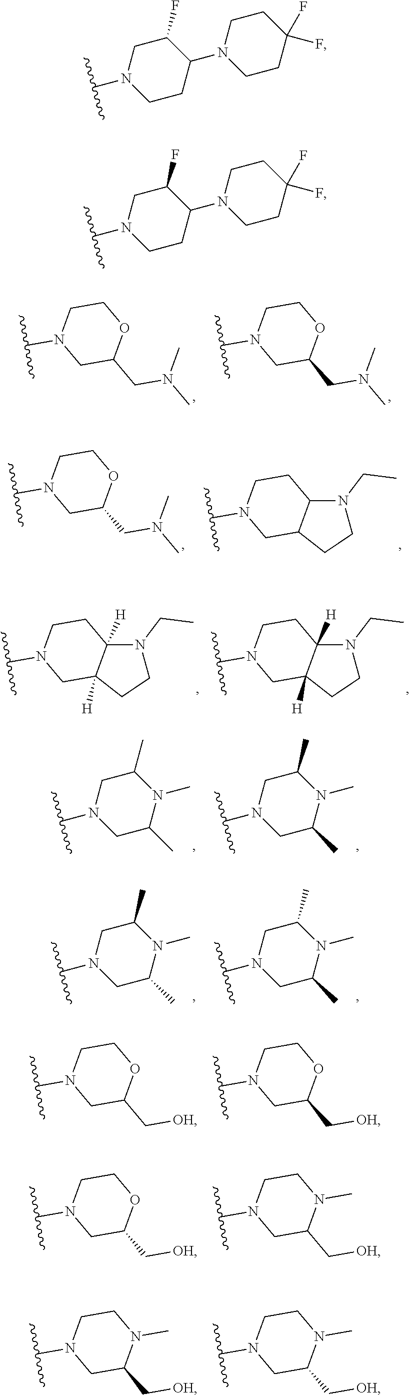

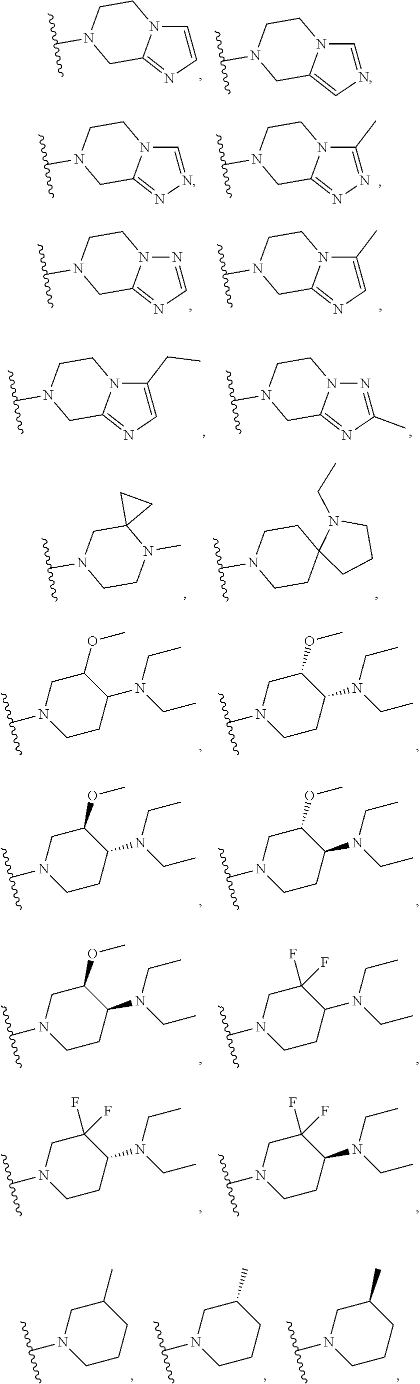

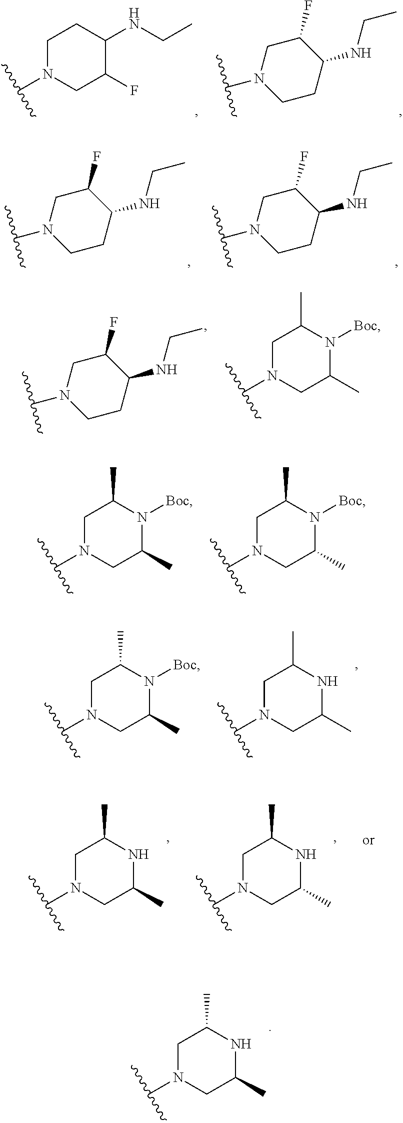

9. The compound of claim 1, wherein, the compound of Formula (I) is: 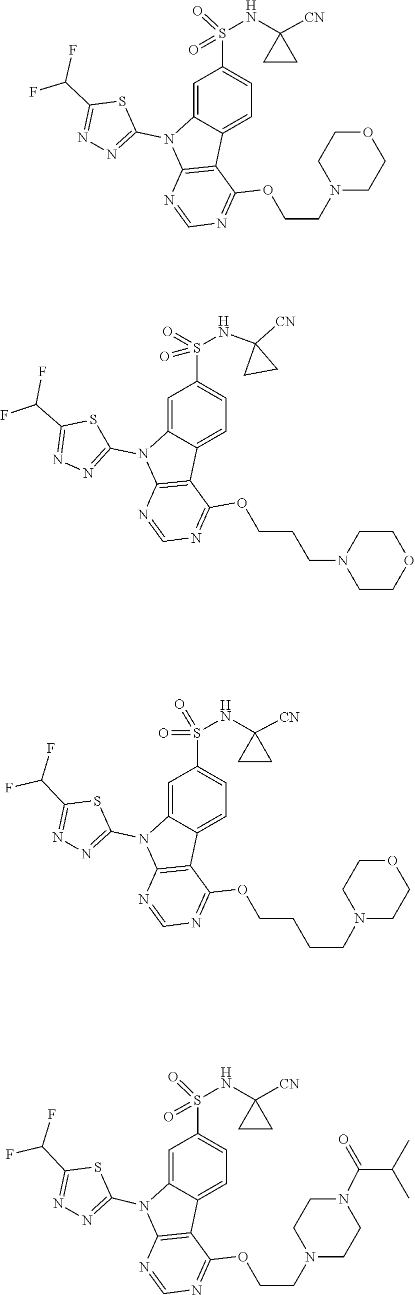 [SMILES: N#CC1(NS(=O)(=O)c2ccc3c4c(OCCN5CCOCC5)ncnc4n(-c4nnc(C(F)F)s4)c3c2)CC1 ; N#CC1(NS(=O)(=O)c2ccc3c4c(OCCCN5CCOCC5)ncnc4n(-c4nnc(C(F)F)s4)c3c2)CC1 ; N#CC1(NS(=O)(=O)c2ccc3c4c(OCCCCN5CCOCC5)ncnc4n(-c4nnc(C(F)F)s4)c3c2)CC1 ; CC(C)C(=O)N1CCN(CCOc2ncnc3c2c2ccc(S(=O)(=O)NC4(C#N)CC4)cc2n3-c2nnc(C(F)F)s2)CC1] 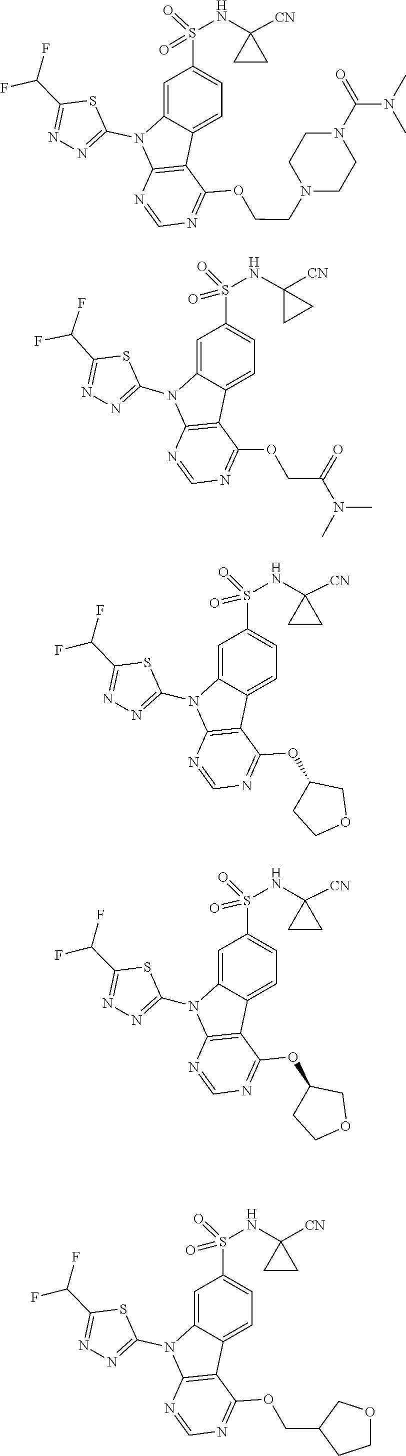 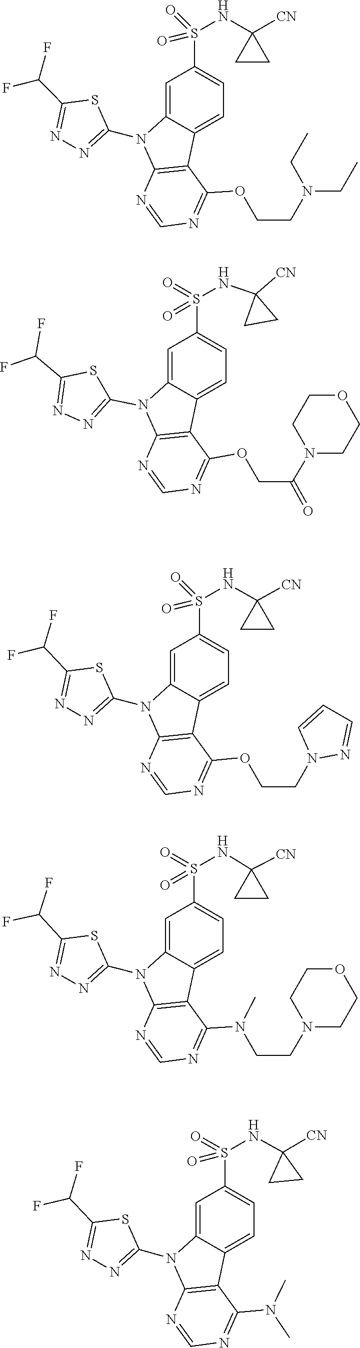 [SMILES: CCN(CC)CCOc1ncnc2c1c1ccc(S(=O)(=O)NC3(C#N)CC3)cc1n2-c1nnc(C(F)F)s1 ; N#CC1(NS(=O)(=O)c2ccc3c4c(OCC(=O)N5CCOCC5)ncnc4n(-c4nnc(C(F)F)s4)c3c2)CC1 ; N#CC1(NS(=O)(=O)c2ccc3c4c(OCCn5cccn5)ncnc4n(-c4nnc(C(F)F)s4)c3c2)CC1 ; CN(CCN1CCOCC1)c1ncnc2c1c1ccc(S(=O)(=O)NC3(C#N)CC3)cc1n2-c1nnc(C(F)F)s1 ; CN(C)c1ncnc2c1c1ccc(S(=O)(=O)NC3(C#N)CC3)cc1n2-c1nnc(C(F)F)s1] 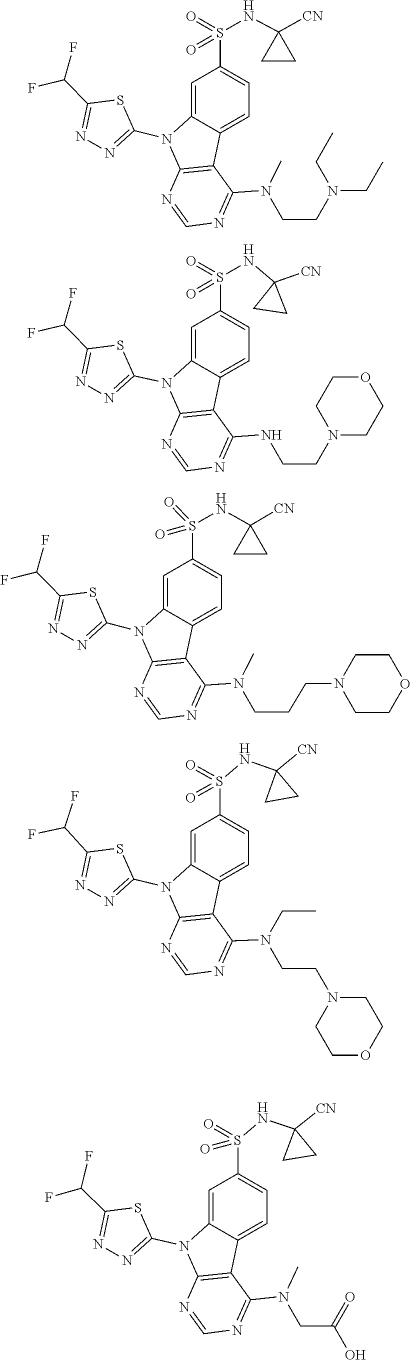 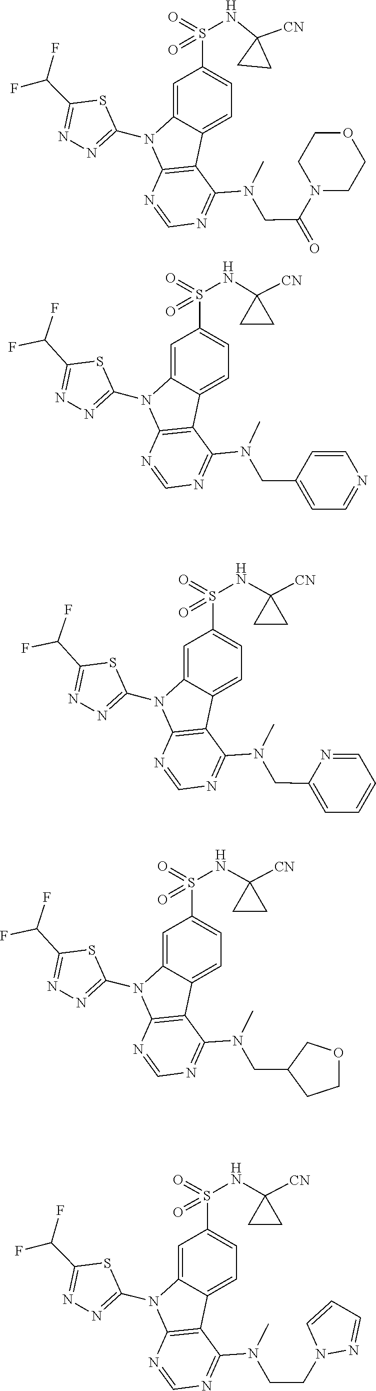 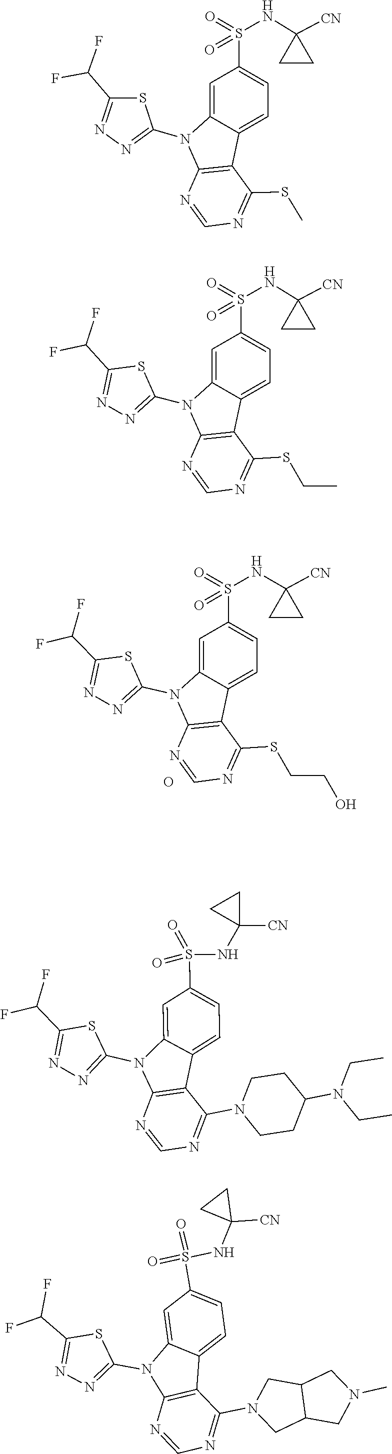 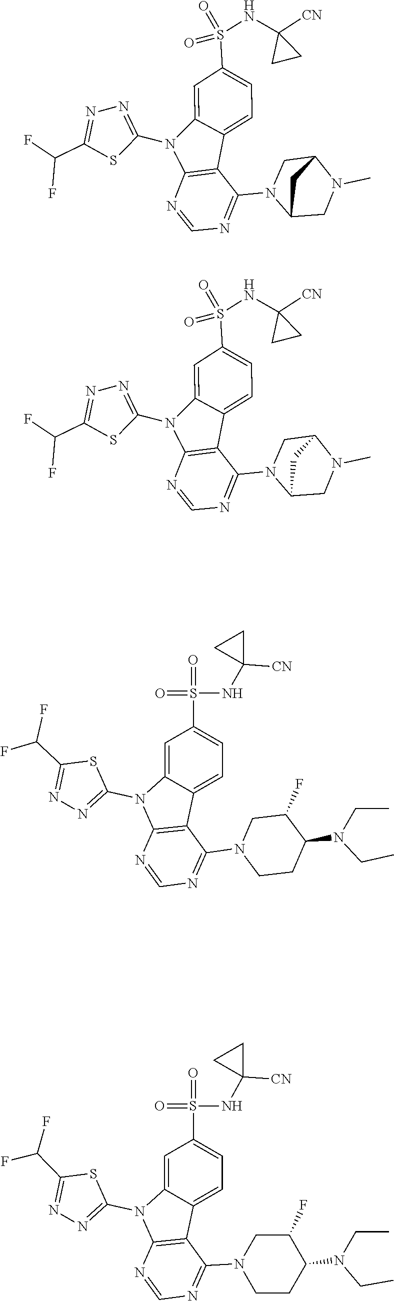 [SMILES: CN1C[C@H]2C[C@@H]1CN2c1ncnc2c1c1ccc(S(=O)(=O)NC3(C#N)CC3)cc1n2-c1nnc(C(F)F)s1 ; CN1C[C@@H]2C[C@H]1CN2c1ncnc2c1c1ccc(S(=O)(=O)NC3(C#N)CC3)cc1n2-c1nnc(C(F)F)s1 ; CCN(CC)[C@H]1CCN(c2ncnc3c2c2ccc(S(=O)(=O)NC4(C#N)CC4)cc2n3-c2nnc(C(F)F)s2)C[C@@H]1F ; CCN(CC)[C@@H]1CCN(c2ncnc3c2c2ccc(S(=O)(=O)NC4(C#N)CC4)cc2n3-c2nnc(C(F)F)s2)C[C@@H]1F] 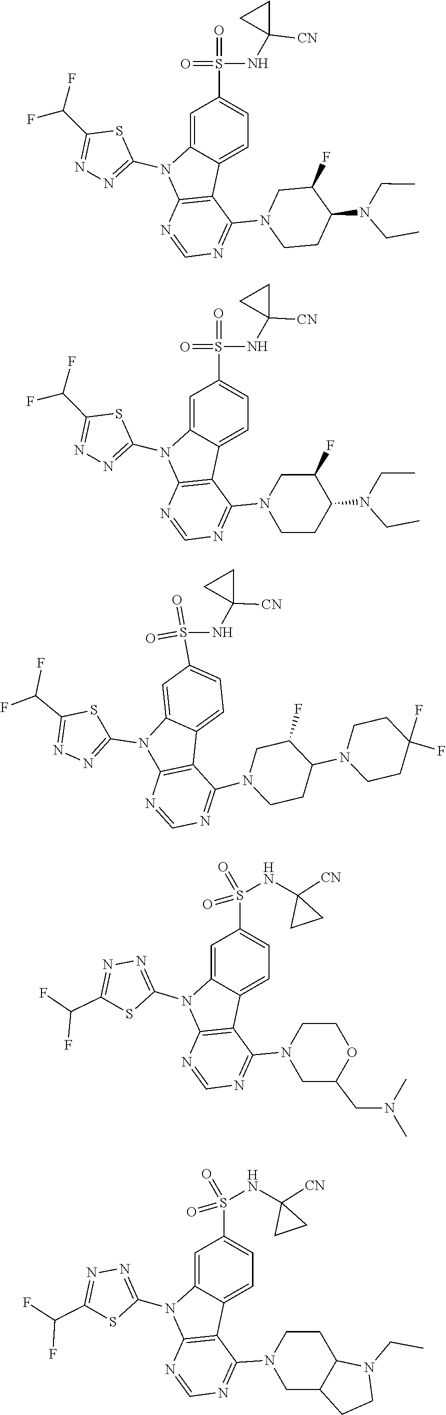 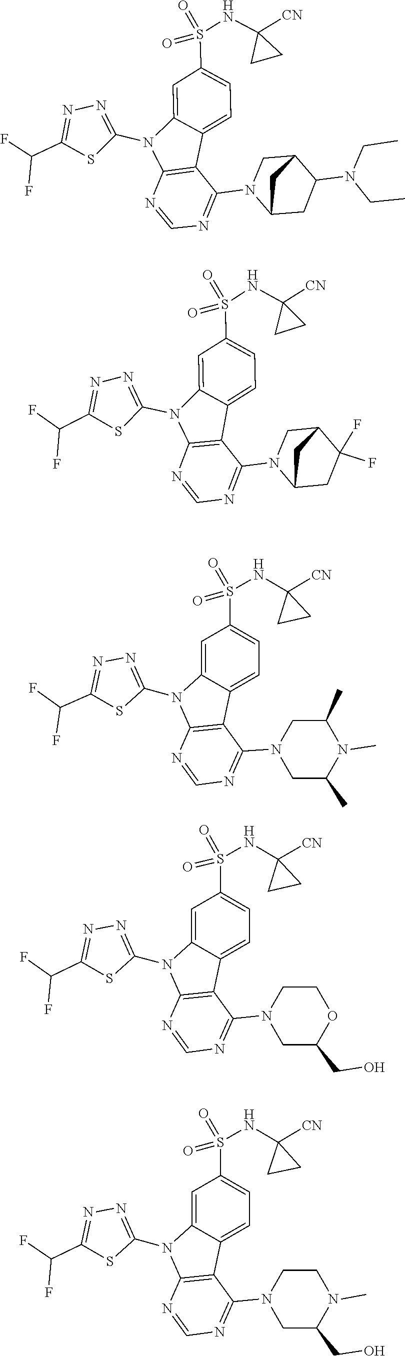 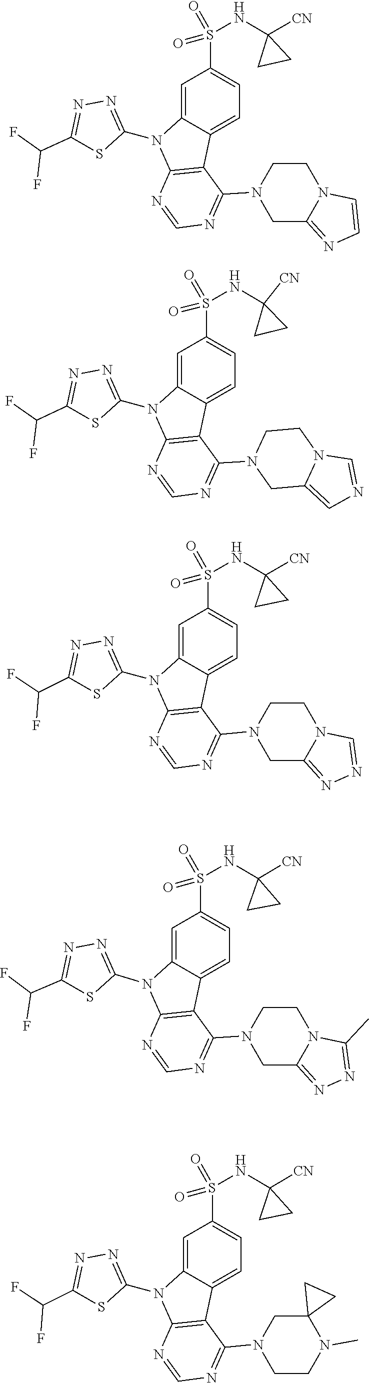 [SMILES: N#CC1(NS(=O)(=O)c2ccc3c4c(N5CCn6ccnc6C5)ncnc4n(-c4nnc(C(F)F)s4)c3c2)CC1 ; N#CC1(NS(=O)(=O)c2ccc3c4c(N5CCn6cncc6C5)ncnc4n(-c4nnc(C(F)F)s4)c3c2)CC1 ; N#CC1(NS(=O)(=O)c2ccc3c4c(N5CCn6cnnc6C5)ncnc4n(-c4nnc(C(F)F)s4)c3c2)CC1 ; Cc1nnc2n1CCN(c1ncnc3c1c1ccc(S(=O)(=O)NC4(C#N)CC4)cc1n3-c1nnc(C(F)F)s1)C2 ; CN1CCN(c2ncnc3c2c2ccc(S(=O)(=O)NC4(C#N)CC4)cc2n3-c2nnc(C(F)F)s2)CC12CC2] 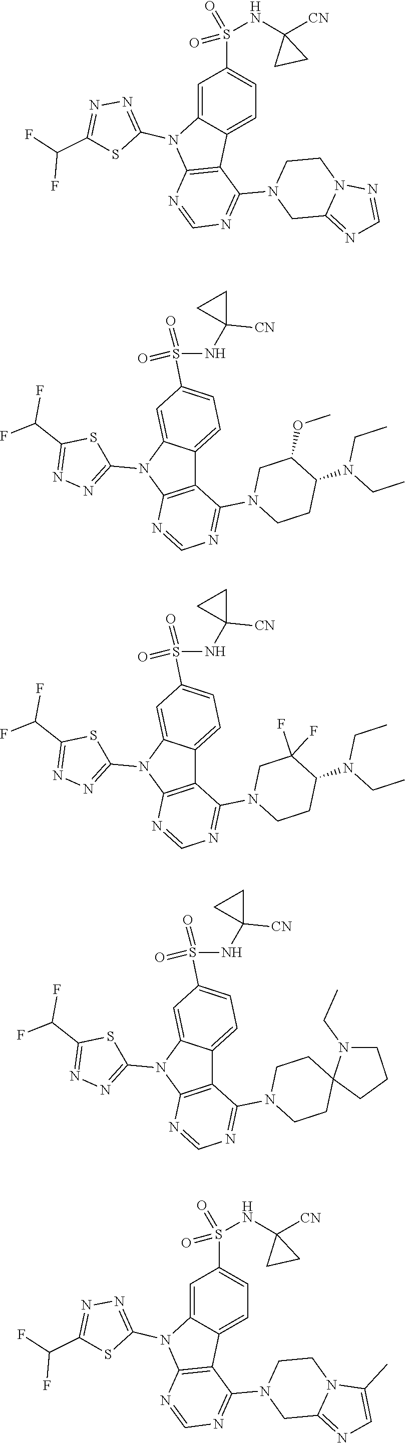 [SMILES: N#CC1(NS(=O)(=O)c2ccc3c4c(N5CCn6ncnc6C5)ncnc4n(-c4nnc(C(F)F)s4)c3c2)CC1 ; CCN(CC)[C@@H]1CCN(c2ncnc3c2c2ccc(S(=O)(=O)NC4(C#N)CC4)cc2n3-c2nnc(C(F)F)s2)C[C@@H]1OC ; CCN(CC)[C@@H]1CCN(c2ncnc3c2c2ccc(S(=O)(=O)NC4(C#N)CC4)cc2n3-c2nnc(C(F)F)s2)CC1(F)F ; CCN1CCCC12CCN(c1ncnc3c1c1ccc(S(=O)(=O)NC4(C#N)CC4)cc1n3-c1nnc(C(F)F)s1)CC2 ; Cc1cnc2n1CCN(c1ncnc3c1c1ccc(S(=O)(=O)NC4(C#N)CC4)cc1n3-c1nnc(C(F)F)s1)C2] 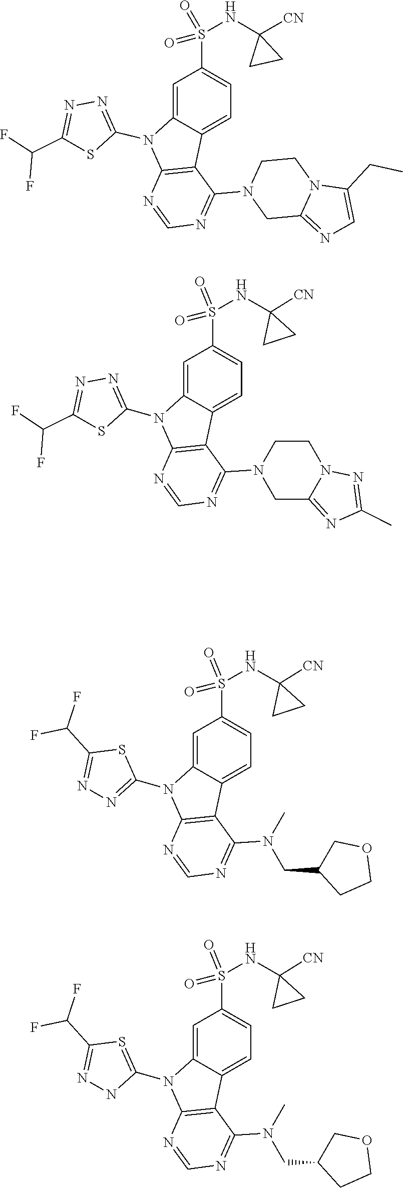 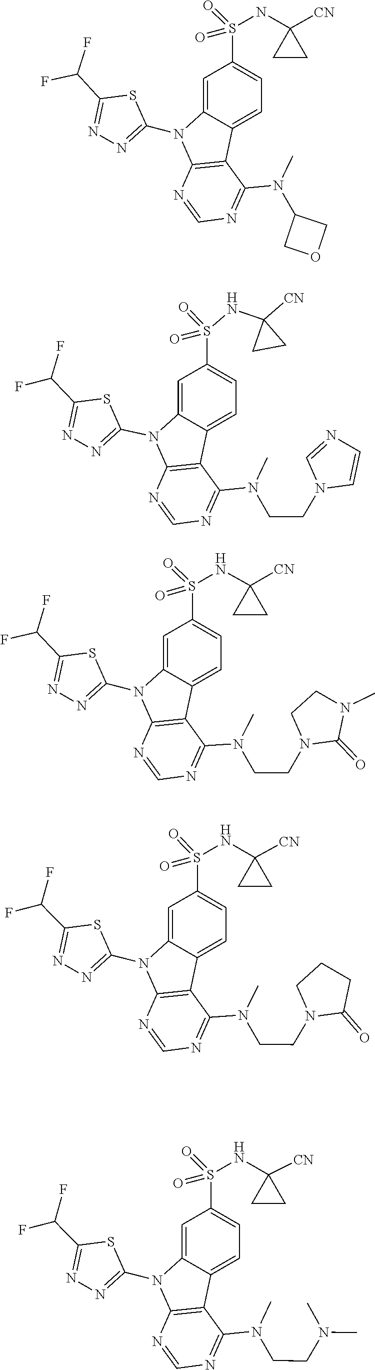 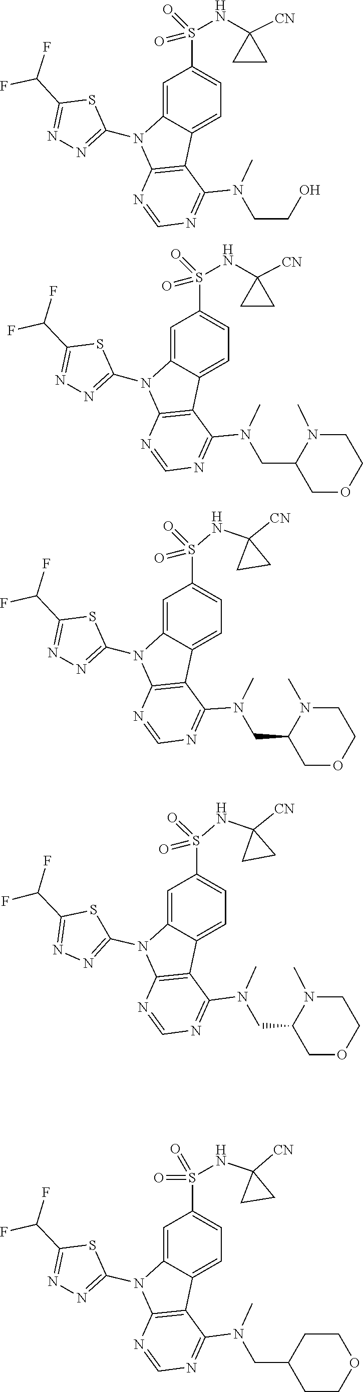 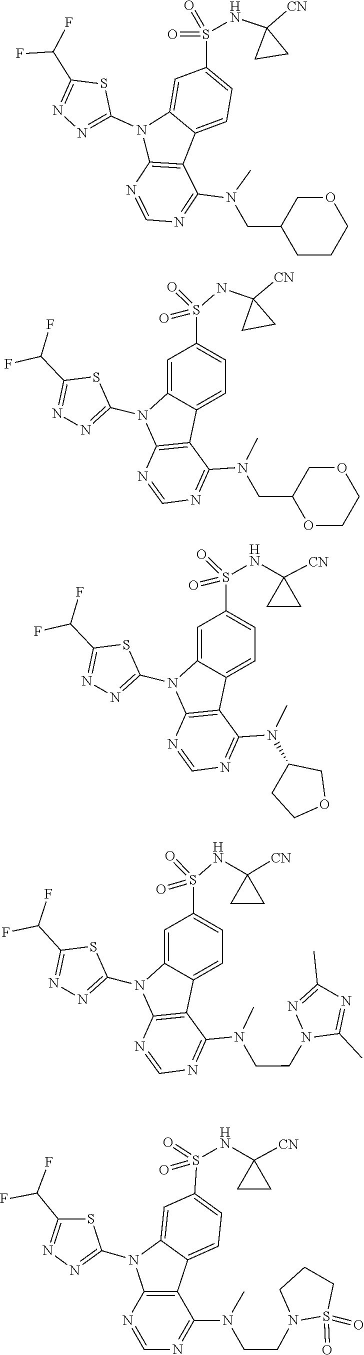 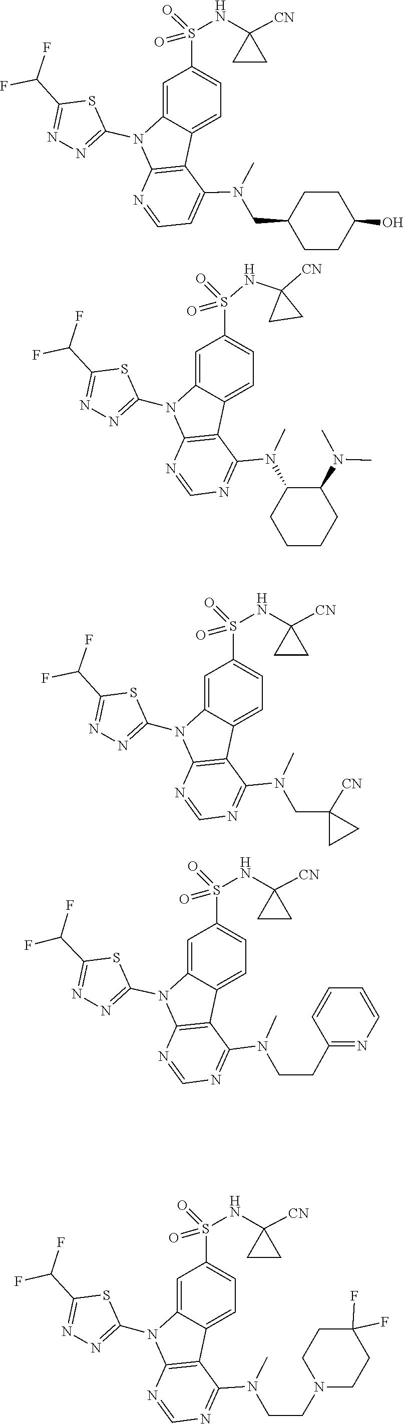 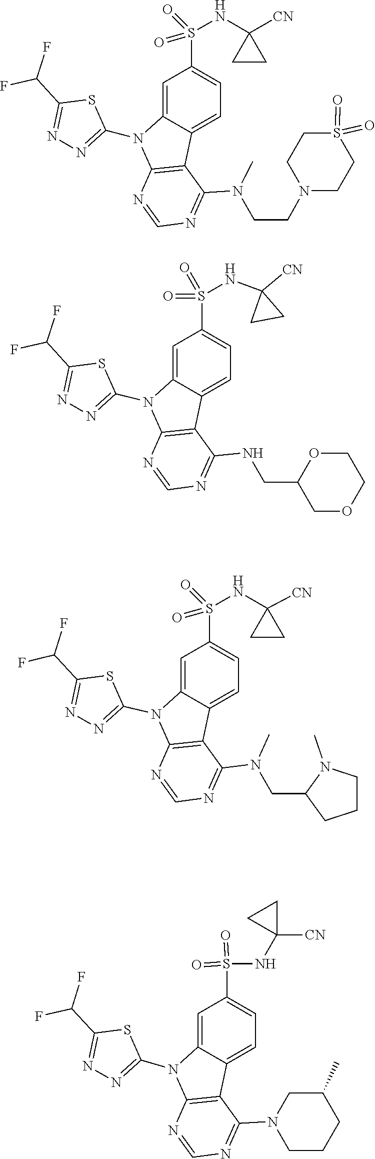 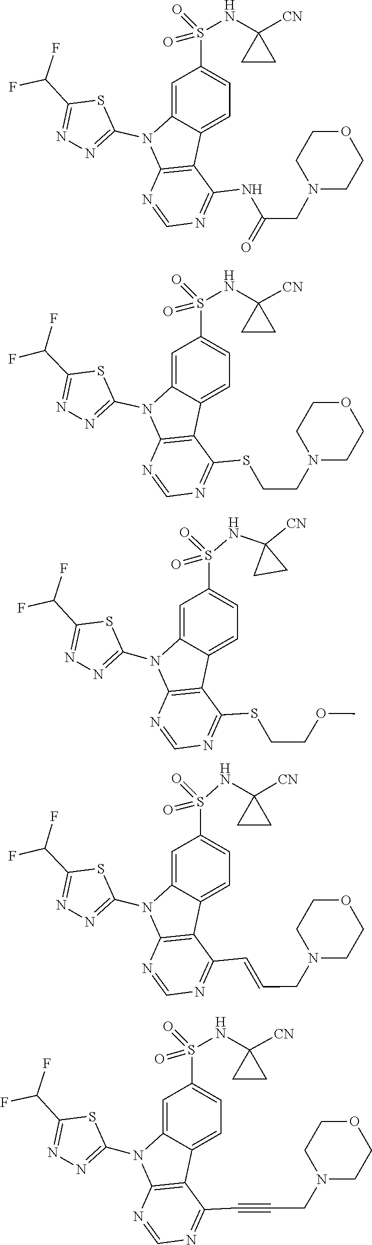 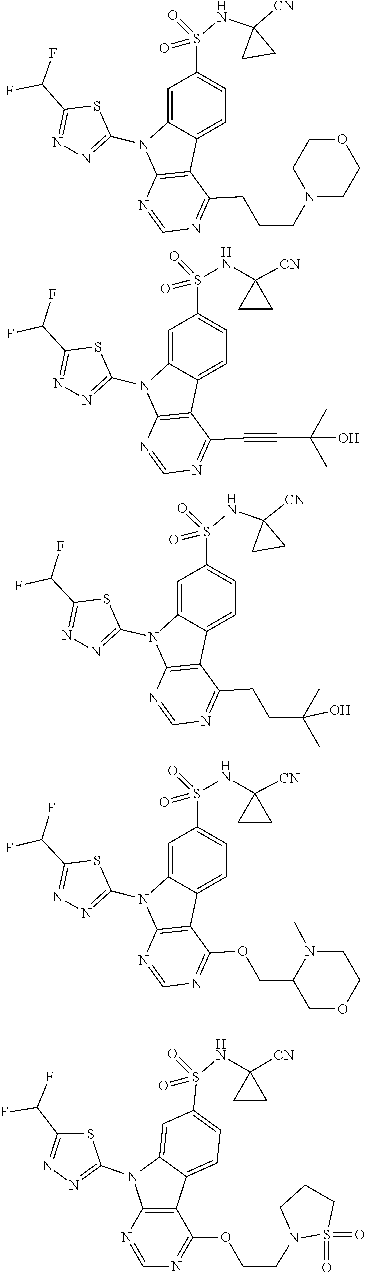 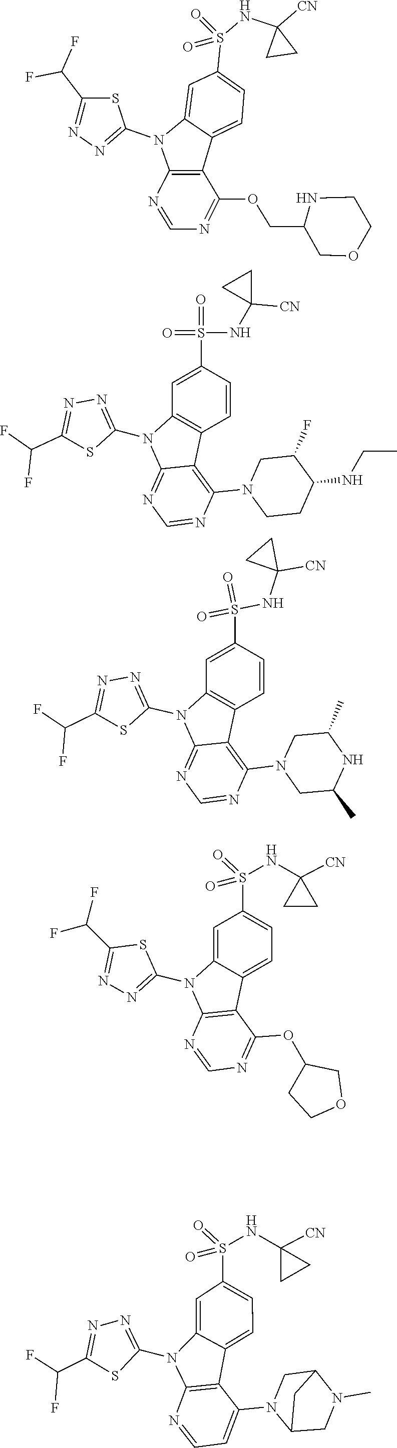 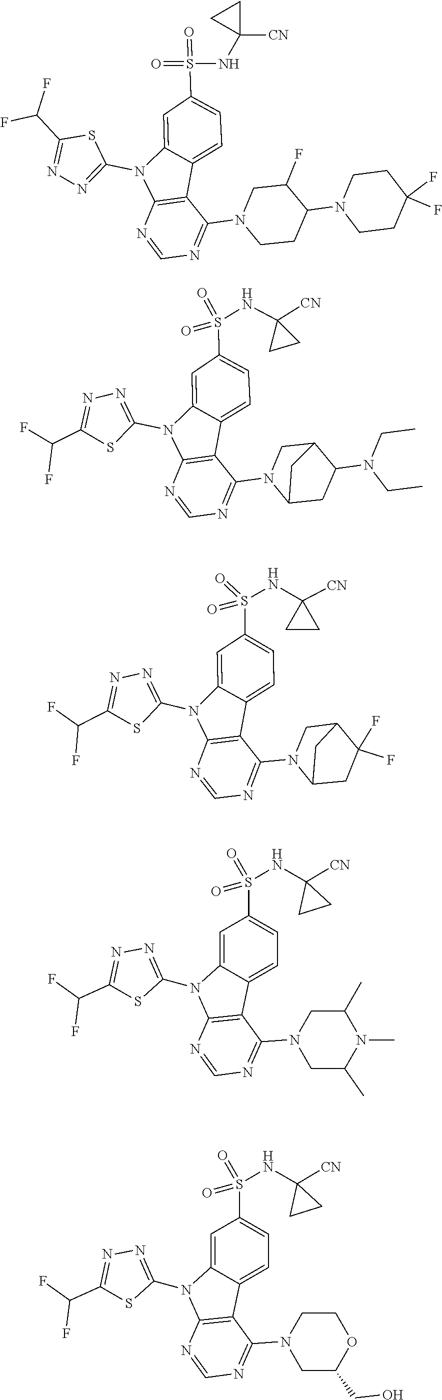 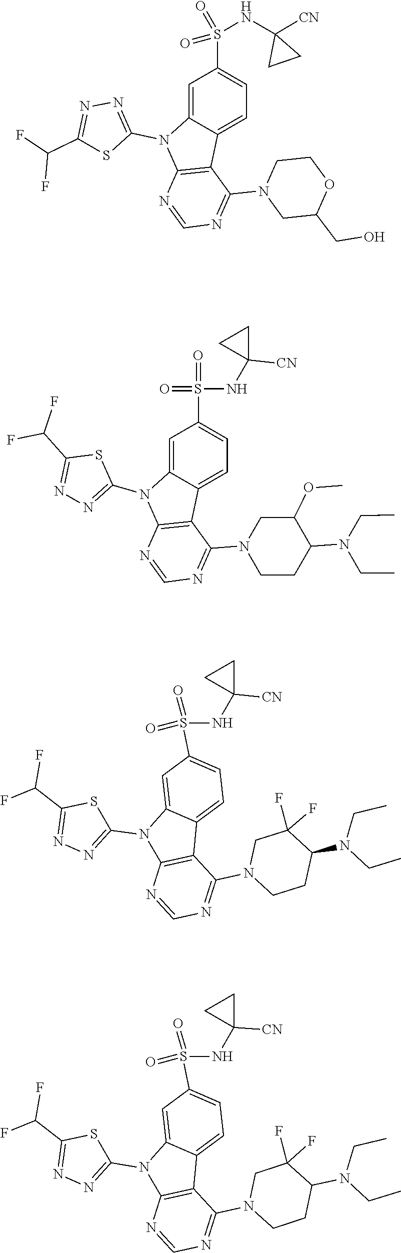 [SMILES: N#CC1(NS(=O)(=O)c2ccc3c4c(N5CCOC(CO)C5)ncnc4n(-c4nnc(C(F)F)s4)c3c2)CC1 ; CCN(CC)C1CCN(c2ncnc3c2c2ccc(S(=O)(=O)NC4(C#N)CC4)cc2n3-c2nnc(C(F)F)s2)CC1OC ; CCN(CC)[C@H]1CCN(c2ncnc3c2c2ccc(S(=O)(=O)NC4(C#N)CC4)cc2n3-c2nnc(C(F)F)s2)CC1(F)F ; CCN(CC)C1CCN(c2ncnc3c2c2ccc(S(=O)(=O)NC4(C#N)CC4)cc2n3-c2nnc(C(F)F)s2)CC1(F)F] 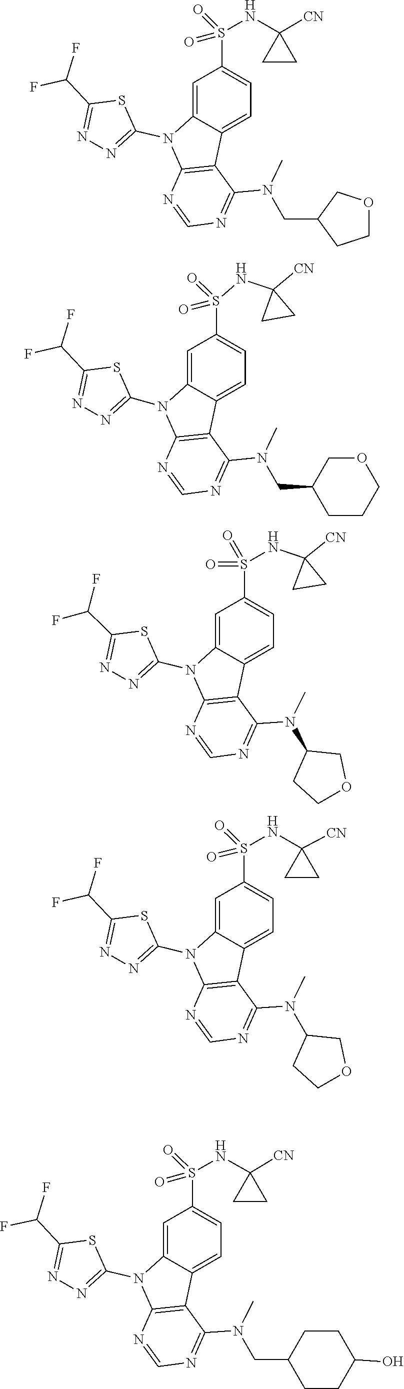 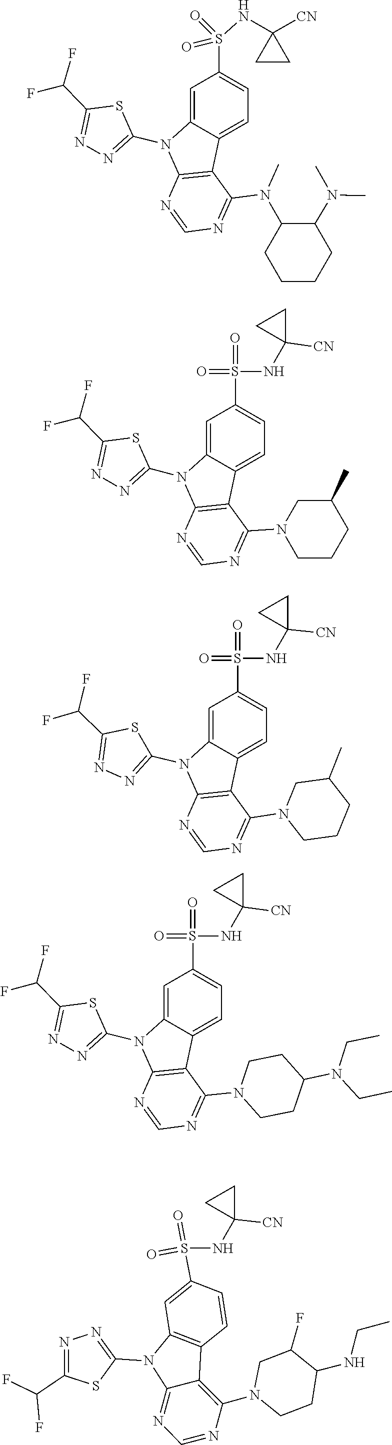 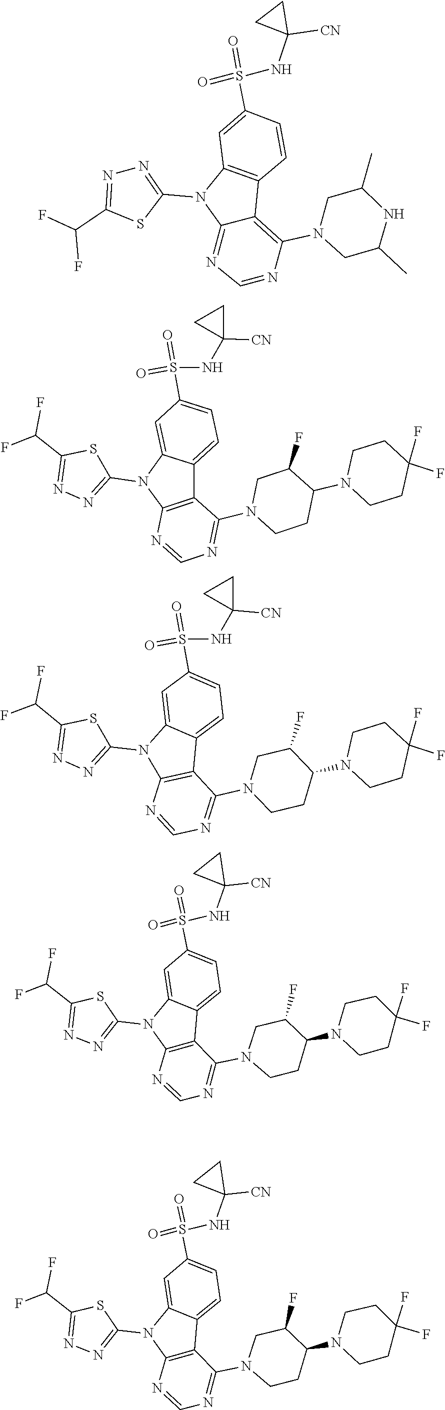 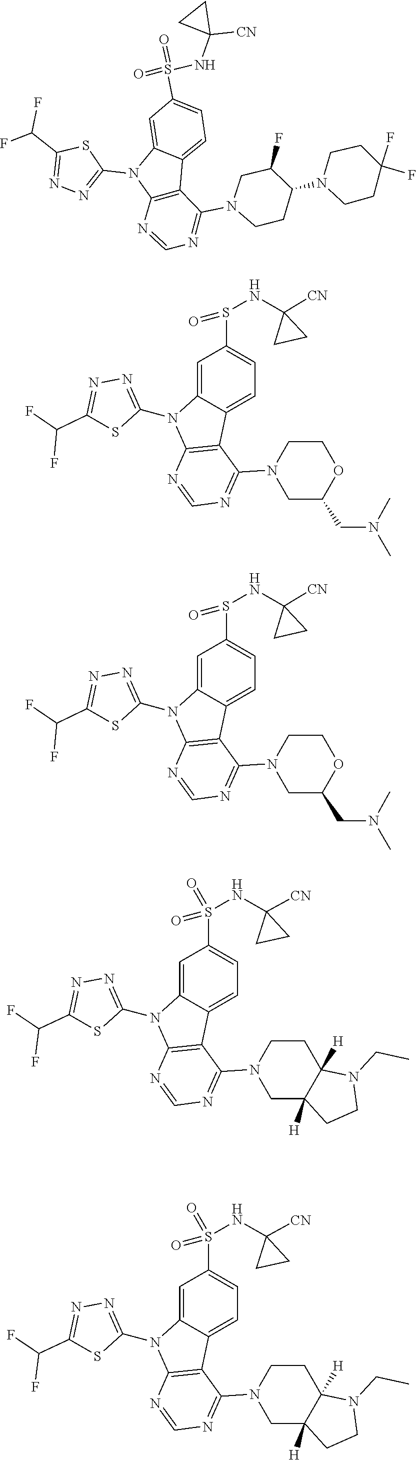 [SMILES: N#CC1(NS(=O)(=O)c2ccc3c4c(N5CC[C@@H](N6CCC(F)(F)CC6)[C@H](F)C5)ncnc4n(-c4nnc(C(F)F)s4)c3c2)CC1 ; CN(C)C[C@@H]1CN(c2ncnc3c2c2ccc(S(=O)NC4(C#N)CC4)cc2n3-c2nnc(C(F)F)s2)CCO1 ; CN(C)C[C@H]1CN(c2ncnc3c2c2ccc(S(=O)NC4(C#N)CC4)cc2n3-c2nnc(C(F)F)s2)CCO1 ; CCN1CC[C@@H]2CN(c3ncnc4c3c3ccc(S(=O)(=O)NC5(C#N)CC5)cc3n4-c3nnc(C(F)F)s3)CC[C@@H]21 ; CCN1CC[C@@H]2CN(c3ncnc4c3c3ccc(S(=O)(=O)NC5(C#N)CC5)cc3n4-c3nnc(C(F)F)s3)CC[C@H]21] 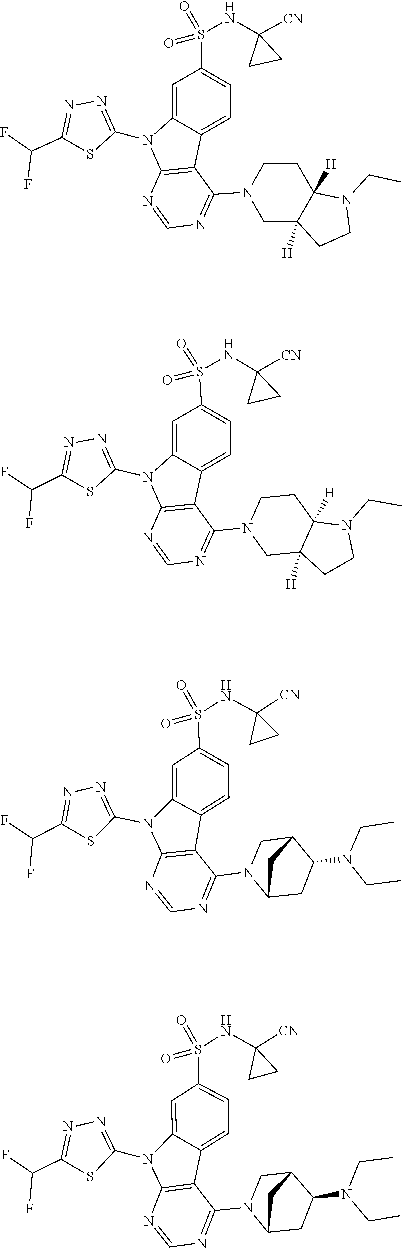 [SMILES: CCN1CC[C@H]2CN(c3ncnc4c3c3ccc(S(=O)(=O)NC5(C#N)CC5)cc3n4-c3nnc(C(F)F)s3)CC[C@@H]21 ; CCN1CC[C@H]2CN(c3ncnc4c3c3ccc(S(=O)(=O)NC5(C#N)CC5)cc3n4-c3nnc(C(F)F)s3)CC[C@H]21 ; CCN(CC)[C@@H]1C[C@H]2C[C@@H]1CN2c1ncnc2c1c1ccc(S(=O)(=O)NC3(C#N)CC3)cc1n2-c1nnc(C(F)F)s1 ; CCN(CC)[C@H]1C[C@H]2C[C@@H]1CN2c1ncnc2c1c1ccc(S(=O)(=O)NC3(C#N)CC3)cc1n2-c1nnc(C(F)F)s1] 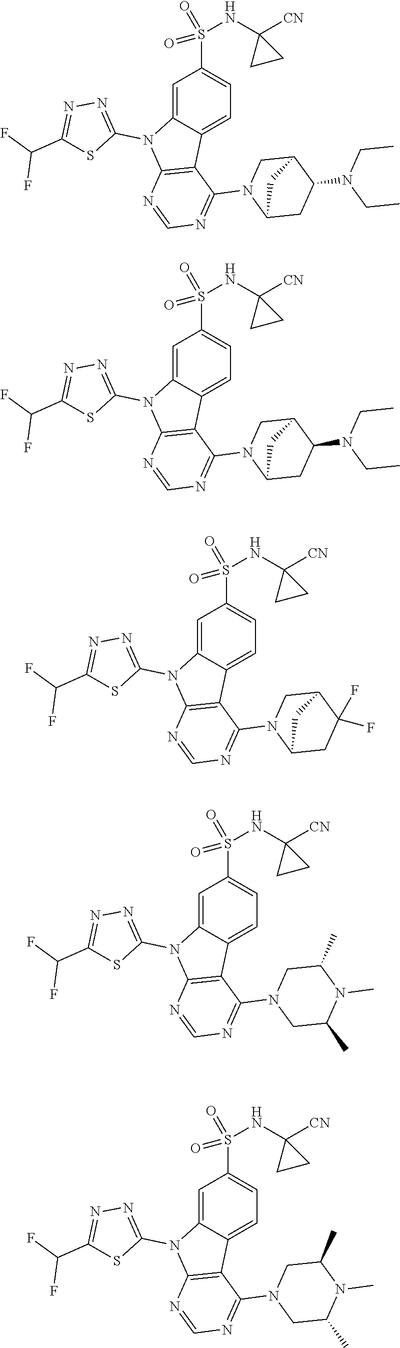 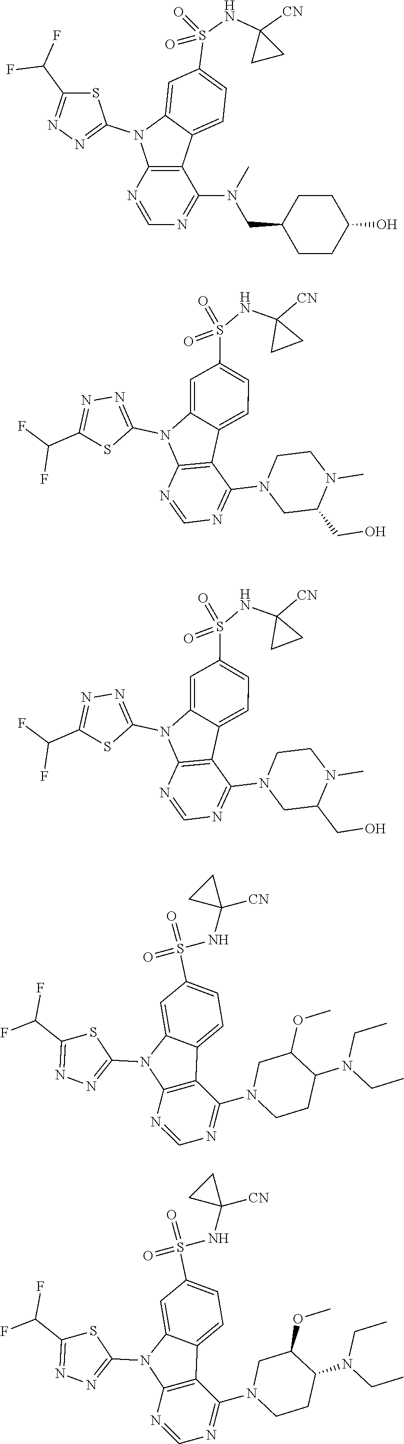 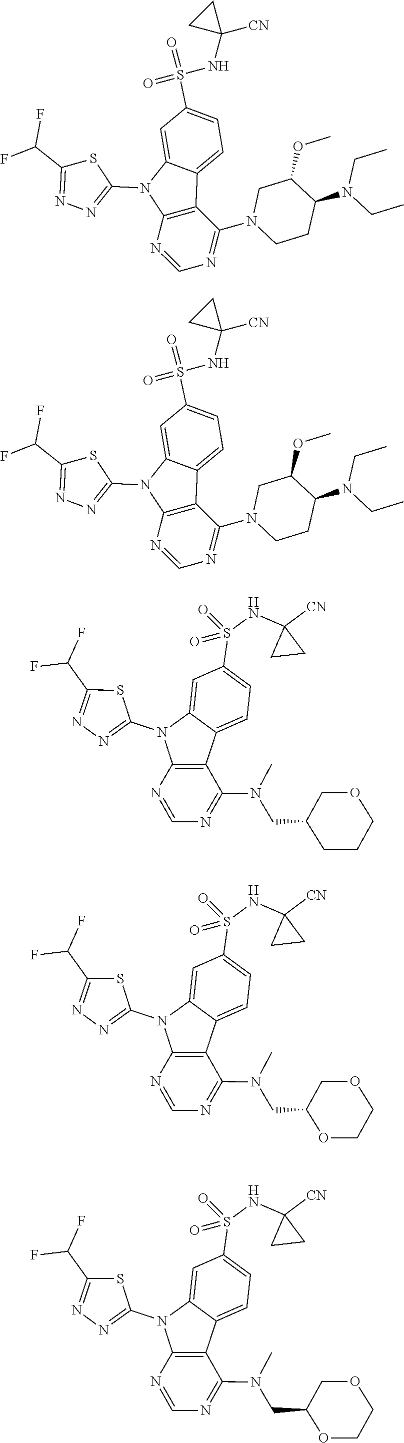 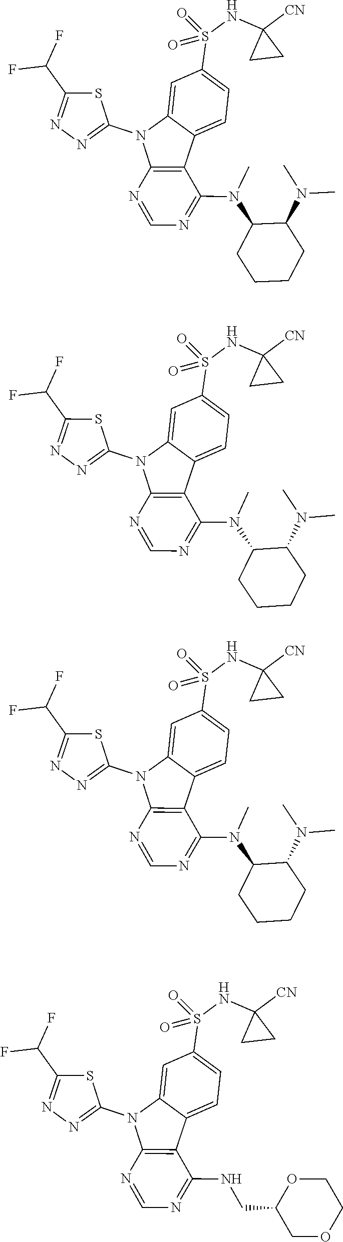 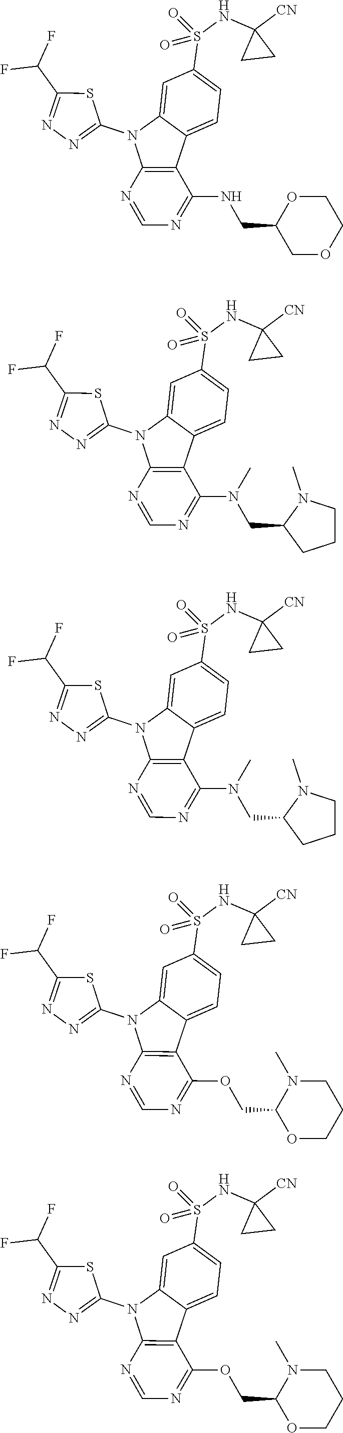 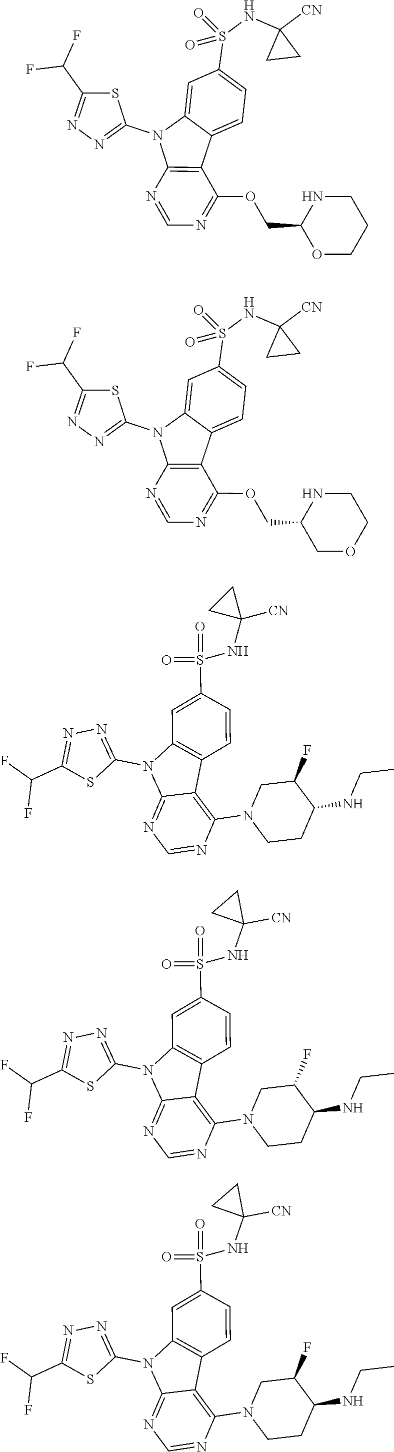 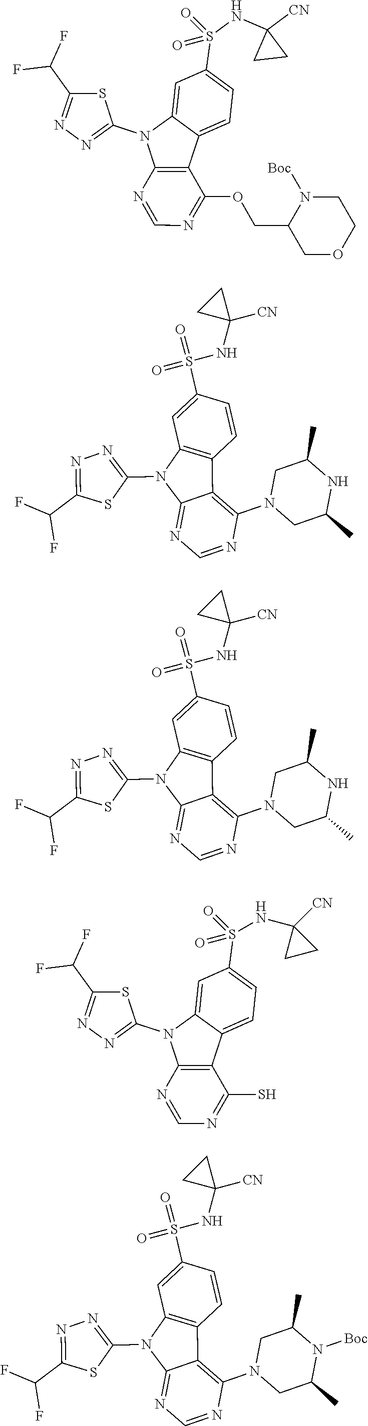 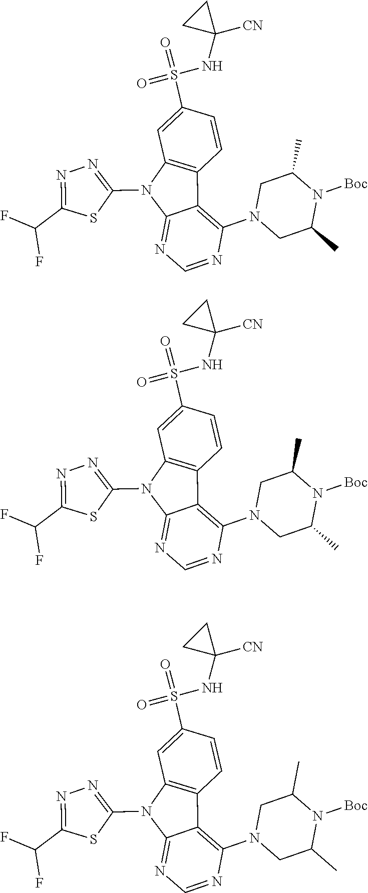
or a pharmaceutically acceptable salt thereof,
preferably, the compound of Formula (I) is: 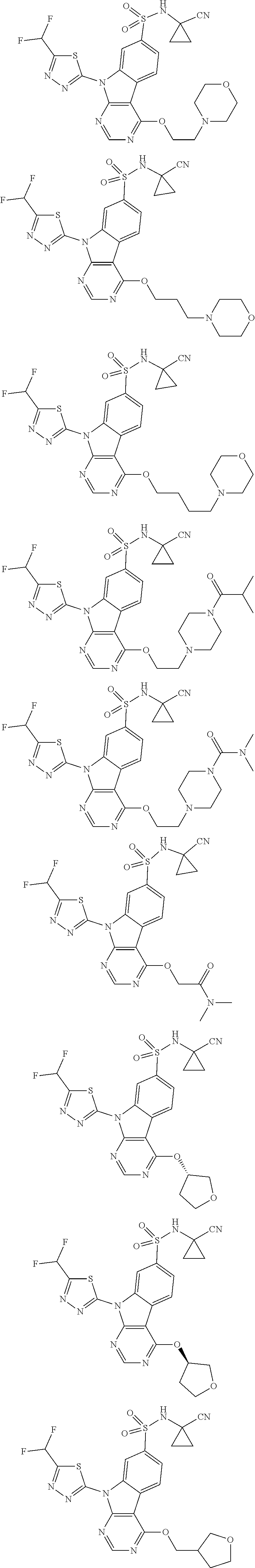 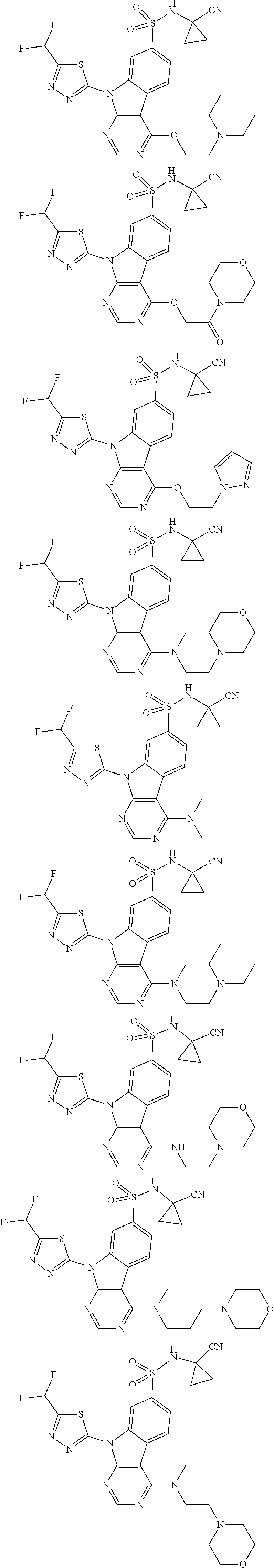 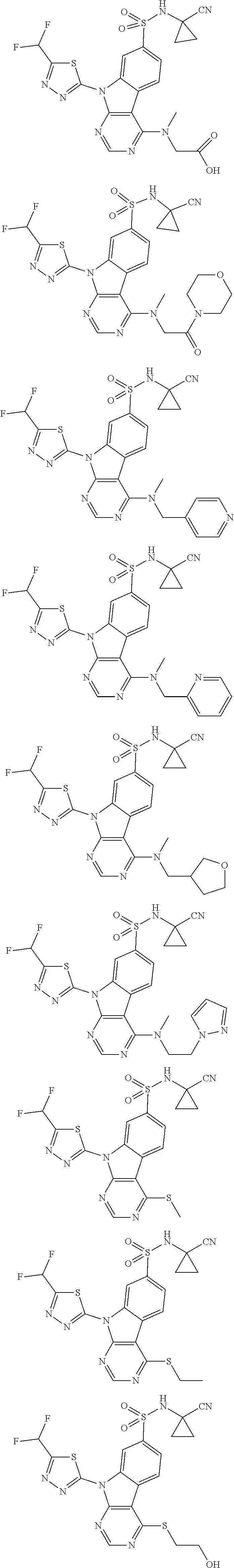 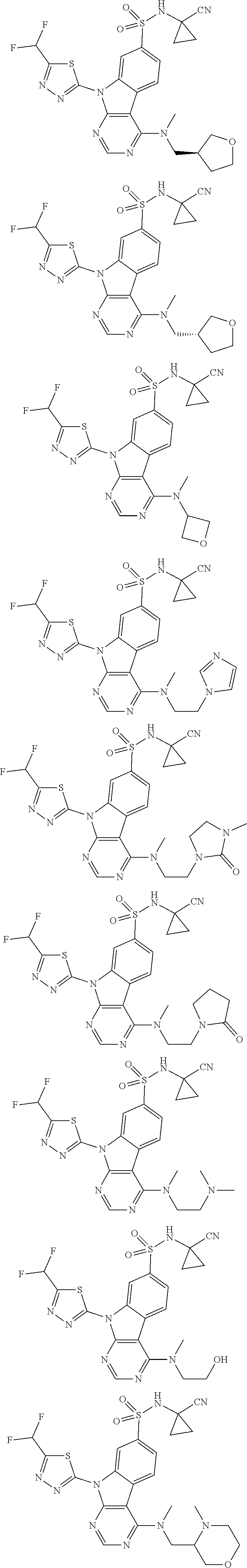 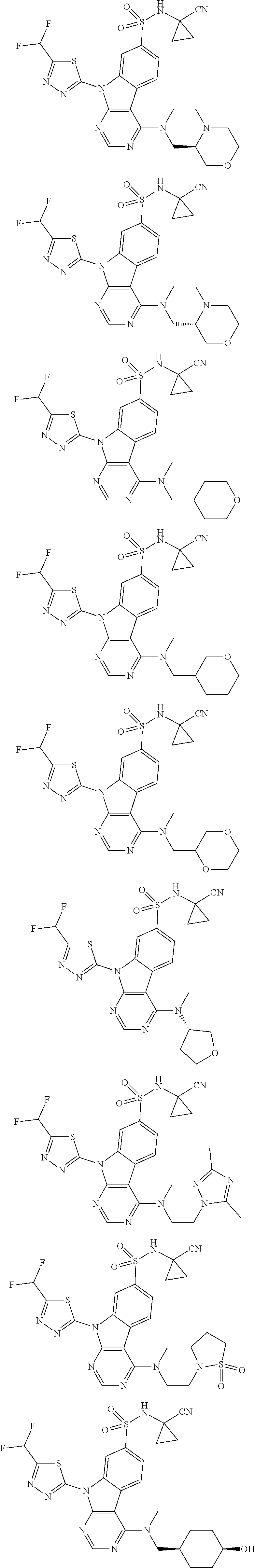 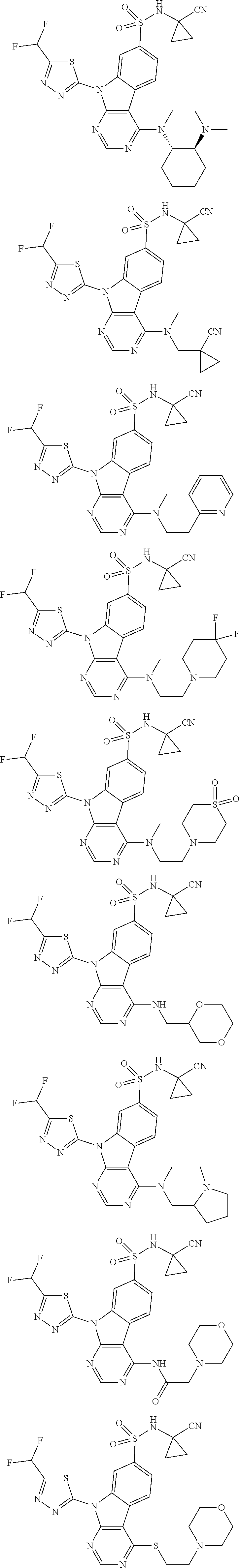 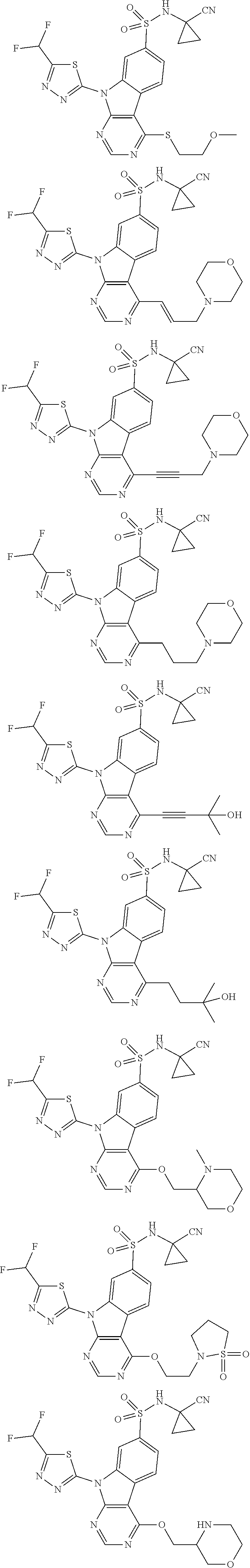 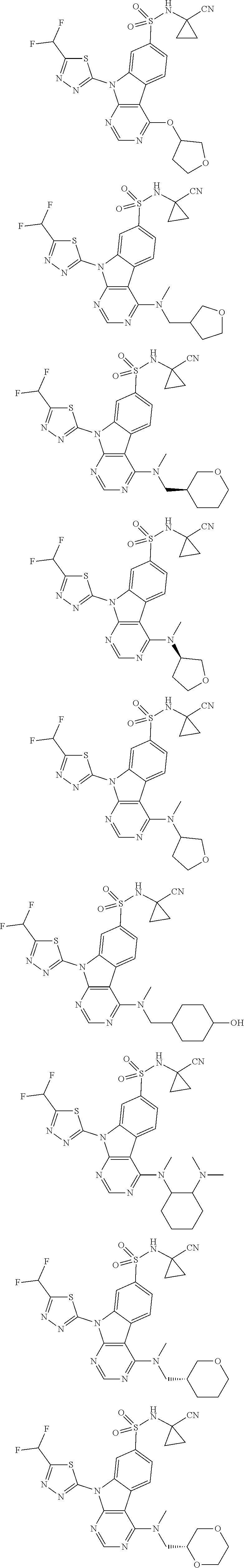 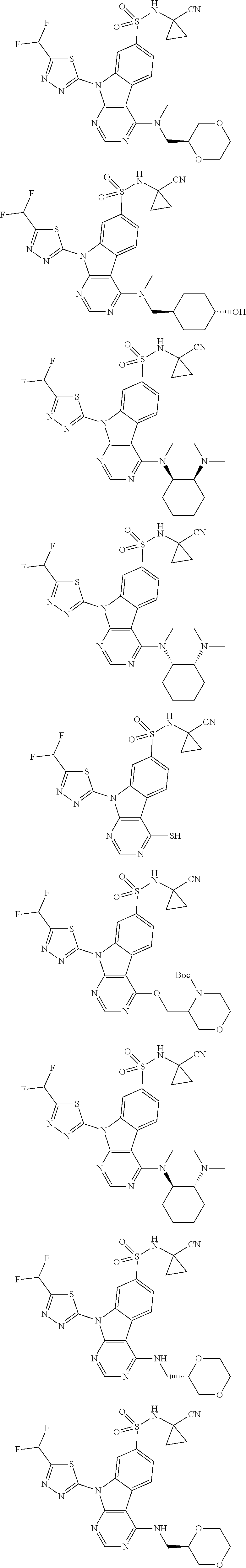 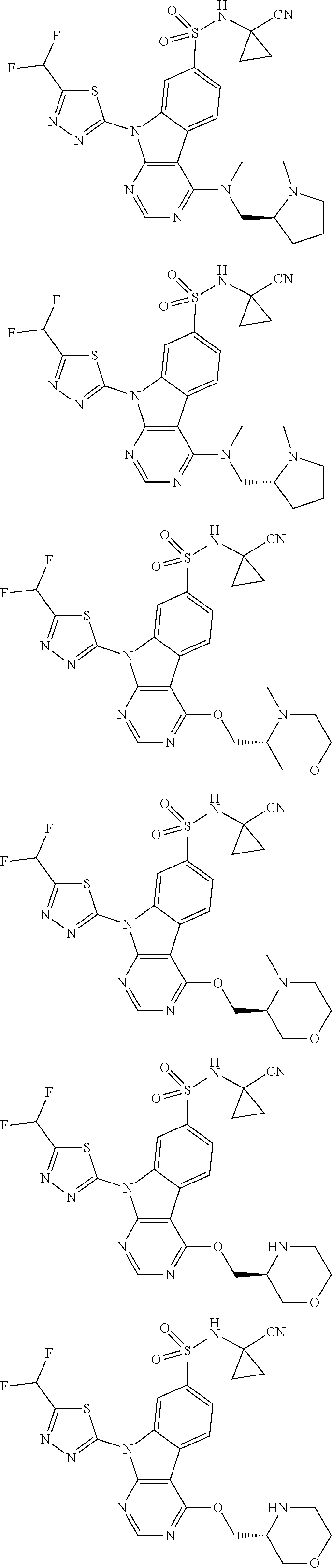
or a pharmaceutically acceptable salt thereof,
preferably, the compound of Formula (I) is: 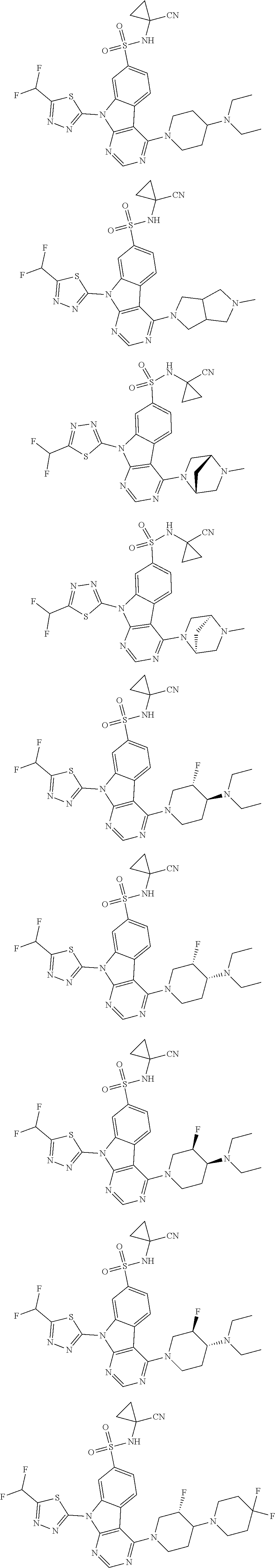 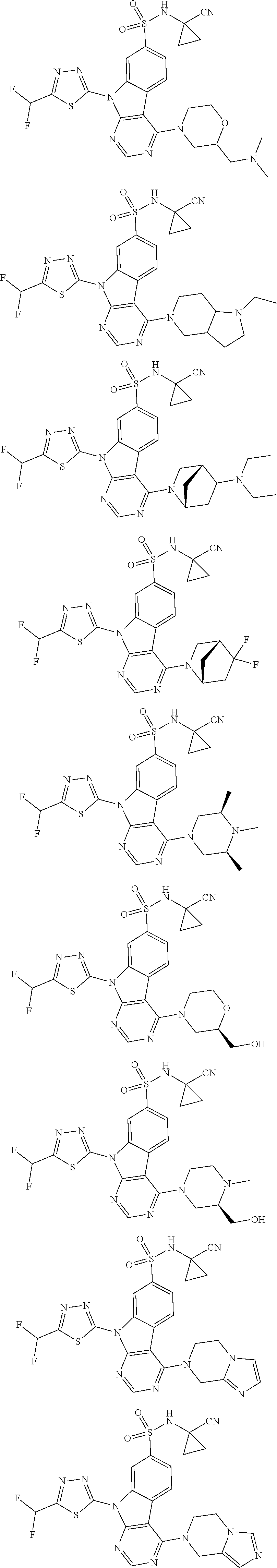 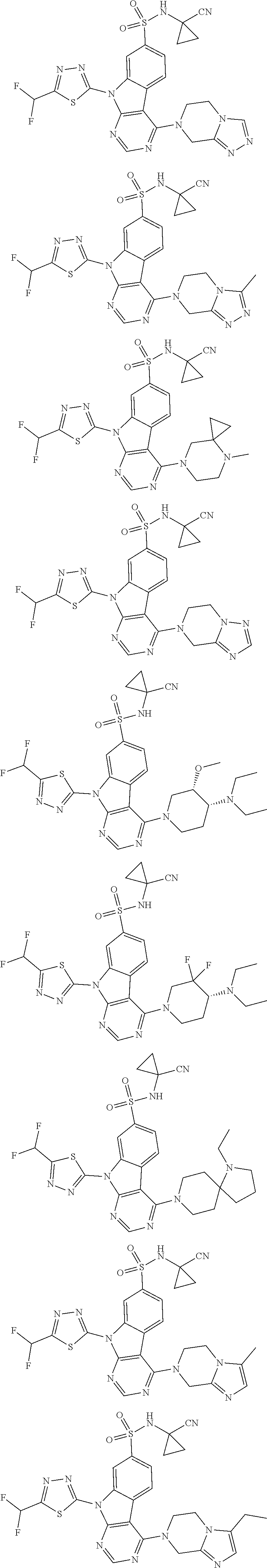 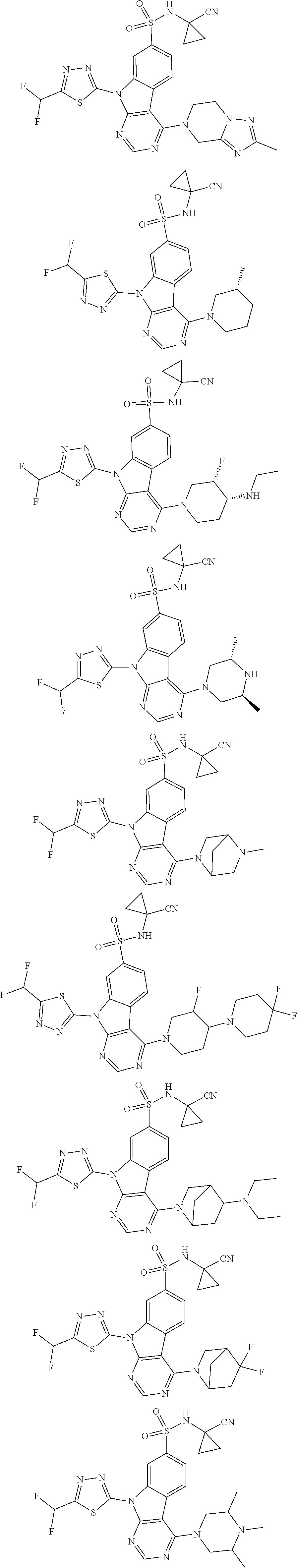 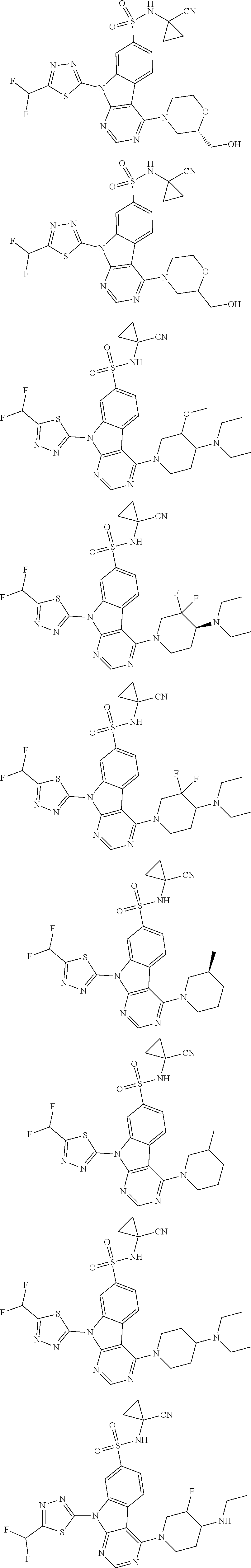 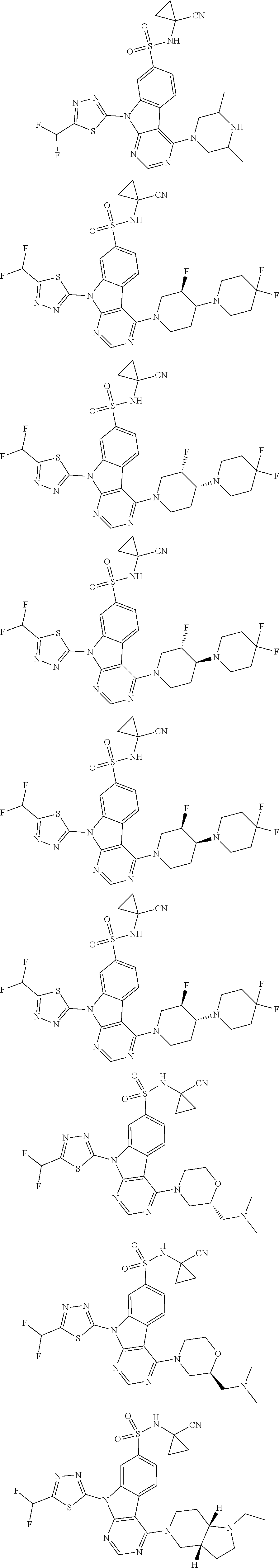 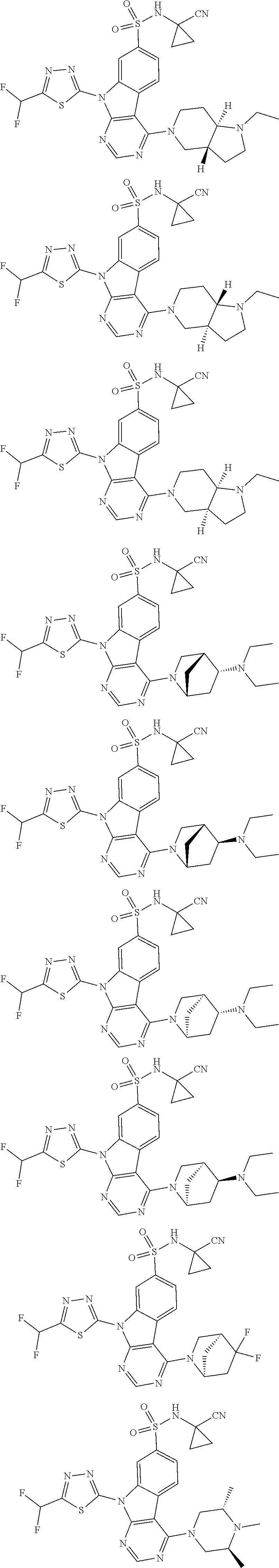 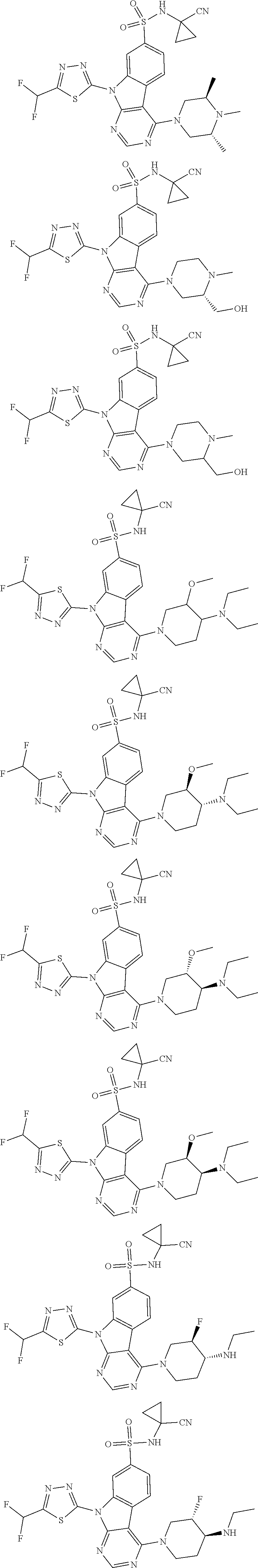 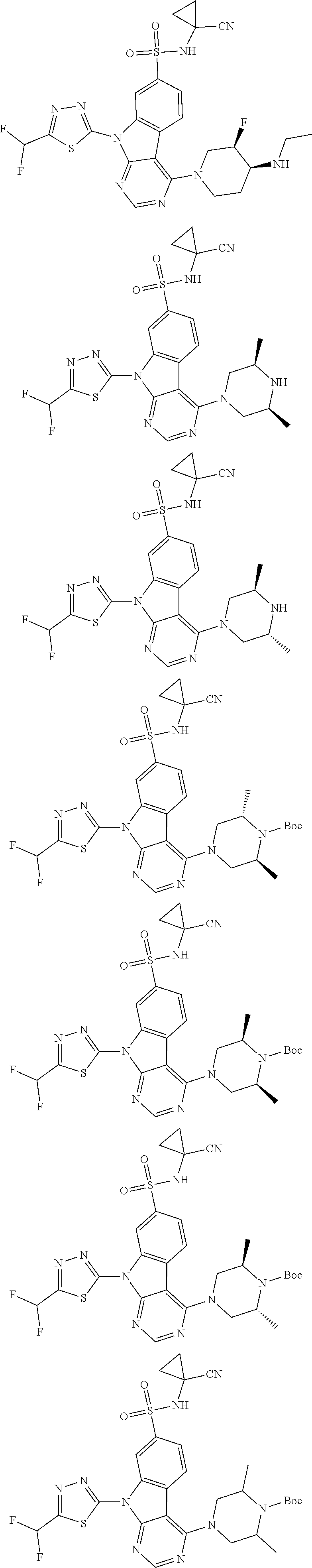
or a pharmaceutically acceptable salt thereof.

10. A pharmaceutical composition comprising the compound of claim 1, a pharmaceutically acceptable salt, stereoisomer, prodrug, chelate, or non-covalent complex, and at least one pharmaceutically acceptable carrier or excipient.

11. A method of inhibiting PARG and/or treating a disease associated with inhibition of PARG, wherein, the method comprising: administering to the patient in need the compound of claim 1, a pharmaceutically acceptable salt or a pharmaceutically acceptable salt, stereoisomer, solvate, N-oxide thereof or prodrugs;
preferably, the disease is a cancer;
preferably, the cancer is breast, ovarian, gastric, prostate, pancreatic, uterine, cervical, endometrial, lung, brain, bile duct and hematological cancer.

12. A method of inhibiting PARG and/or treating a disease associated with inhibition of PARG, wherein, the method comprising: administering to the patient in need the pharmaceutical composition of claim 10;
preferably, the disease is a cancer;
preferably, the cancer is breast, ovarian, gastric, prostate, pancreatic, uterine, cervical, endometrial, lung, brain, bile duct and hematological cancer.

13. Use of the compound of claim 1 in combination with surgery, chemotherapies, radiation therapies, targeted therapy, other DDR modulators, immunotherapies, and gene and cell therapy approaches for the treatment of the cancer;
preferably, the targeted therapy is a kinase inhibitor, growth factor inhibitor, cyclin dependent kinase inhibitor;
preferably, the DDR modulator is DNA-PK inhibitor, ATM inhibitor, ATR inhibitor, CHK1 inhibitor, WEE1 inhibitor, CDK1 inhibitor, LIG4 inhibitor, HIF-1 inhibitor, HDAC inhibitor, RAD51 inhibitor, Polθ inhibitor, WRN inhibitor, PRMT5 inhibitor, MAT2A inhibitor and PKMYT1 inhibitor.

14. A compound of Formula (A), wherein: 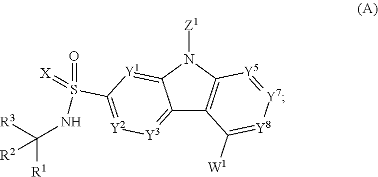
W¹ is a leaving group; preferably, W¹ is halogen, C₁-C₃ alkyl-SO₂—, phenyl-SO₂—, —SC₁-C₄ alkyl, —S-phenyl, —OC₁-C₄ alkyl, —OC₁-C₄ haloalkyl; wherein, the C₁-C₄ alkyl, phenyl is optionally substituted by halo, CN, NO₂, SF₅, OH, C₁-C₄ alkyl, C₁-C₄haloalkyl, —O—C₁-C₄ alkyl, —OC₁-C₄haloalkyl; more preferably, W¹ is F, Cl, Br, I, OTf, OTs, OMs, —SCH₃, —SCH₂CH₃, —S-phenyl, —OCF₂CF₃;
wherein, R¹, R², R³, X, Y¹, Y², Y³, Y⁵, Y⁷, Y⁸, and Z¹ are defined with respect to Formula (I);
preferably, the compound is Formula (Aa): 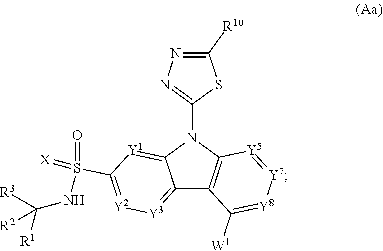
wherein, W¹, R¹, R², R³, R¹⁰, X, Y¹, Y², Y³, Y⁵, Y⁷, and Y⁸ are defined with respect to Formula (A);
preferably, the compound is Formula (Ab): 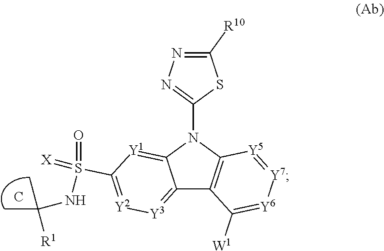
wherein, W¹ is defined with respect to Formula (A), ring C, R¹, R¹⁰, X, Y¹, Y², Y³, Y⁵,
Y⁷, and Y⁸ are defined with respect to Formula (IV);
preferably, wherein, the compound is: 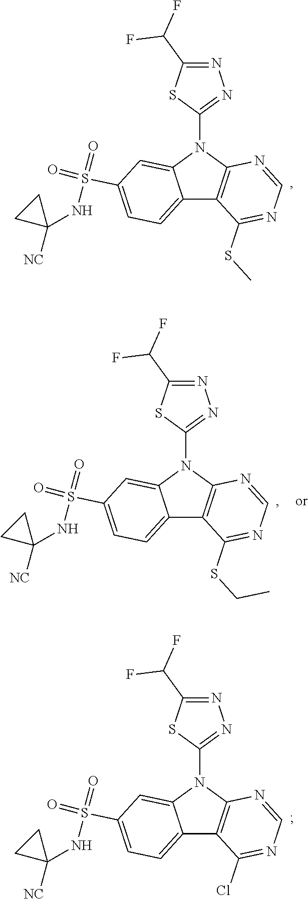 [SMILES: CSc1ncnc2c1c1ccc(S(=O)(=O)NC3(C#N)CC3)cc1n2-c1nnc(C(F)F)s1 ; CCSc1ncnc2c1c1ccc(S(=O)(=O)NC3(C#N)CC3)cc1n2-c1nnc(C(F)F)s1 ; N#CC1(NS(=O)(=O)c2ccc3c4c(Cl)ncnc4n(-c4nnc(C(F)F)s4)c3c2)CC1]
or salts thereof.

15. A compound of Formula (B), wherein: 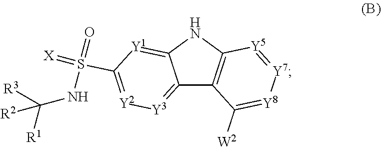
W² is a leaving group; preferably, W² is —SC₁-C₄ alkyl, —S-phenyl, —OC₁-C₄ alkyl, or —OC₁-C₄ haloalkyl; wherein, the C₁-C₄ alkyl, phenyl is optionally substituted by halo, CN, NO₂, SF₅, OH, C₁-C₄ alkyl, C₁-C₄haloalkyl, —O—C₁-C₄ alkyl, —OC₁-C₄haloalkyl; more preferably, W² is —SCH₃, —SCH₂CH₃, —S-phenyl, or —OCF₂CF₃;
wherein, R¹, R², R³, X, Y¹, Y², Y³, Y⁵, Y⁷, and Y⁸ are defined with respect to Formula (I);
preferably, the compound is Formula (Ba): 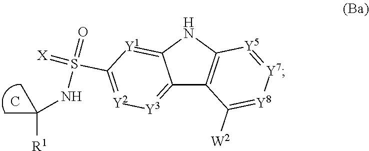
wherein, W² is defined with respect to Formula (B), ring C, R¹, X, Y¹, Y², Y³, Y⁵, Y⁷, and Y⁸ are defined with respect to Formula (IV);
preferably, the compound is: 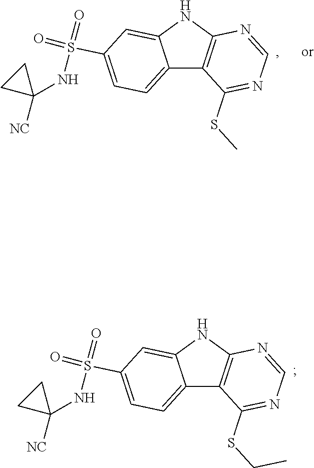 [SMILES: CSc1ncnc2[nH]c3cc(S(=O)(=O)NC4(C#N)CC4)ccc3c12 ; CCSc1ncnc2[nH]c3cc(S(=O)(=O)NC4(C#N)CC4)ccc3c12]
or salts thereof.
